SEQUENCE LISTING (647 sequences)

SEQ ID NO: 1 - DNA, 32306 bp, synthetic construct
  source 1..32306: mol_type = other DNA
gcatttcctctctagctgttcctgaggctggcatctgggtaagtccagctccgcggggcagtgtgggcgacaggaccgggagagcagtgggggagattgtgttcagagggcagaacttgtcctggttaatgcactaggaatttcttctggcgtctccaaggttgtgcttcaacaacatttctggaatgttcttttatcattatttgggctctacgtgtgcaagttcgtgtgtgtgtatgtggcggggggaagcggggaggacagcggcagcaaccatagtttacacccacgacttcagcagatttgcccagtatccctcccaggggagaagatggatgggatatacagaaggaaacaaacctccttaaagaccaaagggcagcagatttttgtgctggtgttagggtggaaggagcacagaagaaaggcagaatcctgatgcagatggaaggactgatggggcaggagctgggagggtatgcggagctaatagggctgtctggagagcagtcacctcaggatgggcaggagctgggagggtatgcggagctaatagggctgtctggagagcagtcacctcaggatgcacagggctgggtttgcagggctcaggaggcgaggagtagataggcaggaatggagatgggacgggagagatgagtgagtctgaggcatcttgacattgaactgcagtcacagaaattccaatttggagagctccaccgttttccaggctctctgcctcccttgagtgttatttatggctttaggttgaggtgctttgccaaatagagtataatcaagggatcaaggagggaacaattccaaacaaagccaacttgttcatccatccggactctaggagatatggaagatctaaagatacagcaggtgattgctagtttttgtggaactcacagtctagttgggaagactatgttggtgaatgctgcagtgatacacctgtgataaatgctggggaagtgtgtcttttagtcatctattttgttgtctttgtctttgtctgactgaaacaagtgatggaagacatggtctcatatctctggtcaaaatcctggctctgttccttcctggttctaaacccctcggcaggcttcctagtccccctaagactcagtttatgcatctataaaatggggctaggaataggtgcacctcatagagctctgtgcaggttagatggggaaaaacatcagtgagtgcctagcctgtggaaagctctaaattaatgtgaattataatgataacaattgcgaattttgcaaggagacattaaaggatgtatgtttttatttattttattttattctattttattttttgagatggagttttgctcttattgcccaggctggagtgcagtggtgtgatcttggctcatcgcaacctccgcctcctgggttcaagcgattctcctgcctcagcctcccgagtagctgggattacaggtgcctggtaccatgcccggttaatttttgtatttttagtagagacgggatttctccctgttgtttggtgagcctggtctccaactcctgacctcaggtgatccgcccacctcggcctcccaaagtgctgggattacagggatgtatgtttttattattctccattatgcactcccagcttcatctcacttcaatccactggttgatagaagtgcagtcaaagactgtcccctcctctgcaagcttttatgttctctctcctctctccctagccacgtcctgttctgttatcaacacacttactcacctcctggccttctccttctattgtctctcatccctcttccctcacaaaaacagaagcaaagagccagagccttcagtttggaggaactgaaaacattctcttctgctttctcattttgtagatgaggaaactgaagttgaggaatagtgaagagtttgtccaatgtcatagccccgtaatcaacgggacaaaaattttcttgctgatgggtcaagatggcatcgtgaagtggttgttcaccgtaaactgtaatacaatcctgtttatggatttgtttgcatatttttccctccatagggaaacctttcttccatggctcaggacacactcctggatcgagccaacaggagaactttctggtaagcatttggctaacttttttttttttgagatggagtcttgctgtgtcgcctaggctggagtgcagtggcgtgatcttggctcactgcagcctccacttcccgggttcaatcaattctcctacctcaacttcctgagtagctgggattacaggcgcccgccaccacacccggctcatttttgtacttttagtagagacacagttttgccatgttggccaggctggtcttgaattcctcagctcaggtgatctgcctgccttggcctctcaaagtgctgggattacaggcgtgagccactgtgcccggccttggctaacttttcaaaattaaagattttgacttgttacagtcatgtgacatttttttctttctgtttgctgagtttttgataatttatatctctcaaagtggagactttaaaaaagactcatccgtgtgccgtgttcactgcctggtatcttagtgtggaccgaagcctaaggaccctgaaaacagctgcagatgaagatggcaagcacccgctgcaagctggccaggtacctggaggacctggaggatgtggacttgaagaaatttaagatgcacttagaggactatcctccccagaagggctgcatccccctcccgaggggtcagacagagaaggcagaccatgtggatctagccacgctaatgatcgacttcaatggggaggagaaggcgtgggccatggccgtgtggatcttcgctgcgatcaacaggagagacctttatgagaaagcaaaaagagatgagccgaagtggggtgagtggaaggaagacttttaaaaaaaattgtggccaagtgcacatagtgtacaattttccatctttatatatatatatatatatatttttttttgagacggagttgctcttgttgcccaggctggagtgcaatggtgagatcttggctcactgcaacctccacctcccgggttcaagcgattttcctgcctcagcctcccaagtagctgggattacaggcattgcacctccacgcccggctaattttgtatttttagtagagatggggtttctccatgttggtcaggttggtctcaaactcccgacctcaggtgatccacccgcctcggcctcgcaaagtgctggaattacaggtgtgagccaccgcgcccggccgtaataatttttaaatgtacagttcagtagtgttaggtacactcacattgtgcaaccagtctccagaactgttttcatcttgcaaaactgaaactcctcacccattgaatgataactcctcattgcctcattctcccagccctggtaaccaccattctatttgccatctctgtgaatcttactactctgggtacctcatataagtggaaacatacagcattttttcctttatgagtagtttatttcactcagcataatgtcctccgggttcaactatgttgtagcatgtgtcggaattcccttcctttaaaaaagactgtataaatactccatggtaccatataccaaattttgtttagttatctgttgatgaacacctggattacttctacctttttgttattgttaatgctgttatgagtgttggtgcacaaatatctcttcaagaccctgctttgaattctctggggtatatactcagaagtgaaatttctgaatcatacggtaattctgttaaatattttgaagaactgctctactgttttagataccatctgcaccatttcccaccaacagcacacaagggttccaatttctccatatcctaagaacacgttatttttatttattttttcttttttatagtagccattctaatgagtgtgaggtggtatctcactgtagttttgatttccgtttccctaatgataagtgatgctgagcacattttcactgcttgttggccatttatagagcttcttcagagaaagttctgttcaagttcttttctcattttcagaccaggtttggcttttgttgttgccttgtaggagttctttatgtattctagatattaaccccatatcagatatatgatttacaaatattttctcctattctgtgggttgtcttttcatttggttaactgtgtcctttgatgcacagaagttagtatgatttttttttttttaaatcatggccgggcatggtggctcatgcctgtaattccagcactttgtgaggctgaggtgggtggatcacctgaggtcaggagttcaacaccagcctggccaacatggcaaaaccccatctctactaaaaatacaaaaaaaattagccgggtgtggtggtgggcacctgtaatcccagctactcaggaggctgaggtaggagaattgcttgaacatggggagtggaggttgcagtgagctgagattgcaccacggcactccagcctgggctaaagagcaagaccctgtttcaaaaaaaaatttttttttcttgtattgtcttggttttttctcttttcccctctcacggtagtgctatgagaaggtggaggcaaactgtttgctttctctaactcctttaaaaagctgatgcagactgtacctaccccctgggcacctgccatgattagtccctctttatctgaatctcagcagtgcctagacctgcctccaattctccatccagcttctcagaccacatgttttgtccagtatctgctgtgtggtcagcaggcttgggcctctgcttcctatggcccagagggcagcctctctccttgaagaaccgtggagtttggggcagaagcctttcctttcaccaccactttgggaattggaatgtaaaagaaattctcttaatccccatgtacacattggggataaggttctttcttttgtcttgtgttctaggcaaaggtagggctcagagaagggcagggtattgcaggatgatgatgtagagctggtggtgatgggaaggaggagtacaaatgagtcagtggatagggagacacatgttaaggcagggcagcagtgtgggtgtaatgacactggggaaacagacagcagtgtgcagccgggggaggtcagaaaccagttgctgcaatggctctcacctgtagtcccagctactcaggaggctaaggcaggaggatcgcttgagcccaggagctcaaaaccagcctgagcaacatagggagacctcatctctaagtaaataaagcaattatcattgtgtcagtacttatatactcacatacccaaatgaaagtgtcataatcacaactgtagaagtactttgctaagcagtttttatggattttatggcacattcaagaaacccatgaaacagcttctggtattatccctcctgcaagtgaatgaggtgagcctggaagagatcctaactggcccatggtttatttctagccataaccagagctggggtaaaaagcagggcatgtctctgatcaaagcctggctcttaaccactacaccgtctttgcttccatatctggattttgaggcagaaaagttataactagggttagaagtttgtttgtttgtttcctggggtggggttaggttttaggtaatagactttggctaatttactgtcatggctcctacctgagtcccagttactcaggtggctgaagtgggtgcatcacttgagctcaggagttccaggctggagtgagctatgaccatgccactgccctccagctttggaaacaaagcaagactctgtctcttaaaaaaaaagaataagattttggttttggcttagattttttggattttattttgtgatatggttaccaagtagacagctgtcagaggtcttttatgggctgggcgcagtggcttatgcctgtaatcccagcacttcaggaggccaagatgggaggattgcttgagcccaggagtttgagaccagcctgggcaacatagtgagaacctgtctctacaaaaaatgcaaacattagttaggtgtggtggcacgtgtcaatagtcccagctgttctggaggctgaggtgggaggatcacttgagcccaggaggttgaggctgcagggagccatgattgtgtcattgcactccagcctgggcaatagagtgaggccctgtctcaaaaaaaaaaaaaaaatcatttatgttacttgtagattcctgcccacctgaagttatctgtcttatatatttcccctagtgactaactcttgttttacctaacctgaatttctcctatagaatgccttcttaagatatgctatctagtgactgtttggatttgtgattctcaacacagatgcatgttggaatcacctgggagcttttagtatgtgatggttcctgggcctcacctcagagggactgaaaaagaacctctgggcgtgcagtcctgaagtccatatttttaaagcttcctgggtgattctaaagtcagccaggattcccaaccactagattaggcacgaaatagactggtcgttctcactcttgaagaaccagacatctagacttgacaatagaagacgtgtgtaaaatggattgcagggaactgcggtcagggaagggtgacctttcttagcccacagccgtgttggggatataggagagcttggagatgccctcacaatcaggctgccaggggcaggcaaaagcaaacgagggtaaaagaaacttagcctgcttcgcatgtttgctgattgatttccaggcctgaggaagagattctgagactggggacagtgggaacacatgcttggcaggtggacagcagaagttgttttgaaactaggagtgcagaaatgctcccaaccagacttttgactggatacctgtcctctgcaactcagagccatgttttgatttgggggatgtttggggtctcctctctcatgccaaatattaggccaggtttcaattgcatcctctgtgataatagttctgggttttgacaccttttttttcccttttaggttcagataatgcacgtgtttcgaatcccactgtgatatgccaggaagacagcattgaagaggagtggatgggtttactggagtacctttcgagaatctctatttgtaaaatgaagaaaggtaagcgactggggtggtgcttctagttgagttttaagacctggttcaggaggctctgggaagctgagcagctgggtaacttggccatactataggttcctgctctttggaatgcaaaggcctgtctcaggaaatccattgtcagttgaagaaacaaattcatgtttgtgtggctcttgcagtcccagagccagcggcggtgattaagcgtttcaagtttgataaggcataggatgcagattacttaaaggaataagattgattcaaatgatctttgggctggtagctacacatagagatgggccttctgctcagctgtgtatcttaaaaacttgacaccagagattctcggcacctttcctacccagtggggaggagctgcaccttccatttctccattccggttaccactcgcttccgatgacaggccccagctgagagcgcatctcaggtggatgtgtgtatactttccccctaacttcctgtctttgccgtagattaccgtaagaagtacagaaagtacgtgagaagcagattccagtgcattgaagacaggaatgcccgtctgggtgagagtgtgagcctcaacaaacgctacacacgactgcgtctcatcaaggagcaccggagccagcaggagagggagcaggagcttctggccatcggcaagaccaagacgtgtgagagccccgtgagtcccattaagatggagttgctgtttgaccccgatgatgagcattctgagcctgtgcacaccgtggtgttccagggggcggcagggattgggaaaacaatcctggccaggaagatgatgttggactgggcgtcggggacactctaccaagacaggtttgactatctgttctatatccactgtcgggaggtgagccttgtgacacagaggagcctgggggacctgatcatgagctgctgccccgacccaaacccacccatccacaagatcgtgagaaaaccctccagaatcctcttcctcatggacggcttcgatgagctgcaaggtgcctttgacgagcacataggaccgctctgcactgactggcagaaggccgagcggggagacattctcctgagcagcctcatcagaaagaagctgcttcccgaggcctctctgctcatcaccacgagacctgtggccctggagaaactgcagcacttgctggaccatcctcggcatgtggagatcctgggtttctccgaggccaaaaggaaagagtacttcttcaagtacttctctgatgaggcccaagccagggcagccttcagtctgattcaggagaacgaggtcctcttcaccatgtgcttcatccccctggtctgctggatcgtgtgcactggactgaaacagcagatggagagtggcaagagccttgcccagacatccaagaccaccaccgcggtgtacgtcttcttcctttccagtttgctgcagccccggggagggagccaggagcacggcctctgcgcccacctctgggggctctgctctttggctgcagatggaatctggaaccagaaaatcctgtttgaggagtccgacctcaggaatcatggactgcagaaggcggatgtgtctgctttcctgaggatgaacctgttccaaaaggaagtggactgcgagaagttctacagcttcatccacatgactttccaggagttctttgccgccatgtactacctgctggaagaggaaaaggaaggaaggacgaacgttccagggagtcgtttgaagcttcccagccgagacgtgacagtccttctggaaaactatggcaaattcgaaaaggggtatttgatttttgttgtacgtttcctctttggcctggtaaaccaggagaggacctcctacttggagaagaaattaagttgcaagatctctcagcaaatcaggctggagctgctgaaatggattgaagtgaaagccaaagctaaaaagctgcagatccagcccagccagctggaattgttctactgtttgtacgagatgcaggaggaggacttcgtgcaaagggccatggactatttccccaagattgagatcaatctctccaccagaatggaccacatggtttcttccttttgcattgagaactgtcatcgggtggagtcactgtccctggggtttctccataacatgcccaaggaggaagaggaggaggaaaaggaaggccgacaccttgatatggtgcagtgtgtcctcccaagctcctctcatgctgcctgttctcatgggtaaggaaactcggcttccaggtgcttcctcctgcttcctcgccagcttcttcttggcgcttgcctcctctcatctcttttcaactatcttccaaatactgttgccacagctacatcataatgccaccactgtctgtttgagactccttcatgagcaaagattgatgtatggtaggtggataaatgggatgaggaaaaaaaaaataaaacaaggaacaaatgtttggggaatgccagtttagcacaaggtattaagtaggatgtaggattgcctacaaatatttgtcaactgaaatttcttgtaagctacttggagcactagtgcctaagagtcagtcaatgtctgtggggcagaacaatgccagagagtctgtgtcccaggagcgaggggacagagggcctcagagctggaggctggggggatgtttcttagaaatgtgcaatctaagcctcctttaaggaatggtgggacctgggcgcatagagaagaaattaaaggaattctgcactgaagactggtatgagccaaatcatgaatgcagtctgagaaaaagtgtgtgcaggtgacagtggagagagacacagtttccttggaaaaaaacatgatgaaggtaaatcaagggaaatagagcagaaatgtggagaccctctttctaggcccccatgttgctctgcactgggcattgccaaaaccccgttttcgggtctgttcacacacttgctgggtggttggaagctagtggccaagtctgtgcaatgaagtggttgagtcacctgacttctgagtcccttttgggtttatcgttcagtgcctcctgcagaaggaccacccacagtggttgtaggaaggtggccaccctgtgatgtgtgagtttagcctggatagaagacacttgacgtggggcaaaattaagggaaacaggaggcttggcccttccacaggcaacaatgagtttcagctttcttgatccgttgacattaccatgaccaggaccaccttcctagtcatggtttggccatgaaggactgactgcccacagggccgcagtggtggtgaagacccaggtcctggtcacagggacttgaggtctggctgtgggacagtccggaaacaagtgcagatgtcggggagggggtcatgggctctgacaaggaaacttgcagagtatccaaagcacaaggggacgaacctgttcaccagcggggcacgggtgggtgtccccgctcagccagcttccggaggatgggcagtgagagagtgagagaaggaaaggggcagccaggctgggggcttacggtgttgagagagagggcaggtgtgggaaagcagggtacggtttctacggcagcaaacagttccgagtgactggaaaaaagacagttgtcttcattcatcaggttgccataacaaaacactgcaggctggttggcttataagcaaaaggaaggttcaccttacggttctggaggctgggaagtccatgatccaggctctggcagatctggtgtttggtgagggcctgttccctggtcgatggtgccttctcactgtgtccttacatggtgaagaggcaaggcagctctctggggtccctttcctaaggacagcaatcccactcctgagggctccaccctcataacttaaccacacctcaaaggccccatcgcctgatacgtcatgttgagggttaggatttcagcgtatggattttggggcacacaaacattcagaccatagaaatgggagaagaagagtaagacagagagaaaagtaggcagaactactcattaagagtgaggtggaaggtggagagggcagggagaccttatgaagatcacacatctgtgaggaaggtttgcacactctcactgaagccccggtaggaggctcagcacccatttctctagatagcaccttccttcctctgaggacagattctgactggagccctggtagggggctcagcacccatccctctagatggcacctgccttactctgaggacagactctcactgaagccccggtaggaggctcagcacccatttctctagatagcaccttccttcctctgaggacagactctgattggagccctggcagggggctcagcacccatttctctagatagcaccttccttcctctgaggacagactctcactgaagccccggtaggaggctcagcacccatttctctagatagcaccttccttcctctgaggacagactctgattggagccctggcagggggctcagcacccatccctctagatggcacctgccttactctgaggacagactctcactgaagccccggtaggaggctcagcaaccatttctctagatagcaccttccttcctctgaggacagactctgattggagccctggcagggggctcagcacccatttctctagatagcaccttccttcctctgaggacagactctcactgaagccccggtaggaggctcagcacccatttctctagatagcaccttccttcctcaggacagactctgactggaaccctggtagggggctcagcacccatccctctagatggcacctgccttactctgaggacagactctgacgggagccctggtagggggctcagcacccatccctctagatggcacctgccttactctgaggacagactctgactggagccctggtagggggctcagcacccatctttctagatggcagctgccttactctgagggcaggagcttgctttgtatgttccagtcccttctgagaaccagaagcctgagcatctattagaaatgcaggattgtggacccaatcttagacctagagagctagactttgcatttgaatgagagctacaggtgattcaaaggtgactgcatgtgtgggtgcagtggctcttgcctgtaatcctaggttttgggaggccaagatggggggattgcttgaagccaggaattcaagtcctacctgagcagcaaagtgagagcacctctctacagaaaaacaaaagcaaaaatgtgactgcatgttggagccacctggctttttttaaataaaaaagatccatatagctggacacagtgactcgtgcctgtaacctcagctactcgggaggctgaggcaagaggattgcttgagcccaggagtttgaggctgcagtgagctatgatcatggcactgcattccagcttgggtttcatctgaaaaaaaatttaaaaaattcatctagttccacctcagagcttctgattttatttccatggatgtggcctgggcttgcgattttctttttcttttttttttttttgagatgaagtcttgctctgttgcccaggctggagtgctgtggcatgatcttggctcactgcaacttccacctcccaggttcaagtgattctcctgcctcagcctccctaatagctgggatgacaggctcgtgccaccacacccaattaatttttgtatttttagtagagagagggtttcaccatgttggccaggctgatcttgaactcctgacctcaggtgatctgcccgcctcggcctcccaaagtgctgggattacaggtgtgagccaccatgcccagcctggcctgaggatttttgtgagcactcctgatgctgttgtgtagccaggagtgaggactgccctctgatcacccagcacagaacgttcagggccatgttgggacagtggactggtaagggcgagaggaggacactggggagcaggctgtagggtgccacagtccgccacagtcacaggctcatgctctccgtccgagaggaggcagaactgcttttaccaaggctcatccagccctcttcatttctctgggaacaatttttggtttcgtgacccatttcttaatccccggaaggcatttctctgaactggtgccaggcaccccggcccccagctccagttagttattattcgaggctgatttcttttctgtctgtcttccttctaattcctagattggtgaacagccacctcacttccagtttttgccggggcctcttttcagttctgagcaccagccagagtctaactgaattggacctcagtgacaattctctgggggacccagggatgagagtgttgtgtgaaacgctccagcatcctggctgtaacattcggagattgtggtgagtccccgtgcatgtgatctgtgtgagtgcaagttcatatgagagagagagagagaaacagactggagaaagatcttcaggaccaggttgctggtgtggtttctttcttttctttttctttttttctttttttgaggcagagttttgctcttattgcccaggctggagtgcaatgccgtgacctcggctcactgcaatctctgcctcctgggttcaagtgattctcctgcctcagcctcctgagtagctgggattacaggcacccaccaccatacccagctaattttttgtatttttagtagagatggggtttcatcatgttgtccaggctagtctcgaagtcctgacctcaggtgatccgcccgcctcggcctcccaaaatgctgggattacaggcatgagccaccgcacccggccggtggtgtggtttctgctgtaacaaagtgccacagactgaatggcttcaacaacaaaaattcattttcttgcagttctgaggccgaaagtctaagatcaaggtgtcagcagggttggtttcttctgaggccgccctccttggtttgtagatgcccccttctccctgtgtactgatgccatcttgccttggggggaagagtggcctagtctcctcttcttacagggacagcagtcctgatggattaggcacagtcaggcatgactacacaagacctaatgttaatctagtcactcctttaaagaccccatttcccaaaacagccacattatgaggtcttgaggattagggcttcaacatgtaaatttttgggggggcacagttcagcccagaacaagtgagaaggtagaatgggttggaggccagctggggaagttttgtgtggggaagaatgaatgaggaactaaaaaaaaagtcatttcttgaaacaaccatgatgctcgatatattccatttcctcatgggccatgttcatagatgagagttggagaatttgccatttacaacaatggcaacctcacctaaaaactatgaaaatactttttttttttttctttgagacaggacctctctctgtcacccaggctggagtgtgtgatcacagcttactgcagcctcgacctcccaggctcaagcaatcctcccacctcagctcccccgagtagctgggactacaggcatgcaccaccatgctggctaatttttgtattttttttgtagagatgtggtctcatcacgttgcccagcctggtcagaatgctttgtatgggttgaaatgtcacagggaatctcctgtgactttattaataatgtgcttgctcacacctgtagtcccagctactcgggaggctgaggcaggagaactgtgtgtacccgggaggcggagatggcagtgagccgagatcgtgccattgcactccagcctgggtgacagagcgagattccatttcaaaataataataataataataataataaaaaatgtgcttgctttctcccacaccatgaatcagattcacacgccccaccgcgctggtgcctgggccgcctcactagctctgtttcattccctgctctgtggacgcatgcaggcgtctgcatttttgaggagctgcacagcaagtgttaaagtccgccactctaggacattttgctacctccctctccggctcaacacactcaccttgtcaccagtcttatcaatttttcctcctacccgagctgttttccacttttcctcgtcctcaccccaccagcccaggtccagcaactgtcaccacttgcctggacacgcagtgacctcctagaccatcagctccccggggatggggtttgcgtttgtttgtcttgctgtcatctttgccacgcctggacagacatcttagtacccatcagtggtcaaaagcacgcactgaatgaatgttggctctttggtctccctgtgtctgcctttttccctcacccgaccattctctgcacaaaagtaagatggtcctttagaaaattctgattcgattgtgctgttcggtggcttaaatgctttctgtctgctctgctctttattttttactttatgtatttatttttgtagatgtagggggcgcaagagcagctttgtttcatggatatgttactgcacagtggtagtgtctgggcttttactgtaactacccctttttttttgagatggactctcgccgtgttgcccaggctggagtacagtggagtgatctcggctcactgcaacatccacctcctgggttcaagcaattcttcgagtagttgggattacaggcatgggccaccacgcccagctgatttttgtatttttagtagggacaaggtttcaccgtattggctaggctggtctcaaactcctgacctcgtgatccacccgcctcggcctcccaaagtgctgggattacaggcgtgagccaccgtgcccggccttagtgtaactcttacccaaatagtgaacattgtacccaataaataattttccagccctcacctggctcccaccctcccacccttctgaggctctagtgtctatcattccatgctctatggatgttaccctttgtttagctcccacttgtaagtgagaacgtgtggtatttgactctgtttctgagttgtttcacttaaggtaatggcctccagttccatctgtgtggcttcaaagatgtgacttcattcttttctatggctgagcagtattccattatgtatatagatcacactcttcatctttgctcttttggtaaatttcaaatcgtagtgccatggagactggttgtttgggacatttaaccttatttctccaacttcatgcccaaattctctccccactgccattgcccctgtatgattctggagaggcagacacaaacagggaagggtaggggacagtgagatgcttcgcagagaagtagagatccatttgtccatttgcttgaagttggtgtaaggatagggacagaaaggaaggtggtctaacaggctccaggacctatgaactattgccctggaaagaagctagaatgttgttcttgcccccatgacagggataaagtatagtttttgttgctggtggtggtggtgacggtggtagtgatgtgcgtgtgtgtttgtgtgcatgtgtgtgacggggaaactaatcaagaatcagagctgcagcgggatgtggaatcccagcactttgagaggctgaggtgggcagatcacctgagcccaggagttcaaaaccagcctgagcaagctggtgagaccttgtctttagaagataataataataataaaaataaaatataaataaaataaaatggctaagcatggtagcacatgcctgtatttgcagccacttgggaggctgaggcaggtggatcccttgagcccaggagttcgaggtggcagtgagctatgatctcgccagtgcactccagcctgtgcaacaaagtgagaccctgtctttaaaaaaagaaaagaaaagaaaagaaaaagaatcagagctgcttttcctttcaatatagatggagggctgggatgaaatcgtcaaacacaatggggtgacaatgttggtgaagacaagaaatagctgagtgcagcactggcgagggcagtgccgactgcgggcacgtcaatggttaggaacacgagggaaggaggtgggtgcacaggcccacaccgcccgcttcacttacaaaccaggagcgcaccattcacatgtctggggggtgatctcaatgcacccggcactgtttacggccgggagttcactgacaggtgatccctctgctgtgactggggagactgggggctgacctcccaatgtgccttgtgctgtattccggagctctctggtcaggtgtattctgatgctttctctattccagagctctctggtcaggtgtgtcctgatgctttctctattccagagctctctggtcaggtgtgtcctgatgctttctctattccggagctctctggtcaggtgtgtcctgatgctttctctattccggagctctctggtcaggtgtgtcctgatgcattctctattccagagctctctggtcaggtgtgtcctgatgctttctctattccagagctctcttgtcaggtgtgtcctgatgctttctctattctggagctctctggtcagatgtgttctgatgctttctctattccggagctctctggtcaggtgtgttctgatgctttctgtattccggagctctctgatcagatgtgttctgatgctttctctattccggagcttcctgatcaggtgtgtcctgatgcttcctctgttctggagctctctggtcaggtgtgttctgatgctttctgcctctgttcttggcatgaaggttggggcgctgtggcctctcgcatgagtgctgcttcgacatctccttggtcctcagcagcaaccagaagctggtggagctggacctgagtgacaacgccctcggtgacttcggaatcagacttctgtgtgtgggactgaagcacctgttgtgcaatctgaagaagctctggtgagtcgagcccgttcccctaaggaagttctgccagcgaggcgtgctgcgcactctggcttcagtgaggcccggtgggctggggtttgagtgagaatggccttggcggctcgggtgactgcgtgtgcttgtcttggggtgtctagcattgcggtcagtgagtgtttgtcgtggggaaagccacacactcaaccctgacaaggagcataaataataacaaaaagaagatgtctgcctttagggtgcctactgcctagctgacaaaaagaaaaaatcagtgtaatgcattgctttagaggagttttagaacttcatgacaagccctgggtcctcatattgtataagaaaaatggtgctaaagcaatgcagaccctcgatgttgtatgcagcccttccctgagtgcttctgggagacttgcattttgaaggagagaaaaaaccacttaaaaatggaagggccatttctcacccattaggatggctattatcaaacaaaagcaagaaaactagaaaacaacaagtattggtgtgggtgcggagaaatggaagccattgtgtgttgctggtgggaatgtaaatgatgcagccactatggaaaacagcacagcaattcctaaaaaataaatacagaattactactggagccagcatttccactgctgggtatatctgcaaaagagttcaaagcaggaactcagccaggagcggtggctcacacctgtaatcccagcattttgggaggctgaggcaggtggatcactggaggtcgggagttcgagactagcctggccatcatagtaaaaccttgtctctactaaaaatacaaaaattagccaggcatggtggtgcatgcctgtaatcccagttactcggggtggaggcaagagaatcttttgaacctgggaggcgggggttgcagtgagtcaagatcccgtcactgtactcccgcctgggtgacagaggaagcattaaaaaaaacagaaggcaggaactcaaacatgttatttgtacatctatgtccatagcaacactattagaaatagccaaaaggtggaaataaccaaacgtccttcaacggatgaagggtgttgaaaagtgctatatatgtacattcagctttaaaaagaattctagtacacactacaacatggatgagcctcggggacattatgctaagtaagataagctagactcgaaagggcaaatactctgattgtccttataggaggtgtctagaacagacagattcatagagacaggaagaaacgtggttgcaggcaatgggagggcggagagtgggaagacattgtttaatggttatggagtttcagttctgcaagatgaaaggagttctggagatggtggtggtgatggttgcacaacaatgtgaagtgcttaaggccattaattgtgcacttaaaaatggttaaaatggtaaattttattttacacgtattttaccacaataaagctgaatgtagggagctgggaagatgtagtattggtgggagcttggagaatgcttacttgttattccaccaaatggaaaaggaaatcagaagtgtacatagagcttgtgtccactccctttccatgtgtaaactggagtagaggcagtggcaggtacgggtgcttccttgtccatggtggagcgtgggtgagagacatgactgacattctgccatctctatggaaggttggtcagctgctgcctcacatcagcatgttgtcaggatcttgcatcagtattgagcaccagccattccctgaccagactctatgtgggggagaatgccttgggagactcaggagtcgcaattttatgtgaaaaagccaagaatccacagtgtaacctgcagaaactggggtaagtcttcatgggtgtcttaccagaaaagtaacttcttttcagaggtgaattatttggaaaatgtggatgggggttactttggtcaggcagagctgaaggtagagtaaggaggtaaaaacatcagaaatgctttgaaggactggaggcatataatgtatactgctgctaataagataatttatgctaatttatgattatagaactagagtttcttagtgttggaagaaaccttagagatggtgcagcccaattgcctgttgagggaaaaacttgcttccaagacactcacatgaatagtcacccattttatgtgtgcatgcttgcaagcatgcaagttatcgcatagtgcatgcagcctctacactaggaaagagctctcaccctgcagcctctacactaggagaggactcttacactgcagcctctatatcctcaacagtgtcatggctcagcaccttcacctatgtagccatgctctcccaggtacacaggctggtttgttagtgtctgtctttgaaatgtggtgcccagacctgatgatgcatggtggatgttttacctcctgttgcagagaactatggtttcactggtggaatctctgatttccttgttttttttggcagttacataagccttttgattcatgttggaattctgcagtaaacagtagtagggaatacccactaataatcatgcactttccctgtaccactttcctgtatcactttccctgtatcacctgctctttcatgttcttacatggattttctcagttggcttcagaaagatgctatagggtagggtatctgtgtctccaatttaaagaggagaaaataaaattcagggagcctcataactttgtccaaggacactcagagagaaggtagtggaactcagtgtagacaaacgtctgtgacctcccaagccactgccttaattcccagtgcattgccttaggagatgttgctaatgggctctctacagggcttcttcatctggctctcagcttaagggtgggacctccaatttagctttgctaaacctcatcttacctgtcttttgacccattgactaggcagttttgaatcttcattctgacaataatatctacaaatacaataagcaaattggataggtcttcattcattcacattattaatatatataccagccagatcagcattcaagacaaaatttggcaggaaaggtcttcccagaggagtaatccactaatccataattaacaaccaacctctgttcacaaagctctgatacttccttggacctctcccatcagccaatcctcagatgtgaccatatctctttctatgaagtcttcctgcagtgccttcatttcctcgccttagggtattacccttccttagcaaatcgccttagaatattactcttccttggcaatttgacttctgcttctctaagtttgttcaacagacatgattttgtcccttcattttgcaggccctctgggggctctgtggtgggcactgacagcagcagtgagtaaaacagaccaaaatcctgttttcacaaccgttcttttagggaagtgataacagataatagacatgctaaacaatataccctgtgtataagagctataagaaaaactggagtgacggcgaggggtgcttctctgctcacgtttggcgagtgcctctccgagcgggtgacattggagcaggtgctcagtgaagcgtgggatgggctgtgaaggagccggaagagccctccaggcaaagcagatggaaccgaagaggcccaggcgtaggcactcaggtgactctcagtgatcctggttccaaattccacatgacaaacaagccaattacaaatgtgcctgatgcatctgtaatatgctctatgtagttactattgtcaatacctaacctggtagagctgctgtaataaagtatcacaaacagagcggcctaaacaacaagagtttattgcctctcagttcaggaggccaaagtctgagactgaggcatcagcaggctgggtccttataggtcgtacaggtccttcccagtccttactttagttgacatctgctgcctttaattcatttcactcttcctctttggaagccttcttcgctcttatttttaggtcactacttagtctttcctgctaatgttatagcttccccctccctggatcctgcattggcttctcttcctgtggtctggggatttcacgactggtttagttgtgtttttttttttttttttttttttgagttggagtcttactctgtcacccaggctaaaatgcagtggcgcaatcttggctcactgcaacctctgcctcccgggttccaacaattcttctgcctcagccttctgagtagcagggactacaggtgtgcactgccatgcctggctaatttttgtatttttagtagatacggggtttcaccatgtttgccaggctggtctcgaatttctgacctcaggtgatctgcctgccttggcctcccaaagtgttgggattacaggcgtgagccacacacccgcctggtttaccttttttgttgttgttgttactaaatatttctaaactccctgagaattctcatatacctctcaatttcaacaacagtaggtctcaaacccagatatcattcacaaactttttattagaaaagtaacacatgcttggtgtaaacatatcatagtacagacaaagtggaaagtgaaagctccttctcagccctcttctgagagagccatttacaatagtctgatgaacaaccatccagactttttttaatacaatagattgttatataatccattcatctgtccatccatccatccatccatcatccatccctctattttccatccgtcaatccatccatccatccatccatccatccatccatccatccatcaatccatccaagggagtatcagagctttgtgaacatatattggttgctaattatggacaaatggatttcctgtatatgctgatctgggtggtatttatatcagtaatgtgaatgaagacctatgtgattttcatgttgcatttgtaatacattgttatgtagtattttaaatgcctagtctgttagagctactgtaataaagtatcacaaacagagcagcttaaacaacagaaatttattgtccctcagttctggaggccagaagtgtgagattaagatgctagcaggctgggtccttctgagggctgtggggaatctgttcatgcttctctttggcttctggtggaatgtcatcttgctgacacattttgccattccttgacctgtagatgcctctgccacaccattgcctttatctctacgtggtgtcttccctgtgtctgtgtgtgtctttctccatgtcccaagtttccctgtgaaagaacacaagtcatgttagatttgtaacgacctgatcttaactaattacattttcagtaaccgtatcaccaaataaggtcacaatctgaagtactggggcctaggacttccacatataaagtcttagaggaaacaattcaatccataacaccctatctattgaaactagaaccataatataaatatttttctgtgatatccttttttcatttatcatggataaatttccatgctgatttgtgtagatctatcgattggggcataatatcccatagtgtgctttaccataatttatttaatcaaatcatgcattttgggataactgttcttttccactattacaaacaactctgcaaaaagcacatttgtatatatagttttatgcatgtgtatttgctttgagataggtatctataagtggcattaatcagtcgagacttttatgcattttatgtgttaggtacagccatacatgtggctgtgtgttaggtatagcctatgggttaggcatagcctgtgtgttaggtatagcctatgggttaggtatagcctacgtgttaggtatagcctatgtgttaggtatagcctatgtgttaggtatagctacatttttttctctcgaactttgggtcctcatctctggctgcatattgtgtactgcctgtctcaatactgctcaagggtcacacatgctgagcatttgcaacattggtttaattatcataccccaccttccacccccgttctctgacctgctcctgccctcttcctcctgcctcattgaatagtggggctttccaggaggctgctgagtgagtaaacgggggacggtcctaaatgctctgctttcggttccctctaccactcatcactacaccagagggaactttttaagatgcaaatttgaaaatattgaccctttgttcaaaaatcttcaaaagttcttctctgcagtcagagtgacatcttagcttggcacacaaggctgtccctgatactgccctctctcctgccataccttgcactccattttgagtcatattggatgagttttgttttgctgttttagagatggggacttgctatgatgctcaggctgaatttgaattcctgggttcatctacagccataccacccagagtgcctgatctcatctgcactcctgggctcaagcaatcctcccgtctcagccgcccaagtagctgggactataggtggacatcactgtgcccagctatgaattagttttgaaacatcttggtggttcttttaatcttccttattcttgctactcttttccttttgcctggaaggttacccccactttcccactttctactatccatctaagaagctcctacttattttttaattactgtgatcctcattttgtccatttaaaagcagggataaaactaacattcccatcttgaggttgtgatcattcaatgagataaccctgtttaaagtcgtcatccatgtcaagcacacaataaatactcaaaaaatgtttgatcttcttcttcagattccttccttccttccttccttcttcctcctcctcctttctttctctctttttctttttctctttctttctttctttctctctctctctctttctttctttctctttctcccttcccttcccctcccccccccctttccctttcccctttcctttccttattttcctttcttttcttttctttttttaagacagggttttgctctgtggctcagactggagtgcagtggtaggatcatagctcactgtagccttgaactctcctgggctcaagcaatcttcctgcctcagccttctgactagctaggactacaggtgtgtactaccatgcctgggtaatgtttaaaatgtttaaaattttttatgtagagacagggtcttgctatgttgcccaggctggtctcaaactcctggcctcaagtggtcctcccaccttggcctcccaaagcactgggattcagttttcattatgagacagttttgtgacataggcaggtagttaataaataaaaatccccaaccaattaacttaatacagtacatggcaagatatccgtgcttggtaaatgtagagctcagttaggtggattggttgaaatcgagatgtcaatcacattcctctaatccaaggattggttggtgggacatggttgtttgattctttaaacattttattatgtgttcagtgtaaccgttgcttaatagttaccagcattttcacaatttggatatgacgttttcttaacaaaactgcaccgtctgttgcaatttctactactactgtgtagtgctccccaaacatctgttgaataatccattctactcctttttggaaatagtggtaaagcctgcataactttaattacataatttaaaaaatattttctcttttcctctttgaagaccatccccattctactagaatgtccctggcctttgccaggaatgctactaatcccttctgcatgttaattaacaacaaccctctgatagaattcttaccggggatgagagcttagtagaagccactcaaaacttgctttgtttttgtctgtctctcttgggcttcatttgcctattagtcttacttactgtgtcttttcagtttgactcttccttaattgggaagatagaaaccagtggatggataatcactttttcctgctggtcatctgttcatgctttactgtctgaatacactttttctgtatttctatttttcctgcctcgtagacaccaaagagcttaaaaaacatgccattgccaaggactctggcaggaaggaggcaatcacctgcgctattcctggcttcatggaatagaagcatctgtgctcctaggaattacgccaccagtgattttagagctcttatgctcttcctgggtgatagaatgggcttaatttgactcaagaatagtacatcaaaactcttaattccatcgagattgatgcatttttggggttttatgctctaaaaggccttttgctgtgcagaaggaagagagaggtggacagaaagggcagtggggagaagatgagactaaaaggtggaaagtccaggtctggatccctccccaccacacacctgctctgtggccttgagagaaacatgtaatttttataagccttagatttctaatatgtaaaatgtatgtaatagttttagctgcactccttccttgccaggttgatataataatcaaataagactggtctctgggtcaggcacagtggctcatgcctgtaatcccagcactttgggaggccaaggtgggtggattgtctgagcccaggagtttaagactgcagtgagctgtgcacttggcctgggtgactgagcaagagcctggctttttctatttatttatttatttttgagacagagtctcactttgttgcccaggttgaagtgcagtggcaatctcggctcactgcaacctctgcctcccaggttcaagcaattctcctgcctcagcctcccaagtagctgggattacaggtgcccaccaccaccctaactaatttttatatttttagtagagatgggctttcaccatgttggccaggctggttttgaactcctgacctcgtgatccaccctcccaaagtgctggacctcccaaagtgctgggattacaggcataagccactgcacctggccttatttatttatttatttaggcagggtctcactctgtcacccaggctagagtgtggcagcgcaatcatggctcactgcagcctcgacttcctggactcaagcgatcctcccacctctccaacgtggttgagggctgcagcgagaaagatgtgaaaggggcatttgggggcgcaggaaggcgggaggtggcaattgctaagggccccgccgtgttcctgtcacagccctagctacagcttagaaggtcacaatggacgttcacctgttgtctttgatgtttaaatcagcaccgacttcccatgaggaggatttttttttcctgtctctcttaaaaaaaaaaaaaaaggcccctgcagtgtgggtgtctgcagtgttacggggattattagttgaggataaccactgggatgtgtttctggaagaagtggtgctgaggagcactcctggccggaggaaagctgtgtgaggaggaggaggagtatggggtaactggctcagtttgccttggctctttctgtcggactcatacagcaaagcatctgctgagacatcatttgtgtctcctgtgacagccagagacagcaggtcttgctctccccagatcatatctgagatgctggctcctgctctctgagctgaaggctgcagctgctgagagaggacgaggcactgagtcaaagcagctgcacaatgttggggaacagctgggtactgaggactcttctccctgtccttctacaggttggtgaattctggccttacgtcagtctgttgttcagctttgtcctcggtactcagcactaatcagaatctcacgcacctttacctgcgaggcaacactctcggagacaaggggatcaaactactctgtgagggactcttgcaccccgactgcaagcttcaggtgttggagtaagtcctttggtttattacagcaatgagaacacatggtggccaagggtggagggacgtttaggcagagtggccacaagataatctgtatcttagaagtgaggtgtcttcttagtgtttgccttgttacaggatcatgggactgggaggagtccttccagatgatatagatgtaggaacttgagtccttaccatcacagttttctgtaccacaaaacaaggagggcagtatgacacttacattttatggaaatgtagggaaaggcacagaatttggtaaatcaatgaacaaaccactgttactaatttgatctgtgcaatgcagtggatttgaaaaagagagagaagaataattaccagcatacaaggctgcttgaagctagttagtcctgtgctcctgtgctttttttttttccacctgaaacaagacaggctaagatgctaaaatcttggggagctagggggatggttaaggggacattttctttaaatcaccccctttttgcagattagacaactgcaacctcacgtcacactgctgctgggatctttccacacttctgacctccagccagagcctgcgaaagctgagcctgggcaacaatgacctgggcgacctgggggtcatgatgttctgtgaagtgctgaaacagcagagctgcctcctgcagaacctggggtgagtgtgctctgcagagatgcccgtggtgggactctgagttctcaggaaacgttgactgttatcaaaatccaggatggctctcgcttcatttgcagccactcaagattaggttgggcctgggtcagttgtggtggatattttaaaatagagaatatggggttaaactacacattccacttcactgagatggttagaatcaccaaggctaagctggatttcacccacattccccaaagaaacacaagccctcatgcgatcatggccaaggaggattctgttctacacaaggaaccgaacttcgtagaactcgtctatgggtcatccaaattggaagggtctttacatagtggggatcttgttgttttgtttctgaggatttattttttttttccttgagacagagtcttgctctgtcgcccaggctggagtgcagtggcatgatctcggctcactgcaagctccgcctcccgggttcacgccattctcctgcctcggcctcccaagtagctgggaccacaggcgcctgccaccacacccggctaattttttgtatttttagtagagttggggtttcactgtgttagccaggatggtctccatctcctgacctcgtgatctgcctgcctcggcctccccaagtgctgggattacaggtgtgagcctccgtgcctggcggtttctgaggatttctaaatgggaaaagagtgctccaggaatagcaagatgggaaattacacgaaattcgacagtgtgtcaggatattgccagcccacctctgtgatactactgaggtagtcgtctcctctctagcaagagcttcctggcgagtcccacggttctgtcagccgtgatgattgcacttttccctgaagacgttttcactgcttggctttaaggccaacatgctctcctgggttttctcctatttcatcagatccttctactcagtctcctttctggcctcttcttcatcatcctgacccttatgtgttgaatcctcagagcttagtcgctgggctaatttttcttccctacactttctccctcatttaaattatctggtcttctggcttcatgatatgtgtgctgtggcctccagattatatgtccataaattctgtgtctccgtgcttggctgatctttcttcttgaaggtttaataaacctctcaaacgtcacatctagatccacacccttgctactctcttctacccgaaacctgattcttccccatttcagtcgatggtcacttcctccaagttgctcgggccaaaacatttctggttctgtcgtcactgtctctttttccttataatcatgtccaatgtgtcagcacatcctgtgagctctgccttcccagcttggccactccctgaccacttcaggccagcttcacggcttcttccttggctcaagccaccatcacctggtgtctggattgtcatggtggcattctgacaggtcaccctgcatctgttattctgattttagcagacaagggattcagctaaaacgtaactcgcatcatatcactcttgtcaacatcgtcttcttatcactttcataatcagagcaacaaactcatggtggcttccatgccccacataatccagcctctcctgcttccgaagattccgcttctcccaacccttgcttattctgccccagtacagtgatctccttgctgttttgcaagaacactgtcatctctggatttttgcacttgcttttcttctggcctagaactctctccccttcagtatccacctgcattattccctgtaccttcctaaattctctgcttaaaggtcttctctgcgaggctgtctttgactattctactgaaaatagaagcactttttcttctcttctcattctcctctgcagtgtagcacatcttatgtgctatgtcattttcttgtttatttattatctatctctgctcccaaaatataaattccattagtccagggacttctgtccatattaatgatctcaaggctgagaacttgttggaatgcaggtttgaataaatacaagtttgaatcagccttgcatccttgtgccaacataatggagcacatttgctgcaggacaagccaatattcccagagctaagagagatgggggaacaaacaaaggattggtcaggtcaaagctggggacactgctacatgtagcatcactgacctagccctgagggacattctggacccaggctgagggacaggctcaaaatagattgaagaatccatgaatgcaatcaacccgttttctcaggctgcgataacaagctgctttagactgtgtgactcaaacacagatttattatctcacaatactggaggctagaaagtccaaaatcaagtctctgatgaatttggttgctggcgagagtcctcttcctgatttgcagatggctaccttcttgttatgtgattgcatggagggaggggaggcaagggagagaagacaggggagaaagaacaaactctctgatatctctcctgtaagggcactcaccccatactgagggccccaccctcacaacttcatctaactctaactacttcccaagggcctcacctttaagtaccgtacattaggggttcgggcttcatcatgggaattgttaaggaagacaaacattcagtatataacaccaccaaattgagatggttataaatggtgcaggacaaatgggtggatttattagttaagttgcaagggaaactttattaaagaggaggaagaacttacatcgaagatgtgagaggcatcctgggcaaatgcaatgtgtagaattgatcaggttagtgactaaatgaacataaggtaaaaaaatttacaagacagttgaggaagcgtaaatgctgactggacatacagtgatactaagaaattattgttaaacgattttggtgtgatagtgacattgtaattatattttagtgtcacaaagtgaattattgatagatgcattaaatgcagtatttaaaataatccatgggtgggaggagtgggtgggcaatagaagaaacaagttgacatgacctgtttaattgtcgaacttgggtaatggacacagggggttcattatactattttctatacttttatgtatgcttaacattttcccaactagatagttaaaaatataacaggaaccacattgggcacttagagcttttttttttttttccccacatttttattgggagccgtgggaggggcctcctctgtcattggaggtgctcacagtttcttcagccactccaggctggggtcctgggggtctgaggctcccagtgacgaaggacacgacgagcaccggctccttggcccaggcattcttgaagaaggtgcggagtctcgcggccatcttggtctcggcggcggcgacggcggcgaggacgcggagcactctgggagttgtggtccctcttttttttttttttttttttttacattttagaagtgtgtggagtttagggaaatgaagagatgagacttttaaggttaacgactctgacagagttatctgaagagtgcaacccaggctttctatttgcttttacaggttgtctgaaatgtatttcaattatgagacaaaaagtgcgttagaaacacttcaaga

SEQ ID NO: 2 - DNA, 4187 bp, synthetic construct
  source 1..4187: mol_type = other DNA
gttcctgaggctggcatctggatgaggaaactgaagttgaggaatagtgaagagtttgtccaatgtcatagccccgtaatcaacgggacaaaaattttcttgctgatgggtcaagatggcatcgtgaagtggttgttcaccgtaaactgtaatacaatcctgtttatggatttgtttgcatatttttccctccatagggaaacctttcttccatggctcaggacacactcctggatcgagccaacaggagaactttctggtaagcatttggctaacttttttttttttgagatggagtcttgctgtgtcgcctaggctggagtgcagtggcgtgatcttggctcactgcagcctccacttcccgggttcaatcaattctcctacctcaacttcctgagtagctgggattacaggcgcccgccaccacacccggctcatttttgtacttttagtagagacacagttttgccatgttggccaggctggtcttgaattcctcagctcaggtgatctgcctgccttggcctctcaaagtgctgggattacaggcgtgagccactgtgcccggccttggctaacttttcaaaattaaagattttgacttgttacagtcatgtgacatttttttctttctgtttgctgagtttttgataatttatatctctcaaagtggagactttaaaaaagactcatccgtgtgccgtgttcactgcctggtatcttagtgtggaccgaagcctaaggaccctgaaaacagctgcagatgaagatggcaagcacccgctgcaagctggccaggtacctggaggacctggaggatgtggacttgaagaaatttaagatgcacttagaggactatcctccccagaagggctgcatccccctcccgaggggtcagacagagaaggcagaccatgtggatctagccacgctaatgatcgacttcaatggggaggagaaggcgtgggccatggccgtgtggatcttcgctgcgatcaacaggagagacctttatgagaaagcaaaaagagatgagccgaagtggggttcagataatgcacgtgtttcgaatcccactgtgatatgccaggaagacagcattgaagaggagtggatgggtttactggagtacctttcgagaatctctatttgtaaaatgaagaaagattaccgtaagaagtacagaaagtacgtgagaagcagattccagtgcattgaagacaggaatgcccgtctgggtgagagtgtgagcctcaacaaacgctacacacgactgcgtctcatcaaggagcaccggagccagcaggagagggagcaggagcttctggccatcggcaagaccaagacgtgtgagagccccgtgagtcccattaagatggagttgctgtttgaccccgatgatgagcattctgagcctgtgcacaccgtggtgttccagggggcggcagggattgggaaaacaatcctggccaggaagatgatgttggactgggcgtcggggacactctaccaagacaggtttgactatctgttctatatccactgtcgagaggtgagccttgtgacacagaggagcctgggggacctgatcatgagctgctgccccgacccaaacccacccatccacaagatcgtgagaaaaccctccagaatcctcttcctcatggacggcttcgatgagctgcaaggtgcctttgacgagcacataggaccgctctgcactgactggcagaaggccgagcggggagacattctcctgagcagcctcatcagaaagaagctgcttcccgaggcctctctgctcatcaccacgagacctgtggccctggagaaactgcagcacttgctggaccatcctcggcatgtggagatcctgggtttctccgaggccaaaaggaaagagtacttcttcaagtacttctctgatgaggcccaagccagggcagccttcagtctgattcaggagaacgaggtcctcttcaccatgtgcttcatccccctggtctgctggatcgtgtgcactggactgaaacagcagatggagagtggcaagagccttgcccagacatccaagaccaccaccgcggtgtacgtcttcttcctttccagtttgctgcagccccggggagggagccaggagcacggcctctgcgcccacctctgggggctctgctctttggctgcagatggaatctggaaccagaaaatcctgtttgaggagtccgacctcaggaatcatggactgcagaaggcggatgtgtctgctttcctgaggatgaacctgttccaaaaggaagtggactgcgagaagttctacagcttcatccacatgactttccaggagttctttgccgccatgtactacctgctggaagaggaaaaggaaggaaggacgaacgttccagggagtcgtttgaagcttcccagccgagacgtgacagtccttctggaaaactatggcaaattcgaaaaggggtatttgatttttgttgtacgtttcctctttggcctggtaaaccaggagaggacctcctacttggagaagaaattaagttgcaagatctctcagcaaatcaggctggagctgctgaaatggattgaagtgaaagccaaagctaaaaagctgcagatccagcccagccagctggaattgttctactgtttgtacgagatgcaggaggaggacttcgtgcaaagggccatggactatttccccaagattgagatcaatctctccaccagaatggaccacatggtttcttccttttgcattgagaactgtcatcgggtggagtcactgtccctggggtttctccataacatgcccaaggaggaagaggaggaggaaaaggaaggccgacaccttgatatggtgcagtgtgtcctcccaagctcctctcatgctgcctgttctcatggattggtgaacagccacctcacttccagtttttgccggggcctcttttcagttctgagcaccagccagagtctaactgaattggacctcagtgacaattctctgggggacccagggatgagagtgttgtgtgaaacgctccagcatcctggctgtaacattcggagattgtggttggggcgctgtggcctctcgcatgagtgctgcttcgacatctccttggtcctcagcagcaaccagaagctggtggagctggacctgagtgacaacgccctcggtgacttcggaatcagacttctgtgtgtgggactgaagcacctgttgtgcaatctgaagaagctctggttggtcagctgctgcctcacatcagcatgttgtcaggatcttgcatcagtattgagcaccagccattccctgaccagactctatgtgggggagaatgccttgggagactcaggagtcgcaattttatgtgaaaaagccaagaatccacagtgtaacctgcagaaactggggttggtgaattctggccttacgtcagtctgttgttcagctttgtcctcggtactcagcactaatcagaatctcacgcacctttacctgcgaggcaacactctcggagacaaggggatcaaactactctgtgagggactcttgcaccccgactgcaagcttcaggtgttggaattagacaactgcaacctcacgtcacactgctgctgggatctttccacacttctgacctccagccagagcctgcgaaagctgagcctgggcaacaatgacctgggcgacctgggggtcatgatgttctgtgaagtgctgaaacagcagagctgcctcctgcagaacctggggttgtctgaaatgtatttcaattatgagacaaaaagtgcgttagaaacacttcaagaagaaaagcctgagctgaccgtcgtctttgagccttcttggtaggagtggaaacggggctgccagacgccagtgttctccggtccctccagctgggggccctcaggtggagagagctgcgatccatccaggccaagaccacagctctgtgatccttccggtggagtgtcggagaagagagcttgccgacgatgccttcctgtgcagagcttgggcatctcctttacgccagggtgaggaagacaccaggacaatgacagcatcgggtgttgttgtcatcacagcgcctcagttagaggatgttcctcttggtgacctcatgtaattagctcattcaataaagcactttctttatttt

SEQ ID NO: 3 - DNA, 3845 bp, synthetic construct
  source 1..3845: mol_type = other DNA
gttcctgaggctggcatctggatgaggaaactgaagttgaggaatagtgaagagtttgtccaatgtcatagccccgtaatcaacgggacaaaaattttcttgctgatgggtcaagatggcatcgtgaagtggttgttcaccgtaaactgtaatacaatcctgtttatggatttgtttgcatatttttccctccatagggaaacctttcttccatggctcaggacacactcctggatcgagccaacaggagaactttctggtaagcatttggctaacttttttttttttgagatggagtcttgctgtgtcgcctaggctggagtgcagtggcgtgatcttggctcactgcagcctccacttcccgggttcaatcaattctcctacctcaacttcctgagtagctgggattacaggcgcccgccaccacacccggctcatttttgtacttttagtagagacacagttttgccatgttggccaggctggtcttgaattcctcagctcaggtgatctgcctgccttggcctctcaaagtgctgggattacaggcgtgagccactgtgcccggccttggctaacttttcaaaattaaagattttgacttgttacagtcatgtgacatttttttctttctgtttgctgagtttttgataatttatatctctcaaagtggagactttaaaaaagactcatccgtgtgccgtgttcactgcctggtatcttagtgtggaccgaagcctaaggaccctgaaaacagctgcagatgaagatggcaagcacccgctgcaagctggccaggtacctggaggacctggaggatgtggacttgaagaaatttaagatgcacttagaggactatcctccccagaagggctgcatccccctcccgaggggtcagacagagaaggcagaccatgtggatctagccacgctaatgatcgacttcaatggggaggagaaggcgtgggccatggccgtgtggatcttcgctgcgatcaacaggagagacctttatgagaaagcaaaaagagatgagccgaagtggggttcagataatgcacgtgtttcgaatcccactgtgatatgccaggaagacagcattgaagaggagtggatgggtttactggagtacctttcgagaatctctatttgtaaaatgaagaaagattaccgtaagaagtacagaaagtacgtgagaagcagattccagtgcattgaagacaggaatgcccgtctgggtgagagtgtgagcctcaacaaacgctacacacgactgcgtctcatcaaggagcaccggagccagcaggagagggagcaggagcttctggccatcggcaagaccaagacgtgtgagagccccgtgagtcccattaagatggagttgctgtttgaccccgatgatgagcattctgagcctgtgcacaccgtggtgttccagggggcggcagggattgggaaaacaatcctggccaggaagatgatgttggactgggcgtcggggacactctaccaagacaggtttgactatctgttctatatccactgtcgagaggtgagccttgtgacacagaggagcctgggggacctgatcatgagctgctgccccgacccaaacccacccatccacaagatcgtgagaaaaccctccagaatcctcttcctcatggacggcttcgatgagctgcaaggtgcctttgacgagcacataggaccgctctgcactgactggcagaaggccgagcggggagacattctcctgagcagcctcatcagaaagaagctgcttcccgaggcctctctgctcatcaccacgagacctgtggccctggagaaactgcagcacttgctggaccatcctcggcatgtggagatcctgggtttctccgaggccaaaaggaaagagtacttcttcaagtacttctctgatgaggcccaagccagggcagccttcagtctgattcaggagaacgaggtcctcttcaccatgtgcttcatccccctggtctgctggatcgtgtgcactggactgaaacagcagatggagagtggcaagagccttgcccagacatccaagaccaccaccgcggtgtacgtcttcttcctttccagtttgctgcagccccggggagggagccaggagcacggcctctgcgcccacctctgggggctctgctctttggctgcagatggaatctggaaccagaaaatcctgtttgaggagtccgacctcaggaatcatggactgcagaaggcggatgtgtctgctttcctgaggatgaacctgttccaaaaggaagtggactgcgagaagttctacagcttcatccacatgactttccaggagttctttgccgccatgtactacctgctggaagaggaaaaggaaggaaggacgaacgttccagggagtcgtttgaagcttcccagccgagacgtgacagtccttctggaaaactatggcaaattcgaaaaggggtatttgatttttgttgtacgtttcctctttggcctggtaaaccaggagaggacctcctacttggagaagaaattaagttgcaagatctctcagcaaatcaggctggagctgctgaaatggattgaagtgaaagccaaagctaaaaagctgcagatccagcccagccagctggaattgttctactgtttgtacgagatgcaggaggaggacttcgtgcaaagggccatggactatttccccaagattgagatcaatctctccaccagaatggaccacatggtttcttccttttgcattgagaactgtcatcgggtggagtcactgtccctggggtttctccataacatgcccaaggaggaagaggaggaggaaaaggaaggccgacaccttgatatggtgcagtgtgtcctcccaagctcctctcatgctgcctgttctcatgggttggggcgctgtggcctctcgcatgagtgctgcttcgacatctccttggtcctcagcagcaaccagaagctggtggagctggacctgagtgacaacgccctcggtgacttcggaatcagacttctgtgtgtgggactgaagcacctgttgtgcaatctgaagaagctctggttggtgaattctggccttacgtcagtctgttgttcagctttgtcctcggtactcagcactaatcagaatctcacgcacctttacctgcgaggcaacactctcggagacaaggggatcaaactactctgtgagggactcttgcaccccgactgcaagcttcaggtgttggaattagacaactgcaacctcacgtcacactgctgctgggatctttccacacttctgacctccagccagagcctgcgaaagctgagcctgggcaacaatgacctgggcgacctgggggtcatgatgttctgtgaagtgctgaaacagcagagctgcctcctgcagaacctggggttgtctgaaatgtatttcaattatgagacaaaaagtgcgttagaaacacttcaagaagaaaagcctgagctgaccgtcgtctttgagccttcttggtaggagtggaaacggggctgccagacgccagtgttctccggtccctccagctgggggccctcaggtggagagagctgcgatccatccaggccaagaccacagctctgtgatccttccggtggagtgtcggagaagagagcttgccgacgatgccttcctgtgcagagcttgggcatctcctttacgccagggtgaggaagacaccaggacaatgacagcatcgggtgttgttgtcatcacagcgcctcagttagaggatgttcctcttggtgacctcatgtaattagctcattcaataaagcactttctttatttt

SEQ ID NO: 4 - DNA, 3545 bp, synthetic construct
  source 1..3545: mol_type = other DNA
gttcctgaggctggcatctggggaaacctttcttccatggctcaggacacactcctggatcgagccaacaggagaactttctgtgtggaccgaagcctaaggaccctgaaaacagctgcagatgaagatggcaagcacccgctgcaagctggccaggtacctggaggacctggaggatgtggacttgaagaaatttaagatgcacttagaggactatcctccccagaagggctgcatccccctcccgaggggtcagacagagaaggcagaccatgtggatctagccacgctaatgatcgacttcaatggggaggagaaggcgtgggccatggccgtgtggatcttcgctgcgatcaacaggagagacctttatgagaaagcaaaaagagatgagccgaagtggggttcagataatgcacgtgtttcgaatcccactgtgatatgccaggaagacagcattgaagaggagtggatgggtttactggagtacctttcgagaatctctatttgtaaaatgaagaaagattaccgtaagaagtacagaaagtacgtgagaagcagattccagtgcattgaagacaggaatgcccgtctgggtgagagtgtgagcctcaacaaacgctacacacgactgcgtctcatcaaggagcaccggagccagcaggagagggagcaggagcttctggccatcggcaagaccaagacgtgtgagagccccgtgagtcccattaagatggagttgctgtttgaccccgatgatgagcattctgagcctgtgcacaccgtggtgttccagggggcggcagggattgggaaaacaatcctggccaggaagatgatgttggactgggcgtcggggacactctaccaagacaggtttgactatctgttctatatccactgtcgagaggtgagccttgtgacacagaggagcctgggggacctgatcatgagctgctgccccgacccaaacccacccatccacaagatcgtgagaaaaccctccagaatcctcttcctcatggacggcttcgatgagctgcaaggtgcctttgacgagcacataggaccgctctgcactgactggcagaaggccgagcggggagacattctcctgagcagcctcatcagaaagaagctgcttcccgaggcctctctgctcatcaccacgagacctgtggccctggagaaactgcagcacttgctggaccatcctcggcatgtggagatcctgggtttctccgaggccaaaaggaaagagtacttcttcaagtacttctctgatgaggcccaagccagggcagccttcagtctgattcaggagaacgaggtcctcttcaccatgtgcttcatccccctggtctgctggatcgtgtgcactggactgaaacagcagatggagagtggcaagagccttgcccagacatccaagaccaccaccgcggtgtacgtcttcttcctttccagtttgctgcagccccggggagggagccaggagcacggcctctgcgcccacctctgggggctctgctctttggctgcagatggaatctggaaccagaaaatcctgtttgaggagtccgacctcaggaatcatggactgcagaaggcggatgtgtctgctttcctgaggatgaacctgttccaaaaggaagtggactgcgagaagttctacagcttcatccacatgactttccaggagttctttgccgccatgtactacctgctggaagaggaaaaggaaggaaggacgaacgttccagggagtcgtttgaagcttcccagccgagacgtgacagtccttctggaaaactatggcaaattcgaaaaggggtatttgatttttgttgtacgtttcctctttggcctggtaaaccaggagaggacctcctacttggagaagaaattaagttgcaagatctctcagcaaatcaggctggagctgctgaaatggattgaagtgaaagccaaagctaaaaagctgcagatccagcccagccagctggaattgttctactgtttgtacgagatgcaggaggaggacttcgtgcaaagggccatggactatttccccaagattgagatcaatctctccaccagaatggaccacatggtttcttccttttgcattgagaactgtcatcgggtggagtcactgtccctggggtttctccataacatgcccaaggaggaagaggaggaggaaaaggaaggccgacaccttgatatggtgcagtgtgtcctcccaagctcctctcatgctgcctgttctcatggattggtgaacagccacctcacttccagtttttgccggggcctcttttcagttctgagcaccagccagagtctaactgaattggacctcagtgacaattctctgggggacccagggatgagagtgttgtgtgaaacgctccagcatcctggctgtaacattcggagattgtggttggggcgctgtggcctctcgcatgagtgctgcttcgacatctccttggtcctcagcagcaaccagaagctggtggagctggacctgagtgacaacgccctcggtgacttcggaatcagacttctgtgtgtgggactgaagcacctgttgtgcaatctgaagaagctctggttggtcagctgctgcctcacatcagcatgttgtcaggatcttgcatcagtattgagcaccagccattccctgaccagactctatgtgggggagaatgccttgggagactcaggagtcgcaattttatgtgaaaaagccaagaatccacagtgtaacctgcagaaactggggttggtgaattctggccttacgtcagtctgttgttcagctttgtcctcggtactcagcactaatcagaatctcacgcacctttacctgcgaggcaacactctcggagacaaggggatcaaactactctgtgagggactcttgcaccccgactgcaagcttcaggtgttggaattagacaactgcaacctcacgtcacactgctgctgggatctttccacacttctgacctccagccagagcctgcgaaagctgagcctgggcaacaatgacctgggcgacctgggggtcatgatgttctgtgaagtgctgaaacagcagagctgcctcctgcagaacctggggttgtctgaaatgtatttcaattatgagacaaaaagtgcgttagaaacacttcaagaagaaaagcctgagctgaccgtcgtctttgagccttcttggtaggagtggaaacggggctgccagacgccagtgttctccggtccctccagctgggggccctcaggtggagagagctgcgatccatccaggccaagaccacagctctgtgatccttccggtggagtgtcggagaagagagcttgccgacgatgccttcctgtgcagagcttgggcatctcctttacgccagggtgaggaagacaccaggacaatgacagcatcgggtgttgttgtcatcacagcgcctcagttagaggatgttcctcttggtgacctcatgtaattagctcattcaataaagcactttctttatttt

SEQ ID NO: 5 - DNA, 4016 bp, synthetic construct
  source 1..4016: mol_type = other DNA
gttcctgaggctggcatctggatgaggaaactgaagttgaggaatagtgaagagtttgtccaatgtcatagccccgtaatcaacgggacaaaaattttcttgctgatgggtcaagatggcatcgtgaagtggttgttcaccgtaaactgtaatacaatcctgtttatggatttgtttgcatatttttccctccatagggaaacctttcttccatggctcaggacacactcctggatcgagccaacaggagaactttctggtaagcatttggctaacttttttttttttgagatggagtcttgctgtgtcgcctaggctggagtgcagtggcgtgatcttggctcactgcagcctccacttcccgggttcaatcaattctcctacctcaacttcctgagtagctgggattacaggcgcccgccaccacacccggctcatttttgtacttttagtagagacacagttttgccatgttggccaggctggtcttgaattcctcagctcaggtgatctgcctgccttggcctctcaaagtgctgggattacaggcgtgagccactgtgcccggccttggctaacttttcaaaattaaagattttgacttgttacagtcatgtgacatttttttctttctgtttgctgagtttttgataatttatatctctcaaagtggagactttaaaaaagactcatccgtgtgccgtgttcactgcctggtatcttagtgtggaccgaagcctaaggaccctgaaaacagctgcagatgaagatggcaagcacccgctgcaagctggccaggtacctggaggacctggaggatgtggacttgaagaaatttaagatgcacttagaggactatcctccccagaagggctgcatccccctcccgaggggtcagacagagaaggcagaccatgtggatctagccacgctaatgatcgacttcaatggggaggagaaggcgtgggccatggccgtgtggatcttcgctgcgatcaacaggagagacctttatgagaaagcaaaaagagatgagccgaagtggggttcagataatgcacgtgtttcgaatcccactgtgatatgccaggaagacagcattgaagaggagtggatgggtttactggagtacctttcgagaatctctatttgtaaaatgaagaaagattaccgtaagaagtacagaaagtacgtgagaagcagattccagtgcattgaagacaggaatgcccgtctgggtgagagtgtgagcctcaacaaacgctacacacgactgcgtctcatcaaggagcaccggagccagcaggagagggagcaggagcttctggccatcggcaagaccaagacgtgtgagagccccgtgagtcccattaagatggagttgctgtttgaccccgatgatgagcattctgagcctgtgcacaccgtggtgttccagggggcggcagggattgggaaaacaatcctggccaggaagatgatgttggactgggcgtcggggacactctaccaagacaggtttgactatctgttctatatccactgtcgagaggtgagccttgtgacacagaggagcctgggggacctgatcatgagctgctgccccgacccaaacccacccatccacaagatcgtgagaaaaccctccagaatcctcttcctcatggacggcttcgatgagctgcaaggtgcctttgacgagcacataggaccgctctgcactgactggcagaaggccgagcggggagacattctcctgagcagcctcatcagaaagaagctgcttcccgaggcctctctgctcatcaccacgagacctgtggccctggagaaactgcagcacttgctggaccatcctcggcatgtggagatcctgggtttctccgaggccaaaaggaaagagtacttcttcaagtacttctctgatgaggcccaagccagggcagccttcagtctgattcaggagaacgaggtcctcttcaccatgtgcttcatccccctggtctgctggatcgtgtgcactggactgaaacagcagatggagagtggcaagagccttgcccagacatccaagaccaccaccgcggtgtacgtcttcttcctttccagtttgctgcagccccggggagggagccaggagcacggcctctgcgcccacctctgggggctctgctctttggctgcagatggaatctggaaccagaaaatcctgtttgaggagtccgacctcaggaatcatggactgcagaaggcggatgtgtctgctttcctgaggatgaacctgttccaaaaggaagtggactgcgagaagttctacagcttcatccacatgactttccaggagttctttgccgccatgtactacctgctggaagaggaaaaggaaggaaggacgaacgttccagggagtcgtttgaagcttcccagccgagacgtgacagtccttctggaaaactatggcaaattcgaaaaggggtatttgatttttgttgtacgtttcctctttggcctggtaaaccaggagaggacctcctacttggagaagaaattaagttgcaagatctctcagcaaatcaggctggagctgctgaaatggattgaagtgaaagccaaagctaaaaagctgcagatccagcccagccagctggaattgttctactgtttgtacgagatgcaggaggaggacttcgtgcaaagggccatggactatttccccaagattgagatcaatctctccaccagaatggaccacatggtttcttccttttgcattgagaactgtcatcgggtggagtcactgtccctggggtttctccataacatgcccaaggaggaagaggaggaggaaaaggaaggccgacaccttgatatggtgcagtgtgtcctcccaagctcctctcatgctgcctgttctcatggattggtgaacagccacctcacttccagtttttgccggggcctcttttcagttctgagcaccagccagagtctaactgaattggacctcagtgacaattctctgggggacccagggatgagagtgttgtgtgaaacgctccagcatcctggctgtaacattcggagattgtggttggggcgctgtggcctctcgcatgagtgctgcttcgacatctccttggtcctcagcagcaaccagaagctggtggagctggacctgagtgacaacgccctcggtgacttcggaatcagacttctgtgtgtgggactgaagcacctgttgtgcaatctgaagaagctctggttggtgaattctggccttacgtcagtctgttgttcagctttgtcctcggtactcagcactaatcagaatctcacgcacctttacctgcgaggcaacactctcggagacaaggggatcaaactactctgtgagggactcttgcaccccgactgcaagcttcaggtgttggaattagacaactgcaacctcacgtcacactgctgctgggatctttccacacttctgacctccagccagagcctgcgaaagctgagcctgggcaacaatgacctgggcgacctgggggtcatgatgttctgtgaagtgctgaaacagcagagctgcctcctgcagaacctggggttgtctgaaatgtatttcaattatgagacaaaaagtgcgttagaaacacttcaagaagaaaagcctgagctgaccgtcgtctttgagccttcttggtaggagtggaaacggggctgccagacgccagtgttctccggtccctccagctgggggccctcaggtggagagagctgcgatccatccaggccaagaccacagctctgtgatccttccggtggagtgtcggagaagagagcttgccgacgatgccttcctgtgcagagcttgggcatctcctttacgccagggtgaggaagacaccaggacaatgacagcatcgggtgttgttgtcatcacagcgcctcagttagaggatgttcctcttggtgacctcatgtaattagctcattcaataaagcactttctttatttt

SEQ ID NO: 6 - DNA, 4016 bp, synthetic construct
  source 1..4016: mol_type = other DNA
gttcctgaggctggcatctggatgaggaaactgaagttgaggaatagtgaagagtttgtccaatgtcatagccccgtaatcaacgggacaaaaattttcttgctgatgggtcaagatggcatcgtgaagtggttgttcaccgtaaactgtaatacaatcctgtttatggatttgtttgcatatttttccctccatagggaaacctttcttccatggctcaggacacactcctggatcgagccaacaggagaactttctggtaagcatttggctaacttttttttttttgagatggagtcttgctgtgtcgcctaggctggagtgcagtggcgtgatcttggctcactgcagcctccacttcccgggttcaatcaattctcctacctcaacttcctgagtagctgggattacaggcgcccgccaccacacccggctcatttttgtacttttagtagagacacagttttgccatgttggccaggctggtcttgaattcctcagctcaggtgatctgcctgccttggcctctcaaagtgctgggattacaggcgtgagccactgtgcccggccttggctaacttttcaaaattaaagattttgacttgttacagtcatgtgacatttttttctttctgtttgctgagtttttgataatttatatctctcaaagtggagactttaaaaaagactcatccgtgtgccgtgttcactgcctggtatcttagtgtggaccgaagcctaaggaccctgaaaacagctgcagatgaagatggcaagcacccgctgcaagctggccaggtacctggaggacctggaggatgtggacttgaagaaatttaagatgcacttagaggactatcctccccagaagggctgcatccccctcccgaggggtcagacagagaaggcagaccatgtggatctagccacgctaatgatcgacttcaatggggaggagaaggcgtgggccatggccgtgtggatcttcgctgcgatcaacaggagagacctttatgagaaagcaaaaagagatgagccgaagtggggttcagataatgcacgtgtttcgaatcccactgtgatatgccaggaagacagcattgaagaggagtggatgggtttactggagtacctttcgagaatctctatttgtaaaatgaagaaagattaccgtaagaagtacagaaagtacgtgagaagcagattccagtgcattgaagacaggaatgcccgtctgggtgagagtgtgagcctcaacaaacgctacacacgactgcgtctcatcaaggagcaccggagccagcaggagagggagcaggagcttctggccatcggcaagaccaagacgtgtgagagccccgtgagtcccattaagatggagttgctgtttgaccccgatgatgagcattctgagcctgtgcacaccgtggtgttccagggggcggcagggattgggaaaacaatcctggccaggaagatgatgttggactgggcgtcggggacactctaccaagacaggtttgactatctgttctatatccactgtcgagaggtgagccttgtgacacagaggagcctgggggacctgatcatgagctgctgccccgacccaaacccacccatccacaagatcgtgagaaaaccctccagaatcctcttcctcatggacggcttcgatgagctgcaaggtgcctttgacgagcacataggaccgctctgcactgactggcagaaggccgagcggggagacattctcctgagcagcctcatcagaaagaagctgcttcccgaggcctctctgctcatcaccacgagacctgtggccctggagaaactgcagcacttgctggaccatcctcggcatgtggagatcctgggtttctccgaggccaaaaggaaagagtacttcttcaagtacttctctgatgaggcccaagccagggcagccttcagtctgattcaggagaacgaggtcctcttcaccatgtgcttcatccccctggtctgctggatcgtgtgcactggactgaaacagcagatggagagtggcaagagccttgcccagacatccaagaccaccaccgcggtgtacgtcttcttcctttccagtttgctgcagccccggggagggagccaggagcacggcctctgcgcccacctctgggggctctgctctttggctgcagatggaatctggaaccagaaaatcctgtttgaggagtccgacctcaggaatcatggactgcagaaggcggatgtgtctgctttcctgaggatgaacctgttccaaaaggaagtggactgcgagaagttctacagcttcatccacatgactttccaggagttctttgccgccatgtactacctgctggaagaggaaaaggaaggaaggacgaacgttccagggagtcgtttgaagcttcccagccgagacgtgacagtccttctggaaaactatggcaaattcgaaaaggggtatttgatttttgttgtacgtttcctctttggcctggtaaaccaggagaggacctcctacttggagaagaaattaagttgcaagatctctcagcaaatcaggctggagctgctgaaatggattgaagtgaaagccaaagctaaaaagctgcagatccagcccagccagctggaattgttctactgtttgtacgagatgcaggaggaggacttcgtgcaaagggccatggactatttccccaagattgagatcaatctctccaccagaatggaccacatggtttcttccttttgcattgagaactgtcatcgggtggagtcactgtccctggggtttctccataacatgcccaaggaggaagaggaggaggaaaaggaaggccgacaccttgatatggtgcagtgtgtcctcccaagctcctctcatgctgcctgttctcatgggttggggcgctgtggcctctcgcatgagtgctgcttcgacatctccttggtcctcagcagcaaccagaagctggtggagctggacctgagtgacaacgccctcggtgacttcggaatcagacttctgtgtgtgggactgaagcacctgttgtgcaatctgaagaagctctggttggtcagctgctgcctcacatcagcatgttgtcaggatcttgcatcagtattgagcaccagccattccctgaccagactctatgtgggggagaatgccttgggagactcaggagtcgcaattttatgtgaaaaagccaagaatccacagtgtaacctgcagaaactggggttggtgaattctggccttacgtcagtctgttgttcagctttgtcctcggtactcagcactaatcagaatctcacgcacctttacctgcgaggcaacactctcggagacaaggggatcaaactactctgtgagggactcttgcaccccgactgcaagcttcaggtgttggaattagacaactgcaacctcacgtcacactgctgctgggatctttccacacttctgacctccagccagagcctgcgaaagctgagcctgggcaacaatgacctgggcgacctgggggtcatgatgttctgtgaagtgctgaaacagcagagctgcctcctgcagaacctggggttgtctgaaatgtatttcaattatgagacaaaaagtgcgttagaaacacttcaagaagaaaagcctgagctgaccgtcgtctttgagccttcttggtaggagtggaaacggggctgccagacgccagtgttctccggtccctccagctgggggccctcaggtggagagagctgcgatccatccaggccaagaccacagctctgtgatccttccggtggagtgtcggagaagagagcttgccgacgatgccttcctgtgcagagcttgggcatctcctttacgccagggtgaggaagacaccaggacaatgacagcatcgggtgttgttgtcatcacagcgcctcagttagaggatgttcctcttggtgacctcatgtaattagctcattcaataaagcactttctttatttt

SEQ ID NO: 7 - DNA, 4187 bp, synthetic construct; identical to SEQ ID NO: 2
  source 1..4187: mol_type = other DNA

SEQ ID NO: 8 - DNA, 5610 bp, synthetic construct
  source 1..5610: mol_type = other DNA
gttgactaggcgctgttcttgctggctggtgccccagggcctggagaggtctgaagaaacctgggagccagcagcccggggctccactctgggttctgaaagcccattccctgctctgcggctcctcccaccccacctcttctcagccttgcagctcaagggttgatctcaggagtccaggacccaggagagggaagaatctgaggaacacagaacagtgagcgttgcccacaccccatctcccgtcaccacatctcccctcaccctcaccctccctgcctggccctggaccccatcccaggacctccctatcagctgacttcttccagtgtcttgcaggcccctctgggctcctccctcccctggcttttcctaccactccccctctatcggcgtctatctgtaggtgccctgggatttataaaactgggttccgaatgctgaataagagacggtaagagccaaggcaaaggacagcactgttctctgcctgcctgataccctcaccacctgggaacatcccccagacaccctcttaactccgggacagagatggctggcggagcctggggccgcctggcctgttacttggagttcctgaagaaggaggagctgaaggagttccagcttctgctcgccaataaagcgcactccaggagctcttcgggtgagacacccgctcagccagagaagacgagtggcatggaggtggcctcgtacctggtggctcagtatggggagcagcgggcctgggacctagccctccatacctgggagcagatggggctgaggtcactgtgcgcccaagcccaggaaggggcaggccactctccctcattcccctacagcccaagtgaaccccacctggggtctcccagccaacccacctccaccgcagtgctaatgccctggatccatgaattgccggcggggtgcacccagggctcagagagaagggttttgagacagctgcctgacacatctggacgccgctggagagaaatctctgcctcactcctctaccaagctcttccaagctccccagaccatgagtctccaagccaggagtcacccaacgcccccacatccacagcagtgctggggagctggggatccccacctcagcccagcctagcacccagagagcaggaggctcctgggacccaatggcctctggatgaaacgtcaggaatttactacacagaaatcagagaaagagagagagagaaatcagagaaaggcaggcccccatgggcagcggtggtaggaacgcccccacaggcgcacaccagcctacagccccaccaccacccatgggagccttctgtgagagagagcctctgttccacatggccctggaaaaatgaggattttaaccaaaaattcacacagctgctacttctacaaagacctcaccccagaagccaagatcccctggtcaagagaagctggcctgattatgtggaggagaatcgaggacatttaattgagatcagagacttatttggcccaggcctggatacccaagaacctcgcatagtcatactgcagggggctgctggaattgggaagtcaacactggccaggcaggtgaaggaagcctgggggagaggccagctgtatggggaccgcttccagcatgtcttctacttcagctgcagagagctggcccagtccaaggtggtgagtctcgctgagctcatcggaaaagatgggacagccactccggctcccattagacagatcctgtctaggccagagcggctgctcttcatcctcgatggtgtagatgagccaggatgggtcttgcaggagccgagttctgagctctgtctgcactggagccagccacagccggcggatgcactgctgggcagtttgctggggaaaactatacttcccgaggcatccttcctgatcacggctcggaccacagctctgcagaacctcattccttctttggagcaggcacgttgggtagaggtcctggggttctctgagtccagcaggaaggaatatttctacagatatttcacagatgaaaggcaagcaattagagcctttaggttggtcaaatcaaacaaagagctctgggccctgtgtcttgtgccctgggtgtcctggctggcctgcacttgcctgatgcagcagatgaagcggaaggaaaaactcacactgacttccaagaccaccacaaccctctgtctacattaccttgcccaggctctccaagctcagccattgggaccccagctcagagacctctgctctctggctgctgagggcatctggcaaaaaaagacccttttcagtccagatgacctcaggaagcatgggttagatggggccatcatctccaccttcttgaagatgggtattcttcaagagcaccccatccctctgagctacagcttcattcacctctgtttccaagagttctttgcagcaatgtcctatgtcttggaggatgagaaggggagaggtaaacattctaattgcatcatagatttggaaaagacgctagaagcatatggaatacatggcctgtttggggcatcaaccacacgtttcctattgggcctgttaagtgatgagggggagagagagatggagaacatctttcactgccggctgtctcaggggaggaacctgatgcagtgggtcccgtccctgcagctgctgctgcagccacactctctggagtccctccactgcttgtacgagactcggaacaaaacgttcctgacacaagtgatggcccatttcgaagaaatgggcatgtgtgtagaaacagacatggagctcttagtgtgcactttctgcattaaattcagccgccacgtgaagaagcttcagctgattgagggcaggcagcacagatcaacatggagccccaccatggtagtcctgttcaggtgggtcccagtcacagatgcctattggcagattctcttctccgtcctcaaggtcaccagaaacctgaaggagctggacctaagtggaaactcgctgagccactctgcagtgaagagtctttgtaagaccctgagacgccctcgctgcctcctggagaccctgcggttggctggctgtggcctcacagctgaggactgcaaggaccttgcctttgggctgagagccaaccagaccctgaccgagctggacctgagcttcaatgtgctcacggatgctggagccaaacacctttgccagagactgagacagccgagctgcaagctacagcgactgcagctggtcagctgtggcctcacgtctgactgctgccaggacctggcctctgtgcttagtgccagccccagcctgaaggagctagacctgcagcagaacaacctggatgacgttggcgtgcgactgctctgtgaggggctcaggcatcctgcctgcaaactcatacgcctggggctggaccagacaactctgagtgatgagatgaggcaggaactgagggccctggagcaggagaaacctcagctgctcatcttcagcagacggaaaccaagtgtgatgacccctactgagggcctggatacgggagagatgagtaatagcacatcctcactcaagcggcagagactcggatcagagagggcggcttcccatgttgctcaggctaatctcaaactcctggacgtgagcaagatcttcccaattgctgagattgcagaggaaagctccccagaggtagtaccggtggaactcttgtgcgtgccttctcctgcctctcaaggggacctgcatacgaagcctttggggactgacgatgacttctggggccccacggggcctgtggctactgaggtagttgacaaagaaaagaacttgtaccgagttcacttccctgtagctggctcctaccgctggcccaacacgggtctctgctttgtgatgagagaagcggtgaccgttgagattgaattctgtgtgtgggaccagttcctgggtgagatcaacccacagcacagctggatggtggcagggcctctgctggacatcaaggctgagcctggagctgtggaagctgtgcacctccctcactttgtggctctccaagggggccatgtggacacatccctgttccaaatggcccactttaaagaggaggggatgctcctggagaagccagccagggtggagctgcatcacatagttctggaaaaccccagcttctcccccttgggagtcctcctgaaaatgatccataatgccctgcgcttcattcccgtcacctctgtggtgttgctttaccaccgcgtccatcctgaggaagtcaccttccacctctacctgatcccaagtgactgctccattcggaaggccatagatgatctagaaatgaaattccagtttgtgcgaatccacaagccacccccgctgaccccactttatatgggctgtcgttacactgtgtctgggtctggttcagggatgctggaaatactccccaaggaactggagctctgctatcgaagccctggagaagaccagctgttctcggagttctacgttggccacttgggatcagggatcaggctgcaagtgaaagacaagaaagatgagactctggtgtgggaggccttggtgaaaccaggagatctcatgcctgcaactactctgatccctccagcccgcatagccgtaccttcacctctggatgccccgcagttgctgcactttgtggaccagtatcgagagcagctgatagcccgagtgacatcggtggaggttgtcttggacaaactgcatggacaggtgctgagccaggagcagtacgagagggtgctggctgagaacacgaggcccagccagatgcggaagctgttcagcttgagccagtcctgggaccggaagtgcaaagatggactctaccaagccctgaaggagacccatcctcacctcattatggaactctgggagaagggcagcaaaaagggactcctgccactcagcagctgaagtatcaacaccagcccttgacccttgagtcctggctttggctgacccttctttgggtctcagtttctttctctgcaaacaagttgccatctggtttgccttccagcactaaagtaatggaactttgatgatgcctttgctgggcattatgtgtccatgccagggatgccacagggggccccagtccaggtggcctaacagcatctcagggaatgtccatctggagctggcaagacccctgcagacctcatagagcctcatctggtggccacagcagccaagcctagagccctccggatcccatccaggcgcaaagaggaataggagggacatggaaccatttgcctctggctgtgtcacagggtgagccccaaaattggggttcagcgtgggaggccacgtggattcttggctttgtacaggaagatctacaagagcaagccaacagagtaaagtggaaggaagtttattcagaaaataaaggagtatcacagctcttttagaatttgtctagcaggctttccagtttttaccagaaaacccctataaattaaaaattttttacttaaatttaagaattaaaaaaatacaaaaaagaaaaaatgaaaataaaggaataagaagttacctactcca

SEQ ID NO: 9 - DNA, 5610 bp, synthetic construct; identical to SEQ ID NO: 8
  source 1..5610: mol_type = other DNA

SEQ ID NO: 10 - DNA, 5478 bp, synthetic construct
  source 1..5478: mol_type = other DNA
gttgactaggcgctgttcttgctggctggtgccccagggcctggagaggtctgaagaaacctgggagccagcagcccggggctccactctgggttctgaaagcccattccctgctctgcggctcctcccaccccacctcttctcagccttgcagctcaagggttgatctcaggagtccaggacccaggagagggaagaatctgaggaacacagaacagtgagcgttgcccacaccccatctcccgtcaccacatctcccctcaccctcaccctccctgcctggccctggaccccatcccaggacctccctatcagctgacttcttccagtgtcttgcaggcccctctgggctcctccctcccctggcttttcctaccactccccctctatcggcgtctatctgtaggtgccctgggatttataaaactgggttccgaatgctgaataagagacggtaagagccaaggcaaaggacagcactgttctctgcctgcctgataccctcaccacctgggaacatcccccagacaccctcttaactccgggacagagatggctggcggagcctggggccgcctggcctgttacttggagttcctgaagaaggaggagctgaaggagttccagcttctgctcgccaataaagcgcactccaggagctcttcgggtgagacacccgctcagccagagaagacgagtggcatggaggtggcctcgtacctggtggctcagtatggggagcagcgggcctgggacctagccctccatacctgggagcagatggggctgaggtcactgtgcgcccaagcccaggaaggggcaggccactctccctcattcccctacagcccaagtgaaccccacctggggtctcccagccaacccacctccaccgcagtgctaatgccctggatccatgaattgccggcggggtgcacccagggctcagagagaagggttttgagacagctgcctgacacatctggacgccgctggagagaaatctctgcctcactcctctaccaagctcttccaagctccccagaccatgagtctccaagccaggagtcacccaacgcccccacatccacagcagtgctggggagctggggatccccacctcagcccagcctagcacccagagagcaggaggctcctgggacccaatggcctctggatgaaacgtcaggaatttactacacagaaatcagagaaagagagagagagaaatcagagaaaggcaggcccccatgggcagcggtggtaggaacgcccccacaggcgcacaccagcctacagccccaccaccacccatgggagccttctgtgagagagagcctctgttccacatggccctggaaaaatgaggattttaaccaaaaattcacacagctgctacttctacaaagacctcaccccagaagccaagatcccctggtcaagagaagctggcctgattatgtggaggagaatcgaggacatttaattgagatcagagacttatttggcccaggcctggatacccaagaacctcgcatagtcatactgcagggggctgctggaattgggaagtcaacactggccaggcaggtgaaggaagcctgggggagaggccagctgtatggggaccgcttccagcatgtcttctacttcagctgcagagagctggcccagtccaaggtggtgagtctcgctgagctcatcggaaaagatgggacagccactccggctcccattagacagatcctgtctaggccagagcggctgctcttcatcctcgatggtgtagatgagccaggatgggtcttgcaggagccgagttctgagctctgtctgcactggagccagccacagccggcggatgcactgctgggcagtttgctggggaaaactatacttcccgaggcatccttcctgatcacggctcggaccacagctctgcagaacctcattccttctttggagcaggcacgttgggtagaggtcctggggttctctgagtccagcaggaaggaatatttctacagatatttcacagatgaaaggcaagcaattagagcctttaggttggtcaaatcaaacaaagagctctgggccctgtgtcttgtgccctgggtgtcctggctggcctgcacttgcctgatgcagcagatgaagcggaaggaaaaactcacactgacttccaagaccaccacaaccctctgtctacattaccttgcccaggctctccaagctcagccattgggaccccagctcagagacctctgctctctggctgctgagggcatctggcaaaaaaagacccttttcagtccagatgacctcaggaagcatgggttagatggggccatcatctccaccttcttgaagatgggtattcttcaagagcaccccatccctctgagctacagcttcattcacctctgtttccaagagttctttgcagcaatgtcctatgtcttggaggatgagaaggggagaggtaaacattctaattgcatcatagatttggaaaagacgctagaagcatatggaatacatggcctgtttggggcatcaaccacacgtttcctattgggcctgttaagtgatgagggggagagagagatggagaacatctttcactgccggctgtctcaggggaggaacctgatgcagtgggtcccgtccctgcagctgctgctgcagccacactctctggagtccctccactgcttgtacgagactcggaacaaaacgttcctgacacaagtgatggcccatttcgaagaaatgggcatgtgtgtagaaacagacatggagctcttagtgtgcactttctgcattaaattcagccgccacgtgaagaagcttcagctgattgagggcaggcagcacagatcaacatggagccccaccatggtagtcctgttcaggtgggtcccagtcacagatgcctattggcagattctcttctccgtcctcaaggtcaccagaaacctgaaggagctggacctaagtggaaactcgctgagccactctgcagtgaagagtctttgtaagaccctgagacgccctcgctgcctcctggagaccctgcggttggctggctgtggcctcacagctgaggactgcaaggaccttgcctttgggctgagagccaaccagaccctgaccgagctggacctgagcttcaatgtgctcacggatgctggagccaaacacctttgccagagactgagacagccgagctgcaagctacagcgactgcagctggtcagctgtggcctcacgtctgactgctgccaggacctggcctctgtgcttagtgccagccccagcctgaaggagctagacctgcagcagaacaacctggatgacgttggcgtgcgactgctctgtgaggggctcaggcatcctgcctgcaaactcatacgcctggggctggaccagacaactctgagtgatgagatgaggcaggaactgagggccctggagcaggagaaacctcagctgctcatcttcagcagacggaaaccaagtgtgatgacccctactgagggcctggatacgggagagatgagtaatagcacatcctcactcaagcggcagagactcggatcagagagggcggcttcccatgttgctcaggctaatctcaaactcctggacgtgagcaagatcttcccaattgctgagattgcagaggaaagctccccagaggtagtaccggtggaactcttgtgcgtgccttctcctgcctctcaaggggacctgcatacgaagcctttggggactgacgatgacttctggggccccacggggcctgtggctactgaggtagttgacaaagaaaagaacttgtaccgagttcacttccctgtagctggctcctaccgctggcccaacacgggtctctgctttgtgatgagagaagcggtgaccgttgagattgaattctgtgtgtgggaccagttcctgggtgagatcaacccacagcacagctggatggtggcagggcctctgctggacatcaaggctgagcctggagctgtggaagctgtgcacctccctcactttgtggctctccaagggggccatgtggacacatccctgttccaaatggcccactttaaagaggaggggatgctcctggagaagccagccagggtggagctgcatcacatagttctggaaaaccccagcttctcccccttgggagtcctcctgaaaatgatccataatgccctgcgcttcattcccgtcacctctgtggtgttgctttaccaccgcgtccatcctgaggaagtcaccttccacctctacctgatcccaagtgactgctccattcggaaggaactggagctctgctatcgaagccctggagaagaccagctgttctcggagttctacgttggccacttgggatcagggatcaggctgcaagtgaaagacaagaaagatgagactctggtgtgggaggccttggtgaaaccaggagatctcatgcctgcaactactctgatccctccagcccgcatagccgtaccttcacctctggatgccccgcagttgctgcactttgtggaccagtatcgagagcagctgatagcccgagtgacatcggtggaggttgtcttggacaaactgcatggacaggtgctgagccaggagcagtacgagagggtgctggctgagaacacgaggcccagccagatgcggaagctgttcagcttgagccagtcctgggaccggaagtgcaaagatggactctaccaagccctgaaggagacccatcctcacctcattatggaactctgggagaagggcagcaaaaagggactcctgccactcagcagctgaagtatcaacaccagcccttgacccttgagtcctggctttggctgacccttctttgggtctcagtttctttctctgcaaacaagttgccatctggtttgccttccagcactaaagtaatggaactttgatgatgcctttgctgggcattatgtgtccatgccagggatgccacagggggccccagtccaggtggcctaacagcatctcagggaatgtccatctggagctggcaagacccctgcagacctcatagagcctcatctggtggccacagcagccaagcctagagccctccggatcccatccaggcgcaaagaggaataggagggacatggaaccatttgcctctggctgtgtcacagggtgagccccaaaattggggttcagcgtgggaggccacgtggattcttggctttgtacaggaagatctacaagagcaagccaacagagtaaagtggaaggaagtttattcagaaaataaaggagtatcacagctcttttagaatttgtctagcaggctttccagtttttaccagaaaacccctataaattaaaaattttttacttaaatttaagaattaaaaaaatacaaaaaagaaaaaatgaaaataaaggaataagaagttacctactcca

SEQ ID NO: 11 - DNA, 2650 bp, synthetic construct
  source 1..2650: mol_type = other DNA
gcagcacagatcaacatggagccccaccatggtagtcctgttcaggtgggtcccagtcacagatgcctattggcagattctcttctccgtcctcaaggtcaccagaaacctgaaggagctggacctaagtggaaactcgctgagccactctgcagtgaagagtctttgtaagaccctgagacgccctcgctgcctcctggagaccctgcggttggctggctgtggcctcacagctgaggactgcaaggaccttgcctttgggctgagagccaaccagaccctgaccgagctggacctgagcttcaatgtgctcacggatgctggagccaaacacctttgccagagactgagacagccgagctgcaagctacagcgactgcagctggtcagctgtggcctcacgtctgactgctgccaggacctggcctctgtgcttagtgccagccccagcctgaaggagctagacctgcagcagaacaacctggatgacgttggcgtgcgactgctctgtgaggggctcaggcatcctgcctgcaaactcatacgcctggggaaaccaagtgtgatgacccctactgagggcctggatacgggagagatgagtaatagcacatcctcactcaagcggcagagactcggatcagagagggcggcttcccatgttgctcaggctaatctcaaactcctggacgtgagcaagatcttcccaattgctgagattgcagaggaaagctccccagaggtagtaccggtggaactcttgtgcgtgccttctcctgcctctcaaggggacctgcatacgaagcctttggggactgacgatgacttctggggccccacggggcctgtggctactgaggtagttgacaaagaaaagaacttgtaccgagttcacttccctgtagctggctcctaccgctggcccaacacgggtctctgctttgtgatgagagaagcggtgaccgttgagattgaattctgtgtgtgggaccagttcctgggtgagatcaacccacagcacagctggatggtggcagggcctctgctggacatcaaggctgagcctggagctgtggaagctgtgcacctccctcactttgtggctctccaagggggccatgtggacacatccctgttccaaatggcccactttaaagaggaggggatgctcctggagaagccagccagggtggagctgcatcacatagttctggaaaaccccagcttctcccccttgggagtcctcctgaaaatgatccataatgccctgcgcttcattcccgtcacctctgtggtgttgctttaccaccgcgtccatcctgaggaagtcaccttccacctctacctgatcccaagtgactgctccattcggaaggccatagatgatctagaaatgaaattccagtttgtgcgaatccacaagccacccccgctgaccccactttatatgggctgtcgttacactgtgtctgggtctggttcagggatgctggaaatactccccaaggaactggagctctgctatcgaagccctggagaagaccagctgttctcggagttctacgttggccacttgggatcagggatcaggctgcaagtgaaagacaagaaagatgagactctggtgtgggaggccttggtgaaaccaggagatctcatgcctgcaactactctgatccctccagcccgcatagccgtaccttcacctctggatgccccgcagttgctgcactttgtggaccagtatcgagagcagctgatagcccgagtgacatcggtggaggttgtcttggacaaactgcatggacaggtgctgagccaggagcagtacgagagggtgctggctgagaacacgaggcccagccagatgcggaagctgttcagcttgagccagtcctgggaccggaagtgcaaagatggactctaccaagccctgaaggagacccatcctcacctcattatggaactctgggagaagggcagcaaaaagggactcctgccactcagcagctgaagtatcaacaccagcccttgacccttgagtcctggctttggctgacccttctttgggtctcagtttctttctctgcaaacaagttgccatctggtttgccttccagcactaaagtaatggaactttgatgatgcctttgctgggcattatgtgtccatgccagggatgccacagggggccccagtccaggtggcctaacagcatctcagggaatgtccatctggagctggcaagacccctgcagacctcatagagcctcatctggtggccacagcagccaagcctagagccctccggatcccatccaggcgcaaagaggaataggagggacatggaaccatttgcctctggctgtgtcacagggtgagccccaaaattggggttcagcgtgggaggccacgtggattcttggctttgtacaggaagatctacaagagcaagccaacagagtaaagtggaaggaagtttattcagaaaataaaggagtatcacagctcttttagaatttgtctagcaggctttccagtttttaccagaaaacccctataaattaaaaattttttacttaaatttaagaattaaaaaaatacaaaaaagaaaaaatgaaaataaaggaataagaagttacctactcca

SEQ ID NO: 12 - DNA, 5388 bp, synthetic construct
  source 1..5388: mol_type = other DNA
gttgactaggcgctgttcttgctggctggtgccccagggcctggagaggtctgaagaaacctgggagccagcagcccggggctccactctgggttctgaaagcccattccctgctctgcggctcctcccaccccacctcttctcagccttgcagctcaagggttgatctcaggagtccaggacccaggagagggaagaatctgaggaacacagaacagtgagcgttgcccacaccccatctcccgtcaccacatctcccctcaccctcaccctccctgcctggccctggaccccatcccaggacctccctatcagctgacttcttccagtgtcttgcaggcccctctgggctcctccctcccctggcttttcctaccactccccctctatcggcgtctatctgtaggtgccctgggatttataaaactgggttccgaatgctgaataagagacggtaagagccaaggcaaaggacagcactgttctctgcctgcctgataccctcaccacctgggaacatcccccagacaccctcttaactccgggacagagatggctggcggagcctggggccgcctggcctgttacttggagttcctgaagaaggaggagctgaaggagttccagcttctgctcgccaataaagcgcactccaggagctcttcgggtgagacacccgctcagccagagaagacgagtggcatggaggtggcctcgtacctggtggctcagtatggggagcagcgggcctgggacctagccctccatacctgggagcagatggggctgaggtcactgtgcgcccaagcccaggaaggggcaggccactctccctcattcccctacagcccaagtgaaccccacctggggtctcccagccaacccacctccaccgcagtgctaatgccctggatccatgaattgccggcggggtgcacccagggctcagagagaagggttttgagacagctgcctgacacatctggacgccgctggagagaaatctctgcctcactcctctaccaagctcttccaagctccccagaccatgagtctccaagccaggagtcacccaacgcccccacatccacagcagtgctggggagctggggatccccacctcagcccagcctagcacccagagagcaggaggctcctgggacccaatggcctctggatgaaacgtcaggaatttactacacagaaatcagagaaagagagagagagaaatcagagaaaggcaggcccccatgggcagcggtggtaggaacgcccccacaggcgcacaccagcctacagccccaccaccacccatgggagccttctgtgagagagagcctctgttccacatggccctggaaaaatgaggattttaaccaaaaattcacacagctgctacttctacaaagacctcaccccagaagccaagatcccctggtcaagagaagctggcctgattatgtggaggagaatcgaggacatttaattgagatcagagacttatttggcccaggcctggatacccaagaacctcgcatagtcatactgcagggggctgctggaattgggaagtcaacactggccaggcaggtgaaggaagcctgggggagaggccagctgtatggggaccgcttccagcatgtcttctacttcagctgcagagagctggcccagtccaaggtggtgagtctcgctgagctcatcggaaaagatgggacagccactccggctcccattagacagatcctgtctaggccagagcggctgctcttcatcctcgatggtgtagatgagccaggatgggtcttgcaggagccgagttctgagctctgtctgcactggagccagccacagccggcggatgcactgctgggcagtttgctggggaaaactatacttcccgaggcatccttcctgatcacggctcggaccacagctctgcagaacctcattccttctttggagcaggcacgttgggtagaggtcctggggttctctgagtccagcaggaaggaatatttctacagatatttcacagatgaaaggcaagcaattagagcctttaggttggtcaaatcaaacaaagagctctgggccctgtgtcttgtgccctgggtgtcctggctggcctgcacttgcctgatgcagcagatgaagcggaaggaaaaactcacactgacttccaagaccaccacaaccctctgtctacattaccttgcccaggctctccaagctcagccattgggaccccagctcagagacctctgctctctggctgctgagggcatctggcaaaaaaagacccttttcagtccagatgacctcaggaagcatgggttagatggggccatcatctccaccttcttgaagatgggtattcttcaagagcaccccatccctctgagctacagcttcattcacctctgtttccaagagttctttgcagcaatgtcctatgtcttggaggatgagaaggggagaggtaaacattctaattgcatcatagatttggaaaagacgctagaagcatatggaatacatggcctgtttggggcatcaaccacacgtttcctattgggcctgttaagtgatgagggggagagagagatggagaacatctttcactgccggctgtctcaggggaggaacctgatgcagtgggtcccgtccctgcagctgctgctgcagccacactctctggagtccctccactgcttgtacgagactcggaacaaaacgttcctgacacaagtgatggcccatttcgaagaaatgggcatgtgtgtagaaacagacatggagctcttagtgtgcactttctgcattaaattcagccgccacgtgaagaagcttcagctgattgagggcaggcagcacagatcaacatggagccccaccatggtagtcctgttcaggtgggtcccagtcacagatgcctattggcagattctcttctccgtcctcaaggtcaccagaaacctgaaggagctggacctaagtggaaactcgctgagccactctgcagtgaagagtctttgtaagaccctgagacgccctcgctgcctcctggagaccctgcggttggctggctgtggcctcacagctgaggactgcaaggaccttgcctttgggctgagagccaaccagaccctgaccgagctggacctgagcttcaatgtgctcacggatgctggagccaaacacctttgccagagactgagacagccgagctgcaagctacagcgactgcagctggtcagctgtggcctcacgtctgactgctgccaggacctggcctctgtgcttagtgccagccccagcctgaaggagctagacctgcagcagaacaacctggatgacgttggcgtgcgactgctctgtgaggggctcaggcatcctgcctgcaaactcatacgcctggggaaaccaagtgtgatgacccctactgagggcctggatacgggagagatgagtaatagcacatcctcactcaagcggcagagactcggatcagagagggcggcttcccatgttgctcaggctaatctcaaactcctggacgtgagcaagatcttcccaattgctgagattgcagaggaaagctccccagaggtagtaccggtggaactcttgtgcgtgccttctcctgcctctcaaggggacctgcatacgaagcctttggggactgacgatgacttctggggccccacggggcctgtggctactgaggtagttgacaaagaaaagaacttgtaccgagttcacttccctgtagctggctcctaccgctggcccaacacgggtctctgctttgtgatgagagaagcggtgaccgttgagattgaattctgtgtgtgggaccagttcctgggtgagatcaacccacagcacagctggatggtggcagggcctctgctggacatcaaggctgagcctggagctgtggaagctgtgcacctccctcactttgtggctctccaagggggccatgtggacacatccctgttccaaatggcccactttaaagaggaggggatgctcctggagaagccagccagggtggagctgcatcacatagttctggaaaaccccagcttctcccccttgggagtcctcctgaaaatgatccataatgccctgcgcttcattcccgtcacctctgtggtgttgctttaccaccgcgtccatcctgaggaagtcaccttccacctctacctgatcccaagtgactgctccattcggaaggaactggagctctgctatcgaagccctggagaagaccagctgttctcggagttctacgttggccacttgggatcagggatcaggctgcaagtgaaagacaagaaagatgagactctggtgtgggaggccttggtgaaaccaggagatctcatgcctgcaactactctgatccctccagcccgcatagccgtaccttcacctctggatgccccgcagttgctgcactttgtggaccagtatcgagagcagctgatagcccgagtgacatcggtggaggttgtcttggacaaactgcatggacaggtgctgagccaggagcagtacgagagggtgctggctgagaacacgaggcccagccagatgcggaagctgttcagcttgagccagtcctgggaccggaagtgcaaagatggactctaccaagccctgaaggagacccatcctcacctcattatggaactctgggagaagggcagcaaaaagggactcctgccactcagcagctgaagtatcaacaccagcccttgacccttgagtcctggctttggctgacccttctttgggtctcagtttctttctctgcaaacaagttgccatctggtttgccttccagcactaaagtaatggaactttgatgatgcctttgctgggcattatgtgtccatgccagggatgccacagggggccccagtccaggtggcctaacagcatctcagggaatgtccatctggagctggcaagacccctgcagacctcatagagcctcatctggtggccacagcagccaagcctagagccctccggatcccatccaggcgcaaagaggaataggagggacatggaaccatttgcctctggctgtgtcacagggtgagccccaaaattggggttcagcgtgggaggccacgtggattcttggctttgtacaggaagatctacaagagcaagccaacagagtaaagtggaaggaagtttattcagaaaataaaggagtatcacagctcttttagaatttgtctagcaggctttccagtttttaccagaaaacccctataaattaaaaattttttacttaaatttaagaattaaaaaaatacaaaaaagaaaaaatgaaaataaaggaataagaagttacctactcca

SEQ ID NO: 13 - DNA, 5098 bp, synthetic construct
  source 1..5098: mol_type = other DNA
gttgactaggcgctgttcttgctggctggtgccccagggcctggagaggtctgaagaaacctgggagccagcagcccggggctccactctgggttctgaaagcccattccctgctctgcggctcctcccaccccacctcttctcagccttgcagctcaagggttgatctcaggagtccaggacccaggagagggaagaatctgaggaacacagaacagtgagcgttgcccacaccccatctcccgtcaccacatctcccctcaccctcaccctccctgcctggccctggaccccatcccaggacctccctatcagctgacttcttccagtgtcttgcaggcccctctgggctcctccctcccctggcttttcctaccactccccctctatcggcgtctatctgtaggtgccctgggatttataaaactgggttccgaatgctgaataagagacggtaagagccaaggcaaaggacagcactgttctctgcctgcctgataccctcaccacctgggaacatcccccagacaccctcttaactccgggacagagatggctggcggagcctggggccgcctggcctgttacttggagttcctgaagaaggaggagctgaaggagttccagcttctgctcgccaataaagcgcactccaggagctcttcgggtgagacacccgctcagccagagaagacgagtggcatggaggtggcctcgtacctggtggctcagtatggggagcagcgggcctgggacctagccctccatacctgggagcagatggggctgaggtcactgtgcgcccaagcccaggaaggggcaggccactctccctcattcccctacagcccaagtgaaccccacctggggtctcccagccaacccacctccaccgcagtgctaatgccctggatccatgaattgccggcggggtgcacccagggctcagagagaagggttttgagacagctgcctgacacatctggacgccgctggagagaaatctctgcctcactcctctaccaagctcttccaagctccccagaccatgagtctccaagccaggagtcacccaacgcccccacatccacagcagtgctggggagctggggatccccacctcagcccagcctagcacccagagagcaggaggctcctgggacccaatggcctctggatgaaacgtcaggaatttactacacagaaatcagagaaagagagagagagaaatcagagaaaggcaggcccccatgggcagcggtggtaggaacgcccccacaggcgcacaccagcctacagccccaccaccacccatgggagccttctgtgagagagagcctctgttccacatggccctggaaaaatgaggattttaaccaaaaattcacacagctgctacttctacaaagacctcaccccagaagccaagatcccctggtcaagagaagctggcctgattatgtggaggagaatcgaggacatttaattgagatcagagacttatttggcccaggcctggatacccaagaacctcgcatagtcatactgcagggggctgctggaattgggaagtcaacactggccaggcaggtgaaggaagcctgggggagaggccagctgtatggggaccgcttccagcatgtcttctacttcagctgcagagagctggcccagtccaaggtggtgagtctcgctgagctcatcggaaaagatgggacagccactccggctcccattagacagatcctgtctaggccagagcggctgctcttcatcctcgatggtgtagatgagccaggatgggtcttgcaggagccgagttctgagctctgtctgcactggagccagccacagccggcggatgcactgctgggcagtttgctggggaaaactatacttcccgaggcatccttcctgatcacggctcggaccacagctctgcagaacctcattccttctttggagcaggcacgttgggtagaggtcctggggttctctgagtccagcaggaaggaatatttctacagatatttcacagatgaaaggcaagcaattagagcctttaggttggtcaaatcaaacaaagagctctgggccctgtgtcttgtgccctgggtgtcctggctggcctgcacttgcctgatgcagcagatgaagcggaaggaaaaactcacactgacttccaagaccaccacaaccctctgtctacattaccttgcccaggctctccaagctcagccattgggaccccagctcagagacctctgctctctggctgctgagggcatctggcaaaaaaagacccttttcagtccagatgacctcaggaagcatgggttagatggggccatcatctccaccttcttgaagatgggtattcttcaagagcaccccatccctctgagctacagcttcattcacctctgtttccaagagttctttgcagcaatgtcctatgtcttggaggatgagaaggggagaggtaaacattctaattgcatcatagatttggaaaagacgctagaagcatatggaatacatggcctgtttggggcatcaaccacacgtttcctattgggcctgttaagtgatgagggggagagagagatggagaacatctttcactgccggctgtctcaggggaggaacctgatgcagtgggtcccgtccctgcagctgctgctgcagccacactctctggagtccctccactgcttgtacgagactcggaacaaaacgttcctgacacaagtgatggcccatttcgaagaaatgggcatgtgtgtagaaacagacatggagctcttagtgtgcactttctgcattaaattcagccgccacgtgaagaagcttcagctgattgagggcaggcagcacagatcaacatggagccccaccatggtagtcctgttcaggtgggtcccagtcacagatgcctattggcagattctcttctccgtcctcaaggtcaccagaaacctgaaggagctggacctaagtggaaactcgctgagccactctgcagtgaagagtctttgtaagaccctgagacgccctcgctgcctcctggagaccctgcggttggctggctgtggcctcacagctgaggactgcaaggaccttgcctttgggctgagagccaaccagaccctgaccgagctggacctgagcttcaatgtgctcacggatgctggagccaaacacctttgccagagactgagacagccgagctgcaagctacagcgactgcagctggtcagctgtggcctcacgtctgactgctgccaggacctggcctctgtgcttagtgccagccccagcctgaaggagctagacctgcagcagaacaacctggatgacgttggcgtgcgactgctctgtgaggggctcaggcatcctgcctgcaaactcatacgcctggggctggaccagacaactctgagtgatgagatgaggcaggaactgagggccctggagcaggagaaacctcagctgctcatcttcagcagacggaaaccaagtgtgatgacccctactgagggcctggatacgggagagatgagtaatagcacatcctcactcaagcggcagagactcggatcagagagggcggcttcccatgttgctcaggctaatctcaaactcctggacgtgagcaagatcttcccaattgctgagattgcaggcaagagccacgaggaaagctccccagaggtagtaccggtggaactcttgtgcgtgccttctcctgcctctcaaggggacctgcatacgaagcctttggggactgacgatgacttctggggccccacggggcctgtggctactgaggtagttgacaaagaaaagaacttgtaccgagttcacttccctgtagctggctcctaccgctggcccaacacgggtctctgctttgtgatgagagaagcggtgaccgttgagattgaattctgtgtgtgggaccagttcctgggtgagatcaacccacagcacagctggatggtggcagggcctctgctggacatcaaggctgagcctggagctgtggaagctgtgcacctccctcactttgtggctctccaagggggccatgtggacacatccctgttccaaatggcccactttaaagaggaggggatgctcctggagaagccagccagggtggagctgcatcacatagttctggaaaaccccagcttctcccccttgggagtcctcctgaaaatgatccataatgccctgcgcttcattcccgtcacctctgtggtgttgctttaccaccgcgtccatcctgaggaagtcaccttccacctctacctgatcccaagtgactgctccattcggaaggccatagatgatctagaaatgaaattccagtttgtgcgaatccacaagccacccccgctgaccccactttatatgggctgtcgttacactgtgtctgggtctggttcagggatgctggaaatactccccaaggaactggagctctgctatcgaagccctggagaagaccagctgttctcggagttctacgttggccacttgggatcagggatcaggctgcaagtgaaagacaagaaagatgagactctggtgtgggaggccttggtgaaaccaggaaggaacaccagccagccgtggaacctcaggtgcaacagagacgccaggagatactagtgcccagcagcctgcggcagtaccaatgaagccagagagggcttggtggatgacaaggaggcctgagtagaccgcaggtgggtctgagaaatgggcttaggtgaggcaggtctttgaaggatttgttcttaatcatatgcgagatgctcaaaaggctggatgcctgcttttgtgggtgaagagcaagaagagaaaacaggttgtacacatacagatgcagatggagagacagagaaaaaaaaggaagaaggcagagaaatgcaccaattcttgagctgtattatctctggaccttgggattgtgggaggctttattttactactgattttgcctacactgttttctcaatttctagttttctacaaagatgatgtgttagctttttcacgcattaagattaaaatttaaaacagacca

SEQ ID NO: 14-20 - skipped (7 entries)

SEQ ID NO: 21 - DNA, 14 bp, synthetic construct
  source 1..14: mol_type = other DNA
gtcccacacacaga

SEQ ID NO: 22 - DNA, 14 bp, synthetic construct
  source 1..14: mol_type = other DNA
gtggtggtcttgga

SEQ ID NO: 23 - DNA, 14 bp, synthetic construct
  source 1..14: mol_type = other DNA
tcccacacacagaa

SEQ ID NO: 24 - DNA, 14 bp, synthetic construct
  source 1..14: mol_type = other DNA
tggattcttggctt

SEQ ID NO: 25 - DNA, 14 bp, synthetic construct
  source 1..14: mol_type = other DNA
gtggattcttggct

SEQ ID NO: 26 - DNA, 14 bp, synthetic construct
  source 1..14: mol_type = other DNA
ggattcttggcttt

SEQ ID NO: 27 - RNA, 14 nt, synthetic construct
  source 1..14: mol_type = other RNA
gtcccacacacaga

SEQ ID NO: 28 - RNA, 14 nt, synthetic construct
  source 1..14: mol_type = other RNA
gtggtggtcttgga

SEQ ID NO: 29 - RNA, 14 nt, synthetic construct
  source 1..14: mol_type = other RNA
tcccacacacagaa

SEQ ID NO: 30 - RNA, 14 nt, synthetic construct
  source 1..14: mol_type = other RNA
tggattcttggctt

SEQ ID NO: 31 - RNA, 14 nt, synthetic construct
  source 1..14: mol_type = other RNA
gtggattcttggct

SEQ ID NO: 32 - RNA, 14 nt, synthetic construct
  source 1..14: mol_type = other RNA
ggattcttggcttt

SEQ ID NO: 33 - DNA, 14 bp, synthetic construct
  source 1..14: mol_type = other DNA
gtcccacacacaga

SEQ ID NO: 34 - DNA, 14 bp, synthetic construct
  source 1..14: mol_type = other DNA
gtggtggtcttgga

SEQ ID NO: 35 - DNA, 14 bp, synthetic construct
  source 1..14: mol_type = other DNA
tcccacacacagaa

SEQ ID NO: 36 - DNA, 14 bp, synthetic construct
  source 1..14: mol_type = other DNA
tggattcttggctt

SEQ ID NO: 37 - DNA, 14 bp, synthetic construct
  source 1..14: mol_type = other DNA
gtggattcttggct

SEQ ID NO: 38 - DNA, 14 bp, synthetic construct
  source 1..14: mol_type = other DNA
ggattcttggcttt

SEQ ID NO: 39-94 - skipped (56 entries)

SEQ ID NO: 95 - DNA, 14 bp, synthetic construct
  source 1..14: mol_type = other DNA
  modified_base 1: mod_base = OTHER; note = Locked Nucleic Acid
  modified_base 2: mod_base = OTHER; note = Locked Nucleic Acid
  modified_base 2^3: mod_base = OTHER; note = phosphorothioate linkage
  modified_base 3: mod_base = OTHER; note = Locked Nucleic Acid
  modified_base 3^4: mod_base = OTHER; note = phosphorothioate linkage
  modified_base 4^5: mod_base = OTHER; note = Locked Nucleic Acid
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12: mod_base = OTHER; note = Locked Nucleic Acid
  modified_base 12^13: mod_base = OTHER; note = phosphorothioate linkage
  modified_base 13: mod_base = OTHER; note = Locked Nucleic Acid
  modified_base 13^14: mod_base = OTHER; note = phosphorothioate linkage
  modified_base 14: mod_base = OTHER; note = Locked Nucleic Acid
gtcccacacacaga

SEQ ID NO: 96 - DNA, 14 bp, synthetic construct
  source 1..14: mol_type = other DNA
  modified_base 1: mod_base = OTHER; note = Locked Nucleic Acid
  modified_base 2: mod_base = OTHER; note = Locked Nucleic Acid
  modified_base 3: mod_base = OTHER; note = Locked Nucleic Acid
  modified_base 12: mod_base = OTHER; note = Locked Nucleic Acid
  modified_base 13: mod_base = OTHER; note = Locked Nucleic Acid
  modified_base 14: mod_base = OTHER; note = Locked Nucleic Acid
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
gtggtggtcttgga

SEQ ID NO: 97 - DNA, 14 bp, synthetic construct
  source 1..14: mol_type = other DNA
  modified_base 1: mod_base = OTHER; note = Locked Nucleic Acid
  modified_base 1^2: mod_base = OTHER; note = phosphorothioate linkage
  modified_base 2: mod_base = OTHER; note = Locked Nucleic Acid
  modified_base 2^3: mod_base = OTHER; note = phosphorothioate linkage
  modified_base 3: mod_base = OTHER; note = Locked Nucleic Acid
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12: mod_base = OTHER; note = Locked Nucleic Acid
  modified_base 12^13: mod_base = OTHER; note = phosphorothioate linkage
  modified_base 13: mod_base = OTHER; note = Locked Nucleic Acid
  modified_base 13^14: mod_base = OTHER; note = phosphorothioate linkage
  modified_base 14: mod_base = OTHER; note = Locked Nucleic Acid
tcccacacacagaa

SEQ ID NO: 98 - DNA, 14 bp, synthetic construct
  source 1..14: mol_type = other DNA
  modified_base 1: mod_base = OTHER; note = Locked Nucleic Acid
  modified_base 1^2: mod_base = OTHER; note = phosphorothioate linkage
  modified_base 2: mod_base = OTHER; note = Locked Nucleic Acid
  modified_base 2^3: mod_base = OTHER; note = phosphorothioate linkage
  modified_base 3: mod_base = OTHER; note = Locked Nucleic Acid
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12: mod_base = OTHER; note = Locked Nucleic Acid
  modified_base 12^13: mod_base = OTHER; note = phosphorothioate linkage
  modified_base 13: mod_base = OTHER; note = Locked Nucleic Acid
  modified_base 13^14: mod_base = OTHER; note = phosphorothioate linkage
  modified_base 14: mod_base = OTHER; note = Locked Nucleic Acid
tggattcttggctt

SEQ ID NO: 99 - DNA, 14 bp, synthetic construct
  source 1..14: mol_type = other DNA
  modified_base 1: mod_base = OTHER; note = Locked Nucleic Acid
  modified_base 1^2: mod_base = OTHER; note = phosphorothioate linkage
  modified_base 2: mod_base = OTHER; note = Locked Nucleic Acid
  modified_base 2^3: mod_base = OTHER; note = phosphorothioate linkage
  modified_base 3: mod_base = OTHER; note = Locked Nucleic Acid
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12: mod_base = OTHER; note = Locked Nucleic Acid
  modified_base 12^13: mod_base = OTHER; note = phosphorothioate linkage
  modified_base 13: mod_base = OTHER; note = Locked Nucleic Acid
  modified_base 13^14: mod_base = OTHER; note = phosphorothioate linkage
  modified_base 14: mod_base = OTHER; note = Locked Nucleic Acid
gtggattcttggct

SEQ ID NO: 100 - DNA, 14 bp, synthetic construct
  source 1..14: mol_type = other DNA
  modified_base 1: mod_base = OTHER; note = Locked Nucleic Acid
  modified_base 1^2: mod_base = OTHER; note = phosphorothioate linkage
  modified_base 2: mod_base = OTHER; note = Locked Nucleic Acid
  modified_base 2^3: mod_base = OTHER; note = phosphorothioate linkage
  modified_base 3: mod_base = OTHER; note = Locked Nucleic Acid
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12: mod_base = OTHER; note = Locked Nucleic Acid
  modified_base 12^13: mod_base = OTHER; note = phosphorothioate linkage
  modified_base 13: mod_base = OTHER; note = Locked Nucleic Acid
  modified_base 13^14: mod_base = OTHER; note = phosphorothioate linkage
  modified_base 14: mod_base = OTHER; note = Locked Nucleic Acid
ggattcttggcttt

SEQ ID NO: 101 - DNA, 14 bp, synthetic construct
  source 1..14: mol_type = other DNA
  modified_base 1: mod_base = OTHER; note = Locked Nucleic Acid
  modified_base 2: mod_base = OTHER; note = Locked Nucleic Acid
  modified_base 3: mod_base = OTHER; note = Locked Nucleic Acid
  modified_base 12: mod_base = OTHER; note = Locked Nucleic Acid
  modified_base 13: mod_base = OTHER; note = Locked Nucleic Acid
  modified_base 14: mod_base = OTHER; note = Locked Nucleic Acid
  modified_base 13^14: mod_base = OTHER; note = phosphorothioate linkage
  modified_base 12^13: mod_base = OTHER; note = phosphorothioate linkage
  modified_base 11^12: mod_base = OTHER; note = phosphorothioate linkage
  modified_base 10^11: mod_base = OTHER; note = phosphorothioate linkage
  modified_base 9^10: mod_base = OTHER; note = phosphorothioate linkage
  modified_base 8^9: mod_base = OTHER; note = phosphorothioate linkage
  modified_base 7^8: mod_base = OTHER; note = phosphorothioate linkage
  modified_base 6^7: mod_base = OTHER; note = phosphorothioate linkage
  modified_base 5^6: mod_base = OTHER; note = phosphorothioate linkage
  modified_base 4^5: mod_base = OTHER; note = phosphorothioate linkage
  modified_base 3^4: mod_base = OTHER; note = phosphorothioate linkage
  modified_base 2^3: mod_base = OTHER; note = phosphorothioate linkage
  modified_base 1^2: mod_base = OTHER; note = phosphorothioate linkage
acgtctatacacca

SEQ ID NO: 102-104 - skipped (3 entries)

SEQ ID NO: 105 - DNA, 14 bp, synthetic construct
  source 1..14: mol_type = other DNA
  modified_base 1: mod_base = OTHER; note = Locked Nucleic Acid
  modified_base 1^2: mod_base = OTHER; note = phosphorothioate linkage
  modified_base 2: mod_base = OTHER; note = Locked Nucleic Acid
  modified_base 2^3: mod_base = OTHER; note = phosphorothioate linkage
  modified_base 3: mod_base = OTHER; note = Locked Nucleic Acid
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12: mod_base = OTHER; note = Locked Nucleic Acid
  modified_base 12^13: mod_base = OTHER; note = phosphorothioate linkage
  modified_base 13: mod_base = OTHER; note = Locked Nucleic Acid
  modified_base 13^14: mod_base = OTHER; note = phosphorothioate linkage
  modified_base 14: mod_base = OTHER; note = Locked Nucleic Acid
gtggctagatccac

SEQ ID NO: 106 - DNA, 14 bp, synthetic construct
  source 1..14: mol_type = other DNA
  modified_base 1: mod_base = OTHER; note = Locked Nucleic Acid
  modified_base 1^2: mod_base = OTHER; note = phosphorothioate linkage
  modified_base 2: mod_base = OTHER; note = Locked Nucleic Acid
  modified_base 2^3: mod_base = OTHER; note = phosphorothioate linkage
  modified_base 3: mod_base = OTHER; note = Locked Nucleic Acid
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12: mod_base = OTHER; note = Locked Nucleic Acid
  modified_base 12^13: mod_base = OTHER; note = phosphorothioate linkage
  modified_base 13: mod_base = OTHER; note = Locked Nucleic Acid
  modified_base 13^14: mod_base = OTHER; note = phosphorothioate linkage
  modified_base 14: mod_base = OTHER; note = Locked Nucleic Acid
ctgttgatcgcagc

SEQ ID NO: 107 - DNA, 14 bp, synthetic construct
  source 1..14: mol_type = other DNA
  modified_base 1: mod_base = OTHER; note = Locked Nucleic Acid
  modified_base 1^2: mod_base = OTHER; note = phosphorothioate linkage
  modified_base 2: mod_base = OTHER; note = Locked Nucleic Acid
  modified_base 2^3: mod_base = OTHER; note = phosphorothioate linkage
  modified_base 3: mod_base = OTHER; note = Locked Nucleic Acid
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12: mod_base = OTHER; note = Locked Nucleic Acid
  modified_base 12^13: mod_base = OTHER; note = phosphorothioate linkage
  modified_base 13: mod_base = OTHER; note = Locked Nucleic Acid
  modified_base 13^14: mod_base = OTHER; note = phosphorothioate linkage
  modified_base 14: mod_base = OTHER; note = Locked Nucleic Acid
aaacccatccactc

SEQ ID NO: 108 - DNA, 14 bp, synthetic construct
  source 1..14: mol_type = other DNA
  modified_base 1: mod_base = OTHER; note = Locked Nucleic Acid
  modified_base 1^2: mod_base = OTHER; note = phosphorothioate linkage
  modified_base 2: mod_base = OTHER; note = Locked Nucleic Acid
  modified_base 2^3: mod_base = OTHER; note = phosphorothioate linkage
  modified_base 3: mod_base = OTHER; note = Locked Nucleic Acid
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12: mod_base = OTHER; note = Locked Nucleic Acid
  modified_base 12^13: mod_base = OTHER; note = phosphorothioate linkage
  modified_base 13: mod_base = OTHER; note = Locked Nucleic Acid
  modified_base 13^14: mod_base = OTHER; note = phosphorothioate linkage
  modified_base 14: mod_base = OTHER; note = Locked Nucleic Acid
gtgtgcacaggctc

SEQ ID NO: 109 - DNA, 14 bp, synthetic construct
  source 1..14: mol_type = other DNA
  modified_base 1: mod_base = OTHER; note = Locked Nucleic Acid
  modified_base 1^2: mod_base = OTHER; note = phosphorothioate linkage
  modified_base 2: mod_base = OTHER; note = Locked Nucleic Acid
  modified_base 2^3: mod_base = OTHER; note = phosphorothioate linkage
  modified_base 3: mod_base = OTHER; note = Locked Nucleic Acid
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12: mod_base = OTHER; note = Locked Nucleic Acid
  modified_base 12^13: mod_base = OTHER; note = phosphorothioate linkage
  modified_base 13: mod_base = OTHER; note = Locked Nucleic Acid
  modified_base 13^14: mod_base = OTHER; note = phosphorothioate linkage
  modified_base 14: mod_base = OTHER; note = Locked Nucleic Acid
ccctggaacaccac

SEQ ID NO: 110 - DNA, 14 bp, synthetic construct
  source 1..14: mol_type = other DNA
  modified_base 1: mod_base = OTHER; note = Locked Nucleic Acid
  modified_base 1^2: mod_base = OTHER; note = phosphorothioate linkage
  modified_base 2: mod_base = OTHER; note = Locked Nucleic Acid
  modified_base 2^3: mod_base = OTHER; note = phosphorothioate linkage
  modified_base 3: mod_base = OTHER; note = Locked Nucleic Acid
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12: mod_base = OTHER; note = Locked Nucleic Acid
  modified_base 12^13: mod_base = OTHER; note = phosphorothioate linkage
  modified_base 13: mod_base = OTHER; note = phosphorothioate linkage
  modified_base 13^14: mod_base = OTHER; note = phosphorothioate linkage
  modified_base 14: mod_base = OTHER; note = Locked Nucleic Acid
gcccagtccaacat

SEQ ID NO: 111 - DNA, 14 bp, synthetic construct
  source 1..14: mol_type = other DNA
  modified_base 1: mod_base = OTHER; note = Locked Nucleic Acid
  modified_base 1^2: mod_base = OTHER; note = phosphorothioate linkage
  modified_base 2: mod_base = OTHER; note = Locked Nucleic Acid
  modified_base 2^3: mod_base = OTHER; note = phosphorothioate linkage
  modified_base 3: mod_base = OTHER; note = Locked Nucleic Acid
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12: mod_base = OTHER; note = Locked Nucleic Acid
  modified_base 12^13: mod_base = OTHER; note = phosphorothioate linkage
  modified_base 13: mod_base = OTHER; note = Locked Nucleic Acid
  modified_base 13^14: mod_base = OTHER; note = phosphorothioate linkage
  modified_base 14: mod_base = OTHER; note = Locked Nucleic Acid
aggctcacctctcg

SEQ ID NO: 112 - DNA, 14 bp, synthetic construct
  source 1..14: mol_type = other DNA
  modified_base 1: mod_base = OTHER; note = Locked Nucleic Acid
  modified_base 1^2: mod_base = OTHER; note = phosphorothioate linkage
  modified_base 2: mod_base = OTHER; note = Locked Nucleic Acid
  modified_base 2^3: mod_base = OTHER; note = phosphorothioate linkage
  modified_base 3: mod_base = OTHER; note = Locked Nucleic Acid
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12: mod_base = OTHER; note = Locked Nucleic Acid
  modified_base 12^13: mod_base = OTHER; note = phosphorothioate linkage
  modified_base 13: mod_base = OTHER; note = Locked Nucleic Acid
  modified_base 13^14: mod_base = OTHER; note = phosphorothioate linkage
  modified_base 14: mod_base = OTHER; note = Locked Nucleic Acid
ggtgggtttgggtc

SEQ ID NO: 113 - DNA, 14 bp, synthetic construct
  source 1..14: mol_type = other DNA
  modified_base 1: mod_base = OTHER; note = Locked Nucleic Acid
  modified_base 1^2: mod_base = OTHER; note = phosphorothioate linkage
  modified_base 2: mod_base = OTHER; note = Locked Nucleic Acid
  modified_base 2^3: mod_base = OTHER; note = phosphorothioate linkage
  modified_base 3: mod_base = OTHER; note = Locked Nucleic Acid
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12: mod_base = OTHER; note = Locked Nucleic Acid
  modified_base 12^13: mod_base = OTHER; note = phosphorothioate linkage
  modified_base 13: mod_base = OTHER; note = Locked Nucleic Acid
  modified_base 13^14: mod_base = OTHER; note = phosphorothioate linkage
  modified_base 14: mod_base = OTHER; note = Locked Nucleic Acid
catgaggaagagga

SEQ ID NO: 114 - DNA, 14 bp, synthetic construct
  source 1..14: mol_type = other DNA
  modified_base 1: mod_base = OTHER; note = Locked Nucleic Acid
  modified_base 1^2: mod_base = OTHER; note = phosphorothioate linkage
  modified_base 2: mod_base = OTHER; note = Locked Nucleic Acid
  modified_base 2^3: mod_base = OTHER; note = phosphorothioate linkage
  modified_base 3: mod_base = OTHER; note = Locked Nucleic Acid
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12: mod_base = OTHER; note = Locked Nucleic Acid
  modified_base 12^13: mod_base = OTHER; note = phosphorothioate linkage
  modified_base 13: mod_base = OTHER; note = Locked Nucleic Acid
  modified_base 13^14: mod_base = OTHER; note = phosphorothioate linkage
  modified_base 14: mod_base = OTHER; note = Locked Nucleic Acid
gtgctcgtcaaagg

SEQ ID NO: 115 - DNA, 14 bp, synthetic construct
  source 1..14: mol_type = other DNA
  modified_base 1: mod_base = OTHER; note = Locked Nucleic Acid
  modified_base 1^2: mod_base = OTHER; note = phosphorothioate linkage
  modified_base 2: mod_base = OTHER; note = Locked Nucleic Acid
  modified_base 2^3: mod_base = OTHER; note = phosphorothioate linkage
  modified_base 3: mod_base = OTHER; note = Locked Nucleic Acid
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12: mod_base = OTHER; note = Locked Nucleic Acid
  modified_base 12^13: mod_base = OTHER; note = phosphorothioate linkage
  modified_base 13: mod_base = OTHER; note = Locked Nucleic Acid
  modified_base 13^14: mod_base = OTHER; note = phosphorothioate linkage
  modified_base 14: mod_base = OTHER; note = Locked Nucleic Acid
atgtgctcgtcaaa

SEQ ID NO: 116 - DNA, 14 bp, synthetic construct
  source 1..14: mol_type = other DNA
  modified_base 1: mod_base = OTHER; note = Locked Nucleic Acid
  modified_base 1^2: mod_base = OTHER; note = phosphorothioate linkage
  modified_base 2: mod_base = OTHER; note = Locked Nucleic Acid
  modified_base 2^3: mod_base = OTHER; note = phosphorothioate linkage
  modified_base 3: mod_base = OTHER; note = Locked Nucleic Acid
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12: mod_base = OTHER; note = Locked Nucleic Acid
  modified_base 12^13: mod_base = OTHER; note = phosphorothioate linkage
  modified_base 13: mod_base = OTHER; note = Locked Nucleic Acid
  modified_base 13^14: mod_base = OTHER; note = phosphorothioate linkage
  modified_base 14: mod_base = OTHER; note = Locked Nucleic Acid
agaatgtctccccg

SEQ ID NO: 117 - DNA, 14 bp, synthetic construct
  source 1..14: mol_type = other DNA
  modified_base 1: mod_base = OTHER; note = Locked Nucleic Acid
  modified_base 1^2: mod_base = OTHER; note = phosphorothioate linkage
  modified_base 2: mod_base = OTHER; note = Locked Nucleic Acid
  modified_base 2^3: mod_base = OTHER; note = phosphorothioate linkage
  modified_base 3: mod_base = OTHER; note = Locked Nucleic Acid
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 5^6: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12: mod_base = OTHER; note = Locked Nucleic Acid
  modified_base 12^13: mod_base = OTHER; note = phosphorothioate linkage
  modified_base 13: mod_base = OTHER; note = Locked Nucleic Acid
  modified_base 13^14: mod_base = OTHER; note = phosphorothioate linkage
  modified_base 14: mod_base = OTHER; note = Locked Nucleic Acid
atgagcagagaggc

SEQ ID NO: 118 - DNA, 14 bp, synthetic construct
  source 1..14: mol_type = other DNA
  modified_base 1: mod_base = OTHER; note = Locked Nucleic Acid
  modified_base 1^2: mod_base = OTHER; note = phosphorothioate linkage
  modified_base 2: mod_base = OTHER; note = Locked Nucleic Acid
  modified_base 2^3: mod_base = OTHER; note = phosphorothioate linkage
  modified_base 3: mod_base = OTHER; note = Locked Nucleic Acid
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12: mod_base = OTHER; note = Locked Nucleic Acid
  modified_base 12^13: mod_base = OTHER; note = phosphorothioate linkage
  modified_base 13: mod_base = OTHER; note = Locked Nucleic Acid
  modified_base 13^14: mod_base = OTHER; note = phosphorothioate linkage
  modified_base 14: mod_base = OTHER; note = Locked Nucleic Acid
gctgcagtttctcc

SEQ ID NO: 119 - DNA, 14 bp, synthetic construct
  source 1..14: mol_type = other DNA
  modified_base 1: mod_base = OTHER; note = Locked Nucleic Acid
  modified_base 1^2: mod_base = OTHER; note = phosphorothioate linkage
  modified_base 2: mod_base = OTHER; note = phosphorothioate linkage
  modified_base 2^3: mod_base = OTHER; note = phosphorothioate linkage
  modified_base 3: mod_base = OTHER; note = Locked Nucleic Acid
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12: mod_base = OTHER; note = Locked Nucleic Acid
  modified_base 12^13: mod_base = OTHER; note = phosphorothioate linkage
  modified_base 13: mod_base = OTHER; note = Locked Nucleic Acid
  modified_base 13^14: mod_base = OTHER; note = phosphorothioate linkage
  modified_base 14: mod_base = OTHER; note = Locked Nucleic Acid
aggatctccacatg

SEQ ID NO: 120 - DNA, 14 bp, synthetic construct
  source 1..14: mol_type = other DNA
  modified_base 1: mod_base = OTHER; note = Locked Nucleic Acid
  modified_base 1^2: mod_base = OTHER; note = phosphorothioate linkage
  modified_base 3: mod_base = OTHER; note = Locked Nucleic Acid
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12: mod_base = OTHER; note = Locked Nucleic Acid
  modified_base 12^13: mod_base = OTHER; note = phosphorothioate linkage
  modified_base 13: mod_base = OTHER; note = Locked Nucleic Acid
  modified_base 13^14: mod_base = OTHER; note = phosphorothioate linkage
  modified_base 14: mod_base = OTHER; note = Locked Nucleic Acid
ttccttttggcctc

SEQ ID NO: 121 - DNA, 14 bp, synthetic construct
  source 1..14: mol_type = other DNA
  modified_base 1: mod_base = OTHER; note = Locked Nucleic Acid
  modified_base 1^2: mod_base = OTHER; note = phosphorothioate linkage
  modified_base 2: mod_base = OTHER; note = phosphorothioate linkage
  modified_base 2^3: mod_base = OTHER; note = phosphorothioate linkage
  modified_base 3: mod_base = OTHER; note = Locked Nucleic Acid
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12: mod_base = OTHER; note = Locked Nucleic Acid
  modified_base 12^13: mod_base = OTHER; note = phosphorothioate linkage
  modified_base 13: mod_base = OTHER; note = Locked Nucleic Acid
  modified_base 13^14: mod_base = OTHER; note = phosphorothioate linkage
  modified_base 14: mod_base = OTHER; note = Locked Nucleic Acid
atgaagcacatggt

SEQ ID NO: 122 - DNA, 14 bp, synthetic construct
  source 1..14: mol_type = other DNA
  modified_base 1: mod_base = OTHER; note = Locked Nucleic Acid
  modified_base 1^2: mod_base = OTHER; note = phosphorothioate linkage
  modified_base 2: mod_base = OTHER; note = Locked Nucleic Acid
  modified_base 2^3: mod_base = OTHER; note = phosphorothioate linkage
  modified_base 3: mod_base = OTHER; note = Locked Nucleic Acid
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12: mod_base = OTHER; note = Locked Nucleic Acid
  modified_base 12^13: mod_base = OTHER; note = phosphorothioate linkage
  modified_base 13: mod_base = OTHER; note = Locked Nucleic Acid
  modified_base 13^14: mod_base = OTHER; note = phosphorothioate linkage
  modified_base 14: mod_base = OTHER; note = Locked Nucleic Acid
gggggatgaagcac

SEQ ID NO: 123 - DNA, 14 bp, synthetic construct
  source 1..14: mol_type = other DNA
  modified_base 1: mod_base = OTHER; note = Locked Nucleic Acid
  modified_base 1^2: mod_base = OTHER; note = phosphorothioate linkage
  modified_base 2: mod_base = OTHER; note = Locked Nucleic Acid
  modified_base 2^3: mod_base = OTHER; note = phosphorothioate linkage
  modified_base 3: mod_base = OTHER; note = Locked Nucleic Acid
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12: mod_base = OTHER; note = Locked Nucleic Acid
  modified_base 12^13: mod_base = OTHER; note = phosphorothioate linkage
  modified_base 13: mod_base = OTHER; note = Locked Nucleic Acid
  modified_base 13^14: mod_base = OTHER; note = phosphorothioate linkage
  modified_base 14: mod_base = OTHER; note = Locked Nucleic Acid
cagggggatgaagc

SEQ ID NO: 124 - DNA, 14 bp, synthetic construct
  source 1..14: mol_type = other DNA
  modified_base 1: mod_base = OTHER; note = Locked Nucleic Acid
  modified_base 1^2: mod_base = OTHER; note = phosphorothioate linkage
  modified_base 2: mod_base = OTHER; note = Locked Nucleic Acid
  modified_base 2^3: mod_base = OTHER; note = phosphorothioate linkage
  modified_base 3: mod_base = OTHER; note = Locked Nucleic Acid
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12: mod_base = OTHER; note = Locked Nucleic Acid
  modified_base 12^13: mod_base = OTHER; note = phosphorothioate linkage
  modified_base 13: mod_base = OTHER; note = Locked Nucleic Acid
  modified_base 13^14: mod_base = OTHER; note = phosphorothioate linkage
  modified_base 14: mod_base = OTHER; note = Locked Nucleic Acid
gaccagggggatga

SEQ ID NO: 125 - DNA, 14 bp, synthetic construct
  source 1..14: mol_type = other DNA
  modified_base 1: mod_base = OTHER; note = Locked Nucleic Acid
  modified_base 1^2: mod_base = OTHER; note = phosphorothioate linkage
  modified_base 2: mod_base = OTHER; note = Locked Nucleic Acid
  modified_base 2^3: mod_base = OTHER; note = phosphorothioate linkage
  modified_base 3: mod_base = OTHER; note = Locked Nucleic Acid
  N_region 3^4
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12: mod_base = OTHER; note = Locked Nucleic Acid
  modified_base 12^13: mod_base = OTHER; note = phosphorothioate linkage
  modified_base 13: mod_base = OTHER; note = Locked Nucleic Acid
  modified_base 13^14: mod_base = OTHER; note = phosphorothioate linkage
  modified_base 14: mod_base = OTHER; note = Locked Nucleic Acid
gcagaccaggggga

SEQ ID NO: 126 - DNA, 14 bp, synthetic construct
  source 1..14: mol_type = other DNA
  modified_base 1: mod_base = OTHER; note = Locked Nucleic Acid
  modified_base 1^2: mod_base = OTHER; note = phosphorothioate linkage
  modified_base 2: mod_base = OTHER; note = Locked Nucleic Acid
  modified_base 2^3: mod_base = OTHER; note = phosphorothioate linkage
  modified_base 3: mod_base = OTHER; note = Locked Nucleic Acid
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12: mod_base = OTHER; note = Locked Nucleic Acid
  modified_base 12^13: mod_base = OTHER; note = phosphorothioate linkage
  modified_base 13: mod_base = OTHER; note = Locked Nucleic Acid
  modified_base 13^14: mod_base = OTHER; note = phosphorothioate linkage
  modified_base 14: mod_base = OTHER; note = Locked Nucleic Acid
cagcagaccagggg

SEQ ID NO: 127 - DNA, 14 bp, synthetic construct
  source 1..14: mol_type = other DNA
  modified_base 1: mod_base = OTHER; note = Locked Nucleic Acid
  modified_base 1^2: mod_base = OTHER; note = phosphorothioate linkage
  modified_base 2: mod_base = OTHER; note = Locked Nucleic Acid
  modified_base 2^3: mod_base = OTHER; note = phosphorothioate linkage
  modified_base 3: mod_base = OTHER; note = Locked Nucleic Acid
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12: mod_base = OTHER; note = Locked Nucleic Acid
  modified_base 12^13: mod_base = OTHER; note = phosphorothioate linkage
  modified_base 13: mod_base = OTHER; note = Locked Nucleic Acid
  modified_base 13^14: mod_base = OTHER; note = phosphorothioate linkage
  modified_base 14: mod_base = OTHER; note = Locked Nucleic Acid
atccagcagaccag

SEQ ID NO: 128 - DNA, 14 bp, synthetic construct
  source 1..14: mol_type = other DNA
  modified_base 1: mod_base = OTHER; note = Locked Nucleic Acid
  modified_base 1^2: mod_base = OTHER; note = phosphorothioate linkage
  modified_base 2: mod_base = OTHER; note = Locked Nucleic Acid
  modified_base 2^3: mod_base = OTHER; note = phosphorothioate linkage
  modified_base 3: mod_base = OTHER; note = Locked Nucleic Acid
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12: mod_base = OTHER; note = Locked Nucleic Acid
  modified_base 12^13: mod_base = OTHER; note = phosphorothioate linkage
  modified_base 13: mod_base = OTHER; note = Locked Nucleic Acid
  modified_base 13^14: mod_base = OTHER; note = phosphorothioate linkage
  modified_base 14: mod_base = OTHER; note = Locked Nucleic Acid
aggaagaagacgta

SEQ ID NO: 129 - DNA, 14 bp, synthetic construct
  source 1..14: mol_type = other DNA
  modified_base 1: mod_base = OTHER; note = Locked Nucleic Acid
  modified_base 1^2: mod_base = OTHER; note = phosphorothioate linkage
  modified_base 2: mod_base = OTHER; note = Locked Nucleic Acid
  modified_base 2^3: mod_base = OTHER; note = phosphorothioate linkage
  modified_base 3: mod_base = OTHER; note = Locked Nucleic Acid
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12: mod_base = OTHER; note = Locked Nucleic Acid
  modified_base 12^13: mod_base = OTHER; note = phosphorothioate linkage
  modified_base 13: mod_base = OTHER; note = Locked Nucleic Acid
  modified_base 13^14: mod_base = OTHER; note = phosphorothioate linkage
  modified_base 14: mod_base = OTHER; note = Locked Nucleic Acid
gaaaggaagaagac

SEQ ID NO: 130 - DNA, 14 bp, synthetic construct
  source 1..14: mol_type = other DNA
  modified_base 1: mod_base = OTHER; note = Locked Nucleic Acid
  modified_base 1^2: mod_base = OTHER; note = phosphorothioate linkage
  modified_base 2: mod_base = OTHER; note = Locked Nucleic Acid
  modified_base 2^3: mod_base = OTHER; note = phosphorothioate linkage
  modified_base 3: mod_base = OTHER; note = Locked Nucleic Acid
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12: mod_base = OTHER; note = Locked Nucleic Acid
  modified_base 12^13: mod_base = OTHER; note = phosphorothioate linkage
  modified_base 13: mod_base = OTHER; note = Locked Nucleic Acid
  modified_base 13^14: mod_base = OTHER; note = phosphorothioate linkage
  modified_base 14: mod_base = OTHER; note = Locked Nucleic Acid
gattttctggttcc

SEQ ID NO: 131 - DNA, 14 bp, synthetic construct
  source 1..14: mol_type = other DNA
  modified_base 1: mod_base = OTHER; note = Locked Nucleic Acid
  modified_base 1^2: mod_base = OTHER; note = phosphorothioate linkage
  modified_base 2: mod_base = OTHER; note = Locked Nucleic Acid
  modified_base 2^3: mod_base = OTHER; note = phosphorothioate linkage
  modified_base 3: mod_base = OTHER; note = Locked Nucleic Acid
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12: mod_base = OTHER; note = Locked Nucleic Acid
  modified_base 12^13: mod_base = OTHER; note = phosphorothioate linkage
  modified_base 13: mod_base = OTHER; note = Locked Nucleic Acid
  modified_base 13^14: mod_base = OTHER; note = phosphorothioate linkage
  modified_base 14: mod_base = OTHER; note = Locked Nucleic Acid
aggattttctggtt

SEQ ID NO: 132 - DNA, 14 bp, synthetic construct
  source 1..14: mol_type = other DNA
  modified_base 1: mod_base = OTHER; note = Locked Nucleic Acid
  modified_base 1^2: mod_base = OTHER; note = phosphorothioate linkage
  modified_base 2: mod_base = OTHER; note = phosphorothioate linkage
  modified_base 2^3: mod_base = OTHER; note = phosphorothioate linkage
  modified_base 3: mod_base = OTHER; note = Locked Nucleic Acid
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12: mod_base = OTHER; note = Locked Nucleic Acid
  modified_base 12^13: mod_base = OTHER; note = phosphorothioate linkage
  modified_base 13: mod_base = OTHER; note = Locked Nucleic Acid
  modified_base 13^14: mod_base = OTHER; note = phosphorothioate linkage
  modified_base 14: mod_base = OTHER; note = Locked Nucleic Acid
atcctcaggaaagc

SEQ ID NO: 133 - DNA, 14 bp, synthetic construct
  source 1..14: mol_type = other DNA
  modified_base 1: mod_base = OTHER; note = Locked Nucleic Acid
  modified_base 1^2: mod_base = OTHER; note = phosphorothioate linkage
  modified_base 2: mod_base = OTHER; note = Locked Nucleic Acid
  modified_base 2^3: mod_base = OTHER; note = phosphorothioate linkage
  modified_base 3: mod_base = OTHER; note = Locked Nucleic Acid
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12: mod_base = OTHER; note = Locked Nucleic Acid
  modified_base 12^13: mod_base = OTHER; note = phosphorothioate linkage
  modified_base 13: mod_base = OTHER; note = Locked Nucleic Acid
  modified_base 13^14: mod_base = OTHER; note = phosphorothioate linkage
  modified_base 14: mod_base = OTHER; note = Locked Nucleic Acid
gttcatcctcagga

SEQ ID NO: 134 - DNA, 14 bp, synthetic construct
  source 1..14: mol_type = other DNA
  modified_base 1: mod_base = OTHER; note = Locked Nucleic Acid
  modified_base 1^2: mod_base = OTHER; note = phosphorothioate linkage
  modified_base 2: mod_base = OTHER; note = Locked Nucleic Acid
  modified_base 2^3: mod_base = OTHER; note = phosphorothioate linkage
  modified_base 3: mod_base = OTHER; note = Locked Nucleic Acid
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12: mod_base = OTHER; note = Locked Nucleic Acid
  modified_base 13: mod_base = OTHER; note = Locked Nucleic Acid
  modified_base 12^13: mod_base = OTHER; note = phosphorothioate linkage
  modified_base 13^14: mod_base = OTHER; note = phosphorothioate linkage
  modified_base 14: mod_base = OTHER; note = Locked Nucleic Acid
gttcatcctcagga

SEQ ID NO: 135 - DNA, 14 bp, synthetic construct
  source 1..14: mol_type = other DNA
  modified_base 1: mod_base = OTHER; note = Locked Nucleic Acid
  modified_base 1^2: mod_base = OTHER; note = phosphorothioate linkage
  modified_base 2: mod_base = OTHER; note = Locked Nucleic Acid
  modified_base 2^3: mod_base = OTHER; note = phosphorothioate linkage
  modified_base 3: mod_base = OTHER; note = Locked Nucleic Acid
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12: mod_base = OTHER; note = Locked Nucleic Acid
  modified_base 12^13: mod_base = OTHER; note = phosphorothioate linkage
  modified_base 13: mod_base = OTHER; note = Locked Nucleic Acid
  modified_base 13^14: mod_base = OTHER; note = phosphorothioate linkage
  modified_base 14: mod_base = OTHER; note = Locked Nucleic Acid
tcgcagtccacttc

SEQ ID NO: 136 - DNA, 14 bp, synthetic construct
  source 1..14: mol_type = other DNA
  modified_base 1: mod_base = OTHER; note = Locked Nucleic Acid
  modified_base 1^2: mod_base = OTHER; note = phosphorothioate linkage
  modified_base 2^3: mod_base = OTHER; note = phosphorothioate linkage
  modified_base 3: mod_base = OTHER; note = Locked Nucleic Acid
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12: mod_base = OTHER; note = Locked Nucleic Acid
  modified_base 12^13: mod_base = OTHER; note = phosphorothioate linkage
  modified_base 13: mod_base = OTHER; note = Locked Nucleic Acid
  modified_base 13^14: mod_base = OTHER; note = phosphorothioate linkage
  modified_base 14: mod_base = OTHER; note = Locked Nucleic Acid
tctcgcagtccact

SEQ ID NO: 137 - DNA, 14 bp, synthetic construct
  source 1..14: mol_type = other DNA
  modified_base 1: mod_base = OTHER; note = Locked Nucleic Acid
  modified_base 1^2: mod_base = OTHER; note = phosphorothioate linkage
  modified_base 2: mod_base = OTHER; note = Locked Nucleic Acid
  modified_base 2^3: mod_base = OTHER; note = phosphorothioate linkage
  modified_base 3: mod_base = OTHER; note = Locked Nucleic Acid
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12: mod_base = OTHER; note = Locked Nucleic Acid
  modified_base 12^13: mod_base = OTHER; note = phosphorothioate linkage
  modified_base 13: mod_base = OTHER; note = Locked Nucleic Acid
  modified_base 13^14: mod_base = OTHER; note = phosphorothioate linkage
  modified_base 14: mod_base = OTHER; note = Locked Nucleic Acid
tgaagctgtagaac

SEQ ID NO: 138 - DNA, 14 bp, synthetic construct
  source 1..14: mol_type = other DNA
  modified_base 1: mod_base = OTHER; note = Locked Nucleic Acid
  modified_base 1^2: mod_base = OTHER; note = phosphorothioate linkage
  modified_base 2: mod_base = OTHER; note = Locked Nucleic Acid
  modified_base 2^3: mod_base = OTHER; note = phosphorothioate linkage
  modified_base 3: mod_base = OTHER; note = Locked Nucleic Acid
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12: mod_base = OTHER; note = Locked Nucleic Acid
  modified_base 12^13: mod_base = OTHER; note = phosphorothioate linkage
  modified_base 13: mod_base = OTHER; note = Locked Nucleic Acid
  modified_base 13^14: mod_base = OTHER; note = phosphorothioate linkage
  modified_base 14: mod_base = OTHER; note = Locked Nucleic Acid
tggaaagtcatgtg

SEQ ID NO: 139 - DNA, 14 bp, synthetic construct
  source 1..14: mol_type = other DNA
  modified_base 1: mod_base = OTHER; note = Locked Nucleic Acid
  modified_base 1^2: mod_base = OTHER; note = phosphorothioate linkage
  modified_base 2: mod_base = OTHER; note = Locked Nucleic Acid
  modified_base 2^3: mod_base = OTHER; note = phosphorothioate linkage
  modified_base 3: mod_base = OTHER; note = Locked Nucleic Acid
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12: mod_base = OTHER; note = Locked Nucleic Acid
  modified_base 12^13: mod_base = OTHER; note = phosphorothioate linkage
  modified_base 13: mod_base = OTHER; note = Locked Nucleic Acid
  modified_base 13^14: mod_base = OTHER; note = phosphorothioate linkage
  modified_base 14: mod_base = OTHER; note = Locked Nucleic Acid
cctggaaagtcatg

SEQ ID NO: 140 - DNA, 14 bp, synthetic construct
  source 1..14: mol_type = other DNA
  modified_base 1: mod_base = OTHER; note = Locked Nucleic Acid
  modified_base 1^2: mod_base = OTHER; note = phosphorothioate linkage
  modified_base 2: mod_base = OTHER; note = Locked Nucleic Acid
  modified_base 2^3: mod_base = OTHER; note = phosphorothioate linkage
  modified_base 3: mod_base = OTHER; note = Locked Nucleic Acid
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12: mod_base = OTHER; note = Locked Nucleic Acid
  modified_base 12^13: mod_base = OTHER; note = phosphorothioate linkage
  modified_base 13: mod_base = OTHER; note = Locked Nucleic Acid
  modified_base 13^14: mod_base = OTHER; note = phosphorothioate linkage
  modified_base 14: mod_base = OTHER; note = Locked Nucleic Acid
ctcctggaaagtca

SEQ ID NO: 141 - DNA, 14 bp, synthetic construct
  source 1..14: mol_type = other DNA
  modified_base 1: mod_base = OTHER; note = Locked Nucleic Acid
  modified_base 1^2: mod_base = OTHER; note = phosphorothioate linkage
  modified_base 2: mod_base = OTHER; note = Locked Nucleic Acid
  modified_base 2^3: mod_base = OTHER; note = phosphorothioate linkage
  modified_base 3: mod_base = OTHER; note = Locked Nucleic Acid
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12: mod_base = OTHER; note = Locked Nucleic Acid
  modified_base 12^13: mod_base = OTHER; note = phosphorothioate linkage
  modified_base 13: mod_base = OTHER; note = Locked Nucleic Acid
  modified_base 13^14: mod_base = OTHER; note = phosphorothioate linkage
  modified_base 14: mod_base = OTHER; note = Locked Nucleic Acid
aactcctggaaagt

SEQ ID NO: 142 - DNA, 14 bp, synthetic construct
  source 1..14: mol_type = other DNA
  modified_base 1: mod_base = OTHER; note = Locked Nucleic Acid
  modified_base 1^2: mod_base = OTHER; note = phosphorothioate linkage
  modified_base 2: mod_base = OTHER; note = Locked Nucleic Acid
  modified_base 2^3: mod_base = OTHER; note = phosphorothioate linkage
  modified_base 3: mod_base = OTHER; note = Locked Nucleic Acid
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12: mod_base = OTHER; note = Locked Nucleic Acid
  modified_base 12^13: mod_base = OTHER; note = phosphorothioate linkage
  modified_base 13: mod_base = OTHER; note = Locked Nucleic Acid
  modified_base 13^14: mod_base = OTHER; note = phosphorothioate linkage
  modified_base 14: mod_base = OTHER; note = Locked Nucleic Acid
agaactcctggaaa

SEQ ID NO: 143 - DNA, 14 bp, synthetic construct
  source 1..14: mol_type = other DNA
  modified_base 1: mod_base = OTHER; note = Locked Nucleic Acid
  modified_base 1^2: mod_base = OTHER; note = phosphorothioate linkage
  modified_base 2: mod_base = OTHER; note = Locked Nucleic Acid
  modified_base 2^3: mod_base = OTHER; note = phosphorothioate linkage
  modified_base 3: mod_base = OTHER; note = Locked Nucleic Acid
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12: mod_base = OTHER; note = Locked Nucleic Acid
  modified_base 12^13: mod_base = OTHER; note = phosphorothioate linkage
  modified_base 13: mod_base = OTHER; note = Locked Nucleic Acid
  modified_base 13^14: mod_base = OTHER; note = phosphorothioate linkage
  modified_base 14: mod_base = OTHER; note = Locked Nucleic Acid
  modified_base 3^4: mod_base = OTHER; note = phosphorothioate linkage
tacaacaaaaatca

SEQ ID NO: 144 - DNA, 14 bp, synthetic construct
  source 1..14: mol_type = other DNA
  modified_base 1: mod_base = OTHER; note = Locked Nucleic Acid
  modified_base 1^2: mod_base = OTHER; note = phosphorothioate linkage
  modified_base 2: mod_base = OTHER; note = Locked Nucleic Acid
  modified_base 2^3: mod_base = OTHER; note = phosphorothioate linkage
  modified_base 3: mod_base = OTHER; note = Locked Nucleic Acid
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12: mod_base = OTHER; note = Locked Nucleic Acid
  modified_base 12^13: mod_base = OTHER; note = phosphorothioate linkage
  modified_base 13: mod_base = OTHER; note = Locked Nucleic Acid
  modified_base 13^14: mod_base = OTHER; note = phosphorothioate linkage
  modified_base 14: mod_base = OTHER; note = Locked Nucleic Acid
aggccaaagaggaa

SEQ ID NO: 145 - DNA, 14 bp, synthetic construct
  source 1..14: mol_type = other DNA
  modified_base 1: mod_base = OTHER; note = Locked Nucleic Acid
  modified_base 1^2: mod_base = OTHER; note = phosphorothioate linkage
  modified_base 2: mod_base = OTHER; note = Locked Nucleic Acid
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12: mod_base = OTHER; note = Locked Nucleic Acid
  modified_base 12^13: mod_base = OTHER; note = phosphorothioate linkage
  modified_base 13: mod_base = OTHER; note = Locked Nucleic Acid
  modified_base 13^14: mod_base = OTHER; note = phosphorothioate linkage
  modified_base 14: mod_base = OTHER; note = Locked Nucleic Acid
ctctcctggtttac

SEQ ID NO: 146 - DNA, 14 bp, synthetic construct
  source 1..14: mol_type = other DNA
  modified_base 1: mod_base = OTHER; note = Locked Nucleic Acid
  modified_base 1^2: mod_base = OTHER; note = phosphorothioate linkage
  modified_base 2: mod_base = OTHER; note = Locked Nucleic Acid
  modified_base 2^3: mod_base = OTHER; note = phosphorothioate linkage
  modified_base 3: mod_base = OTHER; note = Locked Nucleic Acid
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12: mod_base = OTHER; note = Locked Nucleic Acid
  modified_base 12^13: mod_base = OTHER; note = phosphorothioate linkage
  modified_base 13: mod_base = OTHER; note = Locked Nucleic Acid
  modified_base 13^14: mod_base = OTHER; note = phosphorothioate linkage
  modified_base 14: mod_base = OTHER; note = Locked Nucleic Acid
gatcttgcaactta

SEQ ID NO: 147 - DNA, 14 bp, synthetic construct
  source 1..14: mol_type = other DNA
  modified_base 1: mod_base = OTHER; note = Locked Nucleic Acid
  modified_base 1^2: mod_base = OTHER; note = phosphorothioate linkage
  modified_base 2: mod_base = OTHER; note = Locked Nucleic Acid
  modified_base 2^3: mod_base = OTHER; note = phosphorothioate linkage
  modified_base 3: mod_base = OTHER; note = Locked Nucleic Acid
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12: mod_base = OTHER; note = Locked Nucleic Acid
  modified_base 12^13: mod_base = OTHER; note = phosphorothioate linkage
  modified_base 13: mod_base = OTHER; note = Locked Nucleic Acid
  modified_base 13^14: mod_base = OTHER; note = phosphorothioate linkage
  modified_base 14: mod_base = OTHER; note = Locked Nucleic Acid
gagatcttgcaact

SEQ ID NO: 148 - DNA, 14 bp, synthetic construct
  source 1..14: mol_type = other DNA
  modified_base 1: mod_base = OTHER; note = Locked Nucleic Acid
  modified_base 1^2: mod_base = OTHER; note = phosphorothioate linkage
  modified_base 2: mod_base = OTHER; note = Locked Nucleic Acid
  modified_base 2^3: mod_base = OTHER; note = phosphorothioate linkage
  modified_base 3: mod_base = OTHER; note = Locked Nucleic Acid
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12: mod_base = OTHER; note = Locked Nucleic Acid
  modified_base 12^13: mod_base = OTHER; note = phosphorothioate linkage
  modified_base 13: mod_base = OTHER; note = Locked Nucleic Acid
  modified_base 13^14: mod_base = OTHER; note = phosphorothioate linkage
  modified_base 14: mod_base = OTHER; note = Locked Nucleic Acid
gagagatcttgcaa

SEQ ID NO: 149 - DNA, 14 bp, synthetic construct
  source 1..14: mol_type = other DNA
  modified_base 1: mod_base = OTHER; note = Locked Nucleic Acid
  modified_base 1^2: mod_base = OTHER; note = phosphorothioate linkage
  modified_base 2: mod_base = OTHER; note = Locked Nucleic Acid
  modified_base 2^3: mod_base = OTHER; note = phosphorothioate linkage
  modified_base 3: mod_base = OTHER; note = Locked Nucleic Acid
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12: mod_base = OTHER; note = Locked Nucleic Acid
  modified_base 12^13: mod_base = OTHER; note = phosphorothioate linkage
  modified_base 13: mod_base = OTHER; note = Locked Nucleic Acid
  modified_base 13^14: mod_base = OTHER; note = phosphorothioate linkage
  modified_base 14: mod_base = OTHER; note = Locked Nucleic Acid
ctgagagatcttgc

SEQ ID NO: 150 - DNA, 14 bp, synthetic construct
  source 1..14: mol_type = other DNA
  modified_base 1: mod_base = OTHER; note = Locked Nucleic Acid
  modified_base 1^2: mod_base = OTHER; note = phosphorothioate linkage
  modified_base 2: mod_base = OTHER; note = Locked Nucleic Acid
  modified_base 2^3: mod_base = OTHER; note = phosphorothioate linkage
  modified_base 3: mod_base = OTHER; note = Locked Nucleic Acid
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12: mod_base = OTHER; note = Locked Nucleic Acid
  modified_base 12^13: mod_base = OTHER; note = phosphorothioate linkage
  modified_base 13: mod_base = OTHER; note = Locked Nucleic Acid
  modified_base 13^14: mod_base = OTHER; note = phosphorothioate linkage
  modified_base 14: mod_base = OTHER; note = Locked Nucleic Acid
tgctgagagatctt

SEQ ID NO: 151 - DNA, 14 bp, synthetic construct
  source 1..14: mol_type = other DNA
  modified_base 1: mod_base = OTHER; note = Locked Nucleic Acid
  modified_base 1^2: mod_base = OTHER; note = phosphorothioate linkage
  modified_base 2: mod_base = OTHER; note = Locked Nucleic Acid
  modified_base 2^3: mod_base = OTHER; note = phosphorothioate linkage
  modified_base 3: mod_base = OTHER; note = Locked Nucleic Acid
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12: mod_base = OTHER; note = Locked Nucleic Acid
  modified_base 12^13: mod_base = OTHER; note = phosphorothioate linkage
  modified_base 13: mod_base = OTHER; note = Locked Nucleic Acid
  modified_base 13^14: mod_base = OTHER; note = phosphorothioate linkage
  modified_base 14: mod_base = OTHER; note = Locked Nucleic Acid
tttcacttcaatcc

SEQ ID NO: 152 - DNA, 14 bp, synthetic construct
  source 1..14: mol_type = other DNA
  modified_base 1: mod_base = OTHER; note = Locked Nucleic Acid
  modified_base 1^2: mod_base = OTHER; note = phosphorothioate linkage
  modified_base 2: mod_base = OTHER; note = Locked Nucleic Acid
  modified_base 2^3: mod_base = OTHER; note = phosphorothioate linkage
  modified_base 3: mod_base = OTHER; note = Locked Nucleic Acid
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12: mod_base = OTHER; note = Locked Nucleic Acid
  modified_base 12^13: mod_base = OTHER; note = phosphorothioate linkage
  modified_base 13: mod_base = OTHER; note = Locked Nucleic Acid
  modified_base 13^14: mod_base = OTHER; note = phosphorothioate linkage
  modified_base 14: mod_base = OTHER; note = Locked Nucleic Acid
ctttcacttcaatc

SEQ ID NO: 153 - DNA, 14 bp, synthetic construct
  source 1..14: mol_type = other DNA
  modified_base 1: mod_base = OTHER; note = Locked Nucleic Acid
  modified_base 1^2: mod_base = OTHER; note = Locked Nucleic Acid
  modified_base 2: mod_base = OTHER; note = phosphorothioate linkage
  modified_base 2^3: mod_base = OTHER; note = phosphorothioate linkage
  modified_base 3: mod_base = OTHER; note = Locked Nucleic Acid
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12: mod_base = OTHER; note = Locked Nucleic Acid
  modified_base 12^13: mod_base = OTHER; note = phosphorothioate linkage
  modified_base 13: mod_base = OTHER; note = Locked Nucleic Acid
  modified_base 13^14: mod_base = OTHER; note = phosphorothioate linkage
  modified_base 14: mod_base = OTHER; note = Locked Nucleic Acid
ctgcatctcgtaca

SEQ ID NO: 154 - DNA, 14 bp, synthetic construct
  source 1..14: mol_type = other DNA
  modified_base 1: mod_base = OTHER; note = Locked Nucleic Acid
  modified_base 1^2: mod_base = OTHER; note = phosphorothioate linkage
  modified_base 2^3: mod_base = OTHER; note = phosphorothioate linkage
  modified_base 2: mod_base = OTHER; note = Locked Nucleic Acid
  modified_base 3: mod_base = OTHER; note = Locked Nucleic Acid
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12: mod_base = OTHER; note = Locked Nucleic Acid
  modified_base 12^13: mod_base = OTHER; note = phosphorothioate linkage
  modified_base 13: mod_base = OTHER; note = Locked Nucleic Acid
  modified_base 13^14: mod_base = OTHER; note = phosphorothioate linkage
  modified_base 14: mod_base = OTHER; note = Locked Nucleic Acid
tcctgcatctcgta

SEQ ID NO: 155 - DNA, 14 bp, synthetic construct
  source 1..14: mol_type = other DNA
  modified_base 1: mod_base = OTHER; note = Locked Nucleic Acid
  modified_base 1^2: mod_base = OTHER; note = phosphorothioate linkage
  modified_base 2: mod_base = OTHER; note = Locked Nucleic Acid
  modified_base 2^3: mod_base = OTHER; note = phosphorothioate linkage
  modified_base 3: mod_base = OTHER; note = Locked Nucleic Acid
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12: mod_base = OTHER; note = Locked Nucleic Acid
  modified_base 12^13: mod_base = OTHER; note = phosphorothioate linkage
  modified_base 13: mod_base = OTHER; note = Locked Nucleic Acid
  modified_base 13^14: mod_base = OTHER; note = phosphorothioate linkage
  modified_base 14: mod_base = OTHER; note = Locked Nucleic Acid
tggtccattctggt

SEQ ID NO: 156 - DNA, 14 bp, synthetic construct
  source 1..14: mol_type = other DNA
  modified_base 1: mod_base = OTHER; note = Locked Nucleic Acid
  modified_base 3: mod_base = OTHER; note = Locked Nucleic Acid
  modified_base 2: mod_base = OTHER; note = Locked Nucleic Acid
  modified_base 12: mod_base = OTHER; note = Locked Nucleic Acid
  modified_base 13: mod_base = OTHER; note = Locked Nucleic Acid
  modified_base 14: mod_base = OTHER; note = Locked Nucleic Acid
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
gtcactgaggtcca

SEQ ID NO: 157 - DNA, 14 bp, synthetic construct
  source 1..14: mol_type = other DNA
  modified_base 1: mod_base = OTHER; note = Locked Nucleic Acid
  modified_base 2: mod_base = OTHER; note = Locked Nucleic Acid
  modified_base 3: mod_base = OTHER; note = Locked Nucleic Acid
  modified_base 12: mod_base = OTHER; note = Locked Nucleic Acid
  modified_base 13: mod_base = OTHER; note = Locked Nucleic Acid
  modified_base 14: mod_base = OTHER; note = Locked Nucleic Acid
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
attgtcactgaggt

SEQ ID NO: 158 - DNA, 14 bp, synthetic construct
  source 1..14: mol_type = other DNA
  modified_base 1: mod_base = OTHER; note = Locked Nucleic Acid
  modified_base 2: mod_base = OTHER; note = Locked Nucleic Acid
  modified_base 3: mod_base = OTHER; note = Locked Nucleic Acid
  modified_base 12: mod_base = OTHER; note = Locked Nucleic Acid
  modified_base 13: mod_base = OTHER; note = Locked Nucleic Acid
  modified_base 14: mod_base = OTHER; note = Locked Nucleic Acid
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
gaatgttacagcca

SEQ ID NO: 159 - DNA, 14 bp, synthetic construct
  source 1..14: mol_type = other DNA
  modified_base 1: mod_base = OTHER; note = Locked Nucleic Acid
  modified_base 2: mod_base = OTHER; note = Locked Nucleic Acid
  modified_base 3: mod_base = OTHER; note = Locked Nucleic Acid
  modified_base 12: mod_base = OTHER; note = Locked Nucleic Acid
  modified_base 13: mod_base = OTHER; note = Locked Nucleic Acid
  modified_base 14: mod_base = OTHER; note = Locked Nucleic Acid
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
gcgccccaaccaca

SEQ ID NO: 160 - DNA, 14 bp, synthetic construct
  source 1..14: mol_type = other DNA
  modified_base 1: mod_base = OTHER; note = Locked Nucleic Acid
  modified_base 2: mod_base = OTHER; note = Locked Nucleic Acid
  modified_base 3: mod_base = OTHER; note = Locked Nucleic Acid
  modified_base 12: mod_base = OTHER; note = Locked Nucleic Acid
  modified_base 13: mod_base = OTHER; note = Locked Nucleic Acid
  modified_base 14: mod_base = OTHER; note = Locked Nucleic Acid
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
cagcgccccaacca

SEQ ID NO: 161 - DNA, 14 bp, synthetic construct
  source 1..14: mol_type = other DNA
  modified_base 1: mod_base = OTHER; note = Locked Nucleic Acid
  modified_base 2: mod_base = OTHER; note = Locked Nucleic Acid
  modified_base 3: mod_base = OTHER; note = Locked Nucleic Acid
  modified_base 12: mod_base = OTHER; note = Locked Nucleic Acid
  modified_base 14: mod_base = OTHER; note = Locked Nucleic Acid
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
atgtcgaagcagca

SEQ ID NO: 162 - DNA, 14 bp, synthetic construct
  source 1..14: mol_type = other DNA
  modified_base 1: mod_base = OTHER; note = Locked Nucleic Acid
  modified_base 2: mod_base = OTHER; note = Locked Nucleic Acid
  modified_base 3: mod_base = OTHER; note = Locked Nucleic Acid
  modified_base 12: mod_base = OTHER; note = Locked Nucleic Acid
  modified_base 13: mod_base = OTHER; note = Locked Nucleic Acid
  modified_base 14: mod_base = OTHER; note = Locked Nucleic Acid
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
gagatgtcgaagca

SEQ ID NO: 163 - DNA, 14 bp, synthetic construct
  source 1..14: mol_type = other DNA
  modified_base 1: mod_base = OTHER; note = Locked Nucleic Acid
  modified_base 2: mod_base = OTHER; note = Locked Nucleic Acid
  modified_base 3: mod_base = OTHER; note = Locked Nucleic Acid
  modified_base 13: mod_base = OTHER; note = Locked Nucleic Acid
  modified_base 14: mod_base = OTHER; note = Locked Nucleic Acid
  modified_base 12: mod_base = OTHER; note = Locked Nucleic Acid
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
aggagatgtcgaag

SEQ ID NO: 164 - DNA, 14 bp, synthetic construct
  source 1..14: mol_type = other DNA
  modified_base 1: mod_base = OTHER; note = Locked Nucleic Acid
  modified_base 2: mod_base = OTHER; note = Locked Nucleic Acid
  modified_base 3: mod_base = OTHER; note = Locked Nucleic Acid
  modified_base 12: mod_base = OTHER; note = Locked Nucleic Acid
  modified_base 13: mod_base = OTHER; note = Locked Nucleic Acid
  modified_base 14: mod_base = OTHER; note = Locked Nucleic Acid
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
agctccaccagctt

SEQ ID NO: 165 - DNA, 14 bp, synthetic construct
  source 1..14: mol_type = other DNA
  modified_base 1: mod_base = OTHER; note = Locked nucleic acid
  modified_base 2: mod_base = OTHER; note = Locked Nucleic Acid
  modified_base 3: mod_base = OTHER; note = Locked Nucleic Acid
  modified_base 12: mod_base = OTHER; note = Locked Nucleic Acid
  modified_base 13: mod_base = OTHER; note = Locked Nucleic Acid
  modified_base 14: mod_base = OTHER; note = Locked Nucleic Acid
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
cagtcccacacaca

SEQ ID NO: 166 - DNA, 14 bp, synthetic construct
  source 1..14: mol_type = other DNA
  modified_base 1: mod_base = OTHER; note = Locked Nucleic Acid
  modified_base 2: mod_base = OTHER; note = Locked Nucleic Acid
  modified_base 3: mod_base = OTHER; note = Locked Nucleic Acid
  modified_base 12: mod_base = OTHER; note = Locked Nucleic Acid
  modified_base 13: mod_base = OTHER; note = Locked Nucleic Acid
  modified_base 14: mod_base = OTHER; note = Locked Nucleic Acid
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
gcttcagtcccaca

SEQ ID NO: 167 - DNA, 14 bp, synthetic construct
  source 1..14: mol_type = other DNA
  modified_base 1: mod_base = OTHER; note = Locked Nucleic Acid
  modified_base 2: mod_base = OTHER; note = Locked Nucleic Acid
  modified_base 3: mod_base = OTHER; note = Locked Nucleic Acid
  modified_base 12: mod_base = OTHER; note = Locked Nucleic Acid
  modified_base 13: mod_base = OTHER; note = Locked Nucleic Acid
  modified_base 14: mod_base = OTHER; note = Locked Nucleic Acid
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
gtgcttcagtccca

SEQ ID NO: 168 - DNA, 14 bp, synthetic construct
  source 1..14: mol_type = other DNA
  modified_base 1: mod_base = OTHER; note = Locked Nucleic Acid
  modified_base 2: mod_base = OTHER; note = Locked Nucleic Acid
  modified_base 3: mod_base = OTHER; note = Locked Nucleic Acid
  modified_base 13: mod_base = OTHER; note = Locked Nucleic Acid
  modified_base 14: mod_base = OTHER; note = Locked Nucleic Acid
  modified_base 12: mod_base = OTHER; note = Locked Nucleic Acid
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
aggtgcttcagtcc

SEQ ID NO: 169 - DNA, 14 bp, synthetic construct
  source 1..14: mol_type = other DNA
  modified_base 1: mod_base = OTHER; note = Locked Nucleic Acid
  modified_base 2: mod_base = OTHER; note = Locked Nucleic Acid
  modified_base 3: mod_base = OTHER; note = Locked Nucleic Acid
  modified_base 12: mod_base = OTHER; note = Locked Nucleic Acid
  modified_base 13: mod_base = OTHER; note = Locked Nucleic Acid
  modified_base 14: mod_base = OTHER; note = Locked Nucleic Acid
  modified_base 13^14: mod_base = OTHER; note = phosphorothioate linkage
  modified_base 12^13: mod_base = OTHER; note = phosphorothioate linkage
  modified_base 11^12: mod_base = OTHER; note = phosphorothioate linkage
  modified_base 10^11: mod_base = OTHER; note = phosphorothioate linkage
  modified_base 9^10: mod_base = OTHER; note = phosphorothioate linkage
  modified_base 8^9: mod_base = OTHER; note = phosphorothioate linkage
  modified_base 7^8: mod_base = OTHER; note = phosphorothioate linkage
  modified_base 6^7: mod_base = OTHER; note = phosphorothioate linkage
  modified_base 5^6: mod_base = OTHER; note = phosphorothioate linkage
  modified_base 4^5: mod_base = OTHER; note = phosphorothioate linkage
  modified_base 3^4: mod_base = OTHER; note = phosphorothioate linkage
  modified_base 2^3: mod_base = OTHER; note = phosphorothioate linkage
  modified_base 1^2: mod_base = OTHER; note = phosphorothioate linkage
gtctcccaaggcat

SEQ ID NO: 170 - DNA, 14 bp, synthetic construct
  source 1..14: mol_type = other DNA
  modified_base 1: mod_base = OTHER; note = Locked Nucleic Acid
  modified_base 2: mod_base = OTHER; note = Locked Nucleic Acid
  modified_base 3: mod_base = OTHER; note = Locked Nucleic Acid
  modified_base 12: mod_base = OTHER; note = Locked Nucleic Acid
  modified_base 13: mod_base = OTHER; note = Locked Nucleic Acid
  modified_base 14: mod_base = OTHER; note = Locked Nucleic Acid
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
ctcctgagtctccc

SEQ ID NO: 171 - DNA, 14 bp, synthetic construct
  source 1..14: mol_type = other DNA
  modified_base 1: mod_base = OTHER; note = Locked Nucleic Acid
  modified_base 2: mod_base = OTHER; note = Locked Nucleic Acid
  modified_base 3: mod_base = OTHER; note = Locked Nucleic Acid
  modified_base 12: mod_base = OTHER; note = Locked Nucleic Acid
  modified_base 13: mod_base = OTHER; note = Locked Nucleic Acid
  modified_base 14: mod_base = OTHER; note = Locked Nucleic Acid
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
gactcctgagtctc

SEQ ID NO: 172 - DNA, 14 bp, synthetic construct
  source 1..14: mol_type = other DNA
  modified_base 1: mod_base = OTHER; note = Locked Nucleic Acid
  modified_base 2: mod_base = OTHER; note = Locked Nucleic Acid
  modified_base 3: mod_base = OTHER; note = Locked Nucleic Acid
  modified_base 12: mod_base = OTHER; note = Locked Nucleic Acid
  modified_base 13: mod_base = OTHER; note = Locked Nucleic Acid
  modified_base 14: mod_base = OTHER; note = Locked Nucleic Acid
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
attcaccaacccca

SEQ ID NO: 173 - DNA, 14 bp, synthetic construct
  source 1..14: mol_type = other DNA
  modified_base 1: mod_base = OTHER; note = Locked Nucleic Acid
  modified_base 2: mod_base = OTHER; note = Locked Nucleic Acid
  modified_base 3: mod_base = OTHER; note = Locked Nucleic Acid
  modified_base 12: mod_base = OTHER; note = Locked Nucleic Acid
  modified_base 13: mod_base = OTHER; note = Locked Nucleic Acid
  modified_base 14: mod_base = OTHER; note = Locked Nucleic Acid
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
gaattcaccaaccc

SEQ ID NO: 174 - DNA, 14 bp, synthetic construct
  source 1..14: mol_type = other DNA
  modified_base 1: mod_base = OTHER; note = Locked Nucleic Acid
  modified_base 2: mod_base = OTHER; note = Locked Nucleic Acid
  modified_base 3: mod_base = OTHER; note = Locked Nucleic Acid
  modified_base 12: mod_base = OTHER; note = Locked Nucleic Acid
  modified_base 13: mod_base = OTHER; note = Locked Nucleic Acid
  modified_base 14: mod_base = OTHER; note = Locked Nucleic Acid
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
agctgaacaacaga

SEQ ID NO: 175 - DNA, 14 bp, synthetic construct
  source 1..14: mol_type = other DNA
  modified_base 1: mod_base = OTHER; note = Locked Nucleic Acid
  modified_base 2: mod_base = OTHER; note = Locked Nucleic Acid
  modified_base 3: mod_base = OTHER; note = Locked Nucleic Acid
  modified_base 12: mod_base = OTHER; note = Locked Nucleic Acid
  modified_base 13: mod_base = OTHER; note = Locked Nucleic Acid
  modified_base 14: mod_base = OTHER; note = Locked Nucleic Acid
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
gttgtctaattcca

SEQ ID NO: 176 - DNA, 14 bp, synthetic construct
  source 1..14: mol_type = other DNA
  modified_base 1: mod_base = OTHER; note = Locked Nucleic Acid
  modified_base 2: mod_base = OTHER; note = Locked Nucleic Acid
  modified_base 3: mod_base = OTHER; note = Locked Nucleic Acid
  modified_base 12: mod_base = OTHER; note = Locked Nucleic Acid
  modified_base 13: mod_base = OTHER; note = Locked Nucleic Acid
  modified_base 14: mod_base = OTHER; note = Locked Nucleic Acid
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
cagttgtctaattc

SEQ ID NO: 177 - DNA, 14 bp, synthetic construct
  source 1..14: mol_type = other DNA
  modified_base 1: mod_base = OTHER; note = Locked Nucleic Acid
  modified_base 2: mod_base = OTHER; note = Locked Nucleic Acid
  modified_base 3: mod_base = OTHER; note = Locked Nucleic Acid
  modified_base 12: mod_base = OTHER; note = Locked Nucleic Acid
  modified_base 13: mod_base = OTHER; note = Locked Nucleic Acid
  modified_base 14: mod_base = OTHER; note = Locked Nucleic Acid
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
tgcagttgtctaat

SEQ ID NO: 178 - DNA, 14 bp, synthetic construct
  source 1..14: mol_type = other DNA
  modified_base 1: mod_base = OTHER; note = Locked Nucleic Acid
  modified_base 2: mod_base = OTHER; note = Locked Nucleic Acid
  modified_base 3: mod_base = OTHER; note = Locked Nucleic Acid
  modified_base 12: mod_base = OTHER; note = Locked Nucleic Acid
  modified_base 13: mod_base = OTHER; note = Locked Nucleic Acid
  modified_base 14: mod_base = OTHER; note = Locked Nucleic Acid
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 13^14: mod_base = OTHER; note = phosphorothioate linkage
  modified_base 12^13: mod_base = OTHER; note = phosphorothioate linkage
cattgttgcccagg

SEQ ID NO: 179 - DNA, 14 bp, synthetic construct
  source 1..14: mol_type = other DNA
  modified_base 1: mod_base = OTHER; note = Locked Nucleic Acid
  modified_base 2: mod_base = OTHER; note = Locked Nucleic Acid
  modified_base 3: mod_base = OTHER; note = Locked Nucleic Acid
  modified_base 12: mod_base = OTHER; note = Locked Nucleic Acid
  modified_base 13: mod_base = OTHER; note = Locked Nucleic Acid
  modified_base 14: mod_base = OTHER; note = Locked Nucleic Acid
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
tgctgtttcagcac

SEQ ID NO: 180 - DNA, 14 bp, synthetic construct
  source 1..14: mol_type = other DNA
  modified_base 1: mod_base = OTHER; note = Locked Nucleic Acid
  modified_base 2: mod_base = OTHER; note = Locked Nucleic Acid
  modified_base 3: mod_base = OTHER; note = Locked Nucleic Acid
  modified_base 12: mod_base = OTHER; note = Locked Nucleic Acid
  modified_base 13: mod_base = OTHER; note = Locked Nucleic Acid
  modified_base 14: mod_base = OTHER; note = Locked Nucleic Acid
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
ctcaggcttttctt

SEQ ID NO: 181 - DNA, 14 bp, synthetic construct
  source 1..14: mol_type = other DNA
  modified_base 1: mod_base = OTHER; note = Locked Nucleic Acid
  modified_base 2: mod_base = OTHER; note = Locked Nucleic Acid
  modified_base 3: mod_base = OTHER; note = Locked Nucleic Acid
  modified_base 12: mod_base = OTHER; note = Locked Nucleic Acid
  modified_base 13: mod_base = OTHER; note = Locked Nucleic Acid
  modified_base 14: mod_base = OTHER; note = Locked Nucleic Acid
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
agctcaggcttttc

SEQ ID NO: 182 - DNA, 14 bp, synthetic construct
  source 1..14: mol_type = other DNA
  modified_base 1: mod_base = OTHER; note = Locked Nucleic Acid
  modified_base 2: mod_base = OTHER; note = Locked Nucleic Acid
  modified_base 3: mod_base = OTHER; note = Locked Nucleic Acid
  modified_base 12: mod_base = OTHER; note = Locked Nucleic Acid
  modified_base 13: mod_base = OTHER; note = Locked Nucleic Acid
  modified_base 14: mod_base = OTHER; note = Locked Nucleic Acid
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
tcagctcaggcttt

SEQ ID NO: 183 - DNA, 14 bp, synthetic construct
  source 1..14: mol_type = other DNA
  modified_base 1: mod_base = OTHER; note = Locked Nucleic Acid
  modified_base 2: mod_base = OTHER; note = Locked Nucleic Acid
  modified_base 3: mod_base = OTHER; note = Locked Nucleic Acid
  modified_base 12: mod_base = OTHER; note = Locked Nucleic Acid
  modified_base 13: mod_base = OTHER; note = Locked Nucleic Acid
  modified_base 14: mod_base = OTHER; note = Locked Nucleic Acid
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
cgtggctagatcca

SEQ ID NO: 184 - DNA, 14 bp, synthetic construct
  source 1..14: mol_type = other DNA
  modified_base 1: mod_base = OTHER; note = Locked Nucleic Acid
  modified_base 2: mod_base = OTHER; note = Locked Nucleic Acid
  modified_base 3: mod_base = OTHER; note = Locked Nucleic Acid
  modified_base 12: mod_base = OTHER; note = Locked Nucleic Acid
  modified_base 13: mod_base = OTHER; note = Locked Nucleic Acid
  modified_base 14: mod_base = OTHER; note = Locked Nucleic Acid
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
tcctcttccagcag

SEQ ID NO: 185 - DNA, 14 bp, synthetic construct
  source 1..14: mol_type = other DNA
  modified_base 1: mod_base = OTHER; note = Locked Nucleic Acid
  modified_base 2: mod_base = OTHER; note = Locked Nucleic Acid
  modified_base 3: mod_base = OTHER; note = Locked Nucleic Acid
  modified_base 12: mod_base = OTHER; note = Locked Nucleic Acid
  modified_base 13: mod_base = OTHER; note = Locked Nucleic Acid
  modified_base 14: mod_base = OTHER; note = Locked Nucleic Acid
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
ttcctcttccagca

SEQ ID NO: 186 - DNA, 14 bp, synthetic construct
  source 1..14: mol_type = other DNA
  modified_base 1: mod_base = OTHER; note = Locked Nucleic Acid
  modified_base 2: mod_base = OTHER; note = Locked Nucleic Acid
  modified_base 3: mod_base = OTHER; note = Locked Nucleic Acid
  modified_base 12: mod_base = OTHER; note = Locked Nucleic Acid
  modified_base 13: mod_base = OTHER; note = Locked Nucleic Acid
  modified_base 14: mod_base = OTHER; note = Locked Nucleic Acid
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
acaacaaaaatcaa

SEQ ID NO: 187 - DNA, 14 bp, synthetic construct
  source 1..14: mol_type = other DNA
  modified_base 1: mod_base = OTHER; note = Locked Nucleic Acid
  modified_base 2: mod_base = OTHER; note = Locked Nucleic Acid
  modified_base 3: mod_base = OTHER; note = Locked Nucleic Acid
  modified_base 12: mod_base = OTHER; note = Locked Nucleic Acid
  modified_base 13: mod_base = OTHER; note = Locked Nucleic Acid
  modified_base 14: mod_base = OTHER; note = Locked Nucleic Acid
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
agagatcttgcaac

SEQ ID NO: 188 - DNA, 14 bp, synthetic construct
  source 1..14: mol_type = other DNA
  modified_base 1: mod_base = OTHER; note = Locked Nucleic Acid
  modified_base 2: mod_base = OTHER; note = Locked Nucleic Acid
  modified_base 3: mod_base = OTHER; note = Locked Nucleic Acid
  modified_base 12: mod_base = OTHER; note = Locked Nucleic Acid
  modified_base 13: mod_base = OTHER; note = Locked Nucleic Acid
  modified_base 14: mod_base = OTHER; note = Locked Nucleic Acid
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
tgcttcagtcccac

SEQ ID NO: 189 - DNA, 14 bp, synthetic construct
  source 1..14: mol_type = other DNA
  modified_base 1: mod_base = OTHER; note = Locked Nucleic Acid
  modified_base 2: mod_base = OTHER; note = Locked Nucleic Acid
  modified_base 3: mod_base = OTHER; note = Locked Nucleic Acid
  modified_base 12: mod_base = OTHER; note = Locked Nucleic Acid
  modified_base 13: mod_base = OTHER; note = Locked Nucleic Acid
  modified_base 14: mod_base = OTHER; note = Locked Nucleic Acid
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
gcagttgtctaatt

SEQ ID NO: 190-645 - skipped (456 entries)

SEQ ID NO: 646 - DNA, 14 bp, synthetic construct
  source 1..14: mol_type = other DNA
  modified_base 1: mod_base = OTHER; note = Locked Nucleic Acid
  modified_base 2: mod_base = OTHER; note = Locked Nucleic Acid
  modified_base 3: mod_base = OTHER; note = Locked Nucleic Acid
  modified_base 12: mod_base = OTHER; note = Locked Nucleic Acid
  modified_base 14: mod_base = OTHER; note = Locked Nucleic Acid
  modified_base 13: mod_base = OTHER; note = Locked Nucleic Acid
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 12^13: mod_base = OTHER; note = phosphorothioate linkage
  modified_base 13^14: mod_base = OTHER; note = phosphorothioate linkage
ctatgtgctcgtca

SEQ ID NO: 647 - DNA, 16 bp, synthetic construct
  source 1..16: mol_type = other DNA
  modified_base 1: mod_base = OTHER; note = Locked Nucleic Acid
  modified_base 2: mod_base = OTHER; note = Locked Nucleic Acid
  modified_base 3: mod_base = OTHER; note = Locked Nucleic Acid
  modified_base 14: mod_base = OTHER; note = Locked Nucleic Acid
  modified_base 15: mod_base = OTHER; note = Locked Nucleic Acid
  modified_base 16: mod_base = OTHER; note = Locked Nucleic Acid
  modified_base 1^2: mod_base = OTHER; note = phosphorothioate linkage
  modified_base 2^3: mod_base = OTHER; note = phosphorothioate linkage
  modified_base 3^4: mod_base = OTHER; note = phosphorothioate linkage
  modified_base 4^5: mod_base = OTHER; note = phosphorothioate linkage
  modified_base 5^6: mod_base = OTHER; note = phosphorothioate linkage
  modified_base 6^7: mod_base = OTHER; note = phosphorothioate linkage
  modified_base 7^8: mod_base = OTHER; note = phosphorothioate linkage
  modified_base 8^9: mod_base = OTHER; note = phosphorothioate linkage
  modified_base 9^10: mod_base = OTHER; note = phosphorothioate linkage
  modified_base 10^11: mod_base = OTHER; note = phosphorothioate linkage
  modified_base 11^12: mod_base = OTHER; note = phosphorothioate linkage
  modified_base 13^14: mod_base = OTHER; note = phosphorothioate linkage
  modified_base 12^13: mod_base = OTHER; note = phosphorothioate linkage
  modified_base 14^15: mod_base = OTHER; note = phosphorothioate linkage
  modified_base 15^16: mod_base = OTHER; note = phosphorothioate linkage
  modified_base 13^14: mod_base = OTHER; note = phosphorothioate linkage
cctatgtgctcgtcaa

(19) United States
(12) Patent Application Publication
(10) Pub. No.: US 2024/0409931 A1
(43) Pub. Date: Dec. 12, 2024

(54) COMPOSITIONS AND METHODS FOR MODULATING NLRP3 OR NLRP1 EXPRESSION

(71) Applicant: Molecular Axiom, LLC, San Diego, CA (US)

(72) Inventors: Lishan CHEN, San Diego, CA (US); Bohan JIN, San Diego, CA (US); Timothy Preston WONG, San Diego, CA (US)

(21) Appl. No.: 18/688,512

(22) PCT Filed: Sep. 1, 2022
(86) PCT No.: PCT/US2022/042394

§ 371 Date: Mar. 1, 2024

Related U.S. Application Data
(60) Provisional application No. 63/340,192, filed on May 10, 2022.
(60) Provisional application No. 63/240,219, filed on Sep. 2, 2021.

(51) Int. Cl. C12N 15/113

(52) U.S. Cl. CPC C12N 15/113; C12N 2310/11; C12N 2310/315; C12N 2310/3231

(57) ABSTRACT

Described herein are compositions of an antisense oligonucleotide capable of binding to NACHT, LRR and PYD domains-containing protein 3 (NLRP3) or NLRP1 mRNA for modulating gene expressions. Also described herein are methods for using the compositions described herein for modulating gene expressions or for treating inflammasome-associate disease.

### CROSS-REFERENCE

This application claims the benefit of U.S. Provisional Application Ser. No. 63/240,219 filed on Sep. 2, 2021, and U.S. Provisional Application Ser. No. 63/340,192 filed on May 10, 2022, the entirety of which is hereby incorporated by reference herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BACKGROUND

Certain diseases or conditions are caused by inflammation or over- or under-expression of one or more genes of pathway associated with inflammation. To treat such diseases or conditions, one of the most sought-after treatment option involves direct editing of the genes associated with inflammation, or transcriptional/translational regulation using gene silencing tools or methods. RNA-induced gene silencing controls RNA expression of target genes in various aspects including transcription inactivation, mRNA degradation, transcriptional attenuation. Therefore, there remains a need for compositions and methods for effective editing gene expression at RNA levels.

### SUMMARY

Described herein, in some aspects, is a composition comprising an antisense oligonucleotide capable of binding to NACHT, LRR and PYD domains-containing protein 3 (NLRP3) or NLRP1 mRNA. In some aspects, the antisense oligonucleotide specifically binds to the NLRP3 mRNA. In some aspects, the antisense oligonucleotide comprises a nucleic acid sequence that is complementary to a nucleic acid sequence encoding NLRP3 mRNA. In some aspects, the antisense oligonucleotide comprises a nucleic acid sequence that is at least 80%, at least 90%, or at least 95% complementary to a mRNA encoded by SEQ ID NOs: 1-7. In some aspects, the antisense oligonucleotide specifically binds to the NLRP1 mRNA. In some aspects, the antisense oligonucleotide comprises a nucleic acid sequence that is complementary to a nucleic acid sequence encoding NLRP1 mRNA. In some aspects, the antisense oligonucleotide comprises a nucleic acid sequence that is at least 80%, at least 90%, or at least 95% complementary to a mRNA encoded by SEQ ID NOs: 8-13. In some aspects, the antisense oligonucleotide specifically binds to the NLRP3 mRNA and NLRP1 mRNA. In some aspects, the antisense oligonucleotide comprises a nucleic acid sequence that is complementary to a nucleic acid sequence encoding NLRP3 mRNA and NLRP1 mRNA. In some aspects, the antisense oligonucleotide comprises a nucleic acid sequence that is complementary to mRNA encoded by SEQ ID NO: 1 and SEQ ID NO: 8. In some aspects, the antisense oligonucleotide comprises a nucleic acid sequence that has at least 80%, 85%, 90%, 95%, or 99% sequence similarity to one of the following sequences: SEQ ID NOs: 21-38. In some aspects, the antisense oligonucleotide comprises 12-30 nucleic acid bases in length. In some aspects, the antisense oligonucleotide comprises a gap segment and a wing segment. In some aspects, the antisense oligonucleotide comprises 5′-wing segment and 3′-wing segment. In some aspects, the each of the 5′-wing segment and 3′-wing segment comprises three nucleic acid bases in length. In some aspects, the antisense oligonucleotide comprises at least one 2′-modified nucleoside, at least one modified internucleotide linkage, or at least one inverted abasic moiety. In some aspects, the at least one 2′ modified nucleotide comprises 2′-O-methyl, 2′-O-methoxyethyl (2′-0-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA) modified nucleotide, locked nucleic acid (LNA), constrained ethyl (cEt) sugar, thiomorpholino, ethylene nucleic acid (ENA), or a combination thereof. In some aspects, the at least one modified internucleotide linkage comprises a phosphorothioate linkage or a phosphorodithioate linkage. In some aspects, the antisense oligonucleotide comprises a phosphorodiamidate morpholino oligomer (PMO), thiomorpholino, locked nucleic acid (LNA), or constrained ethyl (cEt) sugar. In some aspects, the antisense oligonucleotide is conjugated with a peptide, antibody, lipid, carbohydrates, or a polymer. In some aspects, the antisense oligonucleotide is conjugated with a peptide, antibody, lipid, carbohydrates, or a polymer via a linker. In some aspects, the composition comprises a combination of an antisense oligonucleotide specifically binds to the NLRP3 mRNA and an antisense oligonucleotide specifically binds to the NLRP1 mRNA. In some aspects, the composition comprises an antisense oligonucleotide capable of binding to both NLRP3 mRNA and NLRP1 mRNA. In some aspects, the composition further comprises an excipient. In some aspects, the composition is formulated for parenteral or nasal administration. In some embodiments, the antisense oligonucleotide comprises a nucleic acid sequence that is at least 80%, 85%, 90%, 95%, or 99% identical to any one of SEQ ID NOs: 95, 96, 97, 98, 99, 100, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, or 646. In some embodiments, the antisense oligonucleotide comprises the nucleic acid sequence that is at least 80%, 85%, 90%, 95%, or 99% identical to any one of SEQ ID NOs: 95, 121, 139, 144, 146, 147, or 172. In some embodiments, the antisense oligonucleotide comprises the nucleic acid sequence that is at least 80%, 85%, 90%, 95%, or 99% identical to SEQ ID NO: 95. In some embodiments, the antisense oligonucleotide comprises the nucleic acid sequence that is at least 80%, 85%, 90%, 95%, or 99% identical to SEQ ID NO: 121. In some embodiments, the antisense oligonucleotide comprises the nucleic acid sequence that is at least 80%, 85%, 90%, 95%, or 99% identical to SEQ ID NO: 139. In some embodiments, the antisense oligonucleotide comprises the nucleic acid sequence that is at least 80%, 85%, 90%, 95%, or 99% identical to SEQ ID NO: 144. In some embodiments, the antisense oligonucleotide comprises the nucleic acid sequence that is at least 80%, 85%, 90%, 95%, or 99% identical to SEQ ID NO: 146. In some embodiments, the antisense oligonucleotide comprises the nucleic acid sequence that is at least 80%, 85%, 90%, 95%, or 99% identical to SEQ ID NO: 147. In some embodiments, the antisense oligonucleotide comprises the nucleic acid sequence that is at least 80%, 85%, 90%, 95%, or 99% identical to SEQ ID NO: 172. In some embodiments, the antisense oligonucleotide is any one of SEQ ID NOs: 95, 121, 139, 144, 146, 147, or 172. In some embodiments, the antisense oligonucleotide is SEQ ID NO: 95. In some embodiments, the antisense oligonucleotide is SEQ ID NO: 121. In some embodiments, the antisense oligonucleotide is SEQ ID NO: 139. In some embodiments, the antisense oligonucleotide is SEQ ID NO: 144. In some embodiments, the antisense oligonucleotide is SEQ ID NO: 146. In some embodiments, the antisense oligonucleotide is SEQ ID NO: 147. In some embodiments, the antisense oligonucleotide is SEQ ID NO: 172.

Described herein, in some aspects, is a method of modulating an inflammasome pathway in a cell, comprising: treating the cell with a composition comprising antisense oligonucleotide capable of binding to NLRP3 or NLRP1 mRNA, thereby reducing expression of NLRP3, NLRP1, or inflammasome in the cell. In some aspects, the cell is associated with an inflammasome disease or condition. In some aspects, the antisense oligonucleotide comprises at least one 2′-modified nucleotide, at least one modified internucleotide linkage, or at least one inverted abasic moiety. In some aspects, the at least one 2′ modified nucleotide: comprises 2-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA) modified nucleotide; comprises locked nucleic acid (LNA), locked nucleic acid (LNA), constrained ethyl (cEt) sugar or ethylene nucleic acid (ENA); or comprises a combination thereof. In some aspects, the expression of NLRP3 or NLRP1 protein or mRNA is reduced at least 30%, at least 40%, at least 50% after the treatment. In some aspects, the expression of NLRP3 or NLRP1 protein or mRNA is reduced at least 30%, at least 40%, at least 50% compared to an untreated cell. In some aspects, the cells treated with the composition increase at least one cytokine expression.

Described herein, in some aspects, is a method of treating a disease or condition associated with inflammasome in a subject in need thereof, the method comprising: administering the subject a composition of any one of claims 1-25, thereby treating the disease or condition associated with inflammasome in the subject. In some aspects, the disease or condition associated with inflammasome comprises autoinflammatory disease, autoimmune disease, neurodegenerative disease. In some aspects, the autoimmune disease comprises a diabetes or inflammatory bowel syndrome (IBD). In some aspects, the neurodegenerative disease comprises migraine, amyotrophic lateral sclerosis (ALS), Parkinson's disease, Alzheimer's disease or Huntington's disease. In some aspects, the disease or condition comprises a nerve injury. In some aspects, the nerve injury comprises a peripheral nerve injury. In some aspects, the nerve injury comprises a spinal cord injury.

### BRIEF DESCRIPTION OF THE DRAWINGS

This patent application contains at least one drawing executed in color. Copies of this patent or patent application with color drawing(s) will be provided by the Office upon request and payment of the necessary fee.

FIG. 1 illustrates NLRP3 mRNA knockdown in U87 human glioblastoma cells 24 hours after transfection.

FIG. 2 illustrates NLRP3 mRNA knockdown in LPS+ATP stimulated mouse microglia.

FIG. 3 illustrates IL-1 beta suppression following 72 hour ASO treatment in LPS+ATP stimulated mouse microglia.

FIG. 4 illustrates IL-1 beta suppression in LPS+ATP stimulated human PBMC.

The novel features of the disclosure are set forth with particularity in the appended claims. A better understanding of the features and advantages of the present disclosure will be obtained by reference to the following detailed description that sets forth illustrative embodiments.

### DETAILED DESCRIPTION

### Overview

Described herein are compositions and methods for modulating gene expression or pathway associated with inflammation. Also described herein are composition and methods for treating an inflammatory disease or condition by modulating the gene expression or pathway associated with the inflammatory disease or condition. In some aspects, the composition comprises at least one oligonucleotide that, upon delivered into a cell, binds to an endogenous nucleic acid, which leads to the degradation of the target nucleic acid. In some aspects, described herein is a method for utilizing the composition or the oligonucleotide described herein. In some aspects, the methods treat inflammatory diseases or conditions by contacting a cell with the oligonucleotide to decrease the gene expression or pathway associated with the inflammatory diseases or conditions.

In some aspects, the oligonucleotide is an antisense oligonucleotide, where the oligonucleotide is complementary and binds to at least one endogenous nucleic acid (e.g., a pre-mRNA or a mRNA). In some aspects, the binding of the oligonucleotide to the endogenous nucleic acid leads to degradation of the endogenous nucleic acid or blocking of translation of the target protein encoded from the endogenous nucleic acid, hence decreasing the expression of the gene encoded by the endogenous nucleic acid. For example, the binding of the oligonucleotide to the endogenous nucleic acid comprising an mRNA creates a duplex nucleic acid molecule, which can then recruit endogenous nuclease for degradation of the mRNA. In some cases, the binding of the oligonucleotide to the endogenous nucleic acid leads to degradation of the endogenous mRNA encoding the target protein, where the decreased translation of the target protein leads to decreased assembly of inflammasome.

In some aspects, the gene modulated by the oligonucleotide is part of the pathway. In some aspects, the pathway is an inflammation pathway. As such, In some aspects, the decreasing of the expression of the gene due to the binding of the oligonucleotide to the endogenous nucleic acid can further decrease an inflammasome pathway expression comprising the gene modulated by the oligonucleotide. In some aspects, the decreasing gene or pathway expression leads to therapeutic effects for treating the inflammatory disease or condition. In some aspects, the inflammatory disease or condition is caused by increased gene expression or inflammasome pathway.

In some aspects, the oligonucleotide comprises at least one gap segment. In some aspects, the oligonucleotide comprises at least one wing segment. In some aspects, the oligonucleotide comprises at least one gap segment flanked by two wing segments. For example, the oligonucleotide comprises a gap segment flanked by a 5′-wing segment and a 3′-wing segment. In some aspects, the gap segment or the wing segment comprises at least one chemical modification. In some aspects, the at least one chemical modification increases specificity of the oligonucleotide binding to the endogenous nucleic acid. In some aspects, the at least one chemical modification increases affinity of the oligonucleotide binding to the endogenous nucleic acid. In some aspects, the at least one chemical modification increases resistance of the oligonucleotide to hydrolysis. In some aspects, the at least one chemical modification increases resistance of the oligonucleotide to nuclease digestion. In some aspects, the at least one chemical modification increases half-life of the oligonucleotide in vivo. In some aspects, the at least one chemical modification decreases immunogenicity. In some aspects, the at least one chemical modification decreases innate immune response.

### Compositions

Described herein, In some aspects, are compositions comprising at least one oligonucleotide described herein. In some aspects, the composition comprises at least two, three, four, five, six, seven, eight, nine, ten, or more oligonucleotides. In some aspects, the oligonucleotides comprise same or difference nucleic acid sequences. In some aspects, the oligonucleotide described herein is an antisense oligonucleotide for targeting and bind to an endogenous nucleic acid. In some aspects, the binding of the oligonucleotide to the endogenous nucleic acid recruits endogenous nuclease for degrading the endogenous nucleic acid. In some aspects, the degradation of the endogenous nucleic acid decreases expression of the gene encoded by the endogenous nucleic acid. In some aspects, the degradation of the endogenous nucleic acid can treat a disease or condition described herein.

In some aspects, the oligonucleotide comprises a length of at least five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, or more nucleic acid bases. In some aspects, the oligonucleotide comprises a length of at least 10, 11, 12, 13, 14, 15, 16, 17, 18, 19, or 20 nucleic acid bases. In some aspects, the oligonucleotide comprises 10 nucleic acid bases. In some aspects, the oligonucleotide comprises 11 nucleic acid bases. In some aspects, the oligonucleotide comprises 12 nucleic acid bases. In some aspects, the oligonucleotide comprises 13 nucleic acid bases. In some aspects, the oligonucleotide comprises 14 nucleic acid bases. In some aspects, the oligonucleotide comprises 15 nucleic acid bases. In some aspects, the oligonucleotide comprises 16 nucleic acid bases. In some aspects, the oligonucleotide comprises 17 nucleic acid bases. In some aspects, the oligonucleotide comprises 18 nucleic acid bases. In some aspects, the oligonucleotide comprises 19 nucleic acid bases. In some aspects, the oligonucleotide comprises 20 nucleic acid bases.

In some aspects, the oligonucleotide comprises at least one gap segment. In some aspects, the gap segment comprises at least one, two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, or more nucleic acid bases. In some aspects, the gap segment comprises at least one four, five, six, seven, eight, nine, 10, 11, 12, 13, or 14 nucleic acid bases. In some aspects, the gap segment comprises four nucleic acid bases. In some aspects, the gap segment comprises five nucleic acid bases. In some aspects, the gap segment comprises six nucleic acid bases. In some aspects, the gap segment comprises seven nucleic acid bases. In some aspects, the gap segment comprises eight nucleic acid bases. In some aspects, the gap segment comprises nine nucleic acid bases. In some aspects, the gap segment comprises 10 nucleic acid bases. In some aspects, the gap segment comprises 11 nucleic acid bases. In some aspects, the gap segment comprises 12 nucleic acid bases. In some aspects, the gap segment comprises 13 nucleic acid bases. In some aspects, the gap segment comprises 14 nucleic acid bases.

In some aspects, the oligonucleotide comprises at least one wing segment. In some aspects, the at least one wing segment is a 5′-end wing segment that is covalently connected to the gap segment at the 5′-end of the gap segment. In some aspects, the at least one wing segment is a 3′-end wing segment that is covalently connected to the gap segment at the 3′-end of the gap segment. In some aspects, the gap segment is flanked by the wing segments at both the 5′-end and the 3′-end of the gap segment. In some aspects, the wing segment comprises at least one, two, three, four, five, six, seven, eight, nine, 10, 11, 12, or more nucleic acid bases. In some aspects, the wing segment comprises one nucleic acid base. In some aspects, the wing segment comprises two nucleic acid bases. In some aspects, the wing segment comprises three nucleic acid bases. In some aspects, the wing segment comprises four nucleic acid bases. In some aspects, the wing segment comprises five nucleic acid bases. In some aspects, the wing segment comprises six nucleotides. In some aspects, the wing segment comprises seven nucleic acid bases. In some aspects, the wing segment comprises eight nucleic acid bases. In some aspects, the wing segment comprises nine nucleic acid bases. In some aspects, the wing segment comprises 10 nucleic acid bases.

In some aspects, the oligonucleotide comprises a 5′-end wing segment followed by a gap segment followed by a 3′-end wing segment. In such arrangement, the 5′-end wing segment comprises one, two, three, four, five, six, seven, eight, nine, 10, 11, 12, or more nucleic acid bases and the 3′-end wing segment comprises one, two, three, four, five, six, seven, eight, nine, 10, 11, 12, or more nucleic acid bases. In some aspects, the 5′-end wing segment and the 3′end wing segment comprises the same number of nucleic acid base. In some aspects, the 5′-end wing segment and the 3′end wing segment comprises a different number of nucleic acid bases. In some aspects, the 5′-end wing segment comprises one nucleic acid base. In some aspects, the 5′-end wing segment comprises two nucleic acid bases. In some aspects, the 5′-end wing segment comprises three nucleic acid bases. In some aspects, the 5′-end wing segment comprises four nucleic acid bases. In some aspects, the 5′-end wing segment comprises five nucleic acid bases. In some aspects, the 5′-end wing segment comprises six nucleic acid bases. In some aspects, the 5′-end wing segment comprises seven nucleic acid bases. In some aspects, the 5′-end wing segment comprises eight nucleic acid bases. In some aspects, the 5′-end wing segment comprises nine nucleic acid bases. In some aspects, the 5′-end wing segment comprises 10 nucleic acid bases. In some aspects, the 3′-end wing segment comprises one nucleic acid base. In some aspects, the 3′-end wing segment comprises two nucleic acid bases. In some aspects, the 3′-end wing segment comprises three nucleic acid bases. In some aspects, the 3′-end wing segment comprises four nucleic acid bases. In some aspects, the 3′-end wing segment comprises five nucleic acid bases. In some aspects, the 3′-end wing segment comprises six nucleic acid bases. In some aspects, the 3′-end wing segment comprises seven nucleic acid bases. In some aspects, the 3′-end wing segment comprises eight nucleic acid bases. In some aspects, the 3′-end wing segment comprises nine nucleic acid bases. In some aspects, the 3′-end wing segment comprises 10 nucleic acid bases.

In some aspects, the oligonucleotide comprises a 5′-end wing segment comprising one nucleic acid base and a 3′-end wing segment comprising one nucleic acid base. In some aspects, the oligonucleotide comprises a 5′-end wing segment comprising two nucleic acid bases and a 3′-end wing segment comprising two nucleic acid bases. In some aspects, the oligonucleotide comprises a 5′-end wing segment comprising three nucleic acid bases and a 3′-end wing segment comprising three nucleic acid bases. In some aspects, the oligonucleotide comprises a 5′-end wing segment comprising four nucleic acid bases and a 3′-end wing segment comprising four nucleic acid bases. In some aspects, the oligonucleotide comprises a 5′-end wing segment comprising five nucleic acid bases and a 3′-end wing segment comprising five nucleic acid bases. In some aspects, the oligonucleotide comprises a 5′-end wing segment comprising six nucleic acid bases and a 3′-end wing segment comprising six nucleic acid bases. In some aspects, the oligonucleotide comprises a 5′-end wing segment comprising seven nucleic acid bases and a 3′-end wing segment comprising seven nucleic acid bases. In some aspects, the oligonucleotide comprises a 5′-end wing segment comprising eight nucleic acid bases and a 3′-end wing segment comprising eight nucleic acid bases. In some aspects, the oligonucleotide comprises a 5′-end wing segment comprising nine nucleic acid bases and a 3′-end wing segment comprising nine nucleic acid bases. In some aspects, the oligonucleotide comprises a 5′-end wing segment comprising 10 nucleic acid bases and a 3′-end wing segment comprising 10 nucleic acid bases.

In some aspects, the oligonucleotide is an antisense oligonucleotide. In some aspects, the antisense oligonucleotide binds to a target nucleic acid. In some aspects, the target nucleic acid is an endogenous nucleic acid. In some aspects, the target nucleic acid comprises a nuclear RNA, a cytoplasmic RNA, or a mitochondrial RNA. In some aspects, the target RNA comprises an intergenic DNA (including, without limitation, heterochromatic DNA), a messenger RNA (mRNA), a pre-messenger RNA (pre-mRNA), a transfer RNA (tRNA), a ribosomal RNA (rRNA), a ribozyme, cDNA, a recombinant polynucleotide, a branched polynucleotide, a plasmid, a vector, an isolated DNA of a sequence, an isolated RNA of a sequence, a sgRNA, a oligonucleotide, a nucleic acid probe, a primer, an snRNA, a long non-coding RNA, a small RNA, a snoRNA, a siRNA, a miRNA, a tRNA-derived small RNA (tsRNA), an antisense RNA, an shRNA, or a small rDNA-derived RNA (srRNA).

In some aspects, the oligonucleotide comprises a nucleic acid sequence that allows the oligonucleotide to bind to target nucleic acid by base pairing such as Watson Crick base pairing. Compositions and methods provided herein can be utilized to modulate expression of a gene or pathway. Modulation can refer to altering the expression of a gene or portion thereof at one of various stages, with a view to alleviate a disease or condition associated with the gene or a mutation in the gene. Modulation can be mediated at the level of transcription or post-transcriptionally. Modulating transcription can correct aberrant expression of splice variants generated by a mutation in a gene. In some cases, compositions and methods provided herein can be utilized to regulate gene translation of a target. Modulation can refer to decreasing or knocking down the expression of a gene or portion thereof by decreasing the abundance of a transcript. The decreasing the abundance of a transcript can be mediated by decreasing the processing, splicing, turnover or stability of the transcript; or by decreasing the accessibility of the transcript by translational machinery such as ribosome. In some cases, an oligonucleotide described herein can facilitate a knockdown. A knockdown can reduce the expression of a target RNA. In some cases, a knockdown can be accompanied by modulating of an mRNA. In some cases, a knockdown can occur with substantially little to no modulating of an mRNA. In some instances, a knockdown can occur by targeting an untranslated region of the target RNA, such as a 3′ UTR, a 5′ UTR or both. In some cases, a knockdown can occur by targeting a coding region of the target RNA.

In some aspects, the oligonucleotide is an antisense oligonucleotide for targeting and binding any one of the genes described herein. In some aspects, the gene(s) being targeted and bound by the antisense oligonucleotide is NLRP3, NLRC4/NAIP, NLRP1, AIM2, IFI16, Pyrin, or a combination thereof. In some aspects, the antisense oligonucleotide targets and binds to an mRNA that is at least 70%, 75%, 80%, 85%, 90%, 95%, or 99% identical to NLRP3, NLRC4/NAIP, NLRP1, AIM2, IFI16, or Pyrin. In some aspects, the antisense oligonucleotide targets and binds to at least two mRNAs that are at least 70%, 75%, 80%, 85%, 90%, 95%, or 99% identical to mRNA of NLRP3, NLRC4/NAIP, NLRP1, AIM2, IFI16, or Pyrin. In some aspects, the antisense oligonucleotide comprises a nucleic acid sequence that is at least 70%, 75%, 80%, 85%, 90%, 95%, or 99% complementary to the mRNA of NLRP3, NLRC4/NAIP, NLRP1, AIM2, IFI16, Pyrin, or a combination thereof.

In some aspects, the gene(s) being targeted and bound by the antisense oligonucleotide is NLRP3 or NLRP1. In some aspects, the genes being targeted and bound by the antisense oligonucleotide is both NLRP3 and NLRP1. In some aspects, the antisense oligonucleotide targets and binds to a nucleic acid encoding NLRP3 or NLRP1 as shown in Table 1 or mRNA of NLRP3 or NLRP1, also shown in Table 1. In some aspects, the antisense oligonucleotide targets and is capable of binding to an mRNA of NLRP3 encoded by SEQ ID NO: 1. In some aspects, the antisense oligonucleotide targets and is capable of binding to an mRNA of NLRP1 encoded by SEQ ID NO: 8. In some aspects, the antisense oligonucleotide targets and is capable of binding to an mRNA of NLRP3 encoded by SEQ ID NO: 1 or an mRNA of NLRP1 encoded by SEQ ID NO: 8. In some aspects, the antisense oligonucleotide targets and is capable of binding to an mRNA of NLRP3 encoded by SEQ ID NO: 1 and an mRNA of NLRP1 encoded by SEQ ID NO: 8.

In some aspects, the antisense oligonucleotide targets and binds to an mRNA of NLRP3 that is at least 70%, 75%, 80%, 85%, 90%, 95%, or 99% identical to SEQ ID NOs: 2-7. In some aspects, the antisense oligonucleotide targets and binds to an mRNA of NLRP1 that is at least 70%, 75%, 80%, 85%, 90%, 95%, or 99% identical to SEQ ID NOs: 9-13. In some aspects, the antisense oligonucleotide targets and binds to an mRNA of NLRP3 that is at least 70%, 75%, 80%, 85%, 90%, 95%, or 99% identical to SEQ ID NOs: 2-7 or an mRNA of NLRP1 that is at least 70%, 75%, 80%, 85%, 90%, 95%, or 99% identical to SEQ ID NOs: 9-13. In some aspects, the antisense oligonucleotide targets and binds to an mRNA of NLRP3 that is at least 70%, 75%, 80%, 85%, 90%, 95%, or 99% identical to SEQ ID NO: 2-7 and an mRNA of NLRP1 that is at least 70%, 75%, 80%, 85%, 90%, 95%, or 99% identical to SEQ ID NO: 9-13.

In some aspects, the antisense oligonucleotide that targets and binds to an mRNA of NLRP3 comprises a nucleic acid sequence that is at least 70%, 75%, 80%, 85%, 90%, 95%, or 99% identical to SEQ ID NOs: 21-38. In some aspects, the antisense oligonucleotide that targets and binds to an mRNA of NLRP1 comprises a nucleic acid sequence that is at least 70%, 75%, 80%, 85%, 90%, 95%, or 99% identical to SEQ ID NOs: 21-38. In some aspects, the antisense oligonucleotide that targets and binds to an mRNA of NLRP3 or an mRNA of NLRP1 comprises a nucleic acid sequence that is at least 70%, 75%, 80%, 85%, 90%, 95%, or 99% identical to SEQ ID NOs: 21-38. In some aspects, the antisense oligonucleotide that targets and binds to an mRNA of NLRP3 or an mRNA of NLRP1 comprises a nucleic acid sequence that is at least 70%, 75%, 80%, 85%, 90%, 95%, or 99% identical to SEQ ID NOs: 21-38. In some aspects, the antisense oligonucleotide that targets and binds to an mRNA of NLRP3 and an mRNA of NLRP1 comprises a nucleic acid sequence that is at least 70%, 75%, 80%, 85%, 90%, 95%, or 99% identical to SEQ ID NOs: 21-38.

In some aspects, the antisense oligonucleotide that targets and binds to an mRNA of NLRP3 comprises at least 10, at least 11, at least 12, at least 13, at least 14, at least 15 contiguous or consecutive nucleotide from the nucleotide sequence of SEQ ID NOs: 21-38. In some aspects, the antisense oligonucleotide that targets and binds to an mRNA of NLRP3 comprises at least 10, at least 11, at least 12, at least 13, at least 14, at least 15 nucleic acids that is at least 70%, 75%, 80%, 85%, 90%, 95%, or 99% identical to SEQ ID NOs: 21-38. In some aspects, the antisense oligonucleotide that targets and binds to an mRNA of NLRP1 comprises at least 10, at least 11, at least 12, at least 13, at least 14, at least 15 contiguous or consecutive nucleotide from the nucleotide sequence of SEQ ID NOs: 21-38. In some aspects, the antisense oligonucleotide that targets and binds to an mRNA of NLRP3 comprises at least 10, at least 11, at least 12, at least 13, at least 14, at least 15 contiguous or consecutive nucleotide, differing by no more than 2, no more than 3, no more than 4 nucleotides from the nucleotide sequence of SEQ ID NOs: 21-38. In some aspects, the antisense oligonucleotide that targets and binds to an mRNA of NLRP1 comprises at least 10, at least 11, at least 12, at least 13, at least 14, at least 15 contiguous or consecutive nucleic acid bases, differing by no more than 2, no more than 3, no more than 4 nucleic acid bases from the nucleotide sequence of SEQ ID NOs: 21-38.

In some embodiments, the antisense oligonucleotide comprises a nucleic acid sequence that is at least 80%, 85%, 90%, 95%, or 99% identical to any one of SEQ ID NOs: 95, 96, 97, 98, 99, 100, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, or 646. In some embodiments, the antisense oligonucleotide comprises the nucleic acid sequence that is at least 80%, 85%, 90%, 95%, or 99% identical to any one of SEQ ID NOs: 95, 121, 139, 144, 146, 147, or 172. In some embodiments, the antisense oligonucleotide comprises the nucleic acid sequence that is at least 80%, 85%, 90%, 95%, or 99% identical to SEQ ID NO: 95. In some embodiments, the antisense oligonucleotide comprises the nucleic acid sequence that is at least 80%, 85%, 90%, 95%, or 99% identical to SEQ ID NO: 121. In some embodiments, the antisense oligonucleotide comprises the nucleic acid sequence that is at least 80%, 85%, 90%, 95%, or 99% identical to SEQ ID NO: 139. In some embodiments, the antisense oligonucleotide comprises the nucleic acid sequence that is at least 80%, 85%, 90%, 95%, or 99% identical to SEQ ID NO: 144. In some embodiments, the antisense oligonucleotide comprises the nucleic acid sequence that is at least 80%, 85%, 90%, 95%, or 99% identical to SEQ ID NO: 146. In some embodiments, the antisense oligonucleotide comprises the nucleic acid sequence that is at least 80%, 85%, 90%, 95%, or 99% identical to SEQ ID NO: 147. In some embodiments, the antisense oligonucleotide comprises the nucleic acid sequence that is at least 80%, 85%, 90%, 95%, or 99% identical to SEQ ID NO: 172. In some embodiments, the antisense oligonucleotide is any one of SEQ ID NOs: 95, 121, 139, 144, 146, 147, or 172. In some embodiments, the antisense oligonucleotide is SEQ ID NO: 95. In some embodiments, the antisense oligonucleotide is SEQ ID NO: 121. In some embodiments, the antisense oligonucleotide is SEQ ID NO: 139. In some embodiments, the antisense oligonucleotide is SEQ ID NO: 144. In some embodiments, the antisense oligonucleotide is SEQ ID NO: 146. In some embodiments, the antisense oligonucleotide is SEQ ID NO: 147. In some embodiments, the antisense oligonucleotide is SEQ ID NO: 172.

In some aspects, the oligonucleotide described herein targets and binds to an endogenous nucleic acid encoding a gene associated with the inflammasome pathway. In some aspects, the gene associated with the inflammasome pathway is NLRP3, NLRC4/NAIP, NLRP1, AIM2, IFI16, Pyrin, or a combination thereof. In some aspects, the gene associated with the inflammasome pathway is NLRP3. In some aspects, the gene associated with the inflammasome pathway is NLRP1. In some aspects, the gene associated with the inflammasome pathway is NLRP3 or NLRP1. In some aspects, the gene associated with the inflammasome pathway is NLRP3 and NLRP1.

In some aspects, the oligonucleotide, upon binding to the endogenous nucleic acid, forms a duplex with the endogenous nucleic acid and recruits an endogenous nuclease for degrading the endogenous nucleic acid. In some aspects, the endogenous nuclease is a deoxyribonuclease. In some aspects, the endogenous nuclease is a ribonuclease. In some aspects, the ribonuclease is an endoribonuclease. In some aspects, the endoribonuclease comprises endoribonuclease or RNase A, P, H, I, III, T1, T2, U2, V1, PhyM, or V. In some aspects, the ribonuclease is an exoribonuclease. In some aspects, the exoribonuclease comprises RNase PH, II, R, D, or T. In some aspects, the nuclease comprises polynucleotide phosphorylase (PNPase), oligoribonuclease, exoribonuclease I, or exoribonuclease II. In some aspects, the ribonuclease recruited by the oligonucleotide binding to the endogenous nucleic acid is RNase H.

In some aspects, the oligonucleotide comprises at least one, two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more chemical modifications. In some aspects, the oligonucleotide comprises at least one gap segment comprising at least one, two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more chemical modifications. In some aspects, the oligonucleotide comprises at least one wing segment comprising at least one, two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more chemical modifications. In some aspects, the oligonucleotide comprises at least one gap segment and at least one wing segment comprising at least one, two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more chemical modifications.

In some aspects, the oligonucleotide described herein binds to an endogenous nucleic acid (e.g., a mRNA) encoding NLRP3, where the binding of the oligonucleotide to the NLRP3 endogenous nucleic acid decreases the endogenous mRNA or protein expression of NLRP3 in a cell by at least 10%, 20%, 30%, 40%, 50%, 60%, 70%, 80%, 90%, or more compared to the endogenous mRNA or protein expression of NLRP3 not modulated by the oligonucleotide. In some aspects, the oligonucleotide described herein binds to an endogenous nucleic acid (e.g., a mRNA) encoding NLRP1, where the binding of the oligonucleotide to the NLRP1 endogenous nucleic acid decreases the endogenous mRNA or protein expression of NLRP1 in a cell by at least 10%, 20%, 30%, 40%, 50%, 60%, 70%, 80%, 90%, or more compared to the endogenous mRNA or protein expression of NLRP1 not modulated by the oligonucleotide. In some aspects, the oligonucleotide described herein binds to an endogenous nucleic acid (e.g., a mRNA) encoding NLRP3 or NLRP1, where the binding of the oligonucleotide to the NLRP3 or NLRP1 endogenous nucleic acid decreases the endogenous mRNA or protein expression of NLRP3 or NLRP1 in a cell by at least 10%, 20%, 30%, 40%, 50%, 60%, 70%, 80%, 90%, or more compared to the endogenous mRNA or protein expression of NLRP3 or NLRP1 not modulated by the oligonucleotide. In some aspects, the oligonucleotide described herein binds to an endogenous nucleic acid (e.g., a mRNA) encoding NLRP3 or NLRP1, where the binding of the oligonucleotide to the NLRP3 and NLRP1 endogenous nucleic acids decreases the endogenous mRNA or protein expression of NLRP3 and NLRP1 in a cell by at least 10%, 20%, 30%, 40%, 50%, 60%, 70%, 80%, 90%, or more compared to the endogenous mRNA or protein expression of NLRP3 and NLRP1 not modulated by the oligonucleotide.

In some aspects, the oligonucleotide described herein binds to an endogenous nucleic acid (e.g., a mRNA) encoding NLRP3, where the binding of the oligonucleotide to the NLRP3 endogenous nucleic acid increases or decreases the endogenous expression or the activity of a gene or a protein associated or within the inflammasome pathway in a cell by at least 10%, 20%, 30%, 40%, 50%, 60%, 70%, 80%, 90%, or more compared to the endogenous expression or the activity of a gene or a protein associated or within of the inflammasome pathway not modulated by the oligonucleotide. In some aspects, the oligonucleotide described herein binds to an endogenous nucleic acid (e.g., a mRNA) encoding NLRP1, where the binding of the oligonucleotide to the NLRP1 endogenous nucleic acid increases or decreases the endogenous expression or the activity of a gene or a protein associated or within the inflammasome pathway in a cell by at least 10%, 20%, 30%, 40%, 50%, 60%, 70%, 80%, 90%, or more compared to the endogenous expression or the activity of a gene or a protein associated or within of the inflammasome pathway not modulated by the oligonucleotide. In some aspects, the oligonucleotide described herein binds to an endogenous nucleic acid (e.g., a mRNA) encoding NLRP3 or NLRP1, where the binding of the oligonucleotide to the NLRP3 or NLRP1 endogenous nucleic acid increases or decreases the endogenous expression or the activity of a gene or a protein associated or within the inflammasome pathway in a cell by at least 10%, 20%, 30%, 40%, 50%, 60%, 70%, 80%, 90%, or more compared to the endogenous expression or the activity of a gene or a protein associated or within of the inflammasome pathway not modulated by the oligonucleotide.

In some aspects, the composition comprises at least two oligonucleotides, where a first oligonucleotide binds to NLRP3 or NLRP1 endogenous nucleic acid (e.g., a NLRP3 or NLRP1 mRNA) and a second oligonucleotide binds to NLRP3 or NLRP1 endogenous nucleic acid (e.g., a NLRP3 or NLRP1 mRNA), where the first and second oligonucleotides binds to different endogenous nucleic acid or different portions of the same endogenous nucleic acid. In some aspects, the binding of the oligonucleotides to both NLRP3 and NLRP1 endogenous nucleic acids decreases the endogenous expression of a gene or a protein in, or an activity of a protein in, the inflammasome pathway in a cell by at least 10%, 20%, 30%, 40%, 50%, 60%, 70%, 80%, 90%, or more compared to the endogenous expression of a gene or a protein in, or an activity a protein in of the inflammasome pathway not modulated by the oligonucleotide.

In some aspects, the oligonucleotide described herein binds to an endogenous nucleic acid (e.g., a mRNA) encoding NLRP3, where the binding of the oligonucleotide to the NLRP3 endogenous nucleic acid decreases the endogenous expression of at least one cytokines described herein in a cell or in a tissue by at least 10%, 20%, 30%, 40%, 50%, 60%, 70%, 80%, 90%, or more compared to the endogenous expression of the at least one cytokine not modulated by the oligonucleotide. In some aspects, the oligonucleotide described herein binds to an endogenous nucleic acid (e.g., a mRNA) encoding NLRP1, where the binding of the oligonucleotide to the NLRP1 endogenous nucleic acid decreases the endogenous expression of at least one cytokines described herein in a cell or in a tissue by at least 10%, 20%, 30%, 40%, 50%, 60%, 70%, 80%, 90%, or more compared to the endogenous expression of the at least one cytokine not modulated by the oligonucleotide. In some aspects, the oligonucleotide described herein binds to an endogenous nucleic acid (e.g., a mRNA) encoding NLRP3 or NLRP1, where the binding of the oligonucleotide to the NLRP3 or NLRP1 endogenous nucleic acid decreases the endogenous expression of at least one cytokines described herein in a cell or in a tissue by at least 10%, 20%, 30%, 40%, 50%, 60%, 70%, 80%, 90%, or more compared to the endogenous expression of the at least one cytokine not modulated by the oligonucleotide. In some aspects, the oligonucleotide described herein binds to an endogenous nucleic acid (e.g., a mRNA) encoding NLRP3 and NLRP1, where the binding of the oligonucleotide to the NLRP3 and NLRP1 endogenous nucleic acid decreases the endogenous expression of at least one cytokines described herein in a cell or in a tissue by at least 10%, 20%, 30%, 40%, 50%, 60%, 70%, 80%, 90%, or more compared to the endogenous expression of the at least one cytokine not modulated by the oligonucleotide. In some aspects, the oligonucleotide described herein binds to an endogenous nucleic acid (e.g., a mRNA) encoding NLRP3, where the binding of the oligonucleotide to the NLRP3 endogenous nucleic acid increases the endogenous expression of at least one cytokines described herein in a cell or in a tissue by at least 10%, 20%, 30%, 40%, 50%, 60%, 70%, 80%, 90%, or more compared to the endogenous expression of the at least one cytokine not modulated by the oligonucleotide. In some aspects, the oligonucleotide described herein binds to an endogenous nucleic acid (e.g., a mRNA) encoding NLRP1, where the binding of the oligonucleotide to the NLRP1 endogenous nucleic acid increases the endogenous expression of at least one cytokines described herein in a cell or in a tissue by at least 10%, 20%, 30%, 40%, 50%, 60%, 70%, 80%, 90%, or more compared to the endogenous expression of the at least one cytokine not modulated by the oligonucleotide. In some aspects, the oligonucleotide described herein binds to an endogenous nucleic acid (e.g., a mRNA) encoding NLRP3 or NLRP1, where the binding of the oligonucleotide to the NLRP3 or NLRP1 endogenous nucleic acid increases the endogenous expression of at least one cytokines described herein in a cell or in a tissue by at least 10%, 20%, 30%, 40%, 50%, 60%, 70%, 80%, 90%, or more compared to the endogenous expression of the at least one cytokine not modulated by the oligonucleotide. In some aspects, the oligonucleotide described herein binds to an endogenous nucleic acid (e.g., a mRNA) encoding NLRP3 and NLRP1, where the binding of the oligonucleotide to the NLRP3 and NLRP1 endogenous nucleic acid increases the endogenous expression of at least one cytokines described herein in a cell or in a tissue by at least 10%, 20%, 30%, 40%, 50%, 60%, 70%, 80%, 90%, or more compared to the endogenous expression of the at least one cytokine not modulated by the oligonucleotide. Non-limiting examples of the at least one cytokine that can be modulated by the oligonucleotide binding to the endogenous NLRP3 or NLRP1 nucleic acid include: 4-1BBL, acylation stimulating protein, adipokine, albinterferon, APRIL, Arh, BAFF, Bcl-6, CCL1, CCL1/TCA3, CCL11, CCL12/MCP-5, CCL13/MCP-4, CCL14, CCL15, CCL16, CCL17/TARC, CCL18, CCL19, CCL2, CCL2/MCP-1, CCL20, CCL21, CCL22/MDC, CCL23, CCL24, CCL25, CCL26, CCL27, CCL28, CCL3, CCL3L3, CCL4, CCL4L1/LAG-1, CCL5, CCL6, CCL7, CCL8, CCL9, CCR10, CCR3, CCR4, CCR5, CCR6, CCR7, CCR8, CD153, CD154, CD178, CD40LG, CD70, CD95L/CD178, Cerberus (protein), chemokines, CLCF1, CNTF, colony-stimulating factor, common b chain (CD131), common g chain (CD132), CX3CL1, CX3CR1, CXCL1, CXCL10, CXCL11, CXCL12, CXCL13, CXCL14, CXCL15, CXCL16, CXCL17, CXCL2, CXCL2/MIP-2, CXCL3, CXCL4, CXCL5, CXCL6, CXCL7, CXCL9, CXCR3, CXCR4, CXCR5, EDA-A1, Epo, erythropoietin, FAM19A1, FAM19A2, FAM19A3, FAM19A4, FAM19A5, Flt-3L, FMS-like tyrosine kinase 3 ligand, Foxp3, GATA-3, GcMAF, G-CSF, GITRL, GM-CSF, granulocyte colony-stimulating factor, granulocyte-macrophage colony-stimulating factor, hepatocyte growth factor, IFNA1, IFNA10, IFNA13, IFNA14, IFNA2, IFNA4, IFNA5/IFNaG, IFNA7, IFNA8, IFNB1, IFNE, IFNG, IFNZ, IFN-α, IFN-β, IFN-γ, IFNω/IFNW1, IL-1, IL-10, IL-10 family, IL-10-like, IL-11, IL-12, IL-13, IL-14, IL-15, IL-16, IL-17, IL-17 family, IL-17A-F, IL-18, IL-18BP, IL-19, IL-1A, IL-1B, IL-1F10, IL-1F3/IL-1RA, IL-1F5, IL-1F6, IL-1F7, IL-1F8, IL-1F9, IL-1-like, IL-1RA, IL-1RL2, IL-1α, IL-1β, IL-2, IL-20, IL-21, IL-22, IL-23, IL-24, IL-28A, IL-28B, IL-29, IL-3, IL-31, IL-33, IL-35, IL-4, IL-5, IL-6, IL-6-like, IL-7, IL-8/CXCL8, IL-9, inflammasome, interferome, interferon, interferon beta-1a, interferon beta-1b, interferon gamma, interferon type I, interferon type II, interferon type III, interferons, interleukin, interleukin 1 receptor antagonist, Interleukin 8, IRF4, Leptin, leukemia inhibitory factor (LIF), leukocyte-promoting factor, LIGHT, LTA/TNFB, LT-β, lymphokine, lymphotoxin, lymphotoxin alpha, lymphotoxin beta, macrophage colony-stimulating factor, macrophage inflammatory protein, macrophage-activating factor, M-CSF, MHC class III, miscellaneous hematopoietins, monokine, MSP, myokine, myonectin, nicotinamide phosphoribosyltransferase, oncostatin M (OSM), oprelvekin, OX40L, platelet factor 4, promegapoietin, RANKL, SCF, STAT3, STAT4, STAT6, stromal cell-derived factor 1, TALL-1, TBX21, TGF-α, TGF-β, TGF-β1, TGF-β2, TGF-β3, TNF, TNFSF10, TNFSF11, TNFSF12, TNFSF13, TNFSF14, TNFSF15, TNFSF4, TNFSF8, TNF-α, TNF-β, Tpo, TRAIL, TRANCE, TWEAK, vascular endothelial growth inhibitor, XCL1, or XCL2.

In some aspects, the composition is formulated for administration to a subject by appropriate administration routes, including but not limited to, intravenous, intraarterial, oral, parenteral, buccal, topical, transdermal, rectal, intramuscular, subcutaneous, intraosseous, transmucosal, inhalation, or intraperitoneal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations. In some aspects, the composition is formulated into a dosage form. In some aspects, the composition is formulated to include at least one excipient. In some aspects, the excipient is a pharmaceutically acceptable excipient.

In some aspects, the composition comprising the oligonucleotide described herein treats a disease or condition by decreasing the expression of the gene or the pathway associated with the disease or condition. In some aspects, the composition comprising the oligonucleotide describe herein treats the disease or condition described herein by directly decreasing the gene expression associated with disease or condition described herein. In some aspects, the composition comprising the oligonucleotide treats the disease or condition by decreasing the gene expression as part of a pathway described herein. In some aspects, the composition comprising the oligonucleotide described herein treats a disease or condition by decreasing the endogenous NLRP3 expression. In some aspects, the composition comprising the oligonucleotide described herein treats a disease or condition by decreasing the endogenous NLRP1 expression. In some aspects, the composition comprising the oligonucleotide described herein treats a disease or condition by decreasing the endogenous NLRP3 or NLRP1 expression. In some aspects, the composition comprising the oligonucleotide described herein treats a disease or condition by decreasing the endogenous NLRP3 and NLRP1 expression. In some aspects, the composition comprising the oligonucleotide described herein treats a disease or condition by decreasing the endogenous inflammasome expression. In some aspects, the composition comprising the oligonucleotide described herein treats a disease or condition by decreasing the endogenous inflammasome pathway activity. In some aspects, the disease or condition described herein is an inflammatory disease or condition. In some aspects, the disease or condition described herein is a pyroptosis disease or condition.

### Chemical Modifications

Described herein, In some aspects, is an oligonucleotide comprising at least one chemical modification. In some aspects, the oligonucleotide is single-stranded. In some aspects, the oligonucleotide is an antisense oligonucleotide. In some aspects, the oligonucleotide comprises at least one, two, three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more chemical modifications. In some aspects, the oligonucleotide does not have an intramolecular structure feature. In some aspects, the oligonucleotide comprises at least one gap segment comprising at least one, two, three, four, five, six, seven, eight, nine, ten, 11, 12, 13, 14, 15, or more chemically modified nucleic acid bases. In some aspects, the oligonucleotide comprises at least one wing segment comprising at least one, two, three, four, five, six, seven, eight, nine, ten, or more chemically modified nucleic acid bases. In some aspects, the oligonucleotide comprises a 5′-end wing segment comprising at least one, two, three, four, five, six, seven, eight, nine, ten, or more chemically modified nucleic acid bases. In some aspects, the oligonucleotide comprises a 3′-end wing segment comprising at least one, two, three, four, five, six, seven, eight, nine, ten, or more chemically modified nucleic acid bases. In some aspects, the at least one wing segment is covalently fused to the 5′-end of the gap segment. In some aspects, the at least one wing segment is covalently fused to the 3′-end of the gap segment.

In some aspects, the oligonucleotide comprises at least one, two, three, four, five, six, seven, eight, nine, ten, 11, 12, 13, 14, 15, 16, 17, 18, 19, 20 or more chemically modified nucleic acid bases at the 5′ end of the oligonucleotide. In some aspects, the oligonucleotide comprises at least one, two, three, four, five, six, seven, eight, nine, ten, 11, 12, 13, 14, 15, 16, 17, 18, 19, 20 or more chemically modified nucleic acid bases at the 3′ end of the oligonucleotide. In some aspects, the oligonucleotide comprises at least one, two, three, four, five, six, seven, eight, nine, ten, 11, 12, 13, 14, 15, 16, 17, 18, 19, 20 or more chemically modified nucleic acid bases at both the 5′ and the 3′ end of the oligonucleotide. In some aspects, the oligonucleotide comprises at least one chemical modification in the gap segment of the oligonucleotide. In some aspects, the oligonucleotide comprises at least one chemical modification in the nucleic acid base adjacent the gap segment. In some aspects, at least 10%, 20%, 30%, 40%, 50%, 60%, 70%, 80%, 90% of the bases or internucleotide linkage of the oligonucleotide comprises modifications. In some aspects, the oligonucleotide comprises 100% modified nucleotide bases.

In some aspects, chemical modification can occur at 3′OH, group, 5′OH group, at the backbone, at the sugar component, or at the nucleotide base. Chemical modification can include non-naturally occurring linker molecules of interstrand or intrastrand cross links. In one aspect, the chemically modified nucleic acid comprises modification of one or more of the 3′OH or 5′OH group, the backbone, the sugar component, or the nucleotide base, or addition of non-naturally occurring linker molecules. In some aspects, chemically modified backbone comprises a backbone other than a phosphodiester backbone. In some aspects, a modified sugar comprises a sugar other than deoxyribose (in modified DNA) or other than ribose (modified RNA). In some aspects, a modified base comprises a base other than adenine, guanine, cytosine, thymine or uracil. In some aspects, the oligonucleotide comprises at least one chemically modified base. In some instances, the comprises at least one, two, three, four, five, six, seven, eight, nine, 10, 15, 20, or more modified bases. In some cases, chemical modifications to the base moiety include natural and synthetic modifications of adenine, guanine, cytosine, thymine, or uracil, and purine or pyrimidine bases.

In some aspects, the at least one chemical modification of the oligonucleotide comprises a modification of any one of or any combination of: 2′ modified nucleotide comprising 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA); modification of one or both of the non-linking phosphate oxygens in the phosphodiester backbone linkage; modification of one or more of the linking phosphate oxygens in the phosphodiester backbone linkage; modification of a constituent of the ribose sugar; replacement of the phosphate moiety with “dephospho” linkers; modification or replacement of a naturally occurring nucleobase; modification of the ribose-phosphate backbone; modification of 5′ end of polynucleotide; modification of 3′ end of polynucleotide; modification of the deoxyribose phosphate backbone; substitution of the phosphate group; modification of the ribophosphate backbone; modifications to the sugar of a nucleotide; modifications to the base of a nucleotide; or stereopure of nucleotide. Non-limiting examples of chemical modification to the oligonucleotide can include: modification of one or both of non-linking or linking phosphate oxygens in the phosphodiester backbone linkage (e.g., sulfur (S), selenium (Se), BR3 (wherein R can be, e.g., hydrogen, alkyl, or aryl), C (e.g., an alkyl group, an aryl group, and the like), H, NR2, wherein R can be, e.g., hydrogen, alkyl, or aryl, or wherein R can be, e.g., alkyl or aryl); replacement of the phosphate moiety with “dephospho” linkers (e.g., replacement with methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo, or methyleneoxymethylimino); modification or replacement of a naturally occurring nucleobase with nucleic acid analog; modification of deoxyribose-phosphate or ribose-phosphate backbone (e.g., modifying the ribose-phosphate backbone to incorporate phosphorothioate, phosphonothioacetate, phosphoroselenates, boranophosphates, borano phosphate esters, hydrogen phosphonates, phosphonocarboxylate, phosphoroamidates, alkyl or aryl phosphonates, phosphonoacetate, or phosphotriesters; modification of 5′ end (e.g., 5′ cap or modification of 5′ cap —OH) or 3′ end of the nucleic acid sequence (3′ tail or modification of 3′ end —OH); substitution of the phosphate group with methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo, or methyleneoxymethylimino; modification of the ribophosphate backbone to incorporate morpholino (e.g., phosphorodiamidate morpholino oligomer, PMO, or thiomorpholino), cyclobutyl, pyrrolidine, or peptide nucleic acid (PNA) nucleoside surrogates; modifications to the sugar of a nucleotide to incorporate locked nucleic acid (LNA), unlocked nucleic acid (UNA), ethylene nucleic acid (ENA), constrained ethyl (cEt) sugar, or bridged nucleic acid (BNA); modification of a constituent of the ribose sugar (e.g., 2′-O-methyl, 2′-O-methoxy-ethyl (2′-MOE), 2′-fluoro, 2′-aminoethyl, 2′-deoxy-2′-fuloarabinou-cleic acid, 2′-deoxy, 2′-O-methyl, 3′-phosphorothioate, 3′-phosphonoacetate (PACE), or 3′-phosphonothioacetate (thioPACE)); modification to the base of a nucleotide (of A, T, C, G, or U); and stereopure of nucleotide (e.g., S conformation of phosphorothioate or R conformation of phosphorothioate).

In some aspects, the chemical modification of the oligonucleotide comprises at least one substitution of one or both of non-linking phosphate oxygen atoms in a phosphodiester backbone linkage of the oligonucleotide. In some aspects, the at least one chemical modification of the oligonucleotide comprises a substitution of one or more of linking phosphate oxygen atoms in a phosphodiester backbone linkage of the oligonucleotide. A non-limiting example of a chemical modification of a phosphate oxygen atom is a sulfur atom. In some aspects, the chemical modifications of the oligonucleotide comprise at least one chemical modification to a sugar of a nucleotide of the oligonucleotide. In some aspects, the chemical modifications of the oligonucleotide comprise at least one chemical modification to the sugar of the nucleotide, where the chemical modification comprises at least one locked nucleic acid (LNA). In some aspects, the chemical modifications of the oligonucleotide comprise at least one chemical modification to the sugar of the nucleotide of the oligonucleotide comprising at least one unlocked nucleic acid (UNA). In some aspects, the chemical modifications of the oligonucleotide comprise at least one chemical modification to the sugar of the nucleotide of the oligonucleotide comprising at least one ethylene nucleic acid (ENA). In some aspects, the chemical modifications of the oligonucleotide comprise at least one chemical modification to the sugar comprising a modification of a constituent of the sugar, where the sugar is a ribose sugar. In some aspects, the chemical modifications of the oligonucleotide comprise at least one chemical modification to the constituent of the ribose sugar of the nucleotide of the oligonucleotide comprising a 2′-O-Methyl group. In some aspects, the chemical modifications of the oligonucleotide comprise at least one chemical modification comprising replacement of a phosphate moiety of the oligonucleotide with a dephospho linker. In some aspects, the chemical modifications of the oligonucleotide comprise at least one chemical modification of a phosphate backbone of the oligonucleotide. In some aspects, the oligonucleotide comprises a phosphothioate group. In some aspects, the chemical modifications of the oligonucleotide comprise at least one chemical modification comprising a modification to a base of a nucleotide of the oligonucleotide. In some aspects, the chemical modifications of the oligonucleotide comprise at least one chemical modification comprising an unnatural base of a nucleotide. In some aspects, the chemical modifications of the oligonucleotide comprise at least one chemical modification comprising a morpholino group (e.g., phosphorodiamidate morpholino oligomer, PMO, or thiomorpholino), a cyclobutyl group, pyrrolidine group, or peptide nucleic acid (PNA) nucleoside surrogate. In some aspects, the chemical modifications of the oligonucleotide comprise at least one chemical modification comprising at least one stereopure nucleic acid. In some aspects, the at least one chemical modification can be positioned proximal to a 5′ end of the oligonucleotide. In some aspects, the at least one chemical modification can be positioned proximal to a 3′ end of the oligonucleotide. In some aspects, the at least one chemical modification can be positioned proximal to both 5′ and 3′ ends of the oligonucleotide.

In some aspects, an oligonucleotide comprises a backbone comprising a plurality of sugar and phosphate moieties covalently linked together. In some cases, a backbone of an oligonucleotide comprises a phosphodiester bond linkage between a first hydroxyl group in a phosphate group on a 5′ carbon of a deoxyribose in DNA or ribose in RNA and a second hydroxyl group on a 3′ carbon of a deoxyribose in DNA or ribose in RNA.

In some aspects, a backbone of an oligonucleotide can lack a 5′ reducing hydroxyl, a 3′ reducing hydroxyl, or both, capable of being exposed to a solvent. In some aspects, a backbone of an oligonucleotide can lack a 5′ reducing hydroxyl, a 3′ reducing hydroxyl, or both, capable of being exposed to nucleases. In some aspects, a backbone of an oligonucleotide can lack a 5′ reducing hydroxyl, a 3′ reducing hydroxyl, or both, capable of being exposed to hydrolytic enzymes. In some instances, a backbone of an oligonucleotide can be represented as a polynucleotide sequence in a circular 2-dimensional format with one nucleotide after the other. In some instances, a backbone of an oligonucleotide can be represented as a polynucleotide sequence in a looped 2-dimensional format with one nucleotide after the other. In some cases, a 5′ hydroxyl, a 3′ hydroxyl, or both, are joined through a phosphorus-oxygen bond. In some cases, a 5′ hydroxyl, a 3′ hydroxyl, or both, are modified into a phosphoester with a phosphorus-containing moiety.

In some aspects, the oligonucleotide described herein comprises at least one chemical modification. A chemical modification can be a substitution, insertion, deletion, chemical modification, physical modification, stabilization, purification, or any combination thereof. In some cases, a modification is a chemical modification. Suitable chemical modifications comprise any one of: 5′ adenylate, 5′ guanosine-triphosphate cap, 5′N7-Methylguanosine-triphosphate cap, 5′triphosphate cap, 3′phosphate, 3′thiophosphate, 5′phosphate, 5′thiophosphate, Cis-Syn thymidine dimer, trimers, C12 spacer, C3 spacer, C6 spacer, dSpacer, PC spacer, rSpacer, Spacer 18, Spacer 9,3′-3′ modifications, 5′-5′ modifications, abasic, acridine, azobenzene, biotin, biotin BB, biotin TEG, cholesteryl TEG, desthiobiotin TEG, DNP TEG, DNP-X, DOTA, dT-Biotin, dual biotin, PC biotin, psoralen C2, psoralen C6, TINA, 3′DABCYL, black hole quencher 1, black hole quencher 2, DABCYL SE, dT-DABCYL, IRDye QC-1, QSY-21, QSY-35, QSY-7, QSY-9, carboxyl linker, thiol linkers, 2′deoxyribonucleoside analog purine, 2′deoxyribonucleoside analog pyrimidine, ribonucleoside analog, 2′-O-methyl ribonucleoside analog, sugar modified analogs, wobble/universal bases, fluorescent dye label, 2′fluoro RNA, 2′O-methyl RNA, methylphosphonate, phosphodiester DNA, phosphodiester RNA, phosphothioate DNA, phosphorothioate RNA, UNA, LNA, cEt, pseudouridine-5′-triphosphate, 5-methylcytidine-5′-triphosphate, 2′-O-methyl 3phosphorothioate or any combinations thereof.

In some cases, a modification can be permanent. In other cases, a modification can be transient. In some cases, multiple modifications are made to the oligonucleotide. the oligonucleotide modification can alter physio-chemical properties of a nucleotide, such as their conformation, polarity, hydrophobicity, chemical reactivity, base-pairing interactions, or any combination thereof.

A chemical modification can also be a phosphorothioate substitute. In some cases, a natural phosphodiester bond can be susceptible to rapid degradation by cellular nucleases and; a modification of internucleotide linkage using phosphorothioate (PS) bond substitutes can be more stable towards hydrolysis by cellular degradation. A modification can increase stability in a polynucleic acid. A modification can also enhance biological activity. In some cases, a phosphorothioate enhanced RNA polynucleic acid can inhibit RNase A, RNase T1, calf serum nucleases, or any combinations thereof. These properties can allow the use of PS-RNA polynucleic acids to be used in applications where exposure to nucleases is of high probability in vivo or in vitro. For example, phosphorothioate (PS) bonds can be introduced between the last 3-5 nucleotides at the 5′- or 3′-end of a polynucleic acid which can inhibit exonuclease degradation. In some cases, phosphorothioate bonds can be added throughout an entire polynucleic acid to reduce attack by endonucleases.

In some aspects, the oligonucleotide described herein comprises at least one, two, three, four, five, six, seven, eight, nine, 10, 11, 12, 13, 14, 15, 16, 17, 18, 19, 20, 21, 22, 23, 24, 25, 26, 27, 28, 29, 30, 50, 100, or more internucleotide linkage comprising PS bond. In some aspects, the oligonucleotide described herein comprises only PS bond as the internucleotide linkage modification. In some aspects, all internucleotide linkages of the oligonucleotide described herein are fully PS-modified or include phosphorothioate internucleotide linkages. In some aspects, the oligonucleotide comprising PS bond as the internucleotide linkage modification comprises a 5′-end wing segment comprising one nucleic acid base. In some aspects, the oligonucleotide comprising PS bond as the internucleotide linkage modification comprises a 5′-end wing segment comprising two nucleic acid bases. In some aspects, the oligonucleotide comprising PS bond as the internucleotide linkage modification comprises a 5′-end wing segment comprising three nucleic acid bases. In some aspects, the oligonucleotide comprising PS bond as the internucleotide linkage modification comprises a 5′-end wing segment comprising four nucleic acid bases. In some aspects, the oligonucleotide comprising PS bond as the internucleotide linkage modification comprises a 5′-end wing segment comprising five nucleic acid bases. In some aspects, the oligonucleotide comprising PS bond as the internucleotide linkage modification comprises a 5′-end wing segment comprising six nucleic acid bases. In some aspects, the oligonucleotide comprising PS bond as the internucleotide linkage modification comprises a 5′-end wing segment comprising seven nucleic acid bases. In some aspects, the oligonucleotide comprising PS bond as the internucleotide linkage modification comprises a 5′-end wing segment comprising eight nucleic acid bases. In some aspects, the oligonucleotide comprising PS bond as the internucleotide linkage modification comprises a 5′-end wing segment comprising nine nucleic acid bases. In some aspects, the oligonucleotide comprising PS bond as the internucleotide linkage modification comprises a 5′-end wing segment comprising 10 nucleic acid bases. In some aspects, the oligonucleotide comprising PS bond as the internucleotide linkage modification comprises a 3′-end wing segment comprising one nucleic acid base. In some aspects, the oligonucleotide comprising PS bond as the internucleotide linkage modification comprises a 3′-end wing segment comprising two nucleic acid bases. In some aspects, the oligonucleotide comprising PS bond as the internucleotide linkage modification comprises a 3′-end wing segment comprising three nucleic acid bases. In some aspects, the oligonucleotide comprising PS bond as the internucleotide linkage modification comprises a 3′-end wing segment comprising four nucleic acid bases. In some aspects, the oligonucleotide comprising PS bond as the internucleotide linkage modification comprises a 3′-end wing segment comprising five nucleic acid bases. In some aspects, the oligonucleotide comprising PS bond as the internucleotide linkage modification comprises a 3′-end wing segment comprising six nucleic acid bases. In some aspects, the oligonucleotide comprising PS bond as the internucleotide linkage modification comprises a 3′-end wing segment comprising seven nucleic acid bases. In some aspects, the oligonucleotide comprising PS bond as the internucleotide linkage modification comprises a 3′-end wing segment comprising eight nucleic acid bases. In some aspects, the oligonucleotide comprising PS bond as the internucleotide linkage modification comprises a 3′-end wing segment comprising nine nucleic acid bases. In some aspects, the oligonucleotide comprising PS bond as the internucleotide linkage modification comprises a 3′-end wing segment comprising 10 nucleic acid bases.

In some aspects, the oligonucleotide comprising PS bond as the internucleotide linkage modification comprises a 5′-end wing segment comprising one nucleic acid base and a 3′-end wing segment comprising one nucleic acid base. In some aspects, the oligonucleotide comprising PS bond as the internucleotide linkage modification comprises a 5′-end wing segment comprising two nucleic acid bases and a 3′-end wing segment comprising two nucleic acid bases. In some aspects, the oligonucleotide comprising PS bond as the internucleotide linkage modification comprises a 5′-end wing segment comprising three nucleic acid bases and a 3′-end wing segment comprising three nucleic acid bases. In some aspects, the oligonucleotide comprising PS bond as the internucleotide linkage modification comprises a 5′-end wing segment comprising four nucleic acid bases and a 3′-end wing segment comprising four nucleic acid bases. In some aspects, the oligonucleotide comprising PS bond as the internucleotide linkage modification comprises a 5′-end wing segment comprising five nucleic acid bases and a 3′-end wing segment comprising five nucleic acid bases. In some aspects, the oligonucleotide comprising PS bond as the internucleotide linkage modification comprises a 5′-end wing segment comprising six nucleic acid bases and a 3′-end wing segment comprising six nucleic acid bases. In some aspects, the oligonucleotide comprising PS bond as the internucleotide linkage modification comprises a 5′-end wing segment comprising seven nucleic acid bases and a 3′-end wing segment comprising seven nucleic acid bases. In some aspects, the oligonucleotide comprising PS bond as the internucleotide linkage modification comprises a 5′-end wing segment comprising eight nucleic acid bases and a 3′-end wing segment comprising eight nucleic acid bases. In some aspects, the oligonucleotide comprising PS bond as the internucleotide linkage modification comprises a 5′-end wing segment comprising nine nucleic acid bases and a 3′-end wing segment comprising nine nucleic acid bases. In some aspects, the oligonucleotide comprising PS bond as the internucleotide linkage modification comprises a 5′-end wing segment comprising 10 nucleic acid bases and a 3′-end wing segment comprising 10 nucleic acid bases.

In some aspects, the oligonucleotide comprises a 5′-end wing segment comprising one nucleic acid base, a gapmer, and a 3′-end wing segment comprising one nucleic acid base, where the internucleotide linkages of the oligonucleotide joining the 5′-end wing segment, the gapmer, and the 3′-end wing segment comprises only PS bonds. In some aspects, the oligonucleotide comprises a 5′-end wing segment comprising two nucleic acid bases, a gapmer, and a 3′-end wing segment comprising two nucleic acid bases, where the internucleotide linkages of the oligonucleotide joining the 5′-end wing segment, the gapmer, and the 3′-end wing segment comprises only PS bonds. In some aspects, the oligonucleotide comprises a 5′-end wing segment comprising three nucleic acid bases, a gapmer, and a 3′-end wing segment comprising three nucleic acid bases, where the internucleotide linkages of the oligonucleotide joining the 5′-end wing segment, the gapmer, and the 3′-end wing segment comprises only PS bonds. In some aspects, the oligonucleotide comprises a 5′-end wing segment comprising four nucleic acid bases, a gapmer, and a 3′-end wing segment comprising four nucleic acid bases, where the internucleotide linkages of the oligonucleotide joining the 5′-end wing segment, the gapmer, and the 3′-end wing segment comprises only PS bonds. In some aspects, the oligonucleotide comprises a 5′-end wing segment comprising five nucleic acid bases, a gapmer, and a 3′-end wing segment comprising five nucleic acid bases, where the internucleotide linkages of the oligonucleotide joining the 5′-end wing segment, the gapmer, and the 3′-end wing segment comprises only PS bonds. In some aspects, the oligonucleotide comprises a 5′-end wing segment comprising six nucleic acid bases, a gapmer, and a 3′-end wing segment comprising six nucleic acid bases, where the internucleotide linkages of the oligonucleotide joining the 5′-end wing segment, the gapmer, and the 3′-end wing segment comprises only PS bonds. In some aspects, the oligonucleotide comprises a 5′-end wing segment comprising seven nucleic acid bases, a gapmer, and a 3′-end wing segment comprising seven nucleic acid bases, where the internucleotide linkages of the oligonucleotide joining the 5′-end wing segment, the gapmer, and the 3′-end wing segment comprises only PS bonds. In some aspects, the oligonucleotide comprises a 5′-end wing segment comprising eight nucleic acid bases, a gapmer, and a 3′-end wing segment comprising eight nucleic acid bases, where the internucleotide linkages of the oligonucleotide joining the 5′-end wing segment, the gapmer, and the 3′-end wing segment comprises only PS bonds. In some aspects, the oligonucleotide comprises a 5′-end wing segment comprising nine nucleic acid bases, a gapmer, and a 3′-end wing segment comprising nine nucleic acid bases, where the internucleotide linkages of the oligonucleotide joining the 5′-end wing segment, the gapmer, and the 3′-end wing segment comprises only PS bonds. In some aspects, the oligonucleotide comprises a 5′-end wing segment comprising 10 nucleic acid bases, a gapmer, and a 3′-end wing segment comprising 10 nucleic acid bases, where the internucleotide linkages of the oligonucleotide joining the 5′-end wing segment, the gapmer, and the 3′-end wing segment comprises only PS bonds.

In some aspects, the oligonucleotide comprising the 5′-end wing segment, a gapmer, the 3′-end wing segment, and PS bond as internucleotide linkage comprises a nucleic acid sequence that is at least 70%, 75%, 80%, 85%, 90%, 95%, or 99% identical to any one of SEQ ID NOs: 21-38. In some aspects, the oligonucleotide comprises the 5′-end wing segment, a gapmer, the 3′-end wing segment, and PS bond as internucleotide linkage, where the gapmer comprises a nucleic acid sequence that is at least 70%, 75%, 80%, 85%, 90%, 95%, or 99% identical to any one of SEQ ID NOs: 21-38.

In some instances, chemical modifications to enhance guide stability, synthesis, localization, intracellular retention, or lengthen half-lives may not be genetically encodable. An oligonucleotide can be circular, substantially circular, or otherwise linked in a contiguous fashion (e.g., can be arranged as a loop) and can also retain a substantially similar secondary structure as a substantially similar oligonucleotide that may not be circular or may not be a loop.

### Modification of Phosphate Backbone

In some aspects, the chemical modification comprises modification of one or both of the non-linking phosphate oxygens in the phosphodiester backbone linkage or modification of one or more of the linking phosphate oxygens in the phosphodiester backbone linkage. As used herein, “alkyl” is meant to refer to a saturated hydrocarbon group which is straight-chained or branched. Example alkyl groups include methyl (Me), ethyl (Et), propyl (e.g., n-propyl or isopropyl), butyl (e.g., n-butyl, isobutyl, or t-butyl), or pentyl (e.g., n-pentyl, isopentyl, or neopentyl). An alkyl group can contain from 1 to about 20, from 2 to about 20, from 1 to about 12, from 1 to about 8, from 1 to about 6, from 1 to about 4, or from 1 to about 3 carbon atoms. As used herein, “aryl” refers to monocyclic or polycyclic (e.g., having 2, 3, or 4 fused rings) aromatic hydrocarbons such as, for example, phenyl, naphthyl, anthracenyl, phenanthrenyl, indanyl, or indenyl. In some aspects, aryl groups have from 6 to about 20 carbon atoms. As used herein, “alkenyl” refers to an aliphatic group containing at least one double bond. As used herein, “alkynyl” refers to a straight or branched hydrocarbon chain containing 2-12 carbon atoms and characterized in having one or more triple bonds. Examples of alkynyl groups can include ethynyl, propargyl, or 3-hexynyl. “Arylalkyl” or “aralkyl” refers to an alkyl moiety in which an alkyl hydrogen atom is replaced by an aryl group. Aralkyl includes groups in which more than one hydrogen atom has been replaced by an aryl group. Examples of “arylalkyl” or “aralkyl” include benzyl, 2-phenylethyl, 3-phenylpropyl, 9-fluorenyl, benzhydryl, and trityl groups. “Cycloalkyl” refers to a cyclic, bicyclic, tricyclic, or polycyclic non-aromatic hydrocarbon groups having 3 to 12 carbons. Examples of cycloalkyl moieties include, but are not limited to, cyclopropyl, cyclopentyl, and cyclohexyl. “Heterocyclyl” refers to a monovalent radical of a heterocyclic ring system. Representative heterocyclyls include, without limitation, tetrahydrofuranyl, tetrahydrothienyl, pyrrolidinyl, pyrrolidonyl, piperidinyl, pyrrolinyl, piperazinyl, dioxanyl, dioxolanyl, diazepinyl, oxazepinyl, thiazepinyl, and morpholinyl. “Heteroaryl” refers to a monovalent radical of a heteroaromatic ring system. Examples of heteroaryl moieties can include imidazolyl, oxazolyl, thiazolyl, triazolyl, pyrrolyl, furanyl, indolyl, thiophenyl pyrazolyl, pyridinyl, pyrazinyl, pyridazinyl, pyrimidinyl, indolizinyl, purinyl, naphthyridinyl, quinolyl, and pteridinyl.

In some aspects, the phosphate group of a chemically modified nucleotide can be modified by replacing one or more of the oxygens with a different substituent. In some aspects, the chemically modified nucleotide can include replacement of an unmodified phosphate moiety with a modified phosphate as described herein. In some aspects, the modification of the phosphate backbone can include alterations that result in either an uncharged linker or a charged linker with unsymmetrical charge distribution. Examples of modified phosphate groups can include phosphorothioate, phosphonothioacetate, phosphoroselenates, boranophosphates, boranophosphate esters, hydrogen phosphonates, phosphoroamidates, alkyl or aryl phosphonates and phosphotriesters. In some aspects, one of the non-bridging phosphate oxygen atoms in the phosphate backbone moiety can be replaced by any of the following groups: sulfur (S), selenium (Se), BR3 (wherein R can be, e.g., hydrogen, alkyl, or aryl), C (e.g., an alkyl group, an aryl group, and the like), H, NR2 (wherein R can be, e.g., hydrogen, alkyl, or aryl), or (wherein R can be, e.g., alkyl or aryl). The phosphorous atom in an unmodified phosphate group can be achiral. However, replacement of one of the non-bridging oxygens with one of the above atoms or groups of atoms can render the phosphorous atom chiral. A phosphorous atom in a phosphate group modified in this way is a stereogenic center. The stereogenic phosphorous atom can possess either the “R” configuration (herein Rp) or the “S” configuration (herein Sp). In some cases, the oligonucleotide comprises stereopure nucleotides comprising S conformation of phosphorothioate or R conformation of phosphorothioate. In some aspects, the chiral phosphate product is present in a diastereomeric excess of 50%, 60%, 70%, 80%, 90%, or more. In some aspects, the chiral phosphate product is present in a diastereomeric excess of 95%. In some aspects, the chiral phosphate product is present in a diastereomeric excess of 96%. In some aspects, the chiral phosphate product is present in a diastereomeric excess of 97%. In some aspects, the chiral phosphate product is present in a diastereomeric excess of 98%. In some aspects, the chiral phosphate product is present in a diastereomeric excess of 99%. In some aspects, both non-bridging oxygens of phosphorodithioates can be replaced by sulfur. The phosphorus center in the phosphorodithioates can be achiral which precludes the formation of oligoribonucleotide diastereomers. In some aspects, modifications to one or both non-bridging oxygens can also include the replacement of the non-bridging oxygens with a group independently selected from S, Se, B, C, H, N, and OR (R can be, e.g., alkyl or aryl). In some aspects, the phosphate linker can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either or both of the linking oxygens.

In certain embodiments, nucleic acids comprise linked nucleic acids. Nucleic acids can be linked together using any internucleotide linkage. The two main classes of internucleotide linkage groups are defined by the presence or absence of a phosphorus atom. Representative phosphorus containing internucleotide linkage include, but are not limited to, phosphodiesters, phosphotriesters, methylphosphonates, phosphoramidate, and phosphorothioates (P═S). Representative non-phosphorus containing internucleotide linkage groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N*-dimethylhydrazine (—CH₂—N(CH₃)—N(CH₃)). In certain embodiments, internucleotide linkages having a chiral atom can be prepared as a racemic mixture, as separate enantiomers, e.g., alkylphosphonates and phosphorothioates. Unnatural nucleic acids can contain a single modification. Unnatural nucleic acids can contain multiple modifications within one of the moieties or between different moieties.

Backbone phosphate modifications to nucleic acid include, but are not limited to, methyl phosphonate, phosphorothioate, phosphoramidate (bridging or non-bridging), phosphotriester, phosphorodithioate, phosphodithioate, and boranophosphate, and can be used in any combination. Other non-phosphate linkages may also be used.

In some aspects, backbone modifications (e.g., methylphosphonate, phosphorothioate, phosphoroamidate and phosphorodithioate internucleotide linkages) can confer immunomodulatory activity on the modified nucleic acid and/or enhance their stability in vivo.

In some instances, a phosphorous derivative (or modified phosphate group) is attached to the sugar or sugar analog moiety in and can be a monophosphate, diphosphate, triphosphate, alkylphosphonate, phosphorothioate, phosphorodithioate, phosphoramidate or the like.

In some cases, backbone modification comprises replacing the phosphodiester linkage with an alternative moiety such as an anionic, neutral or cationic group. Examples of such modifications include: anionic internucleoside linkage; N3′ to P5′ phosphoramidate modification; boranophosphate DNA; prooligonucleotides; neutral internucleoside linkages such as methylphosphonates; amide linked DNA; methylene(methylimino) linkages; formacetal and thioformacetal linkages; backbones containing sulfonyl groups; morpholino oligos; peptide nucleic acids (PNA); and positively charged deoxyribonucleic guanidine (DNG) oligos. A modified nucleic acid may comprise a chimeric or mixed backbone comprising one or more modifications, e.g. a combination of phosphate linkages such as a combination of phosphodiester and phosphorothioate linkages.

Substitutes for the phosphate include, for example, short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. It is also understood in a nucleotide substitute that both the sugar and the phosphate moieties of the nucleotide can be replaced, by for example an amide type linkage (aminoethylglycine) (PNA). It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethylammonium 1-di-O-hexadecyl-rac-glycero-S—H-phosphonate, a polyamine or a polyethylene glycol chain, or adamantane acetic acid, a palmityl moiety, or an octadecylamine or hexylamino-carbonyl-oxycholesterol moiety.

In some aspects, the chemical modification described herein comprises modification of a phosphate backbone. In some aspects, the oligonucleotide described herein comprises at least one chemically modified phosphate backbone. Exemplary chemically modification of the phosphate group or backbone can include replacing one or more of the oxygens with a different substituent. Furthermore, the modified nucleotide present in the oligonucleotide can include the replacement of an unmodified phosphate moiety with a modified phosphate as described herein. In some aspects, the modification of the phosphate backbone can include alterations resulting in either an uncharged linker or a charged linker with unsymmetrical charge distribution. Exemplary modified phosphate groups can include, phosphorothioate, phosphonothioacetate, phosphoroselenates, borano phosphates, borano phosphate esters, hydrogen phosphonates, phosphoroamidates, alkyl or aryl phosphonates and phosphotriesters. In some aspects, one of the non-bridging phosphate oxygen atoms in the phosphate backbone moiety can be replaced by any of the following groups: sulfur (S), selenium (Se), BR3 (wherein R can be, e.g., hydrogen, alkyl, or aryl), C (e.g., an alkyl group, an aryl group, and the like), H, NR2 (wherein R can be, e.g., hydrogen, alkyl, or aryl), or (wherein R can be, e.g., alkyl or aryl). The phosphorous atom in an unmodified phosphate group is achiral. However, replacement of one of the non-bridging oxygens with one of the above atoms or groups of atoms can render the phosphorous atom chiral; that is to say that a phosphorous atom in a phosphate group modified in this way is a stereogenic center. The stereogenic phosphorous atom can possess either the “R” configuration (herein Rp) or the “S” configuration (herein Sp). In such case, the chemically modified oligonucleotide can be stereopure (e.g. S or R confirmation). In some cases, the chemically modified oligonucleotide comprises stereopure phosphate modification. For example, the chemically modified oligonucleotide comprises S conformation of phosphorothioate or R conformation of phosphorothioate.

Phosphorodithioates have both non-bridging oxygens replaced by sulfur. The phosphorus center in the phosphorodithioates is achiral which precludes the formation of oligoribonucleotide diastereomers. In some aspects, modifications to one or both non-bridging oxygens can also include the replacement of the non-bridging oxygens with a group independently selected from S, Se, B, C, H, N, and OR (R can be, e.g., alkyl or aryl).

The phosphate linker can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens.

### Replacement of Phosphate Moiety

In some aspects, at least one phosphate group of the oligonucleotide can be chemically modified. In some aspects, the phosphate group can be replaced by non-phosphorus containing connectors. In some aspects, the phosphate moiety can be replaced by dephospho linker. In some aspects, the charge phosphate group can be replaced by a neutral group. In some cases, the phosphate group can be replaced by methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino. In some aspects, nucleotide analogs described herein can also be modified at the phosphate group. Modified phosphate group can include modification at the linkage between two nucleotides with phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates (e.g. 3′-amino phosphoramidate and aminoalkylphosphoramidates), thionophosphoramidates, thionoalkylphosphonates, thionoalkylphosphotriesters, and boranophosphates. The phosphate or modified phosphate linkage between two nucleotides can be through a 3′-5′ linkage or a 2′-5′ linkage, and the linkage contains inverted polarity such as 3′-5′ to 5′-3′ or 2′-5′ to 5′-2′.

### Substitution of Phosphate Group

In some aspects, the chemical modification described herein comprises modification by replacement of a phosphate group. In some aspects, the oligonucleotide described herein comprises at least one chemically modification comprising a phosphate group substitution or replacement. Exemplary phosphate group replacement can include non-phosphorus containing connectors. In some aspects, the phosphate group substitution or replacement can include replacing charged phosphate group can by a neutral moiety. Exemplary moieties which can replace the phosphate group can include methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

### Modification of the Ribophosphate Backbone

In some aspects, the chemical modification described herein comprises modifying ribophosphate backbone of the oligonucleotide. In some aspects, the oligonucleotide described herein comprises at least one chemically modified ribophosphate backbone. Exemplary chemically modified ribophosphate backbone can include scaffolds that can mimic nucleic acids can also be constructed wherein the phosphate linker and ribose sugar are replaced by nuclease resistant nucleoside or nucleotide surrogates. In some aspects, the nucleobases can be tethered by a surrogate backbone. Examples can include morpholino such as a phosphorodiamidate morpholino oligomer (PMO), thiomorpholino, cyclobutyl, pyrrolidine and peptide nucleic acid (PNA) nucleoside surrogates.

### Modification of Sugar

In some aspects, the chemical modification described herein comprises modification of sugar. In some aspects, the oligonucleotide described herein comprises at least one chemically modified sugar. Exemplary chemically modified sugar can include 2′ hydroxyl group (OH) modified or replaced with a number of different “oxy” or “deoxy” substituents. In some aspects, modifications to the 2′ hydroxyl group can enhance the stability of the nucleic acid since the hydroxyl can no longer be deprotonated to form a 2′-alkoxide ion. The 2′-alkoxide can catalyze degradation by intramolecular nucleophilic attack on the linker phosphorus atom. Examples of “oxy”-2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH₂CH₂O)ₙCH₂CH₂OR, wherein R can be, e.g., H or optionally substituted alkyl, and n can be an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20). In some aspects, the “oxy”-2′ hydroxyl group modification can include (LNA, in which the 2′ hydroxyl can be connected, e.g., by a Ci-₆ alkylene or Cj-6 heteroalkylene bridge, to the 4′ carbon of the same ribose sugar, where exemplary bridges can include methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy, O(CH₂)ₙ-amino, (wherein amino can be, e.g., NH₂; alkylamino, dialkylamino, heterocyclyl, arylamino, diarylamino, heteroarylamino, or diheteroarylamino, ethylenediamine, or polyamino). In some aspects, the “oxy”-2′ hydroxyl group modification can include the methoxyethyl group (MOE), (OCH₂CH₂OCH₃, e.g., a PEG derivative). In some cases, the deoxy modifications can include hydrogen (i.e., deoxyribose sugars, e.g., at the overhang portions of partially dsRNA); halo (e.g., bromo, chloro, fluoro, or iodo); amino (wherein amino can be, e.g., NH₂; alkylamino, dialkylamino, heterocyclyl, arylamino, diarylamino, heteroarylamino, diheteroarylamino, or amino acid); NH(CH₂CH₂NH)ₙCH₂CH₂-amino (wherein amino can be, e.g., as described herein), NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which can be optionally substituted with e.g., an amino as described herein. In some instances, the sugar group can also contain one or more carbons that possess the opposite stereochemical configuration than that of the corresponding carbon in ribose. Thus, a modified nucleic acid can include nucleotides containing e.g., arabinose, as the sugar. The nucleotide “monomer” can have an alpha linkage at the F position on the sugar, e.g., alpha-nucleosides. The modified nucleic acids can also include “abasic” sugars, which lack a nucleobase at C—. The abasic sugars can also be further modified at one or more of the constituent sugar atoms. The modified nucleic acids can also include one or more sugars that are in the L form, e.g. L-nucleosides. In some aspects, the oligonucleotide described herein includes the sugar group ribose, which is a 5-membered ring having an oxygen.

Exemplary modified nucleosides and modified nucleotides can include replacement of the oxygen in ribose (e.g., with sulfur (S), selenium (Se), or alkylene, such as, e.g., methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for example, anhydrohexitol, altritol, mannitol, cyclohexanyl, cyclohexenyl, and morpholino that also has a phosphoramidate backbone. In some aspects, the modified nucleotides can include multicyclic forms (e.g., tricyclo; and “unlocked” forms, such as glycol nucleic acid (GNA) (e.g., R-GNA or S-GNA, where ribose is replaced by glycol units attached to phosphodiester bonds), threose nucleic acid. In some aspects, the modifications to the sugar of the oligonucleotide comprises modifying the oligonucleotide to include locked nucleic acid (LNA), unlocked nucleic acid (UNA), ethylene nucleic acid (ENA), constrained ethyl (cEt) sugar, or bridged nucleic acid (BNA).

### Modification of a Constituent of the Ribose Sugar

In some aspects, the oligonucleotide described herein comprises at least one chemical modification of a constituent of the ribose sugar. In some aspects, the chemical modification of the constituent of the ribose sugar can include 2′-O-methyl, 2′-O-methoxyethyl (2′-O-MOE), 2′-fluoro, 2′-aminoethyl, 2′-deoxy-2′-fuloarabinou-cleic acid, 2′-deoxy, 2′-deoxy-2′-fluoro, 2′-O-methyl, 3′-phosphorothioate, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), 2′-O—N-methylacetamido (2′-O-NMA) 3′-phosphonoacetate (PACE), or 3′-phosphonothioacetate (thioPACE). In some aspects, the chemical modification of the constituent of the ribose sugar comprises unnatural nucleic acid. In some instances, the unnatural nucleic acids include modifications at the 5′-position and the 2′-position of the sugar ring, such as 5′-CH₂-substituted 2′-O-protected nucleosides. In some cases, unnatural nucleic acids include amide linked nucleoside dimers have been prepared for incorporation into oligonucleotides wherein the 3′ linked nucleoside in the dimer (5′ to 3′) comprises a 2′-OCH₃ and a 5′-(S)—CH₃. Unnatural nucleic acids can include 2′-substituted 5′-CH₂ (or O) modified nucleosides. Unnatural nucleic acids can include 5′-methylenephosphonate DNA and RNA monomers, and dimers. Unnatural nucleic acids can include 5′-phosphonate monomers having a 2′-substitution and other modified 5′-phosphonate monomers. Unnatural nucleic acids can include 5′-modified methylenephosphonate monomers. Unnatural nucleic acids can include analogs of 5′ or 6′-phosphonate ribonucleosides comprising a hydroxyl group at the 5′ and/or 6′-position. Unnatural nucleic acids can include 5′-phosphonate deoxyribonucleoside monomers and dimers having a 5′-phosphate group. Unnatural nucleic acids can include nucleosides having a 6′-phosphonate group wherein the 5′ or/and 6′-position is unsubstituted or substituted with a thio-tert-butyl group (SC(CH₃)₃) (and analogs thereof); a methyleneamino group (CH₂NH₂) (and analogs thereof) or a cyano group (CN) (and analogs thereof).

In some aspects, unnatural nucleic acids also include modifications of the sugar moiety. In some cases, nucleic acids contain one or more nucleosides wherein the sugar group has been modified. Such sugar modified nucleosides may impart enhanced nuclease stability, increased binding affinity, or some other beneficial biological property. In certain embodiments, nucleic acids comprise a chemically modified ribofuranose ring moiety. Examples of chemically modified ribofuranose rings include, without limitation, addition of substituent groups (including 5′ and/or 2′ substituent groups; bridging of two ring atoms to form bicyclic nucleic acids; replacement of the ribosyl ring oxygen atom with S, N(R), or C(R₁)(R₂) (R═H, C₁-C₁₂ alkyl or a protecting group); and combinations thereof.

In some instances, the oligonucleotide described herein comprises modified sugars or sugar analogs. Thus, in addition to ribose and deoxyribose, the sugar moiety can be pentose, deoxypentose, hexose, deoxyhexose, glucose, arabinose, xylose, lyxose, or a sugar “analog” cyclopentyl group. The sugar can be in a pyranosyl or furanosyl form. The sugar moiety can be the furanoside of ribose, deoxyribose, arabinose or 2′-O-alkylribose, and the sugar can be attached to the respective heterocyclic bases either in [alpha] or [beta] anomeric configuration. Sugar modifications include, but are not limited to, 2′-alkoxy-RNA analogs, 2′-amino-RNA analogs, 2′-fluoro-DNA, and 2′-alkoxy- or amino-RNA/DNA chimeras. For example, a sugar modification may include 2′-O-methyl-uridine or 2′-O-methyl-cytidine. Sugar modifications include 2′-O-alkyl-substituted deoxyribonucleosides and 2′-O-ethyleneglycol-like ribonucleosides.

Modifications to the sugar moiety include natural modifications of the ribose and deoxy ribose as well as unnatural modifications. Sugar modifications include, but are not limited to, the following modifications at the 2′ position: OH; F; O—, S—, or N-alkyl; O—, S—, or N-alkenyl; O- , S- or N-alkynyl; or O-alkyl-O-alkyl, wherein the alkyl, alkenyl and alkynyl can be substituted or unsubstituted C₁ to C₁₀, alkyl or C₂ to C₁₀ alkenyl and alkynyl. 2′ sugar modifications also include but are not limited to —O[(CH₂)ₙO]ₘ CH₃, —O(CH₂)ₙOCH₃, —O(CH₂)ₙNH₂, —O(CH₂)ₙCH₃, —O(CH₂)ₙONH₂, and —O(CH₂)ₙON[(CH₂)ₙ CH₃)]2, where n and m are from 1 to about 10. Other chemical modifications at the 2′ position include but are not limited to: C₁ to C₁₀ lower alkyl, substituted lower alkyl, alkaryl, aralkyl, O-alkaryl, O-aralkyl, SH, SCH₃, OCN, Cl, Br, CN, CF₃, OCF₃, SOCH₃, SO₂ CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of the 5′ terminal nucleotide. Chemically modified sugars also include those that contain modifications at the bridging ring oxygen, such as CH₂ and S. Nucleotide sugar analogs can also have sugar mimetics such as cyclobutyl moieties in place of the pentofuranosyl sugar. Examples of nucleic acids having modified sugar moieties include, without limitation, nucleic acids comprising 5′-vinyl, 5′-methyl (R or S), 4′-S, 2′-F, 2′-OCH₃, and 2′-O(CH₂)₂OCH₃ substituent groups. The substituent at the 2′ position can also be selected from allyl, amino, azido, thio, O-allyl, O—(C₁-C₁₀ alkyl), OCF₃, O(CH₂)₂SCH₃, O(CH₂)₂—O—N(Rₘ)(Rₙ), and O-CH₂—C(═O)—N(Rₘ)(Rₙ), where each Rₘ and Rₙ is, independently, H or substituted or unsubstituted C₁-C₁₀ alkyl.

In certain embodiments, nucleic acids described herein include one or more bicyclic nucleic acids. In certain such embodiments, the bicyclic nucleic acid comprises a bridge between the 4′ and the 2′ ribosyl ring atoms. In certain embodiments, nucleic acids provided herein include one or more bicyclic nucleic acids wherein the bridge comprises a 4′ to 2′ bicyclic nucleic acid. Examples of such 4′ to 2′ bicyclic nucleic acids include, but are not limited to, one of the formulae: 4′-(CH₂)—O-2′ (LNA); 4′-(CH₂)—S-2′; 4′—(CH₂)₂—O-2′ (ENA); 4′-CH(CH₃)—O-2′ and 4′-CH(CH₂OCH₃)—O-2′, and analogs thereof; 4′-C(CH₃)(CH₃)—O-2′ and analogs thereof.

### Modifications on the Base of Nucleotide

In some aspects, the chemical modification described herein comprises modification of the base of nucleotide (e.g. the nucleobase). Exemplary nucleobases can include adenine (A), thymine (T), guanine (G), cytosine (C), and uracil (U). These nucleobases can be modified or replaced to in the oligonucleotide described herein. The nucleobase of the nucleotide can be independently selected from a purine, a pyrimidine, a purine or pyrimidine analog. In some aspects, the nucleobase can be naturally occurring or synthetic derivatives of a base.

In some aspects, the chemical modification described herein comprises modifying an uracil. In some aspects, the oligonucleotide described herein comprises at least one chemically modified uracil. Exemplary chemically modified uracil can include pseudouridine, pyridin-4-one ribonucleoside, 5-aza-uridine, 6-aza-uridine, 2-thio-5-aza-uridine, 2-thio-uridine, 4-thio-uridine, 4-thio-pseudouridine, 2-thio-pseudouridine, 5-hydroxy-uridine, 5-aminoallyl-uridine, 5-halo-uridine (e.g., 5-iodo-uridine or 5-bromo-uridine), 3-methyl-uridine, 5-methoxy-uridine, uridine 5-oxyacetic acid, uridine 5-oxyacetic acid methyl ester, 5-carboxymethyl-uridine, 1-carboxymethyl-pseudouridine, 5-carboxyhydroxymethyl-uridine, 5-carboxyhydroxymethyl-uridine methyl ester, 5-methoxycarbonylmethyl-uridine, 5-methoxycarbonylmethyl-2-thio-uridine, 5-aminomethyl-2-thio-uridine, 5-methylaminomethyl-uridine, 5-methylaminomethyl-2-thio-uridine, 5-methylaminomethyl-2-seleno-uridine, 5-carbamoylmethyl-uridine, 5-carboxymethylaminomethyl-uridine, 5-carboxymethylaminomethyl-2-thio-uridine, 5-propynyl-uridine, 1-propynyl-pseudouridine, 5-taurinomethyl-uridine, 1-taurinomethyl-pseudouridine, 5-taurinomethyl-2-thio-uridine, 1-taurinomethyl-4-thio-pseudouridine, 5-methyl-uridine, 1 methyl-pseudouridine, 5-methyl-2-thio-uridine, 1-methyl-4-thio-pseudouridine, 4-thio-1-methyl-pseudouridine, 3-methyl-pseudouridine, 2-thio-1-methyl-pseudouridine, 1-methyl-1-deaza-pseudouridine, 2-thio-1-methyl-1-deaza-pseudouridine, dihydroundine, dihydropseudoundine, 5,6-dihydrouridine, 5-methyl-dihydrouridine, 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl) uridine, 1-methyl-3-(3-amino-3-carboxypropy pseudouridine, 5-(isopentenylaminomethyl) uridine, 5-(isopentenylaminomethy])-2-thio-uridine, a-thio-uridine, 2′-O-methyl-uridine, 5,2′-O-dimethyl-uridine, 2′-O-methyl-pseudouridine, 2-thio-2′-O-methyl-uridine, 5-methoxycarbonylmethyl-2′-O-methyl-uridine, 5-carbamoylmethyl-2′-O-methyl-uridine, 5-carboxymethylaminomethyl-2′-O-methyl-uridine, 3,2′-O-dimethyl-uridine, 5-(isopentenylaminomethyl)-2′-O-methyl-uridine, l-thio-uridine, deoxythymidine, 2′-F-ara-uridine, 2′-F-uridine, 2′-OH-ara-uridine, 5-(2-carbomethoxyvinyl) uridine, 5-[3-(1-E-propenylamino)uridine, pyrazolo[3,4-d]pyrimidines, xanthine, and hypoxanthine.

In some aspects, the chemical modification described herein comprises modifying a cytosine. In some aspects, the oligonucleotide described herein comprises at least one chemically modified cytosine. Exemplary chemically modified cytosine can include 5-aza-cytidine, 6-aza-cytidine, pseudoisocytidine, 3-methyl-cytidine, N4-acetyl-cytidine, 5-formyl-cytidine, N4-methyl-cytidine, 5-methyl-cytidine, 5-halo-cytidine, 5-hydroxymethyl-cytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine, a-thio-cytidine, 2′-O-methyl-cytidine, 5,2′-O-dimethyl-cytidine, N4-acetyl-2′-O-methyl-cytidine, N4,2′-O-dimethyl-cytidine, 5-formyl-2′-O-methyl-cytidine, N4,N4,2′-O-trimethyl-cytidine, 1-thio-cytidine, 2′-F-ara-cytidine, 2′-F-cytidine, and 2′-OH-ara-cytidine.

In some aspects, the chemical modification described herein comprises modifying a adenine. In some aspects, the oligonucleotide described herein comprises at least one chemically modified adenine. Exemplary chemically modified adenine can include 2-amino-purine, 2,6-diaminopurine, 2-amino-6-halo-purine (e.g., 2-amino-6-chloro-purine), 6-halo-purine (e.g., 6-chloi-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine, 2-methyl-adenine, N6-methyl-adenosine, 2-methylthio-N6-methyl-adenosine, N6-isopentenyl-adenosine, 2-methylthio-N6-isopentenyl-adenosine, N6-(cis-hydroxyisopentenyl) adenosine, 2-methylthio-N6-(cis-hydroxyisopentenyl) adenosine, N6-glycinylcarbamoyl-adenosine, N6-threonylcarbamoyl-adenosine, N6-methyl-N6-threonylcarbamoyl-adenosine, 2-methylthio-N6-threonylcarbamoyl-adenosine, N6, N6-dimethyladenosine, N6-hydroxynorvalylcarbamoyl-adenosine, 2-methylthio-N6-hydroxynorvalylcarbamoyl-adenosine, N6-acetyl-adenosine, 7-methyl-adenine, 2-methylthio-adenine, 2-methoxy-adenine, a-thio-adenosine, 2′-O-methyl-adenosine, N6, 2′—O-dimethyladenosine, N6-Methyl-2′-deoxyadenosine, N6, N6, 2′—O-trimethyl-adenosine, 1,2′-O-dimethyladenosine, 2′-O-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

In some aspects, the chemical modification described herein comprises modifying a guanine. In some aspects, the oligonucleotide described herein comprises at least one chemically modified guanine. Exemplary chemically modified guanine can include inosine, 1-methyl-inosine, wyosine, methylwyosine, 4-demethyl-wyosine, isowyosine, wybutosine, peroxywybutosine, hydroxywybutosine, undemriodified hydroxywybutosine, 7-deaza-guanosine, queuosine, epoxyqueuosine, galactosyl-queuosine, mannosyl-queuosine, 7-cyano-7-deaza-guanosine, 7-aminomethyl-7-deaza-guanosine, archaeosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methyl-inosine, 6-methoxy-guanosine, 1-methyl-guanosine, N2-methyl-guanosine, N2, N2-dimethyl-guanosine, N2, 7-dimethyl-guanosine, N2, N2, 7-dimethyl-guanosine, 8-oxo-guanosine, 7-methyl-8-oxo-guanosine, 1-meththio-guanosine, N2-methyl-6-thio-guanosine, N2,N2-dimethyl-6-thio-guanosine, a-thio-guanosine, 2′-O-methyl-guanosine, N2-methyl-2′-O-methyl-guanosine, N2,N2-dimethyl-2′-O-methyl-guanosine, 1-methyl-2′-O-methyl-guanosine, N2, 7-dimethyl-2′-O-methyl-guanosine, 2′-O-methyl-inosine, 1, 2′—O-dimethyl-inosine, 6-O-phenyl-2′-deoxyinosine, 2′-O-ribosylguanosine, 1-thio-guanosine, 6-O-methyguanosine, O⁶-Methyl-2′-deoxyguanosine, 2′-F-ara-guanosine, and 2′-F-guanosine.

In some cases, the chemical modification of the oligonucleotide can include introducing or substituting a nucleic acid analog or an unnatural nucleic acid into the oligonucleotide. In some aspects, nucleic acid analog can be any one of the chemically modified nucleic acid described herein. all of which are expressly incorporated by reference in their entireties. The chemically modified nucleotide described herein can include a variant of guanosine, uridine, adenosine, thymidine, and cytosine, including any natively occurring or non-natively occurring guanosine, uridine, adenosine, thymidine or cytidine that has been altered chemically, for example by acetylation, methylation, hydroxylation. Exemplary chemically modified nucleotide can include 1-methyl-adenosine, 1-methyl-guanosine, 1-methyl-inosine, 2,2-dimethyl-guanosine, 2,6-diaminopurine, 2′-amino-2′-deoxyadenosine, 2′-amino-2′-deoxycytidine, 2′-amino-2′-deoxyguanosine, 2′-amino-2′-deoxyuridine, 2-amino-6-chloropurineriboside, 2-aminopurine-riboside, 2′-araadenosine, 2′-aracytidine, 2′-arauridine, 2′-azido-2′-deoxyadenosine, 2′-azido-2′-deoxycytidine, 2′-azido-2′-deoxyguanosine, 2′-azido-2′-deoxyuridine, 2-chloroadenosine, 2′-fluoro-2′-deoxyadenosine, 2′-fluoro-2′-deoxycytidine, 2′-fluoro-2′-deoxyguanosine, 2′-fluoro-2′-deoxyuridine, 2′-fluorothymidine, 2-methyl-adenosine, 2-methyl-guanosine, 2-methyl-thio-N6-isopenenyl-adenosine, 2′-O-methyl-2-aminoadenosine, 2′-O-methyl-2′-deoxyadenosine, 2′-O-methyl-2′-deoxycytidine, 2′-O-methyl-2′-deoxyguanosine, 2, —O-methyl-2′-deoxyuridine, 2′-O-methyl-5-methyluridine, 2′-O-methylinosine, 2′-O-methylpseudouridine, 2-thiocytidine, 2-thio-cytidine, 3-methyl-cytidine, 4-acetyl-cytidine, 4-thiouridine, 5-(carboxyhydroxymethyl)-uridine, 5,6-dihydrouridine, 5-aminoallylcytidine, 5-aminoallyl-deoxyuridine, 5-bromouridine, 5-carboxymethylaminomethyl-2-thio-uracil, 5-carboxymethylamonomethyl-uracil, 5-chloro-ara-cytosine, 5-fluoro-uridine, 5-iodouridine, 5-methoxycarbonylmethyl-uridine, 5-methoxy-uridine, 5-methyl-2-thio-uridine, 6-Azacytidine, 6-azauridine, 6-chloro-7-deaza-guanosine, 6-chloropurineriboside, 6-mercapto-guanosine, 6-methyl-mercaptopurine-riboside, 7-deaza-2′-deoxyguanosine, 7-deazaadenosine, 7-methyl-guanosine, 8-azaadenosine, 8-bromo-adenosine, 8-bromo-guanosine, 8-mercapto-guanosine, 8-oxoguanosine, benzimidazole-riboside, beta-D-mannosyl-queosine, dihydro-uridine, inosine, N1-methyladenosine, N6-([6-aminohexyl] carbamoylmethyl)-adenosine, N6-isopentenyl-adenosine, N6-methyl-adenosine, N7-methyl-xanthosine, N-uracil-5-oxyacetic acid methyl ester, puromycin, queosine, uracil-5-oxyacetic acid, uracil-5-oxy acetic acid methyl ester, wybutoxosine, xanthosine, and xylo-adenosine. In some aspects, the chemically modified nucleic acid as described herein comprises at least one chemically modified nucleotide selected from 2-amino-6-chloropurineriboside-5′-triphosphate, 2-aminopurine-riboside-5′-triphosphate, 2-aminoadenosine-5′-triphosphate, 2′-amino-2′-deoxycytidine-triphosphate, 2-thiocytidine-5′-triphosphate, 2-thiouridine-5′-triphosphate, 2′-fluorothymidine-5′-triphosphate, 2′—O-methyl-inosine-5′-triphosphate, 4-thiouridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-bromo-2′-deoxycytidine-5′-triphosphate, 5-bromo-2′-deoxyuridine-5′-triphosphate, 5-iodocytidine-5′-triphosphate, 5-iodo-2′-deoxycytidine-5′-triphosphate, 5-iodouridine-5′-triphosphate, 5-iodo-2′-deoxyuridine-5′-triphosphate, 5-methylcytidine-5′-triphosphate, 5-methyluridine-5′-triphosphate, 5-propynyl-2′-deoxycytidine-5′-triphosphate, 5-propynyl-2′-deoxyuridine-5′-triphosphate, 6-azacytidine-5′-triphosphate, 6-azauridine-5′-triphosphate, 6-chloropurineriboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, 6-methylguanosine-5′-triphosphate, pseudouridine-5′-triphosphate, puromycin-5′-triphosphate, or xanthosine-5′-triphosphate. In some aspects, the chemically modified nucleic acid as described herein comprises at least one chemically modified nucleotide selected from pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine. In some aspects, the artificial nucleic acid as described herein comprises at least one chemically modified nucleotide selected from 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine. In some aspects, the chemically modified nucleic acid as described herein comprises at least one chemically modified nucleotide selected from 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine. In other embodiments, the chemically modified nucleic acid as described herein comprises at least one chemically modified nucleotide selected from inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine. In certain embodiments, the chemically modified nucleic acid as described herein comprises at least one chemically modified nucleotide selected from 6-aza-cytidine, 2-thio-cytidine, alpha-thio-cytidine, pseudo-iso-cytidine, 5-aminoallyl-uridine, 5-iodo-uridine, N1-methyl-pseudouridine, 5,6-dihydrouridine, alpha-thio-uridine, 4-thio-uridine, 6-aza-uridine, 5-hydroxy-uridine, deoxy-thymidine, 5-methyl-uridine, pyrrolo-cytidine, inosine, alpha-thio-guanosine, 6-methyl-guanosine, 5-methyl-cytdine, 8-oxo-guanosine, 7-deaza-guanosine, N1-methyl-adenosine, 2-amino-6-chloro-purine, N6-methyl-2-amino-purine, pseudo-iso-cytidine, 6-chloro-purine, N6-methyl-adenosine, alpha-thio-adenosine, 8-azido-adenosine, 7-deaza-adenosine.

A modified base of a unnatural nucleic acid includes, but is not limited to, uracil-5-yl, hypoxanthin-9-yl (I), 2-aminoadenin-9-yl, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Certain unnatural nucleic acids, such as 5-substituted pyrimidines, 6-azapyrimidines and N-2 substituted purines, N-6 substituted purines, 0-6 substituted purines, 2-aminopropyladenine, 5-propynyluracil, 5-propynylcytosine, 5-methylcytosine, those that increase the stability of duplex formation, universal nucleic acids, hydrophobic nucleic acids, promiscuous nucleic acids, size-expanded nucleic acids, fluorinated nucleic acids, 5-substituted pyrimidines, 6-azapyrimidines and N-2, N-6 and 0-6 substituted purines, including 2-aminopropyladenine, 5-propynyluracil and 5-propynylcytosine. 5-methylcytosine (5-me-C), 5-hydroxymethyl cytosine, xanthine, hypoxanthine, 2-aminoadenine, 6-methyl, other alkyl derivatives of adenine and guanine, 2-propyl and other alkyl derivatives of adenine and guanine, 2-thiouracil, 2-thiothymine and 2-thiocytosine, 5-halouracil, 5-halocytosine, 5-propynyl (—C≡C—CH₃) uracil, 5-propynyl cytosine, other alkynyl derivatives of pyrimidine nucleic acids, 6-azo uracil, 6-azo cytosine, 6-azo thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl, other 5-substituted uracils and cytosines, 7-methylguanine, 7-methyladenine, 2-F-adenine, 2-amino-adenine, 8-azaguanine, 8-azaadenine, 7-deazaguanine, 7-deazaadenine, 3-deazaguanine, 3-deazaadenine, tricyclic pyrimidines, phenoxazine cytidine([5,4-b][1,4]benzoxazin-2(3H)-one), phenothiazine cytidine (1H-pyrimido[5,4-b][1,4]benzothiazin-2(3H)-one), G-clamps, phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one), those in which the purine or pyrimidine base is replaced with other heterocycles, 7-deaza-adenine, 7-deazaguanosine, 2-aminopyridine, 2-pyridone, azacytosine, 5-bromocytosine, bromouracil, 5-chlorocytosine, chlorinated cytosine, cyclocytosine, cytosine arabinoside, 5-fluorocytosine, fluoropyrimidine, fluorouracil, 5,6-dihydrocytosine, 5-iodocytosine, hydroxyurea, iodouracil, 5-nitrocytosine, 5-bromouracil, 5-chlorouracil, 5-fluorouracil, and 5-iodouracil, 2-amino-adenine, 6-thio-guanine, 2-thio-thymine, 4-thio-thymine, 5-propynyl-uracil, 4-thio-uracil, N4-ethylcytosine, 7-deazaguanine, 7-deaza-8-azaguanine, 5-hydroxycytosine, 2′-deoxyuridine, or 2-amino-2′-deoxyadenosine.

In some cases, the at least one chemical modification comprises chemically modifying the 5′ or 3′ end such as 5′ cap or 3′ tail of the oligonucleotide. In some aspects, the oligonucleotide comprises a chemical modification comprising 3′ nucleotides which can be stabilized against degradation, e.g., by incorporating one or more of the modified nucleotides described herein. In this embodiment, uridines can be replaced with modified uridines, e.g., 5-(2-amino) propyl uridine, and 5-bromo uridine, or with any of the modified uridines described herein; adenosines and guanosines can be replaced with modified adenosines and guanosines, e.g., with modifications at the 8-position, e.g., 8-bromo guanosine, or with any of the modified adenosines or guanosines described herein. In some aspects, deaza nucleotides, e.g., 7-deaza-adenosine, can be incorporated into the gRNA. In some aspects, O- and N-alkylated nucleotides, e.g., N6-methyladenosine, can be incorporated into the gRNA. In some aspects, sugar-modified ribonucleotides can be incorporated, e.g., wherein the 2′ OH-group is replaced by a group selected from H, —OR, —R (wherein R can be, e.g., alkyl, cycloalkyl, aryl, aralkyl, heteroaryl or sugar), halo, —SH, —SR (wherein R can be, e.g., alkyl, cycloalkyl, aryl, aralkyl, heteroaryl or sugar), amino (wherein amino can be, e.g., NH₂; alkylamino, dialkylamino, heterocyclyl, arylamino, diarylamino, heteroarylamino, diheteroarylamino, or amino acid); or cyano (—CN). In some aspects, the phosphate backbone can be modified as described herein, e.g., with a phosphothioate group. In some aspects, the nucleotides in the overhang region of the gRNA can each independently be a modified or unmodified nucleotide including, but not limited to 2′-sugar modified, such as, 2-F 2′-O-methyl, thymidine (T), 2′-O-methoxyethyl-5-methyluridine (Teo), 2′-O-methoxyethyladenosine (Aeo), 2′-O-methoxyethyl-5-methylcytidine (m5Ceo), or any combinations thereof.

In some aspects, the oligonucleotide comprising at least one chemical modification, upon binding to the target RNA, is more specific in recruiting the endogenous nuclease for decreasing expression the target RNA compared to an oligonucleotide sharing identical nucleic acid sequence, but without any chemical modification, with the oligonucleotide comprising at least one chemical modification. In some aspects, the oligonucleotide comprising at least one chemical modification is at least 10%, 20%, 30%, 40%, 50%, 60%, 70%, 80%, 90%, two fold, three fold, four fold, five fold, six fold, seven fold, eight fold, nine fold, 10 fold, 20 fold, 30 fold, 40 fold, 50 fold, 100 fold, 500 fold, 1000 fold, or more specific in recruiting the endogenous nuclease for decreasing expression the target RNA compared to an oligonucleotide sharing identical nucleic acid sequence, but without any chemical modification, with the oligonucleotide comprising at least one chemical modification.

In some aspects, the oligonucleotide comprising at least one chemical modification comprises an increased resistance towards degradation by hydrolysis compared to an oligonucleotide sharing identical nucleic acid sequence, but without any chemical modification, with the oligonucleotide comprising at least one chemical modification. In some aspects, the oligonucleotide comprising the at least one chemical modification is at least 10%, 20%, 30%, 40%, 50%, 60%, 70%, 80%, 90%, two fold, three fold, four fold, five fold, six fold, seven fold, eight fold, nine fold, 10 fold, 20 fold, 30 fold, 40 fold, 50 fold, 100 fold, 500 fold, 1000 fold, or more resistant towards degradation by hydrolysis compared to an oligonucleotide sharing identical nucleic acid sequence, but without any chemical modification, with the oligonucleotide comprising at least one chemical modification.

In some aspects, the oligonucleotide comprising at least one chemical modification comprises an increased resistance towards degradation by nuclease digestion compared to an oligonucleotide sharing identical nucleic acid sequence, but without any chemical modification, with the oligonucleotide comprising at least one chemical modification. In some aspects, the oligonucleotide comprising the at least one chemical modification is at least 10%, 20%, 30%, 40%, 50%, 60%, 70%, 80%, 90%, two fold, three fold, four fold, five fold, six fold, seven fold, eight fold, nine fold, 10 fold, 20 fold, 30 fold, 40 fold, 50 fold, 100 fold, 500 fold, 1000 fold, or more resistant towards degradation by nuclease digestion compared to an oligonucleotide sharing identical nucleic acid sequence, but without any chemical modification, with the oligonucleotide comprising at least one chemical modification.

In some aspects, the oligonucleotide comprising at least one chemical modification induces less immunogenicity compared an oligonucleotide sharing identical nucleic acid sequence, but without any chemical modification, with the oligonucleotide comprising at least one chemical modification. In some aspects, the oligonucleotide comprising the at least chemical modification is at least 10%, 20%, 30%, 40%, 50%, 60%, 70%, 80%, 90%, two fold, three fold, four fold, five fold, six fold, seven fold, eight fold, nine fold, 10 fold, 20 fold, 30 fold, 40 fold, 50 fold, 100 fold, 500 fold, 1000 fold, or more less likely to induce immunogenicity compared to immunogenicity induced by an oligonucleotide sharing identical nucleic acid sequence, but without any chemical modification, with the oligonucleotide comprising at least one chemical modification.

In some aspects, the oligonucleotide comprising at least one chemical modification induces less innate immune response relative to an oligonucleotide sharing identical nucleic acid sequence, but without any chemical modification, with the oligonucleotide comprising at least one chemical modification. In some aspects, the oligonucleotide comprising the at least one chemical modification is at least 10%, 20%, 30%, 40%, 50%, 60%, 70%, 80%, 90%, two fold, three fold, four fold, five fold, six fold, seven fold, eight fold, nine fold, 10 fold, 20 fold, 30 fold, 40 fold, 50 fold, 100 fold, 500 fold, 1000 fold, or more less likely to induce innate immune response compared to innate immune response induced by an oligonucleotide sharing identical nucleic acid sequence, but without any chemical modification, with the oligonucleotide comprising at least one chemical modification.

In some aspects, the oligonucleotide comprising at least one chemical modification, when contacted with the target RNA, is less likely to induce off-target modulating of the target RNA compared to the off-target modulating of the target RNA induced by an oligonucleotide sharing identical nucleic acid sequence, but without any chemical modification, with the oligonucleotide comprising at least one chemical modification. In some aspects, the oligonucleotide comprising the at least one chemical modification is at least 10%, 20%, 30%, 40%, 50%, 60%, 70%, 80%, 90%, two fold, three fold, four fold, five fold, six fold, seven fold, eight fold, nine fold, 10 fold, 20 fold, 30 fold, 40 fold, 50 fold, 100 fold, 500 fold, 1000 fold, or more less likely to induce off-target modulating compared to off-target modulating induced by an oligonucleotide sharing identical nucleic acid sequence, but without any chemical modification, with the oligonucleotide comprising at least one chemical modification.

Table 2 illustrates examples of the oligonucleotide comprising the at least one chemical modification described herein. Nucleic acid sequences shown in Table 2 are denoted by “+” for referencing to a nucleic acid substitution with LNA; and denoted by “*” for referencing a chemical modification comprising a phosphorothioate linkage. “+N” is N nucleotide modified as LNA. “N*” is phosphothioate linkage coupling with the following nucleoside.

### Methods of Delivery

Described herein, In some aspects, are methods of delivering the oligonucleotide described herein to a cell. In some aspects, the method comprises delivering directly or indirectly an oligonucleotide to the cell. In some aspects, the method comprises contacting the cell with the composition or the oligonucleotide described herein. In some aspects, the method comprises expressing the composition or the oligonucleotide described herein in the cell. In some aspects, the oligonucleotide or vector encoding the oligonucleotide can be delivered into the cell via any of the transfection methods described herein. In some aspects, the oligonucleotide can be delivered into the cell via the use of expression vectors. In the context of an expression vector, the vector can be readily introduced into the cell described herein by any method in the art. For example, the expression vector can be transferred into the cell by physical, chemical, or biological means.

Physical methods for introducing the oligonucleotide or vector encoding the oligonucleotide into the cell can include calcium phosphate precipitation, lipofection, particle bombardment, microinjection, gene gun, electroporation, and the like. Methods for producing cells comprising vectors and/or exogenous nucleic acids are suitable for methods herein. One method for the introduction of oligonucleotide or vector encoding the oligonucleotide into a host cell is calcium phosphate transfection.

Chemical means for introducing the oligonucleotide or vector encoding the oligonucleotide into the cell can include colloidal dispersion systems, such as macromolecule complexes, nanocapsules, microspheres, beads, and lipid-based systems including oil-in-water emulsions, micelles, mixed micelles, spherical nucleic acid (SNA), liposomes, or lipid nanoparticles. An exemplary colloidal system for use as a delivery vehicle in vitro and in vivo is a liposome (e.g., an artificial membrane vesicle). Other methods of state-of-the-art targeted delivery of nucleic acids are available, such as delivery of oligonucleotide or vector encoding the oligonucleotide with targeted nanoparticles or other suitable sub-micron sized delivery system.

In the case where a non-viral delivery system is utilized, an exemplary delivery vehicle is a liposome. The use of lipid formulations is contemplated for the introduction of the oligonucleotide or vector encoding the oligonucleotide into a cell (in vitro, ex vivo, or in vivo). In another aspect, the oligonucleotide or vector encoding the oligonucleotide can be associated with a lipid. The oligonucleotide or vector encoding the oligonucleotide associated with a lipid, In some aspects, is encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, In some aspects, they are present in a bilayer structure, as micelles, or with a “collapsed” structure. Alternately, they are simply be interspersed in a solution, possibly forming aggregates that are not uniform in size or shape. Lipids are fatty substances which are, In some aspects, naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use are obtained from commercial sources. Stock solutions of lipids in chloroform or chloroform/methanol are often stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes are often characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers. However, compositions that have different structures in solution than the normal vesicular structure are also encompassed. For example, the lipids, In some aspects, assume a micellar structure or merely exist as nonuniform aggregates of lipid molecules. Also contemplated are lipofectamine-nucleic acid complexes.

In some cases, non-viral delivery method comprises lipofection, nucleofection, microinjection, biolistics, virosomes, liposomes, immunoliposomes, exosomes, polycation or lipid:cargo conjugates (or aggregates), naked polypeptide (e.g., recombinant polypeptides), naked DNA, artificial virions, and agent-enhanced uptake of polypeptide or DNA. In some aspects, the delivery method comprises conjugating or encapsulating the compositions or the oligonucleotides described herein with at least one polymer such as natural polymer or synthetic materials. The polymer can be biocompatible or biodegradable. Non-limiting examples of suitable biocompatible, biodegradable synthetic polymers can include aliphatic polyesters, poly(amino acids), copoly(ether-esters), polyalkylenes oxalates, polyamides, poly(iminocarbonates), polyorthoesters, polyoxaesters, polyamidoesters, polyoxaesters containing amine groups, and poly(anhydrides). Such synthetic polymers can be homopolymers or copolymers (e.g., random, block, segmented, graft) of a plurality of different monomers, e.g., two or more of lactic acid, lactide, glycolic acid, glycolide, epsilon-caprolactone, trimethylene carbonate, p-dioxanone, etc. In an example, the scaffold can be comprised of a polymer comprising glycolic acid and lactic acid, such as those with a ratio of glycolic acid to lactic acid of 90/10 or 5/95. Non-limiting examples of naturally occurring biocompatible, biodegradable polymers can include glycoproteins, proteoglycans, polysaccharides, glycosamineoglycan (GAG) and fragment(s) derived from these components, elastin, laminins, decrorin, fibrinogen/fibrin, fibronectins, osteopontin, tenascins, hyaluronic acid, collagen, chondroitin sulfate, heparin, heparan sulfate, ORC, carboxymethyl cellulose, and chitin.

In some cases, the oligonucleotide or vector encoding the oligonucleotide described herein can be packaged and delivered to the cell via extracellular vesicles. The extracellular vesicles can be any membrane-bound particles. In some aspects, the extracellular vesicles can be any membrane-bound particles secreted by at least one cell. In some instances, the extracellular vesicles can be any membrane-bound particles synthesized in vitro. In some instances, the extracellular vesicles can be any membrane-bound particles synthesized without a cell. In some cases, the extracellular vesicles can be exosomes, microvesicles, retrovirus-like particles, apoptotic bodies, apoptosomes, oncosomes, exophers, enveloped viruses, exomeres, or other very large extracellular vesicles.

In some cases, the oligonucleotide or vector encoding the oligonucleotide described herein can be administered to the subject in need thereof via the use of the transgenic cells generated by introduction of the oligonucleotide or vector encoding the oligonucleotide first into allogeneic or autologous cells. In some cases, the cell can be isolated. In some aspects, the cell can be isolated from the subject.

In some aspects, the oligonucleotide described herein is conjugated. In some aspects, the oligonucleotide is conjugated to with a peptide, antibody, lipid, carbohydrate, or polymer. In some aspects, the oligonucleotide is conjugated to with a peptide, antibody, lipid, carbohydrate, or polymer at the 5′ end of the oligonucleotide. In some aspects, the oligonucleotide is conjugated to with a peptide, antibody, lipid, carbohydrate, or polymer at the 3′ end of the oligonucleotide. In some aspects, the oligonucleotide is conjugated to with a peptide, antibody, lipid, carbohydrate, or polymer at any nucleic acid residue of the oligonucleotide. In some aspects, the peptide, antibody, lipid, carbohydrate, or polymer conjugated to the oligonucleotide confers therapeutic effect. For example, the peptide, antibody, lipid, carbohydrate, or polymer conjugated to the oligonucleotide can be cytotoxic drug or drug for treating cancer. In some aspects, the peptide, antibody, lipid, carbohydrate, or polymer conjugated to the oligonucleotide increases the efficiency of the oligonucleotide binding to the endogenous nucleic acid. In some aspects, the peptide, antibody, lipid, carbohydrate, or polymer conjugated to the oligonucleotide confers targeting specificity of the oligonucleotide to specific types of cells (e.g., cancer cells, etc.). In some aspects, the peptide, antibody, lipid, carbohydrate, or polymer conjugated to the oligonucleotide confers stability of the oligonucleotide in vitro, ex vivo, or in vivo. For example, the oligonucleotide can be conjugated with polyethylene glycol (PEG) or endosomolytic agent to decrease immunogenicity or degradation. In some aspects, the peptide, antibody, lipid, carbohydrate, or polymer conjugated to the oligonucleotide to facilitate the oligonucleotide for entering cell. In some aspects, the peptide, antibody, lipid, carbohydrate, or polymer conjugated to the oligonucleotide to facilitate and release to the oligonucleotide in the cell. In some aspects, the peptide, antibody, lipid, carbohydrate, or polymer conjugated to the oligonucleotide comprises at least one targeting moiety for targeting the cell. Non-limiting examples of the targeting moiety comprises a signaling peptide, a chemokine, a chemokine receptor, an adhesion molecule, an antigen, or an antibody.

The linker for conjugating the oligonucleotide to the peptide, antibody, lipid, or polymer can be any linker that connects biomolecules. In some aspects, a linker described herein is a cleavable linker or a non-cleavable linker. In some instances, the linker is a cleavable linker. In other instances, the linker is a non-cleavable linker. In some cases, the linker is a non-polymeric linker. A non-polymeric linker refers to a linker that does not contain a repeating unit of monomers generated by a polymerization process. In some aspects, the linker comprises a peptide moiety. In some instances, the peptide moiety comprises at least 2, 3, 4, 5, or 6 more amino acid residues. In some aspects, the linker comprises a benzoic acid group, or its derivatives thereof. In some aspects, the linker can comprise nucleic acid linker such as DNA linker. In such case, the peptide, antibody, lipid, or polymer can be conjugated on one end of the nucleic acid linker or intercalated into the nucleotide pairing of the nucleic acid linker. In some aspects, the linker can be a peptide linker. The peptide linker can be flexible (e.g., poly-glycine linker) or rigid (e.g., EAAAK repeat linker). In some aspects, the peptide linker can be cleaved (e.g., a disulfide bond). In some aspects, the linker comprises polymers such PEG, polylactic acid (PLA), or polyacrylic acid (PAA).

### Methods of Treatment

Disclosed herein, In some aspects, are methods of modulating gene expression of any one of the genes described herein for treating a disease or condition. In some aspects, the gene being modulated by the oligonucleotide, composition, or pharmaceutical composition described herein is associated with inflammasome pathway. In some aspects, the method comprises contacting a cell with the oligonucleotide, the composition, or the pharmaceutical composition described herein to decrease expression of NLRP3. In some aspects, the method comprises contacting a cell with the oligonucleotide, the composition, or the pharmaceutical composition described herein to decrease expression of NLRP1. In some aspects, the method comprises contacting a cell with the oligonucleotide, the composition, or the pharmaceutical composition described herein to decrease expression of NLRP3 or NLRP1. In some aspects, the method comprises contacting a cell with the oligonucleotide, the composition, or the pharmaceutical composition described herein to decrease expression of NLRP3 and NLRP1. In some aspects, the method comprises contacting a cell with the oligonucleotide, the composition, or the pharmaceutical composition described herein to decrease expression of any one of the genes associated with inflammasome.

Also disclosed herein, In some aspects, are methods of treating a subject in need thereof by administrating a therapeutic effective amount of the oligonucleotide, composition, or pharmaceutical composition described herein to the subject. In some aspects, the method treats the subject by modulating gene expression or inflammasome pathway in the subject. In some aspects, the method comprises decreasing gene expression by contacting an endogenous nucleic acid (e.g., endogenous mRNA) with the oligonucleotide described herein. In some aspects, the method comprises decreasing the expression of any one of the genes described herein. In some aspects, the method comprises decreasing expression of NLRP3, NLRC4/NAIP, NLRP1, AIM2, IFI16, Pyrin, or a combination thereof. In some aspects, the method comprises decreasing NLRP3. In some aspects, the method comprises decreasing NLRP1. In some aspects, the method comprises decreasing NLRP3 or NLRP1. In some aspects, the method comprises decreasing NLRP3 and NLRP1 in the subject or in a diseased cell by contacting mRNA of NLRP3 or NLRP1 with the oligonucleotide described herein, where the binding of the oligonucleotide to the mRNA recruits endogenous nuclease for degradation of the mRNA. In some aspects, the method comprises decreasing expression of inflammasome pathway in the diseased cell.

In some embodiments, the oligonucleotide is an antisense oligonucleotide. In some embodiments, the antisense oligonucleotide comprises a nucleic acid sequence that is at least 80%, 85%, 90%, 95%, or 99% identical to any one of SEQ ID NOs: 95, 96, 97, 98, 99, 100, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, or 646. In some embodiments, the antisense oligonucleotide comprises the nucleic acid sequence that is at least 80%, 85%, 90%, 95%, or 99% identical to any one of SEQ ID NOs: 95, 121, 139, 144, 146, 147, or 172. In some embodiments, the antisense oligonucleotide comprises the nucleic acid sequence that is at least 80%, 85%, 90%, 95%, or 99% identical to SEQ ID NO: 95. In some embodiments, the antisense oligonucleotide comprises the nucleic acid sequence that is at least 80%, 85%, 90%, 95%, or 99% identical to SEQ ID NO: 121. In some embodiments, the antisense oligonucleotide comprises the nucleic acid sequence that is at least 80%, 85%, 90%, 95%, or 99% identical to SEQ ID NO: 139. In some embodiments, the antisense oligonucleotide comprises the nucleic acid sequence that is at least 80%, 85%, 90%, 95%, or 99% identical to SEQ ID NO: 144. In some embodiments, the antisense oligonucleotide comprises the nucleic acid sequence that is at least 80%, 85%, 90%, 95%, or 99% identical to SEQ ID NO: 146. In some embodiments, the antisense oligonucleotide comprises the nucleic acid sequence that is at least 80%, 85%, 90%, 95%, or 99% identical to SEQ ID NO: 147. In some embodiments, the antisense oligonucleotide comprises the nucleic acid sequence that is at least 80%, 85%, 90%, 95%, or 99% identical to SEQ ID NO: 172. In some embodiments, the antisense oligonucleotide is any one of SEQ ID NOs: 95, 121, 139, 144, 146, 147, or 172. In some embodiments, the antisense oligonucleotide is SEQ ID NO: 95. In some embodiments, the antisense oligonucleotide is SEQ ID NO: 121. In some embodiments, the antisense oligonucleotide is SEQ ID NO: 139. In some embodiments, the antisense oligonucleotide is SEQ ID NO: 144. In some embodiments, the antisense oligonucleotide is SEQ ID NO: 146. In some embodiments, the antisense oligonucleotide is SEQ ID NO: 147. In some embodiments, the antisense oligonucleotide is SEQ ID NO: 172.

In some aspects, the oligonucleotide, composition, or pharmaceutical composition can be administered to the subject alone (e.g., standalone treatment). In some aspects, the oligonucleotide, composition, or pharmaceutical composition is administered in combination with an additional agent. In some cases, the additional agent as used herein is administered alone. The oligonucleotide, composition, or pharmaceutical composition and the additional agent can be administered together or sequentially. Non-limiting examples of the additional agent comprise N-(2-(4-(4-bis(2-chloroethyl)aminophenyl)butyryl)aminoethyl)-5-(4-amidinophenyl)-2-furanecarboxamide hydrochloride; Allyl isothiocyanate; Benzyl isothiocyanate; Phenethyl isothiocyanate; Belinostat; Berberin; Casticin; Chrysin; Bufalin; Fisetin; Fucoidan; Galic acid; Gemcitabine; Guizhi Fuling Decoction; JOTO1007; Quercetin; Rasfonin; 2,3,7,8-tetrachlorodibenzodioxin; Triptolide; 4-Hydroxybutenolide; or a combination thereof. Other non-limiting examples of additional agent can include Glyburide, 16673-34-0, JC124, FC11A-2, Parthenolide, VX-740, VX-765, Bay 11-7082, BHB, MCC950, MNS, CY-09, Tranilast, OLT1177, Oridonin, Glybenclamide, Ac-YVAD-cmk, Z-VAD-FMK, Isoliquiritigenin, Pralnacasan, or a combination there of. The combination therapies can be administered within the same day, or can be administered one or more days, weeks, months, or years apart. In some aspects, the method comprises, in addition to modulating the gene expression by the oligonucleotide described herein, modulating expression of one or more of IL-6, IL-I β, IL-17, IL-18, IL-1a, IL-37, IL-22, IL-33, and Th17.

In some aspects, the oligonucleotide, composition, or pharmaceutical composition is a first-line treatment for the disease or condition. In some aspects, the oligonucleotide, composition, or pharmaceutical composition is a second-line, third-line, or fourth-line treatment. In some aspects, the oligonucleotide, composition, or pharmaceutical composition comprises at least one, two, three, four, five, six, seven, eight, nine, 10, 20, 30 or more oligonucleotide. In general, method disclosed herein comprises administering the oligonucleotide, composition, or pharmaceutical composition by oral administration. However, in some instances, method comprises administering the oligonucleotide, composition, or pharmaceutical composition by intraperitoneal injection. In some instances, the method comprises administering the pharmaceutical composition in the form of an anal suppository. In some instances, the method comprises administering the oligonucleotide, composition, or pharmaceutical composition by intravenous (“i.v.”) administration. It is conceivable that one can also administer the oligonucleotide, composition, or pharmaceutical composition disclosed herein by other routes, such as subcutaneous injection, intramuscular injection, intradermal injection, transdermal injection percutaneous administration, intranasal administration, intralymphatic injection, rectal administration intragastric administration, or any other suitable parenteral administration. In some aspects, routes for local delivery closer to site of injury or inflammation are preferred over systemic routes. Routes, dosage, time points, and duration of administrating therapeutics can be adjusted. In some aspects, administration of therapeutics is prior to, or after, onset of either, or both, acute and chronic symptoms of the disease or condition.

Suitable dose and dosage administrated to a subject is determined by factors including, but no limited to, the particular the oligonucleotide, composition, or pharmaceutical composition, disease condition and its severity, the identity (e.g., weight, sex, age) of the subject in need of treatment, and can be determined according to the particular circumstances surrounding the case, including, e.g., the specific agent being administered, the route of administration, the condition being treated, and the subject being treated.

In some aspects, the administration of the oligonucleotide, composition, or pharmaceutical composition described herein is hourly, once every 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours 22 hours, 23 hours, 1 day, 2 days, 3 days, 4 days, 5 days, 6 days, 7 days, 8 days, 9 days, 10 days, 11 days, 12 days, 13 days, 14 days, 15 days, 1 month, 2 months, 3 months, 4 months, 5 months, 6 months, 7 months, 8 months, 9 months, 10 months, 11 months, 1 year, 2 years, 3 years, 4 years, or 5 years, or 10 years. The effective dosage ranges can be adjusted based on subject's response to the treatment. Some routes of administration will require higher concentrations of effective amount of therapeutics than other routes.

In some aspects, the administration of the oligonucleotide, composition, or pharmaceutical composition described herein increases survival rate of the subject by at least 1%, 2%, 3%, 4%, 5%, 6%, 7%, 8%, 9%, 10%, 15%, 20%, 30%, 40%, 50%, or more. In some aspects, the administration of the oligonucleotide, composition, or pharmaceutical composition described herein at a dose that increases survival rate of the subject by at least 1%, 2%, 3%, 4%, 5%, 6%, 7%, 8%, 9%, 10%, 15%, 20%, 30%, 40%, 50%, or more. In some aspects, the administration of the oligonucleotide, composition, or pharmaceutical composition described herein at a schedule that increases survival rate of the subject by at least 1%, 2%, 3%, 4%, 5%, 6%, 7%, 8%, 9%, 10%, 15%, 20%, 30%, 40%, 50%, or more. In some aspects, the administration of the oligonucleotide, composition, or pharmaceutical composition described herein at a dose and a schedule that increase survival rate of the subject by at least 1%, 2%, 3%, 4%, 5%, 6%, 7%, 8%, 9%, 10%, 15%, 20%, 30%, 40%, 50%, or more.

In some aspects, the administration of the oligonucleotide, composition, or pharmaceutical composition described herein decreases growth of the tumor by at least 1%, 2%, 3%, 4%, 5%, 6%, 7%, 8%, 9%, 10%, 15%, 20%, 30%, 40%, 50%, or more. In some aspects, the administration of the oligonucleotide, composition, or pharmaceutical composition described herein at a dose that decreases growth of the tumor by at least 1%, 2%, 3%, 4%, 5%, 6%, 7%, 8%, 9%, 10%, 15%, 20%, 30%, 40%, 50%, or more. In some aspects, the administration of the oligonucleotide, composition, or pharmaceutical composition described herein at a schedule that decreases growth of the tumor by at least 1%, 2%, 3%, 4%, 5%, 6%, 7%, 8%, 9%, 10%, 15%, 20%, 30%, 40%, 50%, or more. In some aspects, the administration of the oligonucleotide, composition, or pharmaceutical composition described herein at a dose and a schedule that decreases growth of the tumor by at least 1%, 2%, 3%, 4%, 5%, 6%, 7%, 8%, 9%, 10%, 15%, 20%, 30%, 40%, 50%, or more.

In some aspects, the administration of the oligonucleotide, composition, or pharmaceutical composition described herein decreases inflammation by at least 1%, 2%, 3%, 4%, 5%, 6%, 7%, 8%, 9%, 10%, 15%, 20%, 30%, 40%, 50%, or more. In some aspects, the administration of the oligonucleotide, composition, or pharmaceutical composition described herein at a dose that decreases inflammation by at least 1%, 2%, 3%, 4%, 5%, 6%, 7%, 8%, 9%, 10%, 15%, 20%, 30%, 40%, 50%, or more. In some aspects, the administration of the oligonucleotide, composition, or pharmaceutical composition described herein at a schedule that decreases inflammation by at least 1%, 2%, 3%, 4%, 5%, 6%, 7%, 8%, 9%, 10%, 15%, 20%, 30%, 40%, 50%, or more. In some aspects, the administration of the oligonucleotide, composition, or pharmaceutical composition described herein at a dose and a schedule that decreases inflammation by at least 1%, 2%, 3%, 4%, 5%, 6%, 7%, 8%, 9%, 10%, 15%, 20%, 30%, 40%, 50%, or more.

In some aspects, the administration of the oligonucleotide, composition, or pharmaceutical composition described herein to the subject in a dose that is sufficient to inhibit growth of the tumor. In some aspects, the administration of the oligonucleotide, composition, or pharmaceutical composition described herein to the subject in a schedule that is sufficient to inhibit growth of the tumor. In some aspects, the administration of the oligonucleotide, composition, or pharmaceutical composition described herein to the subject in a dose and a schedule that are sufficient to inhibit growth of the tumor.

In certain embodiments, where the subject's condition does not improve, upon the doctor's discretion the administration of the pharmaceutical composition is administered chronically, that is, for an extended period of time, including throughout the duration of the subject's life in order to ameliorate or otherwise control or limit the symptoms of the subject's disease or condition. In certain embodiments wherein a subject's status does improve, the dose of the pharmaceutical composition being administered can be temporarily reduced or temporarily suspended for a certain length of time (i.e., a “drug holiday”). In specific embodiments, the length of the drug holiday is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug holiday is, by way of example only, by 10%-100%, including by way of example only 10%, 15%, 20%, 25%, 30%, 35%, 40%, 45%, 50%, 55%, 60%, 6%, 70%, 75%, 80%, 85%, 90%, 95%, and 100%. In certain embodiments, the dose of the pharmaceutical composition being administered can be temporarily reduced or temporarily suspended for a certain length of time (i.e., a “drug diversion”). In specific embodiments, the length of the pharmaceutical composition diversion is between 2 days and 1 year, including by way of example only, 2 days, 3 days, 4 days, 5 days, 6 days, 7 days, 10 days, 12 days, 15 days, 20 days, 28 days, or more than 28 days. The dose reduction during the pharmaceutical composition diversion is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%. After a suitable length of time, the normal dosing schedule is optionally reinstated.

In some aspects, once improvement of the subject's conditions has occurred, a maintenance dose is administered if necessary. Subsequently, in specific embodiments, the dosage or the frequency of administration, or both, is reduced, as a function of the symptoms, to a level at which the improved disease, disorder or condition is retained. In certain embodiments, however, the subject requires intermittent treatment on a long-term basis upon any recurrence of symptoms.

Toxicity and therapeutic efficacy of such therapeutic regimens are determined by standard pharmaceutical procedures in cell cultures or experimental animals, including, but not limited to, the determination of the LD₅₀ and the ED₅₀. The dose ratio between the toxic and therapeutic effects is the therapeutic index and it is expressed as the ratio between LD₅₀ and ED₅₀. In certain embodiments, the data obtained from cell culture assays and animal studies are used in formulating the therapeutically effective daily dosage range and/or the therapeutically effective unit dosage amount for use in mammals, including humans. In some aspects, the daily dosage amount of the composition described herein lies within a range of circulating concentrations that include the ED₅₀ with minimal toxicity. In certain embodiments, the daily dosage range and/or the unit dosage amount varies within this range depending upon the dosage form employed and the route of administration utilized.

In some aspects, the disease or condition described herein is associated with inflammation. In some aspects, the disease or condition described herein is associated with pyroptosis. In some aspects, the disease or condition described herein is associated with expression of any one of the genes described herein. In some aspects, the disease or condition described herein is associated with NLRP3, NLRC4/NAIP, NLRP1, AIM2, IFI16, Pyrin, or a combination thereof. In some aspects, the disease or condition described herein is associated with expression of NLRP3. In some aspects, the disease or condition described herein is caused by inflammation associated with expression of NLRP3. In some aspects, the disease or condition described herein is caused by pyroptosis associated with expression of NLRP3. In some aspects, the disease or condition described herein is associated with expression of NLRP1. In some aspects, the disease or condition described herein is caused by inflammation associated with expression of NLRP1. In some aspects, the disease or condition described herein is caused by pyroptosis associated with expression of NLRP1. In some aspects, the disease or condition described herein is associated with expression of NLRP3 or NLRP1. In some aspects, the disease or condition described herein is caused by inflammation associated with expression of NLRP3 or NLRP1. In some aspects, the disease or condition described herein is caused by pyroptosis associated with expression of NLRP3 or NLRP1 In some aspects, the disease or condition described herein is associated with expression of NLRP3 and NLRP1. In some aspects, the disease or condition described herein is caused by inflammation associated with expression of NLRP3 and NLRP1. In some aspects, the disease or condition described herein is caused by pyroptosis associated with expression of NLRP3 and NLRP1.

In some aspects, the disease or condition described herein is associated with expression of inflammasome. In some aspects, the disease or condition caused by inflammasome is associated with expression of NLRP3. In some aspects, the disease or condition caused by inflammasome is associated with expression of NLRP1. In some aspects, the disease or condition caused by inflammasome is associated with expression of NLRP3 or NLRP1. In some aspects, the disease or condition caused by inflammasome is associated with expression of NLRP3 and NLRP1.

In some aspects, the disease or condition associated with expression of NLRP3 or NLRP1 or inflammasome is an autoinflammatory disease, autoimmune disease, neurodegenerative disease. In some aspects, the disease or condition is associated with immune system, cardiovascular system, endocrine system, gastrointestinal tract, renal system, respiratory system, central nervous system, cancer, or pathogen infection (e.g., infection by virus, bacterium, protist, worm, fungus, or any other organism capable of infecting a mammal). Non-limiting examples of viruses include influenza virus, cytomegalovirus, Epstein Barr Virus, human immunodeficiency virus (HIV), alphavirus such as Chikungunya and Ross River virus, flaviviruses such as Dengue virus, Zika virus, or papillomavirus. Non-limiting examples of pathogenic bacteria include Staphylococcus aureus, Helicobacter pylon, Bacillus anthracis, Bordatella pertussis, Corynebacterium dipthenae, Clostridium tetani, Clostridium botulinum, Streptococcus pneumoniae, Streptococcus pyogenes, Listeria monocytogenes. Hemophilus influenzae, Pasteureiia multicida, Shigella dysenteriae, Mycobacterium tuberculosis, Mycobacterium leprae, Mycoplasma pneumoniae. Mycoplasma hominis, Neisseria meningitidis, Neisseria gonorrhoeae, Rickettsia rickettsii, Legionella pneumophila, Klebsiella pneumoniae, Pseudomonas aeruginosa, Propionibacterium acnes, Treponema pallidum, Chlamydia trachomatis, Vibrio cholerae, Salmonella typhimurium, Salmonella typhi, Borrelia burgdorferi, or Yersinia pestis. Non-limiting examples of protists include Plasmodium, Babesia, Giardia, Entamoeba, Leishmania, or Trypanosomas. Non-limiting examples of worms include helminths inclusive of schistisimes, roundworms, tapeworms, or flukes. Non-limiting examples of fungi include Candida or Aspergillus species,

In some aspects, the disease or condition is caused at least partially by constitutively active inflammation, including: cryopyrin-associated periodic syndromes (CAPS); Muckle-Wells syndrome (MWS); familial cold automflammatory syndrome (FCAS); or neonatal-onset multisystem inflammatory disease (NOMID). In some aspects, the disease or condition is an autoinflammatory diseases, including: familial Mediterranean fever (FMF); TNF receptor associated periodic syndrome (TRAPS); mevalonate kinase deficiency (MET); hyperimmunoglobulinemia D and periodic fever syndrome; deficiency of interleukin 1 receptor (DIRA) antagonist; Majeed syndrome; pyogenic arthritis; pyoderma gangrenosum and acne (PAPA); haploinsufficiency of A20 (HA20); pediatric granulomatous arthritis (PGA); PLCG2-associated antibody deficiency and immune dysregulation (PLAID); PLCG2-associated autoinflammation; antibody deficiency and immune dysregulation (APLAID); sideroblastic anemia with B-cell immunodeficiency; periodic fevers, and developmental delay (SIFD); Sweet syndrome; chronic nonbacterial osteomyelitis (CNO); chronic recurrent multifocal osteomyelitis (CRMO); synovitis, acne, pustulosis, hyperostosis, or osteitis syndrome (SAPHO). In some aspects, the disease or condition is an autoimmune disease, including multiple sclerosis (MS); type I diabetes; psoriasis; rheumatoid arthritis; Behcet's disease; Sjogren's syndrome; or Schnitzler syndrome. In some aspects, the disease or condition is a respiratory diseases, including idiopathic pulmonary fibrosis (IFF); chronic obstructive pulmonary disorder (COPD); steroid-resistant asthma; asbestosis; or silicosis and cystic fibrosis. In some aspects, the disease or condition is a central nervous system diseases, including migraine; amyotrophic lateral sclerosis (ALS); Parkinson's disease; Alzheimer's disease; motor neuron disease; Huntington's disease; cerebral malaria; or brain injury from pneumococcal meningitis. In some aspects, the disease or condition comprises a nerve injury. In some aspects, the nerve injury comprises a peripheral nerve injury. In some aspects, the nerve injury comprises a spinal cord injury.

In some aspects, the disease or condition is a metabolic disease, including type II diabetes; atherosclerosis; obesity; gout; or pseudo-gout. In some aspects, the disease or condition is an ocular diseases such as ocular epithelium, age-related macular degeneration (AMD), corneal infection, uveitis, or dry eye. In some aspects, the disease or condition is a kidney disease such as chronic kidney disease, oxalate nephropathy, or diabetic nephropathy. In some aspects, the disease or condition is a liver disease such as non-alcoholic steatohepatitis or alcoholic liver disease. In some aspects, the disease or condition is an inflammatory reactions in skin such as contact hypersensitivity or sunburn. In some aspects, the disease or condition is an inflammatory reactions in the joints such as osteoarthritis, systemic juvenile idiopathic arthritis, adult-onset Still's disease, or relapsing polychondritis. In some aspects, the disease or condition is caused by viral infection such as alpha virus (Chikungunya, Ross River), flavivirus (Dengue and Zika Virus), flu, or HIV. In some aspects, the disease or condition is hidradenitis suppurativa (HS) or other cyst-causing skin diseases. In some aspects, the disease or condition is polymyositis, stroke, myocardial infarction, Graft versus Host Disease, hypertension, colitis, helminth infection, bacterial infection, abdominal aortic aneurism, wound healing, depression, psychological stress, pericarditis including Dressler's syndrome, or ischemia reperfusion injury.

In some aspects, the disease or condition is cancer. In some aspects, the cancer is a lung cancer, a pancreatic cancer, or a colon cancer. Other non-limiting examples of the cancer can include Acute Lymphoblastic Leukemia, Acute Lymphocytic Leukemia (ALL), Acute Myeloid Leukemia (AML), Adenoid Cystic Carcinoma, Adrenal Gland Cancer, Adrenocortical Carcinoma, Adult Leukemia, AIDS-Related Lymphoma, Amyloidosis, Anal Cancer, Astrocytomas, Ataxia Telangiectasia, Atypical Mole Syndrome, Atypical Teratoid/Rhabdoid Tumor, Basal Cell Carcinoma, Bile Duct Cancer, Birt Hogg Dube Syndrome, Bladder Cancer, Bone Cancer, Brain Tumor, Breast Cancer, Bronchial Tumors, Burkitt Lymphoma, Carcinoid Tumor (Gastrointestinal), Carcinoma of Unknown Primary, Cardiac (Heart) Tumors, Cervical Cancer, Cholangiocarcinoma, Chordoma, Chronic Lymphocytic Leukemia (CLL), Chronic Myelogenous Leukemia, Chronic Myeloid Leukemia, Chronic Myeloproliferative Neoplasms, Colorectal Cancer, Craniopharyngioma, Cutaneous T-Cell Lymphoma, Ductal Carcinoma, Embryonal Tumors, Endometrial Cancer, Ependymoma, Esophageal Cancer, Esthesioneuroblastoma, Ewing Sarcoma, Extracranial Germ Cell Tumor, Extragonadal Germ Cell Tumor, Eye Cancer, Fallopian Tube Cancer, Fibrous Histiocytoma of Bone, Malignant, and Osteosarcoma, Gallbladder Cancer, Gastric Cancer, Gastrointestinal Carcinoid Tumor, Gastrontestinal Stromal Tumor (GIST), Germ Cell Tumors, Gestational Trophoblastic Disease, Hairy Cell Leukemia, Head and Neck Cancer, Hepatocellular Cancer, HER2-Positive Breast Cancer, Histiocytosis, Langerhans Cell, Hodgkin's Lymphoma, Hypopharyngeal Cancer, Intraocular Melanoma, Islet Cell Tumor, Juvenile Polyposis Syndrome, Kaposi Sarcoma, Kidney Cancer, Langerhans Cell Histiocytosis, Laryngeal Cancer, Leukemia, Lip and Oral Cavity Cancer, Liver Cancer, Lobular Carcinoma, Lung Cancer (Non-Small Cell and Small Cell), Lymphoma, Malignant Fibrous Histiocytoma of Bone and Osteosarcoma, Malignant Glioma, Melanoma, Intraocular Melanoma, Meningioma, Merkel Cell Carcinoma, Mesothelioma, Malignant, Metastatic Cancer, Metastatic Squamous Neck Cancer with Occult Primary, Midline Tract Carcinoma, Multiple Endocrine Neoplasia Syndromes, Multiple Myeloma, Plasma Cell Neoplasms, Mycosis Fungoides, Myelodysplastic Syndrome (MDS), Myeloproliferative Neoplasms, Chronic, Nasal Cavity and Paranasal Sinus Cancer, Nasopharyngeal Cancer, Neuroblastoma, Neuroendocrine Tumor, Non-Hodgkin Lymphoma, Oral Cancer, Lip and Oral Cavity Cancer and Oropharyngeal Cancer, Oropharyngeal Cancer, Osteosarcoma, Ovarian Cancer, Ovarian Germ Cell Tumors, Pancreatic Cancer, Pancreatic Neuroendocrine Tumors, Papillomatosis, Paraganglioma, Paranasal Sinus and Nasal Cavity Cancer, Parathyroid Cancer, Penile Cancer, Peritoneal Cancer, Peutz-Jeghers Syndrome, Pharyngeal Cancer, Pheochromocytoma, Pituitary Tumor, Plasma Cell Neoplasm/Multiple Myeloma, Pleuropulmonary Blastoma, Polycythemia Vera, Pregnancy and Breast Cancer, Primary Central Nervous System (CNS) Lymphoma, Primary Peritoneal Cancer, Prostate Cancer, Rectal Cancer, Recurrent Cancer, Renal Cell Carcinoma, Retinoblastoma, Rhabdomyosarcoma, Salivary Gland Cancer, Sarcoma, Sézary Syndrome, Skin Cancer, Small Intestine Cancer, Soft Tissue Sarcoma, Solid tumor, Squamous Cell Carcinoma of the Skin, Squamous Neck Cancer with Occult Primary, Metastatic, Stomach Cancer, T-Cell Lymphoma, Testicular Cancer, Throat Cancer, Thymoma, Thymic Carcinoma, Thyroid Cancer, Transitional Cell Cancer of the Renal Pelvis and Ureter, Unusual Cancers of Childhood, Ureter and Renal Pelvis, Transitional Cell Cancer, Urethral Cancer, Uterine (Endometrial) Cancer, Uterine Sarcoma, Vaginal Cancer, Vascular Tumors, Vulvar Cancer, Wilms Tumor, or a combination thereof.

### Pharmaceutical Compositions

Described herein, In some aspects, is a pharmaceutical composition comprising at least one oligonucleotide or the composition described herein. Pharmaceutical composition, as used herein, refers to a mixture of a pharmaceutical composition, with other chemical components (i.e. pharmaceutically acceptable inactive ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. Optionally, the compositions include two or more pharmaceutical composition as discussed herein. In practicing the methods of treatment or use provided herein, therapeutically effective amounts of pharmaceutical compositions described herein are administered in a pharmaceutical composition to a mammal having a disease, disorder, or condition to be treated, e.g., an inflammatory disease, fibrostenotic disease, and/or fibrotic disease. In some aspects, the mammal is a human. A therapeutically effective amount can vary widely depending on the severity of the disease, the age and relative health of the subject, the potency of the pharmaceutical composition used and other factors. The pharmaceutical compositions can be used singly or in combination with one or more pharmaceutical compositions as components of mixtures. The pharmaceutical commotions described herein comprise the oligonucleotide, the compositions, the cells contacted with the oligonucleotide or contacted with the composition comprising the oligonucleotide, or a combination thereof.

The pharmaceutical formulations described herein are administered to a subject by appropriate administration routes, including but not limited to, intravenous, intraarterial, oral, parenteral, buccal, topical, transdermal, rectal, intramuscular, subcutaneous, intraosseous, transmucosal, inhalation, or intraperitoneal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

Pharmaceutical compositions including a pharmaceutical composition are manufactured in a conventional manner, such as, by way of example only, by means of conventional mixing, dissolving, granulating, dragee-making, levigating, emulsifying, encapsulating, entrapping or compression processes.

The pharmaceutical compositions may include at least a pharmaceutical composition as an active ingredient in free-acid or free-base form, or in a pharmaceutically acceptable salt form. In addition, the methods and pharmaceutical compositions described herein include the use of N-oxides (if appropriate), crystalline forms, amorphous phases, as well as active metabolites of these compounds having the same type of activity. In some aspects, pharmaceutical compositions exist in unsolvated form or in solvated forms with pharmaceutically acceptable solvents such as water, ethanol, and the like. The solvated forms of the pharmaceutical compositions are also considered to be disclosed herein.

In some aspects, a pharmaceutical composition exists as a tautomer. All tautomers are included within the scope of the agents presented herein. As such, it is to be understood that a pharmaceutical composition or a salt thereof may exhibit the phenomenon of tautomerism whereby two chemical compounds that are capable of facile interconversion by exchanging a hydrogen atom between two atoms, to either of which it forms a covalent bond. Since the tautomeric compounds exist in mobile equilibrium with each other they can be regarded as different isomeric forms of the same compound.

In some aspects, a pharmaceutical composition exists as an enantiomer, diastereomer, or other steroisomeric form. The agents disclosed herein include all enantiomeric, diastereomeric, and epimeric forms as well as mixtures thereof.

In some aspects, pharmaceutical compositions described herein can be prepared as prodrugs. A “prodrug” refers to an agent that is converted into the parent drug in vivo. Prodrugs are often useful because, in some situations, they can be easier to administer than the parent drug. They may, for instance, be bioavailable by oral administration whereas the parent is not. The prodrug may also have improved solubility in pharmaceutical compositions over the parent drug. An example, without limitation, of a prodrug would be a pharmaceutical composition described herein, which is administered as an ester (the “prodrug”) to facilitate transmittal across a cell membrane where water solubility is detrimental to mobility but which then is metabolically hydrolyzed to the carboxylic acid, the active enzyme, once inside the cell where water-solubility is beneficial. A further example of a prodrug might be a short peptide (polyaminoacid) bonded to an acid group where the peptide is metabolized to reveal the active moiety. In certain embodiments, upon in vivo administration, a prodrug is chemically converted to the biologically, pharmaceutically or therapeutically active form of the pharmaceutical composition. In certain embodiments, a prodrug is enzymatically metabolized by one or more steps or processes to the biologically, pharmaceutically or therapeutically active form of the pharmaceutical composition.

Prodrug forms of the pharmaceutical compositions, wherein the prodrug is metabolized in vivo to produce an agent as set forth herein are included within the scope of the claims. Prodrug forms of the herein described pharmaceutical compositions, wherein the prodrug is metabolized in vivo to produce an agent as set forth herein are included within the scope of the claims. In some cases, some of the pharmaceutical compositions described herein can be a prodrug for another derivative or active compound. In some embodiments described herein, hydrazones are metabolized in vivo to produce a pharmaceutical composition.

### Kits

Described herein, In some aspects, are kits for using the oligonucleotide, the compositions, or the pharmaceutical compositions described herein. In some aspects, the kits disclosed herein may be used to treat a disease or condition in a subject. In some aspects, the kit comprises an assemblage of materials or components apart from the oligonucleotide, the composition, or the pharmaceutical composition. In some aspects, the kit comprises the components for assaying and selecting for suitable oligonucleotide for treating a disease or a condition. In some aspects, the kit comprises components for performing assays such as enzyme-linked immunosorbent assay (ELISA), single-molecular array (Simoa), PCR, or qPCR. The exact nature of the components configured in the kit depends on its intended purpose. For example, some embodiments are configured for the purpose of treating a disease or condition disclosed herein (e.g., cancer) in a subject. In some aspects, the kit is configured particularly for the purpose of treating mammalian subjects. In some aspects, the kit is configured particularly for the purpose of treating human subjects.

Instructions for use may be included in the kit. In some aspects, the kit comprises instructions for administering the composition to a subject in need thereof. In some aspects, the kit comprises instructions for further engineering the oligonucleotide. In some aspects, the kit comprises instructions thawing or otherwise restoring biological activity of the oligonucleotide, which may have been cryopreserved or lyophilized during storage or transportation. In some aspects, the kit comprises instructions for measuring efficacy for its intended purpose (e.g., therapeutic efficacy if used for treating a subject).

Optionally, the kit also contains other useful components, such as, diluents, buffers, pharmaceutically acceptable carriers, syringes, catheters, applicators, pipetting or measuring tools, bandaging materials or other useful paraphernalia. The materials or components assembled in the kit may be provided to the practitioner stored in any convenient and suitable ways that preserve their operability and utility. For example, the oligonucleotide, the composition, or the pharmaceutical composition may be in dissolved, dehydrated, or lyophilized form. The components are typically contained in suitable packaging material(s).

Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit scope of the present embodiments disclosed herein but as exemplary.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

Any systems, methods, software, and platforms described herein are modular. Accordingly, terms such as “first” and “second” do not necessarily imply priority, order of importance, or order of acts.

The term “about” when referring to a number or a numerical range means that the number or numerical range referred to is an approximation within experimental variability (or within statistical experimental error), and the number or numerical range may vary from, for example, from 1% to 15% of the stated number or numerical range. In examples, the term “about” refers to ±10% of a stated number or value.

The terms “increased”, “increasing”, or “increase” are used herein to generally mean an increase by a statically significant amount. In some aspects, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level.

The terms “decreased”, “decreasing”, or “decrease” are used herein generally to mean a decrease by a statistically significant amount. In some aspects, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

TABLE 1
Exemplary nucleic acid sequences

| Seq ID | |
|---|---|
| NO | Nucleic acid sequence |
| 1 | GCATTTCCTCTCTAGCTGTTCCTGAGGCTGGCATCTGGGTAAGTCCAGCTCCGCGGGGCA |
| | GTGTGGGCGACAGGACCGGGAGAGCAGTGGGGGAGATTGTGTTCAGAGGGCAGAACTTGT |
| | CCTGGTTAATGCACTAGGAATTTCTTCTGGCGTCTCCAAGGTTGTGCTTCAACAACATTT |
| | CTGGAATGTTCTTTTATCATTATTTGGGCTCTACGTGTGCAAGTTCGTGTGTGTGTATGT |
| | GGCGGGGGGAAGCGGGGAGGACAGCGGCAGCAACCATAGTTTACACCCACGACTTCAGCA |
| | GATTTGCCCAGTATCCCTCCCAGGGGAGAAGATGGATGGGATATACAGAAGGAAACAAAC |
| | CTCCTTAAAGACCAAAGGGCAGCAGATTTTTGTGCTGGTGTTAGGGTGGAAGGAGCACAG |
| | AAGAAAGGCAGAATCCTGATGCAGATGGAAGGACTGATGGGGCAGGAGCTGGGAGGGTAT |
| | GCGGAGCTAATAGGGCTGTCTGGAGAGCAGTCACCTCAGGATGGGCAGGAGCTGGGAGGG |
| | TATGCGGAGCTAATAGGGCTGTCTGGAGAGCAGTCACCTCAGGATGCACAGGGCTGGGTT |
| | TGCAGGGCTCAGGAGGCGAGGAGTAGATAGGCAGGAATGGAGATGGGACGGGAGAGATGA |
| | GTGAGTCTGAGGCATCTTGACATTGAACTGCAGTCACAGAAATTCCAATTTGGAGAGCTC |
| | CACCGTTTTCCAGGCTCTCTGCCTCCCTTGAGTGTTATTTATGGCTTTAGGTTGAGGTGC |
| | TTTGCCAAATAGAGTATAATCAAGGGATCAAGGAGGGAACAATTCCAAACAAAGCCAACT |
| | TGTTCATCCATCCGGACTCTAGGAGATATGGAAGATCTAAAGATACAGCAGGTGATTGCT |
| | AGTTTTTGTGGAACTCACAGTCTAGTTGGGAAGACTATGTTGGTGAATGCTGCAGTGATA |
| | CACCTGTGATAAATGCTGGGGAAGTGTGTCTTTTAGTCATCTATTTTGTTGTCTTTGTCT |
| | TTGTCTGACTGAAACAAGTGATGGAAGACATGGTCTCATATCTCTGGTCAAAATCCTGGC |
| | TCTGTTCCTTCCTGGTTCTAAACCCCTCGGCAGGCTTCCTAGTCCCCCTAAGACTCAGTT |
| | TATGCATCTATAAAATGGGGCTAGGAATAGGTGCACCTCATAGAGCTCTGTGCAGGTTAG |
| | ATGGGGAAAAACATCAGTGAGTGCCTAGCCTGTGGAAAGCTCTAAATTAATGTGAATTAT |
| | AATGATAACAATTGCGAATTTTGCAAGGAGACATTAAAGGATGTATGTTTTTATTTATTT |
| | TATTTTATTCTATTTTATTTTTTGAGATGGAGTTTTGCTCTTATTGCCCAGGCTGGAGTG |
| | CAGTGGTGTGATCTTGGCTCATCGCAACCTCCGCCTCCTGGGTTCAAGCGATTCTCCTGC |
| | CTCAGCCTCCCGAGTAGCTGGGATTACAGGTGCCTGGTACCATGCCCGGTTAATTTTTGT |
| | ATTTTTAGTAGAGACGGGATTTCTCCCTGTTGTTTGGTGAGCCTGGTCTCCAACTCCTGA |
| | CCTCAGGTGATCCGCCCACCTCGGCCTCCCAAAGTGCTGGGATTACAGGGATGTATGTTT |
| | TTATTATTCTCCATTATGCACTCCCAGCTTCATCTCACTTCAATCCACTGGTTGATAGAA |
| | GTGCAGTCAAAGACTGTCCCCTCCTCTGCAAGCTTTTATGTTCTCTCTCCTCTCTCCCTA |
| | GCCACGTCCTGTTCTGTTATCAACACACTTACTCACCTCCTGGCCTTCTCCTTCTATTGT |
| | CTCTCATCCCTCTTCCCTCACAAAAACAGAAGCAAAGAGCCAGAGCCTTCAGTTTGGAGG |
| | AACTGAAAACATTCTCTTCTGCTTTCTCATTTTGTAGATGAGGAAACTGAAGTTGAGGAA |
| | TAGTGAAGAGTTTGTCCAATGTCATAGCCCCGTAATCAACGGGACAAAAATTTTCTTGCT |
| | GATGGGTCAAGATGGCATCGTGAAGTGGTTGTTCACCGTAAACTGTAATACAATCCTGTT |
| | TATGGATTTGTTTGCATATTTTTCCCTCCATAGGGAAACCTTTCTTCCATGGCTCAGGAC |
| | ACACTCCTGGATCGAGCCAACAGGAGAACTTTCTGGTAAGCATTTGGCTAACTTTTTTTT |
| | TTTTGAGATGGAGTCTTGCTGTGTCGCCTAGGCTGGAGTGCAGTGGCGTGATCTTGGCTC |
| | ACTGCAGCCTCCACTTCCCGGGTTCAATCAATTCTCCTACCTCAACTTCCTGAGTAGCTG |
| | GGATTACAGGCGCCCGCCACCACACCCGGCTCATTTTTGTACTTTTAGTAGAGACACAGT |
| | TTTGCCATGTTGGCCAGGCTGGTCTTGAATTCCTCAGCTCAGGTGATCTGCCTGCCTTGG |
| | CCTCTCAAAGTGCTGGGATTACAGGCGTGAGCCACTGTGCCCGGCCTTGGCTAACTTTTC |
| | AAAATTAAAGATTTTGACTTGTTACAGTCATGTGACATTTTTTTCTTTCTGTTTGCTGAG |
| | TTTTTGATAATTTATATCTCTCAAAGTGGAGACTTTAAAAAAGACTCATCCGTGTGCCGT |
| | GTTCACTGCCTGGTATCTTAGTGTGGACCGAAGCCTAAGGACCCTGAAAACAGCTGCAGA |
| | TGAAGATGGCAAGCACCCGCTGCAAGCTGGCCAGGTACCTGGAGGACCTGGAGGATGTGG |
| | ACTTGAAGAAATTTAAGATGCACTTAGAGGACTATCCTCCCCAGAAGGGCTGCATCCCCC |
| | TCCCGAGGGGTCAGACAGAGAAGGCAGACCATGTGGATCTAGCCACGCTAATGATCGACT |
| | TCAATGGGGAGGAGAAGGCGTGGGCCATGGCCGTGTGGATCTTCGCTGCGATCAACAGGA |
| | GAGACCTTTATGAGAAAGCAAAAAGAGATGAGCCGAAGTGGGGTGAGTGGAAGGAAGACT |
| | TTTAAAAAAAATTGTGGCCAAGTGCACATAGTGTACAATTTTCCATCTTTATATATATAT |
| | ATATATATATTTTTTTTTGAGACGGAGTTGCTCTTGTTGCCCAGGCTGGAGTGCAATGGT |
| | GAGATCTTGGCTCACTGCAACCTCCACCTCCCGGGTTCAAGCGATTTTCCTGCCTCAGCC |
| | TCCCAAGTAGCTGGGATTACAGGCATTGCACCTCCACGCCCGGCTAATTTTGTATTTTTA |
| | GTAGAGATGGGGTTTCTCCATGTTGGTCAGGTTGGTCTCAAACTCCCGACCTCAGGTGAT |
| | CCACCCGCCTCGGCCTCGCAAAGTGCTGGAATTACAGGTGTGAGCCACCGCGCCCGGCCG |
| | TAATAATTTTTAAATGTACAGTTCAGTAGTGTTAGGTACACTCACATTGTGCAACCAGTC |
| | TCCAGAACTGTTTTCATCTTGCAAAACTGAAACTCCTCACCCATTGAATGATAACTCCTC |
| | ATTGCCTCATTCTCCCAGCCCTGGTAACCACCATTCTATTTGCCATCTCTGTGAATCTTA |
| | CTACTCTGGGTACCTCATATAAGTGGAAACATACAGCATTTTTTCCTTTATGAGTAGTTT |
| | ATTTCACTCAGCATAATGTCCTCCGGGTTCAACTATGTTGTAGCATGTGTCGGAATTCCC |
| | TTCCTTTAAAAAAGACTGTATAAATACTCCATGGTACCATATACCAAATTTTGTTTAGTT |
| | ATCTGTTGATGAACACCTGGATTACTTCTACCTTTTTGTTATTGTTAATGCTGTTATGAG |
| | TGTTGGTGCACAAATATCTCTTCAAGACCCTGCTTTGAATTCTCTGGGGTATATACTCAG |
| | AAGTGAAATTTCTGAATCATACGGTAATTCTGTTAAATATTTTGAAGAACTGCTCTACTG |
| | TTTTAGATACCATCTGCACCATTTCCCACCAACAGCACACAAGGGTTCCAATTTCTCCAT |
| | ATCCTAAGAACACGTTATTTTTATTTATTTTTTCTTTTTTATAGTAGCCATTCTAATGAG |
| | TGTGAGGTGGTATCTCACTGTAGTTTTGATTTCCGTTTCCCTAATGATAAGTGATGCTGA |
| | GCACATTTTCACTGCTTGTTGGCCATTTATAGAGCTTCTTCAGAGAAAGTTCTGTTCAAG |
| | TTCTTTTCTCATTTTCAGACCAGGTTTGGCTTTTGTTGTTGCCTTGTAGGAGTTCTTTAT |
| | GTATTCTAGATATTAACCCCATATCAGATATATGATTTACAAATATTTTCTCCTATTCTG |
| | TGGGTTGTCTTTTCATTTGGTTAACTGTGTCCTTTGATGCACAGAAGTTAGTATGATTTT |
| | TTTTTTTTTAAATCATGGCCGGGCATGGTGGCTCATGCCTGTAATTCCAGCACTTTGTGA |
| | GGCTGAGGTGGGTGGATCACCTGAGGTCAGGAGTTCAACACCAGCCTGGCCAACATGGCA |
| | AAACCCCATCTCTACTAAAAATACAAAAAAAATTAGCCGGGTGTGGTGGTGGGCACCTGT |
| | AATCCCAGCTACTCAGGAGGCTGAGGTAGGAGAATTGCTTGAACATGGGGAGTGGAGGTT |
| | GCAGTGAGCTGAGATTGCACCACGGCACTCCAGCCTGGGCTAAAGAGCAAGACCCTGTTT |
| | CAAAAAAAAATTTTTTTTTCTTGTATTGTCTTGGTTTTTTCTCTTTTCCCCTCTCACGGT |
| | AGTGCTATGAGAAGGTGGAGGCAAACTGTTTGCTTTCTCTAACTCCTTTAAAAAGCTGAT |
| | GCAGACTGTACCTACCCCCTGGGCACCTGCCATGATTAGTCCCTCTTTATCTGAATCTCA |
| | GCAGTGCCTAGACCTGCCTCCAATTCTCCATCCAGCTTCTCAGACCACATGTTTTGTCCA |
| | GTATCTGCTGTGTGGTCAGCAGGCTTGGGCCTCTGCTTCCTATGGCCCAGAGGGCAGCCT |
| | CTCTCCTTGAAGAACCGTGGAGTTTGGGGCAGAAGCCTTTCCTTTCACCACCACTTTGGG |
| | AATTGGAATGTAAAAGAAATTCTCTTAATCCCCATGTACACATTGGGGATAAGGTTCTTT |
| | CTTTTGTCTTGTGTTCTAGGCAAAGGTAGGGCTCAGAGAAGGGCAGGGTATTGCAGGATG |
| | ATGATGTAGAGCTGGTGGTGATGGGAAGGAGGAGTACAAATGAGTCAGTGGATAGGGAGA |
| | CACATGTTAAGGCAGGGCAGCAGTGTGGGTGTAATGACACTGGGGAAACAGACAGCAGTG |
| | TGCAGCCGGGGGAGGTCAGAAACCAGTTGCTGCAATGGCTCTCACCTGTAGTCCCAGCTA |
| | CTCAGGAGGCTAAGGCAGGAGGATCGCTTGAGCCCAGGAGCTCAAAACCAGCCTGAGCAA |
| | CATAGGGAGACCTCATCTCTAAGTAAATAAAGCAATTATCATTGTGTCAGTACTTATATA |
| | CTCACATACCCAAATGAAAGTGTCATAATCACAACTGTAGAAGTACTTTGCTAAGCAGTT |
| | TTTATGGATTTTATGGCACATTCAAGAAACCCATGAAACAGCTTCTGGTATTATCCCTCC |
| | TGCAAGTGAATGAGGTGAGCCTGGAAGAGATCCTAACTGGCCCATGGTTTATTTCTAGCC |
| | ATAACCAGAGCTGGGGTAAAAAGCAGGGCATGTCTCTGATCAAAGCCTGGCTCTTAACCA |
| | CTACACCGTCTTTGCTTCCATATCTGGATTTTGAGGCAGAAAAGTTATAACTAGGGTTAG |
| | AAGTTTGTTTGTTTGTTTCCTGGGGGGGGTTAGGTTTTAGGTAATAGACTTTGGCTAATT |
| | TACTGTCATGGCTCCTACCTGAGTCCCAGTTACTCAGGTGGCTGAAGTGGGTGCATCACT |
| | TGAGCTCAGGAGTTCCAGGCTGGAGTGAGCTATGACCATGCCACTGCCCTCCAGCTTTGG |
| | AAACAAAGCAAGACTCTGTCTCTTAAAAAAAAAGAATAAGATTTTGGTTTTGGCTTAGAT |
| | TTTTTGGATTTTATTTTGTGATATGGTTACCAAGTAGACAGCTGTCAGAGGTCTTTTATG |
| | GGCTGGGCGCAGTGGCTTATGCCTGTAATCCCAGCACTTCAGGAGGCCAAGATGGGAGGA |
| | TTGCTTGAGCCCAGGAGTTTGAGACCAGCCTGGGCAACATAGTGAGAACCTGTCTCTACA |
| | AAAAATGCAAACATTAGTTAGGTGTGGTGGCACGTGTCAATAGTCCCAGCTGTTCTGGAG |
| | GCTGAGGTGGGAGGATCACTTGAGCCCAGGAGGTTGAGGCTGCAGGGAGCCATGATTGTG |
| | TCATTGCACTCCAGCCTGGGCAATAGAGTGAGGCCCTGTCTCAAAAAAAAAAAAAAAATC |
| | ATTTATGTTACTTGTAGATTCCTGCCCACCTGAAGTTATCTGTCTTATATATTTCCCCTA |
| | GTGACTAACTCTTGTTTTACCTAACCTGAATTTCTCCTATAGAATGCCTTCTTAAGATAT |
| | GCTATCTAGTGACTGTTTGGATTTGTGATTCTCAACACAGATGCATGTTGGAATCACCTG |
| | GGAGCTTTTAGTATGTGATGGTTCCTGGGCCTCACCTCAGAGGGACTGAAAAAGAACCTC |
| | TGGGCGTGCAGTCCTGAAGTCCATATTTTTAAAGCTTCCTGGGTGATTCTAAAGTCAGCC |
| | AGGATTCCCAACCACTAGATTAGGCACGAAATAGACTGGTCGTTCTCACTCTTGAAGAAC |
| | CAGACATCTAGACTTGACAATAGAAGACGTGTGTAAAATGGATTGCAGGGAACTGCGGTC |
| | AGGGAAGGGTGACCTTTCTTAGCCCACAGCCGTGTTGGGGATATAGGAGAGCTTGGAGAT |
| | GCCCTCACAATCAGGCTGCCAGGGGCAGGCAAAAGCAAACGAGGGTAAAAGAAACTTAGC |
| | CTGCTTCGCATGTTTGCTGATTGATTTCCAGGCCTGAGGAAGAGATTCTGAGACTGGGGA |
| | CAGTGGGAACACATGCTTGGCAGGTGGACAGCAGAAGTTGTTTTGAAACTAGGAGTGCAG |
| | AAATGCTCCCAACCAGACTTTTGACTGGATACCTGTCCTCTGCAACTCAGAGCCATGTTT |
| | TGATTTGGGGGATGTTTGGGGTCTCCTCTCTCATGCCAAATATTAGGCCAGGTTTCAATT |
| | GCATCCTCTGTGATAATAGTTCTGGGTTTTGACACCTTTTTTTTCCCTTTTAGGTTCAGA |
| | TAATGCACGTGTTTCGAATCCCACTGTGATATGCCAGGAAGACAGCATTGAAGAGGAGTG |
| | GATGGGTTTACTGGAGTACCTTTCGAGAATCTCTATTTGTAAAATGAAGAAAGGTAAGCG |
| | ACTGGGGTGGTGCTTCTAGTTGAGTTTTAAGACCTGGTTCAGGAGGCTCTGGGAAGCTGA |
| | GCAGCTGGGTAACTTGGCCATACTATAGGTTCCTGCTCTTTGGAATGCAAAGGCCTGTCT |
| | CAGGAAATCCATTGTCAGTTGAAGAAACAAATTCATGTTTGTGTGGCTCTTGCAGTCCCA |
| | GAGCCAGCGGCGGTGATTAAGCGTTTCAAGTTTGATAAGGCATAGGATGCAGATTACTTA |
| | AAGGAATAAGATTGATTCAAATGATCTTTGGGCTGGTAGCTACACATAGAGATGGGCCTT |
| | CTGCTCAGCTGTGTATCTTAAAAACTTGACACCAGAGATTCTCGGCACCTTTCCTACCCA |
| | GTGGGGAGGAGCTGCACCTTCCATTTCTCCATTCCGGTTACCACTCGCTTCCGATGACAG |
| | GCCCCAGCTGAGAGCGCATCTCAGGTGGATGTGTGTATACTTTCCCCCTAACTTCCTGTC |
| | TTTGCCGTAGATTACCGTAAGAAGTACAGAAAGTACGTGAGAAGCAGATTCCAGTGCATT |
| | GAAGACAGGAATGCCCGTCTGGGTGAGAGTGTGAGCCTCAACAAACGCTACACACGACTG |
| | CGTCTCATCAAGGAGCACCGGAGCCAGCAGGAGAGGGAGCAGGAGCTTCTGGCCATCGGC |
| | AAGACCAAGACGTGTGAGAGCCCCGTGAGTCCCATTAAGATGGAGTTGCTGTTTGACCCC |
| | GATGATGAGCATTCTGAGCCTGTGCACACCGTGGTGTTCCAGGGGGCGGCAGGGATTGGG |
| | AAAACAATCCTGGCCAGGAAGATGATGTTGGACTGGGCGTCGGGGACACTCTACCAAGAC |
| | AGGTTTGACTATCTGTTCTATATCCACTGTCGGGAGGTGAGCCTTGTGACACAGAGGAGC |
| | CTGGGGGACCTGATCATGAGCTGCTGCCCCGACCCAAACCCACCCATCCACAAGATCGTG |
| | AGAAAACCCTCCAGAATCCTCTTCCTCATGGACGGCTTCGATGAGCTGCAAGGTGCCTTT |
| | GACGAGCACATAGGACCGCTCTGCACTGACTGGCAGAAGGCCGAGCGGGGAGACATTCTC |
| | CTGAGCAGCCTCATCAGAAAGAAGCTGCTTCCCGAGGCCTCTCTGCTCATCACCACGAGA |
| | CCTGTGGCCCTGGAGAAACTGCAGCACTTGCTGGACCATCCTCGGCATGTGGAGATCCTG |
| | GGTTTCTCCGAGGCCAAAAGGAAAGAGTACTTCTTCAAGTACTTCTCTGATGAGGCCCAA |
| | GCCAGGGCAGCCTTCAGTCTGATTCAGGAGAACGAGGTCCTCTTCACCATGTGCTTCATC |
| | CCCCTGGTCTGCTGGATCGTGTGCACTGGACTGAAACAGCAGATGGAGAGTGGCAAGAGC |
| | CTTGCCCAGACATCCAAGACCACCACCGCGGTGTACGTCTTCTTCCTTTCCAGTTTGCTG |
| | CAGCCCCGGGGAGGGAGCCAGGAGCACGGCCTCTGCGCCCACCTCTGGGGGCTCTGCTCT |
| | TTGGCTGCAGATGGAATCTGGAACCAGAAAATCCTGTTTGAGGAGTCCGACCTCAGGAAT |
| | CATGGACTGCAGAAGGCGGATGTGTCTGCTTTCCTGAGGATGAACCTGTTCCAAAAGGAA |
| | GTGGACTGCGAGAAGTTCTACAGCTTCATCCACATGACTTTCCAGGAGTTCTTTGCCGCC |
| | ATGTACTACCTGCTGGAAGAGGAAAAGGAAGGAAGGACGAACGTTCCAGGGAGTCGTTTG |
| | AAGCTTCCCAGCCGAGACGTGACAGTCCTTCTGGAAAACTATGGCAAATTCGAAAAGGGG |
| | TATTTGATTTTTGTTGTACGTTTCCTCTTTGGCCTGGTAAACCAGGAGAGGACCTCCTAC |
| | TTGGAGAAGAAATTAAGTTGCAAGATCTCTCAGCAAATCAGGCTGGAGCTGCTGAAATGG |
| | ATTGAAGTGAAAGCCAAAGCTAAAAAGCTGCAGATCCAGCCCAGCCAGCTGGAATTGTTC |
| | TACTGTTTGTACGAGATGCAGGAGGAGGACTTCGTGCAAAGGGCCATGGACTATTTCCCC |
| | AAGATTGAGATCAATCTCTCCACCAGAATGGACCACATGGTTTCTTCCTTTTGCATTGAG |
| | AACTGTCATCGGGTGGAGTCACTGTCCCTGGGGTTTCTCCATAACATGCCCAAGGAGGAA |
| | GAGGAGGAGGAAAAGGAAGGCCGACACCTTGATATGGTGCAGTGTGTCCTCCCAAGCTCC |
| | TCTCATGCTGCCTGTTCTCATGGGTAAGGAAACTCGGCTTCCAGGTGCTTCCTCCTGCTT |
| | CCTCGCCAGCTTCTTCTTGGCGCTTGCCTCCTCTCATCTCTTTTCAACTATCTTCCAAAT |
| | ACTGTTGCCACAGCTACATCATAATGCCACCACTGTCTGTTTGAGACTCCTTCATGAGCA |
| | AAGATTGATGTATGGTAGGTGGATAAATGGGATGAGGAAAAAAAAAATAAAACAAGGAAC |
| | AAATGTTTGGGGAATGCCAGTTTAGCACAAGGTATTAAGTAGGATGTAGGATTGCCTACA |
| | AATATTTGTCAACTGAAATTTCTTGTAAGCTACTTGGAGCACTAGTGCCTAAGAGTCAGT |
| | CAATGTCTGTGGGGCAGAACAATGCCAGAGAGTCTGTGTCCCAGGAGCGAGGGGACAGAG |
| | GGCCTCAGAGCTGGAGGCTGGGGGGATGTTTCTTAGAAATGTGCAATCTAAGCCTCCTTT |
| | AAGGAATGGTGGGACCTGGGCGCATAGAGAAGAAATTAAAGGAATTCTGCACTGAAGACT |
| | GGTATGAGCCAAATCATGAATGCAGTCTGAGAAAAAGTGTGTGCAGGTGACAGTGGAGAG |
| | AGACACAGTTTCCTTGGAAAAAAACATGATGAAGGTAAATCAAGGGAAATAGAGCAGAAA |
| | TGTGGAGACCCTCTTTCTAGGCCCCCATGTTGCTCTGCACTGGGCATTGCCAAAACCCCG |
| | TTTTCGGGTCTGTTCACACACTTGCTGGGTGGTTGGAAGCTAGTGGCCAAGTCTGTGCAA |
| | TGAAGTGGTTGAGTCACCTGACTTCTGAGTCCCTTTTGGGTTTATCGTTCAGTGCCTCCT |
| | GCAGAAGGACCACCCACAGTGGTTGTAGGAAGGTGGCCACCCTGTGATGTGTGAGTTTAG |
| | CCTGGATAGAAGACACTTGACGTGGGGCAAAATTAAGGGAAACAGGAGGCTTGGCCCTTC |
| | CACAGGCAACAATGAGTTTCAGCTTTCTTGATCCGTTGACATTACCATGACCAGGACCAC |
| | CTTCCTAGTCATGGTTTGGCCATGAAGGACTGACTGCCCACAGGGCCGCAGTGGTGGTGA |
| | AGACCCAGGTCCTGGTCACAGGGACTTGAGGTCTGGCTGTGGGACAGTCCGGAAACAAGT |
| | GCAGATGTCGGGGAGGGGGTCATGGGCTCTGACAAGGAAACTTGCAGAGTATCCAAAGCA |
| | CAAGGGGACGAACCTGTTCACCAGCGGGGCACGGGTGGGTGTCCCCGCTCAGCCAGCTTC |
| | CGGAGGATGGGCAGTGAGAGAGTGAGAGAAGGAAAGGGGCAGCCAGGCTGGGGGCTTACG |
| | GTGTTGAGAGAGAGGGCAGGTGTGGGAAAGCAGGGTACGGTTTCTACGGCAGCAAACAGT |
| | TCCGAGTGACTGGAAAAAAGACAGTTGTCTTCATTCATCAGGTTGCCATAACAAAACACT |
| | GCAGGCTGGTTGGCTTATAAGCAAAAGGAAGGTTCACCTTACGGTTCTGGAGGCTGGGAA |
| | GTCCATGATCCAGGCTCTGGCAGATCTGGTGTTTGGTGAGGGCCTGTTCCCTGGTCGATG |
| | GTGCCTTCTCACTGTGTCCTTACATGGTGAAGAGGCAAGGCAGCTCTCTGGGGTCCCTTT |
| | CCTAAGGACAGCAATCCCACTCCTGAGGGCTCCACCCTCATAACTTAACCACACCTCAAA |
| | GGCCCCATCGCCTGATACGTCATGTTGAGGGTTAGGATTTCAGCGTATGGATTTTGGGGC |
| | ACACAAACATTCAGACCATAGAAATGGGAGAAGAAGAGTAAGACAGAGAGAAAAGTAGGC |
| | AGAACTACTCATTAAGAGTGAGGTGGAAGGTGGAGAGGGCAGGGAGACCTTATGAAGATC |
| | ACACATCTGTGAGGAAGGTTTGCACACTCTCACTGAAGCCCCGGTAGGAGGCTCAGCACC |
| | CATTTCTCTAGATAGCACCTTCCTTCCTCTGAGGACAGATTCTGACTGGAGCCCTGGTAG |
| | GGGGCTCAGCACCCATCCCTCTAGATGGCACCTGCCTTACTCTGAGGACAGACTCTCACT |
| | GAAGCCCCGGTAGGAGGCTCAGCACCCATTTCTCTAGATAGCACCTTCCTTCCTCTGAGG |
| | ACAGACTCTGATTGGAGCCCTGGCAGGGGGCTCAGCACCCATTTCTCTAGATAGCACCTT |
| | CCTTCCTCTGAGGACAGACTCTCACTGAAGCCCCGGTAGGAGGCTCAGCACCCATTTCTC |
| | TAGATAGCACCTTCCTTCCTCTGAGGACAGACTCTGATTGGAGCCCTGGCAGGGGGCTCA |
| | GCACCCATCCCTCTAGATGGCACCTGCCTTACTCTGAGGACAGACTCTCACTGAAGCCCC |
| | GGTAGGAGGCTCAGCAACCATTTCTCTAGATAGCACCTTCCTTCCTCTGAGGACAGACTC |
| | TGATTGGAGCCCTGGCAGGGGGCTCAGCACCCATTTCTCTAGATAGCACCTTCCTTCCTC |
| | TGAGGACAGACTCTCACTGAAGCCCCGGTAGGAGGCTCAGCACCCATTTCTCTAGATAGC |
| | ACCTTCCTTCCTCAGGACAGACTCTGACTGGAACCCTGGTAGGGGGCTCAGCACCCATCC |
| | CTCTAGATGGCACCTGCCTTACTCTGAGGACAGACTCTGACGGGAGCCCTGGTAGGGGGC |
| | TCAGCACCCATCCCTCTAGATGGCACCTGCCTTACTCTGAGGACAGACTCTGACTGGAGC |
| | CCTGGTAGGGGGCTCAGCACCCATCTTTCTAGATGGCAGCTGCCTTACTCTGAGGGCAGG |
| | AGCTTGCTTTGTATGTTCCAGTCCCTTCTGAGAACCAGAAGCCTGAGCATCTATTAGAAA |
| | TGCAGGATTGTGGACCCAATCTTAGACCTAGAGAGCTAGACTTTGCATTTGAATGAGAGC |
| | TACAGGTGATTCAAAGGTGACTGCATGTGTGGGTGCAGTGGCTCTTGCCTGTAATCCTAG |
| | GTTTTGGGAGGCCAAGATGGGGGGATTGCTTGAAGCCAGGAATTCAAGTCCTACCTGAGC |
| | AGCAAAGTGAGAGCACCTCTCTACAGAAAAACAAAAGCAAAAATGTGACTGCATGTTGGA |
| | GCCACCTGGCTTTTTTTAAATAAAAAAGATCCATATAGCTGGACACAGTGACTCGTGCCT |
| | GTAACCTCAGCTACTCGGGAGGCTGAGGCAAGAGGATTGCTTGAGCCCAGGAGTTTGAGG |
| | CTGCAGTGAGCTATGATCATGGCACTGCATTCCAGCTTGGGTTTCATCTGAAAAAAAATT |
| | TAAAAAATTCATCTAGTTCCACCTCAGAGCTTCTGATTTTATTTCCATGGATGTGGCCTG |
| | GGCTTGCGATTTTCTTTTTCTTTTTTTTTTTTTTGAGATGAAGTCTTGCTCTGTTGCCCA |
| | GGCTGGAGTGCTGTGGCATGATCTTGGCTCACTGCAACTTCCACCTCCCAGGTTCAAGTG |
| | ATTCTCCTGCCTCAGCCTCCCTAATAGCTGGGATGACAGGCTCGTGCCACCACACCCAAT |
| | TAATTTTTGTATTTTTAGTAGAGAGAGGGTTTCACCATGTTGGCCAGGCTGATCTTGAAC |
| | TCCTGACCTCAGGTGATCTGCCCGCCTCGGCCTCCCAAAGTGCTGGGATTACAGGTGTGA |
| | GCCACCATGCCCAGCCTGGCCTGAGGATTTTTGTGAGCACTCCTGATGCTGTTGTGTAGC |
| | CAGGAGTGAGGACTGCCCTCTGATCACCCAGCACAGAACGTTCAGGGCCATGTTGGGACA |
| | GTGGACTGGTAAGGGCGAGAGGAGGACACTGGGGAGCAGGCTGTAGGGTGCCACAGTCCG |
| | CCACAGTCACAGGCTCATGCTCTCCGTCCGAGAGGAGGCAGAACTGCTTTTACCAAGGCT |
| | CATCCAGCCCTCTTCATTTCTCTGGGAACAATTTTTGGTTTCGTGACCCATTTCTTAATC |
| | CCCGGAAGGCATTTCTCTGAACTGGTGCCAGGCACCCCGGCCCCCAGCTCCAGTTAGTTA |
| | TTATTCGAGGCTGATTTCTTTTCTGTCTGTCTTCCTTCTAATTCCTAGATTGGTGAACAG |
| | CCACCTCACTTCCAGTTTTTGCCGGGGCCTCTTTTCAGTTCTGAGCACCAGCCAGAGTCT |
| | AACTGAATTGGACCTCAGTGACAATTCTCTGGGGGACCCAGGGATGAGAGTGTTGTGTGA |
| | AACGCTCCAGCATCCTGGCTGTAACATTCGGAGATTGTGGTGAGTCCCCGTGCATGTGAT |
| | CTGTGTGAGTGCAAGTTCATATGAGAGAGAGAGAGAGAAACAGACTGGAGAAAGATCTTC |
| | AGGACCAGGTTGCTGGTGTGGTTTCTTTCTTTTCTTTTTCTTTTTTTCTTTTTTTGAGGC |
| | AGAGTTTTGCTCTTATTGCCCAGGCTGGAGTGCAATGCCGTGACCTCGGCTCACTGCAAT |
| | CTCTGCCTCCTGGGTTCAAGTGATTCTCCTGCCTCAGCCTCCTGAGTAGCTGGGATTACA |
| | GGCACCCACCACCATACCCAGCTAATTTTTTGTATTTTTAGTAGAGATGGGGTTTCATCA |
| | TGTTGTCCAGGCTAGTCTCGAAGTCCTGACCTCAGGTGATCCGCCCGCCTCGGCCTCCCA |
| | AAATGCTGGGATTACAGGCATGAGCCACCGCACCCGGCCGGTGGTGTGGTTTCTGCTGTA |
| | ACAAAGTGCCACAGACTGAATGGCTTCAACAACAAAAATTCATTTTCTTGCAGTTCTGAG |
| | GCCGAAAGTCTAAGATCAAGGTGTCAGCAGGGTTGGTTTCTTCTGAGGCCGCCCTCCTTG |
| | GTTTGTAGATGCCCCCTTCTCCCTGTGTACTGATGCCATCTTGCCTTGGGGGGAAGAGTG |
| | GCCTAGTCTCCTCTTCTTACAGGGACAGCAGTCCTGATGGATTAGGCACAGTCAGGCATG |
| | ACTACACAAGACCTAATGTTAATCTAGTCACTCCTTTAAAGACCCCATTTCCCAAAACAG |
| | CCACATTATGAGGTCTTGAGGATTAGGGCTTCAACATGTAAATTTTTGGGGGGGCACAGT |
| | TCAGCCCAGAACAAGTGAGAAGGTAGAATGGGTTGGAGGCCAGCTGGGGAAGTTTTGTGT |
| | GGGGAAGAATGAATGAGGAACTAAAAAAAAAGTCATTTCTTGAAACAACCATGATGCTCG |
| | ATATATTCCATTTCCTCATGGGCCATGTTCATAGATGAGAGTTGGAGAATTTGCCATTTA |
| | CAACAATGGCAACCTCACCTAAAAACTATGAAAATACTTTTTTTTTTTTTCTTTGAGACA |
| | GGACCTCTCTCTGTCACCCAGGCTGGAGTGTGTGATCACAGCTTACTGCAGCCTCGACCT |
| | CCCAGGCTCAAGCAATCCTCCCACCTCAGCTCCCCCGAGTAGCTGGGACTACAGGCATGC |
| | ACCACCATGCTGGCTAATTTTTGTATTTTTTTTGTAGAGATGTGGTCTCATCACGTTGCC |
| | CAGCCTGGTCAGAATGCTTTGTATGGGTTGAAATGTCACAGGGAATCTCCTGTGACTTTA |
| | TTAATAATGTGCTTGCTCACACCTGTAGTCCCAGCTACTCGGGAGGCTGAGGCAGGAGAA |
| | CTGTGTGTACCCGGGAGGCGGAGATGGCAGTGAGCCGAGATCGTGCCATTGCACTCCAGC |
| | CTGGGTGACAGAGCGAGATTCCATTTCAAAATAATAATAATAATAATAATAATAAAAAAT |
| | GTGCTTGCTTTCTCCCACACCATGAATCAGATTCACACGCCCCACCGCGCTGGTGCCTGG |
| | GCCGCCTCACTAGCTCTGTTTCATTCCCTGCTCTGTGGACGCATGCAGGCGTCTGCATTT |
| | TTGAGGAGCTGCACAGCAAGTGTTAAAGTCCGCCACTCTAGGACATTTTGCTACCTCCCT |
| | CTCCGGCTCAACACACTCACCTTGTCACCAGTCTTATCAATTTTTCCTCCTACCCGAGCT |
| | GTTTTCCACTTTTCCTCGTCCTCACCCCACCAGCCCAGGTCCAGCAACTGTCACCACTTG |
| | CCTGGACACGCAGTGACCTCCTAGACCATCAGCTCCCCGGGGATGGGGTTTGCGTTTGTT |
| | TGTCTTGCTGTCATCTTTGCCACGCCTGGACAGACATCTTAGTACCCATCAGTGGTCAAA |
| | AGCACGCACTGAATGAATGTTGGCTCTTTGGTCTCCCTGTGTCTGCCTTTTTCCCTCACC |
| | CGACCATTCTCTGCACAAAAGTAAGATGGTCCTTTAGAAAATTCTGATTCGATTGTGCTG |
| | TTCGGTGGCTTAAATGCTTTCTGTCTGCTCTGCTCTTTATTTTTTACTTTATGTATTTAT |
| | TTTTGTAGATGTAGGGGGCGCAAGAGCAGCTTTGTTTCATGGATATGTTACTGCACAGTG |
| | GTAGTGTCTGGGCTTTTACTGTAACTACCCCTTTTTTTTTGAGATGGACTCTCGCCGTGT |
| | TGCCCAGGCTGGAGTACAGTGGAGTGATCTCGGCTCACTGCAACATCCACCTCCTGGGTT |
| | CAAGCAATTCTTCGAGTAGTTGGGATTACAGGCATGGGCCACCACGCCCAGCTGATTTTT |
| | GTATTTTTAGTAGGGACAAGGTTTCACCGTATTGGCTAGGCTGGTCTCAAACTCCTGACC |
| | TCGTGATCCACCCGCCTCGGCCTCCCAAAGTGCTGGGATTACAGGCGTGAGCCACCGTGC |
| | CCGGCCTTAGTGTAACTCTTACCCAAATAGTGAACATTGTACCCAATAAATAATTTTCCA |
| | GCCCTCACCTGGCTCCCACCCTCCCACCCTTCTGAGGCTCTAGTGTCTATCATTCCATGC |
| | TCTATGGATGTTACCCTTTGTTTAGCTCCCACTTGTAAGTGAGAACGTGTGGTATTTGAC |
| | TCTGTTTCTGAGTTGTTTCACTTAAGGTAATGGCCTCCAGTTCCATCTGTGTGGCTTCAA |
| | AGATGTGACTTCATTCTTTTCTATGGCTGAGCAGTATTCCATTATGTATATAGATCACAC |
| | TCTTCATCTTTGCTCTTTTGGTAAATTTCAAATCGTAGTGCCATGGAGACTGGTTGTTTG |
| | GGACATTTAACCTTATTTCTCCAACTTCATGCCCAAATTCTCTCCCCACTGCCATTGCCC |
| | CTGTATGATTCTGGAGAGGCAGACACAAACAGGGAAGGGTAGGGGACAGTGAGATGCTTC |
| | GCAGAGAAGTAGAGATCCATTTGTCCATTTGCTTGAAGTTGGTGTAAGGATAGGGACAGA |
| | AAGGAAGGTGGTCTAACAGGCTCCAGGACCTATGAACTATTGCCCTGGAAAGAAGCTAGA |
| | ATGTTGTTCTTGCCCCCATGACAGGGATAAAGTATAGTTTTTGTTGCTGGTGGTGGTGGT |
| | GACGGTGGTAGTGATGTGCGTGTGTGTTTGTGTGCATGTGTGTGACGGGGAAACTAATCA |
| | AGAATCAGAGCTGCAGCGGGATGTGGAATCCCAGCACTTTGAGAGGCTGAGGTGGGCAGA |
| | TCACCTGAGCCCAGGAGTTCAAAACCAGCCTGAGCAAGCTGGTGAGACCTTGTCTTTAGA |
| | AGATAATAATAATAATAAAAATAAAATATAAATAAAATAAAATGGCTAAGCATGGTAGCA |
| | CATGCCTGTATTTGCAGCCACTTGGGAGGCTGAGGCAGGTGGATCCCTTGAGCCCAGGAG |
| | TTCGAGGTGGCAGTGAGCTATGATCTCGCCAGTGCACTCCAGCCTGTGCAACAAAGTGAG |
| | ACCCTGTCTTTAAAAAAAGAAAAGAAAAGAAAAGAAAAAGAATCAGAGCTGCTTTTCCTT |
| | TCAATATAGATGGAGGGCTGGGATGAAATCGTCAAACACAATGGGGTGACAATGTTGGTG |
| | AAGACAAGAAATAGCTGAGTGCAGCACTGGCGAGGGCAGTGCCGACTGCGGGCACGTCAA |
| | TGGTTAGGAACACGAGGGAAGGAGGTGGGTGCACAGGCCCACACCGCCCGCTTCACTTAC |
| | AAACCAGGAGCGCACCATTCACATGTCTGGGGGGTGATCTCAATGCACCCGGCACTGTTT |
| | ACGGCCGGGAGTTCACTGACAGGTGATCCCTCTGCTGTGACTGGGGAGACTGGGGGCTGA |
| | CCTCCCAATGTGCCTTGTGCTGTATTCCGGAGCTCTCTGGTCAGGTGTATTCTGATGCTT |
| | TCTCTATTCCAGAGCTCTCTGGTCAGGTGTGTCCTGATGCTTTCTCTATTCCAGAGCTCT |
| | CTGGTCAGGTGTGTCCTGATGCTTTCTCTATTCCGGAGCTCTCTGGTCAGGTGTGTCCTG |
| | ATGCTTTCTCTATTCCGGAGCTCTCTGGTCAGGTGTGTCCTGATGCATTCTCTATTCCAG |
| | AGCTCTCTGGTCAGGTGTGTCCTGATGCTTTCTCTATTCCAGAGCTCTCTTGTCAGGTGT |
| | GTCCTGATGCTTTCTCTATTCTGGAGCTCTCTGGTCAGATGTGTTCTGATGCTTTCTCTA |
| | TTCCGGAGCTCTCTGGTCAGGTGTGTTCTGATGCTTTCTGTATTCCGGAGCTCTCTGATC |
| | AGATGTGTTCTGATGCTTTCTCTATTCCGGAGCTTCCTGATCAGGTGTGTCCTGATGCTT |
| | CCTCTGTTCTGGAGCTCTCTGGTCAGGTGTGTTCTGATGCTTTCTGCCTCTGTTCTTGGC |
| | ATGAAGGTTGGGGCGCTGTGGCCTCTCGCATGAGTGCTGCTTCGACATCTCCTTGGTCCT |
| | CAGCAGCAACCAGAAGCTGGTGGAGCTGGACCTGAGTGACAACGCCCTCGGTGACTTCGG |
| | AATCAGACTTCTGTGTGTGGGACTGAAGCACCTGTTGTGCAATCTGAAGAAGCTCTGGTG |
| | AGTCGAGCCCGTTCCCCTAAGGAAGTTCTGCCAGCGAGGCGTGCTGCGCACTCTGGCTTC |
| | AGTGAGGCCCGGTGGGCTGGGGTTTGAGTGAGAATGGCCTTGGCGGCTCGGGTGACTGCG |
| | TGTGCTTGTCTTGGGGTGTCTAGCATTGCGGTCAGTGAGTGTTTGTCGTGGGGAAAGCCA |
| | CACACTCAACCCTGACAAGGAGCATAAATAATAACAAAAAGAAGATGTCTGCCTTTAGGG |
| | TGCCTACTGCCTAGCTGACAAAAAGAAAAAATCAGTGTAATGCATTGCTTTAGAGGAGTT |
| | TTAGAACTTCATGACAAGCCCTGGGTCCTCATATTGTATAAGAAAAATGGTGCTAAAGCA |
| | ATGCAGACCCTCGATGTTGTATGCAGCCCTTCCCTGAGTGCTTCTGGGAGACTTGCATTT |
| | TGAAGGAGAGAAAAAACCACTTAAAAATGGAAGGGCCATTTCTCACCCATTAGGATGGCT |
| | ATTATCAAACAAAAGCAAGAAAACTAGAAAACAACAAGTATTGGTGTGGGTGCGGAGAAA |
| | TGGAAGCCATTGTGTGTTGCTGGTGGGAATGTAAATGATGCAGCCACTATGGAAAACAGC |
| | ACAGCAATTCCTAAAAAATAAATACAGAATTACTACTGGAGCCAGCATTTCCACTGCTGG |
| | GTATATCTGCAAAAGAGTTCAAAGCAGGAACTCAGCCAGGAGCGGTGGCTCACACCTGTA |
| | ATCCCAGCATTTTGGGAGGCTGAGGCAGGTGGATCACTGGAGGTCGGGAGTTCGAGACTA |
| | GCCTGGCCATCATAGTAAAACCTTGTCTCTACTAAAAATACAAAAATTAGCCAGGCATGG |
| | TGGTGCATGCCTGTAATCCCAGTTACTCGGGGTGGAGGCAAGAGAATCTTTTGAACCTGG |
| | GAGGCGGGGGTTGCAGTGAGTCAAGATCCCGTCACTGTACTCCCGCCTGGGTGACAGAGG |
| | AAGCATTAAAAAAAACAGAAGGCAGGAACTCAAACATGTTATTTGTACATCTATGTCCAT |
| | AGCAACACTATTAGAAATAGCCAAAAGGTGGAAATAACCAAACGTCCTTCAACGGATGAA |
| | GGGTGTTGAAAAGTGCTATATATGTACATTCAGCTTTAAAAAGAATTCTAGTACACACTA |
| | CAACATGGATGAGCCTCGGGGACATTATGCTAAGTAAGATAAGCTAGACTCGAAAGGGCA |
| | AATACTCTGATTGTCCTTATAGGAGGTGTCTAGAACAGACAGATTCATAGAGACAGGAAG |
| | AAACGTGGTTGCAGGCAATGGGAGGGCGGAGAGTGGGAAGACATTGTTTAATGGTTATGG |
| | AGTTTCAGTTCTGCAAGATGAAAGGAGTTCTGGAGATGGTGGTGGTGATGGTTGCACAAC |
| | AATGTGAAGTGCTTAAGGCCATTAATTGTGCACTTAAAAATGGTTAAAATGGTAAATTTT |
| | ATTTTACACGTATTTTACCACAATAAAGCTGAATGTAGGGAGCTGGGAAGATGTAGTATT |
| | GGTGGGAGCTTGGAGAATGCTTACTTGTTATTCCACCAAATGGAAAAGGAAATCAGAAGT |
| | GTACATAGAGCTTGTGTCCACTCCCTTTCCATGTGTAAACTGGAGTAGAGGCAGTGGCAG |
| | GTACGGGTGCTTCCTTGTCCATGGTGGAGCGTGGGTGAGAGACATGACTGACATTCTGCC |
| | ATCTCTATGGAAGGTTGGTCAGCTGCTGCCTCACATCAGCATGTTGTCAGGATCTTGCAT |
| | CAGTATTGAGCACCAGCCATTCCCTGACCAGACTCTATGTGGGGGAGAATGCCTTGGGAG |
| | ACTCAGGAGTCGCAATTTTATGTGAAAAAGCCAAGAATCCACAGTGTAACCTGCAGAAAC |
| | TGGGGTAAGTCTTCATGGGTGTCTTACCAGAAAAGTAACTTCTTTTCAGAGGTGAATTAT |
| | TTGGAAAATGTGGATGGGGGTTACTTTGGTCAGGCAGAGCTGAAGGTAGAGTAAGGAGGT |
| | AAAAACATCAGAAATGCTTTGAAGGACTGGAGGCATATAATGTATACTGCTGCTAATAAG |
| | ATAATTTATGCTAATTTATGATTATAGAACTAGAGTTTCTTAGTGTTGGAAGAAACCTTA |
| | GAGATGGTGCAGCCCAATTGCCTGTTGAGGGAAAAACTTGCTTCCAAGACACTCACATGA |
| | ATAGTCACCCATTTTATGTGTGCATGCTTGCAAGCATGCAAGTTATCGCATAGTGCATGC |
| | AGCCTCTACACTAGGAAAGAGCTCTCACCCTGCAGCCTCTACACTAGGAGAGGACTCTTA |
| | CACTGCAGCCTCTATATCCTCAACAGTGTCATGGCTCAGCACCTTCACCTATGTAGCCAT |
| | GCTCTCCCAGGTACACAGGCTGGTTTGTTAGTGTCTGTCTTTGAAATGTGGTGCCCAGAC |
| | CTGATGATGCATGGTGGATGTTTTACCTCCTGTTGCAGAGAACTATGGTTTCACTGGTGG |
| | AATCTCTGATTTCCTTGTTTTTTTTGGCAGTTACATAAGCCTTTTGATTCATGTTGGAAT |
| | TCTGCAGTAAACAGTAGTAGGGAATACCCACTAATAATCATGCACTTTCCCTGTACCACT |
| | TTCCTGTATCACTTTCCCTGTATCACCTGCTCTTTCATGTTCTTACATGGATTTTCTCAG |
| | TTGGCTTCAGAAAGATGCTATAGGGTAGGGTATCTGTGTCTCCAATTTAAAGAGGAGAAA |
| | ATAAAATTCAGGGAGCCTCATAACTTTGTCCAAGGACACTCAGAGAGAAGGTAGTGGAAC |
| | TCAGTGTAGACAAACGTCTGTGACCTCCCAAGCCACTGCCTTAATTCCCAGTGCATTGCC |
| | TTAGGAGATGTTGCTAATGGGCTCTCTACAGGGCTTCTTCATCTGGCTCTCAGCTTAAGG |
| | GTGGGACCTCCAATTTAGCTTTGCTAAACCTCATCTTACCTGTCTTTTGACCCATTGACT |
| | AGGCAGTTTTGAATCTTCATTCTGACAATAATATCTACAAATACAATAAGCAAATTGGAT |
| | AGGTCTTCATTCATTCACATTATTAATATATATACCAGCCAGATCAGCATTCAAGACAAA |
| | ATTTGGCAGGAAAGGTCTTCCCAGAGGAGTAATCCACTAATCCATAATTAACAACCAACC |
| | TCTGTTCACAAAGCTCTGATACTTCCTTGGACCTCTCCCATCAGCCAATCCTCAGATGTG |
| | ACCATATCTCTTTCTATGAAGTCTTCCTGCAGTGCCTTCATTTCCTCGCCTTAGGGTATT |
| | ACCCTTCCTTAGCAAATCGCCTTAGAATATTACTCTTCCTTGGCAATTTGACTTCTGCTT |
| | CTCTAAGTTTGTTCAACAGACATGATTTTGTCCCTTCATTTTGCAGGCCCTCTGGGGGCT |
| | CTGTGGTGGGCACTGACAGCAGCAGTGAGTAAAACAGACCAAAATCCTGTTTTCACAACC |
| | GTTCTTTTAGGGAAGTGATAACAGATAATAGACATGCTAAACAATATACCCTGTGTATAA |
| | GAGCTATAAGAAAAACTGGAGTGACGGCGAGGGGTGCTTCTCTGCTCACGTTTGGCGAGT |
| | GCCTCTCCGAGCGGGTGACATTGGAGCAGGTGCTCAGTGAAGCGTGGGATGGGCTGTGAA |
| | GGAGCCGGAAGAGCCCTCCAGGCAAAGCAGATGGAACCGAAGAGGCCCAGGCGTAGGCAC |
| | TCAGGTGACTCTCAGTGATCCTGGTTCCAAATTCCACATGACAAACAAGCCAATTACAAA |
| | TGTGCCTGATGCATCTGTAATATGCTCTATGTAGTTACTATTGTCAATACCTAACCTGGT |
| | AGAGCTGCTGTAATAAAGTATCACAAACAGAGCGGCCTAAACAACAAGAGTTTATTGCCT |
| | CTCAGTTCAGGAGGCCAAAGTCTGAGACTGAGGCATCAGCAGGCTGGGTCCTTATAGGTC |
| | GTACAGGTCCTTCCCAGTCCTTACTTTAGTTGACATCTGCTGCCTTTAATTCATTTCACT |
| | CTTCCTCTTTGGAAGCCTTCTTCGCTCTTATTTTTAGGTCACTACTTAGTCTTTCCTGCT |
| | AATGTTATAGCTTCCCCCTCCCTGGATCCTGCATTGGCTTCTCTTCCTGTGGTCTGGGGA |
| | TTTCACGACTGGTTTAGTTGTGTTTTTTTTTTTTTTTTTTTTTTTGAGTTGGAGTCTTAC |
| | TCTGTCACCCAGGCTAAAATGCAGTGGCGCAATCTTGGCTCACTGCAACCTCTGCCTCCC |
| | GGGTTCCAACAATTCTTCTGCCTCAGCCTTCTGAGTAGCAGGGACTACAGGTGTGCACTG |
| | CCATGCCTGGCTAATTTTTGTATTTTTAGTAGATACGGGGTTTCACCATGTTTGCCAGGC |
| | TGGTCTCGAATTTCTGACCTCAGGTGATCTGCCTGCCTTGGCCTCCCAAAGTGTTGGGAT |
| | TACAGGCGTGAGCCACACACCCGCCTGGTTTACCTTTTTTGTTGTTGTTGTTACTAAATA |
| | TTTCTAAACTCCCTGAGAATTCTCATATACCTCTCAATTTCAACAACAGTAGGTCTCAAA |
| | CCCAGATATCATTCACAAACTTTTTATTAGAAAAGTAACACATGCTTGGTGTAAACATAT |
| | CATAGTACAGACAAAGTGGAAAGTGAAAGCTCCTTCTCAGCCCTCTTCTGAGAGAGCCAT |
| | TTACAATAGTCTGATGAACAACCATCCAGACTTTTTTTAATACAATAGATTGTTATATAA |
| | TCCATTCATCTGTCCATCCATCCATCCATCCATCATCCATCCCTCTATTTTCCATCCGTC |
| | AATCCATCCATCCATCCATCCATCCATCCATCCATCCATCCATCAATCCATCCAAGGGAG |
| | TATCAGAGCTTTGTGAACATATATTGGTTGCTAATTATGGACAAATGGATTTCCTGTATA |
| | TGCTGATCTGGGTGGTATTTATATCAGTAATGTGAATGAAGACCTATGTGATTTTCATGT |
| | TGCATTTGTAATACATTGTTATGTAGTATTTTAAATGCCTAGTCTGTTAGAGCTACTGTA |
| | ATAAAGTATCACAAACAGAGCAGCTTAAACAACAGAAATTTATTGTCCCTCAGTTCTGGA |
| | GGCCAGAAGTGTGAGATTAAGATGCTAGCAGGCTGGGTCCTTCTGAGGGCTGTGGGGAAT |
| | CTGTTCATGCTTCTCTTTGGCTTCTGGTGGAATGTCATCTTGCTGACACATTTTGCCATT |
| | CCTTGACCTGTAGATGCCTCTGCCACACCATTGCCTTTATCTCTACGTGGTGTCTTCCCT |
| | GTGTCTGTGTGTGTCTTTCTCCATGTCCCAAGTTTCCCTGTGAAAGAACACAAGTCATGT |
| | TAGATTTGTAACGACCTGATCTTAACTAATTACATTTTCAGTAACCGTATCACCAAATAA |
| | GGTCACAATCTGAAGTACTGGGGCCTAGGACTTCCACATATAAAGTCTTAGAGGAAACAA |
| | TTCAATCCATAACACCCTATCTATTGAAACTAGAACCATAATATAAATATTTTTCTGTGA |
| | TATCCTTTTTTCATTTATCATGGATAAATTTCCATGCTGATTTGTGTAGATCTATCGATT |
| | GGGGCATAATATCCCATAGTGTGCTTTACCATAATTTATTTAATCAAATCATGCATTTTG |
| | GGATAACTGTTCTTTTCCACTATTACAAACAACTCTGCAAAAAGCACATTTGTATATATA |
| | GTTTTATGCATGTGTATTTGCTTTGAGATAGGTATCTATAAGTGGCATTAATCAGTCGAG |
| | ACTTTTATGCATTTTATGTGTTAGGTACAGCCATACATGTGGCTGTGTGTTAGGTATAGC |
| | CTATGGGTTAGGCATAGCCTGTGTGTTAGGTATAGCCTATGGGTTAGGTATAGCCTACGT |
| | GTTAGGTATAGCCTATGTGTTAGGTATAGCCTATGTGTTAGGTATAGCTACATTTTTTTC |
| | TCTCGAACTTTGGGTCCTCATCTCTGGCTGCATATTGTGTACTGCCTGTCTCAATACTGC |
| | TCAAGGGTCACACATGCTGAGCATTTGCAACATTGGTTTAATTATCATACCCCACCTTCC |
| | ACCCCCGTTCTCTGACCTGCTCCTGCCCTCTTCCTCCTGCCTCATTGAATAGTGGGGCTT |
| | TCCAGGAGGCTGCTGAGTGAGTAAACGGGGGACGGTCCTAAATGCTCTGCTTTCGGTTCC |
| | CTCTACCACTCATCACTACACCAGAGGGAACTTTTTAAGATGCAAATTTGAAAATATTGA |
| | CCCTTTGTTCAAAAATCTTCAAAAGTTCTTCTCTGCAGTCAGAGTGACATCTTAGCTTGG |
| | CACACAAGGCTGTCCCTGATACTGCCCTCTCTCCTGCCATACCTTGCACTCCATTTTGAG |
| | TCATATTGGATGAGTTTTGTTTTGCTGTTTTAGAGATGGGGACTTGCTATGATGCTCAGG |
| | CTGAATTTGAATTCCTGGGTTCATCTACAGCCATACCACCCAGAGTGCCTGATCTCATCT |
| | GCACTCCTGGGCTCAAGCAATCCTCCCGTCTCAGCCGCCCAAGTAGCTGGGACTATAGGT |
| | GGACATCACTGTGCCCAGCTATGAATTAGTTTTGAAACATCTTGGTGGTTCTTTTAATCT |
| | TCCTTATTCTTGCTACTCTTTTCCTTTTGCCTGGAAGGTTACCCCCACTTTCCCACTTTC |
| | TACTATCCATCTAAGAAGCTCCTACTTATTTTTTAATTACTGTGATCCTCATTTTGTCCA |
| | TTTAAAAGCAGGGATAAAACTAACATTCCCATCTTGAGGTTGTGATCATTCAATGAGATA |
| | ACCCTGTTTAAAGTCGTCATCCATGTCAAGCACACAATAAATACTCAAAAAATGTTTGAT |
| | CTTCTTCTTCAGATTCCTTCCTTCCTTCCTTCCTTCTTCCTCCTCCTCCTTTCTTTCTCT |
| | CTTTTTCTTTTTCTCTTTCTTTCTTTCTTTCTCTCTCTCTCTCTTTCTTTCTTTCTCTTT |
| | CTCCCTTCCCTTCCCCTCCCCCCCCCCTTTCCCTTTCCCCTTTCCTTTCCTTATTTTCCT |
| | TTCTTTTCTTTTCTTTTTTTAAGACAGGGTTTTGCTCTGTGGCTCAGACTGGAGTGCAGT |
| | GGTAGGATCATAGCTCACTGTAGCCTTGAACTCTCCTGGGCTCAAGCAATCTTCCTGCCT |
| | CAGCCTTCTGACTAGCTAGGACTACAGGTGTGTACTACCATGCCTGGGTAATGTTTAAAA |
| | TGTTTAAAATTTTTTATGTAGAGACAGGGTCTTGCTATGTTGCCCAGGCTGGTCTCAAAC |
| | TCCTGGCCTCAAGTGGTCCTCCCACCTTGGCCTCCCAAAGCACTGGGATTCAGTTTTCAT |
| | TATGAGACAGTTTTGTGACATAGGCAGGTAGTTAATAAATAAAAATCCCCAACCAATTAA |
| | CTTAATACAGTACATGGCAAGATATCCGTGCTTGGTAAATGTAGAGCTCAGTTAGGTGGA |
| | TTGGTTGAAATCGAGATGTCAATCACATTCCTCTAATCCAAGGATTGGTTGGTGGGACAT |
| | GGTTGTTTGATTCTTTAAACATTTTATTATGTGTTCAGTGTAACCGTTGCTTAATAGTTA |
| | CCAGCATTTTCACAATTTGGATATGACGTTTTCTTAACAAAACTGCACCGTCTGTTGCAA |
| | TTTCTACTACTACTGTGTAGTGCTCCCCAAACATCTGTTGAATAATCCATTCTACTCCTT |
| | TTTGGAAATAGTGGTAAAGCCTGCATAACTTTAATTACATAATTTAAAAAATATTTTCTC |
| | TTTTCCTCTTTGAAGACCATCCCCATTCTACTAGAATGTCCCTGGCCTTTGCCAGGAATG |
| | CTACTAATCCCTTCTGCATGTTAATTAACAACAACCCTCTGATAGAATTCTTACCGGGGA |
| | TGAGAGCTTAGTAGAAGCCACTCAAAACTTGCTTTGTTTTTGTCTGTCTCTCTTGGGCTT |
| | CATTTGCCTATTAGTCTTACTTACTGTGTCTTTTCAGTTTGACTCTTCCTTAATTGGGAA |
| | GATAGAAACCAGTGGATGGATAATCACTTTTTCCTGCTGGTCATCTGTTCATGCTTTACT |
| | GTCTGAATACACTTTTTCTGTATTTCTATTTTTCCTGCCTCGTAGACACCAAAGAGCTTA |
| | AAAAACATGCCATTGCCAAGGACTCTGGCAGGAAGGAGGCAATCACCTGCGCTATTCCTG |
| | GCTTCATGGAATAGAAGCATCTGTGCTCCTAGGAATTACGCCACCAGTGATTTTAGAGCT |
| | CTTATGCTCTTCCTGGGTGATAGAATGGGCTTAATTTGACTCAAGAATAGTACATCAAAA |
| | CTCTTAATTCCATCGAGATTGATGCATTTTTGGGGTTTTATGCTCTAAAAGGCCTTTTGC |
| | TGTGCAGAAGGAAGAGAGAGGTGGACAGAAAGGGCAGTGGGGAGAAGATGAGACTAAAAG |
| | GTGGAAAGTCCAGGTCTGGATCCCTCCCCACCACACACCTGCTCTGTGGCCTTGAGAGAA |
| | ACATGTAATTTTTATAAGCCTTAGATTTCTAATATGTAAAATGTATGTAATAGTTTTAGC |
| | TGCACTCCTTCCTTGCCAGGTTGATATAATAATCAAATAAGACTGGTCTCTGGGTCAGGC |
| | ACAGTGGCTCATGCCTGTAATCCCAGCACTTTGGGAGGCCAAGGTGGGTGGATTGTCTGA |
| | GCCCAGGAGTTTAAGACTGCAGTGAGCTGTGCACTTGGCCTGGGTGACTGAGCAAGAGCC |
| | TGGCTTTTTCTATTTATTTATTTATTTTTGAGACAGAGTCTCACTTTGTTGCCCAGGTTG |
| | AAGTGCAGTGGCAATCTCGGCTCACTGCAACCTCTGCCTCCCAGGTTCAAGCAATTCTCC |
| | TGCCTCAGCCTCCCAAGTAGCTGGGATTACAGGTGCCCACCACCACCCTAACTAATTTTT |
| | ATATTTTTAGTAGAGATGGGCTTTCACCATGTTGGCCAGGCTGGTTTTGAACTCCTGACC |
| | TCGTGATCCACCCTCCCAAAGTGCTGGACCTCCCAAAGTGCTGGGATTACAGGCATAAGC |
| | CACTGCACCTGGCCTTATTTATTTATTTATTTAGGCAGGGTCTCACTCTGTCACCCAGGC |
| | TAGAGTGTGGCAGCGCAATCATGGCTCACTGCAGCCTCGACTTCCTGGACTCAAGCGATC |
| | CTCCCACCTCTCCAACGTGGTTGAGGGCTGCAGCGAGAAAGATGTGAAAGGGGCATTTGG |
| | GGGCGCAGGAAGGCGGGAGGTGGCAATTGCTAAGGGCCCCGCCGTGTTCCTGTCACAGCC |
| | CTAGCTACAGCTTAGAAGGTCACAATGGACGTTCACCTGTTGTCTTTGATGTTTAAATCA |
| | GCACCGACTTCCCATGAGGAGGATTTTTTTTTCCTGTCTCTCTTAAAAAAAAAAAAAAAG |
| | GCCCCTGCAGTGTGGGTGTCTGCAGTGTTACGGGGATTATTAGTTGAGGATAACCACTGG |
| | GATGTGTTTCTGGAAGAAGTGGTGCTGAGGAGCACTCCTGGCCGGAGGAAAGCTGTGTGA |
| | GGAGGAGGAGGAGTATGGGGTAACTGGCTCAGTTTGCCTTGGCTCTTTCTGTCGGACTCA |
| | TACAGCAAAGCATCTGCTGAGACATCATTTGTGTCTCCTGTGACAGCCAGAGACAGCAGG |
| | TCTTGCTCTCCCCAGATCATATCTGAGATGCTGGCTCCTGCTCTCTGAGCTGAAGGCTGC |
| | AGCTGCTGAGAGAGGACGAGGCACTGAGTCAAAGCAGCTGCACAATGTTGGGGAACAGCT |
| | GGGTACTGAGGACTCTTCTCCCTGTCCTTCTACAGGTTGGTGAATTCTGGCCTTACGTCA |
| | GTCTGTTGTTCAGCTTTGTCCTCGGTACTCAGCACTAATCAGAATCTCACGCACCTTTAC |
| | CTGCGAGGCAACACTCTCGGAGACAAGGGGATCAAACTACTCTGTGAGGGACTCTTGCAC |
| | CCCGACTGCAAGCTTCAGGTGTTGGAGTAAGTCCTTTGGTTTATTACAGCAATGAGAACA |
| | CATGGTGGCCAAGGGTGGAGGGACGTTTAGGCAGAGTGGCCACAAGATAATCTGTATCTT |
| | AGAAGTGAGGTGTCTTCTTAGTGTTTGCCTTGTTACAGGATCATGGGACTGGGAGGAGTC |
| | CTTCCAGATGATATAGATGTAGGAACTTGAGTCCTTACCATCACAGTTTTCTGTACCACA |
| | AAACAAGGAGGGCAGTATGACACTTACATTTTATGGAAATGTAGGGAAAGGCACAGAATT |
| | TGGTAAATCAATGAACAAACCACTGTTACTAATTTGATCTGTGCAATGCAGTGGATTTGA |
| | AAAAGAGAGAGAAGAATAATTACCAGCATACAAGGCTGCTTGAAGCTAGTTAGTCCTGTG |
| | CTCCTGTGCTTTTTTTTTTTCCACCTGAAACAAGACAGGCTAAGATGCTAAAATCTTGGG |
| | GAGCTAGGGGGATGGTTAAGGGGACATTTTCTTTAAATCACCCCCTTTTTGCAGATTAGA |
| | CAACTGCAACCTCACGTCACACTGCTGCTGGGATCTTTCCACACTTCTGACCTCCAGCCA |
| | GAGCCTGCGAAAGCTGAGCCTGGGCAACAATGACCTGGGCGACCTGGGGGTCATGATGTT |
| | CTGTGAAGTGCTGAAACAGCAGAGCTGCCTCCTGCAGAACCTGGGGTGAGTGTGCTCTGC |
| | AGAGATGCCCGTGGTGGGACTCTGAGTTCTCAGGAAACGTTGACTGTTATCAAAATCCAG |
| | GATGGCTCTCGCTTCATTTGCAGCCACTCAAGATTAGGTTGGGCCTGGGTCAGTTGTGGT |
| | GGATATTTTAAAATAGAGAATATGGGGTTAAACTACACATTCCACTTCACTGAGATGGTT |
| | AGAATCACCAAGGCTAAGCTGGATTTCACCCACATTCCCCAAAGAAACACAAGCCCTCAT |
| | GCGATCATGGCCAAGGAGGATTCTGTTCTACACAAGGAACCGAACTTCGTAGAACTCGTC |
| | TATGGGTCATCCAAATTGGAAGGGTCTTTACATAGTGGGGATCTTGTTGTTTTGTTTCTG |
| | AGGATTTATTTTTTTTTTCCTTGAGACAGAGTCTTGCTCTGTCGCCCAGGCTGGAGTGCA |
| | GTGGCATGATCTCGGCTCACTGCAAGCTCCGCCTCCCGGGTTCACGCCATTCTCCTGCCT |
| | CGGCCTCCCAAGTAGCTGGGACCACAGGCGCCTGCCACCACACCCGGCTAATTTTTTGTA |
| | TTTTTAGTAGAGTTGGGGTTTCACTGTGTTAGCCAGGATGGTCTCCATCTCCTGACCTCG |
| | TGATCTGCCTGCCTCGGCCTCCCCAAGTGCTGGGATTACAGGTGTGAGCCTCCGTGCCTG |
| | GCGGTTTCTGAGGATTTCTAAATGGGAAAAGAGTGCTCCAGGAATAGCAAGATGGGAAAT |
| | TACACGAAATTCGACAGTGTGTCAGGATATTGCCAGCCCACCTCTGTGATACTACTGAGG |
| | TAGTCGTCTCCTCTCTAGCAAGAGCTTCCTGGCGAGTCCCACGGTTCTGTCAGCCGTGAT |
| | GATTGCACTTTTCCCTGAAGACGTTTTCACTGCTTGGCTTTAAGGCCAACATGCTCTCCT |
| | GGGTTTTCTCCTATTTCATCAGATCCTTCTACTCAGTCTCCTTTCTGGCCTCTTCTTCAT |
| | CATCCTGACCCTTATGTGTTGAATCCTCAGAGCTTAGTCGCTGGGCTAATTTTTCTTCCC |
| | TACACTTTCTCCCTCATTTAAATTATCTGGTCTTCTGGCTTCATGATATGTGTGCTGTGG |
| | CCTCCAGATTATATGTCCATAAATTCTGTGTCTCCGTGCTTGGCTGATCTTTCTTCTTGA |
| | AGGTTTAATAAACCTCTCAAACGTCACATCTAGATCCACACCCTTGCTACTCTCTTCTAC |
| | CCGAAACCTGATTCTTCCCCATTTCAGTCGATGGTCACTTCCTCCAAGTTGCTCGGGCCA |
| | AAACATTTCTGGTTCTGTCGTCACTGTCTCTTTTTCCTTATAATCATGTCCAATGTGTCA |
| | GCACATCCTGTGAGCTCTGCCTTCCCAGCTTGGCCACTCCCTGACCACTTCAGGCCAGCT |
| | TCACGGCTTCTTCCTTGGCTCAAGCCACCATCACCTGGTGTCTGGATTGTCATGGTGGCA |
| | TTCTGACAGGTCACCCTGCATCTGTTATTCTGATTTTAGCAGACAAGGGATTCAGCTAAA |
| | ACGTAACTCGCATCATATCACTCTTGTCAACATCGTCTTCTTATCACTTTCATAATCAGA |
| | GCAACAAACTCATGGTGGCTTCCATGCCCCACATAATCCAGCCTCTCCTGCTTCCGAAGA |
| | TTCCGCTTCTCCCAACCCTTGCTTATTCTGCCCCAGTACAGTGATCTCCTTGCTGTTTTG |
| | CAAGAACACTGTCATCTCTGGATTTTTGCACTTGCTTTTCTTCTGGCCTAGAACTCTCTC |
| | CCCTTCAGTATCCACCTGCATTATTCCCTGTACCTTCCTAAATTCTCTGCTTAAAGGTCT |
| | TCTCTGCGAGGCTGTCTTTGACTATTCTACTGAAAATAGAAGCACTTTTTCTTCTCTTCT |
| | CATTCTCCTCTGCAGTGTAGCACATCTTATGTGCTATGTCATTTTCTTGTTTATTTATTA |
| | TCTATCTCTGCTCCCAAAATATAAATTCCATTAGTCCAGGGACTTCTGTCCATATTAATG |
| | ATCTCAAGGCTGAGAACTTGTTGGAATGCAGGTTTGAATAAATACAAGTTTGAATCAGCC |
| | TTGCATCCTTGTGCCAACATAATGGAGCACATTTGCTGCAGGACAAGCCAATATTCCCAG |
| | AGCTAAGAGAGATGGGGGAACAAACAAAGGATTGGTCAGGTCAAAGCTGGGGACACTGCT |
| | ACATGTAGCATCACTGACCTAGCCCTGAGGGACATTCTGGACCCAGGCTGAGGGACAGGC |
| | TCAAAATAGATTGAAGAATCCATGAATGCAATCAACCCGTTTTCTCAGGCTGCGATAACA |
| | AGCTGCTTTAGACTGTGTGACTCAAACACAGATTTATTATCTCACAATACTGGAGGCTAG |
| | AAAGTCCAAAATCAAGTCTCTGATGAATTTGGTTGCTGGCGAGAGTCCTCTTCCTGATTT |
| | GCAGATGGCTACCTTCTTGTTATGTGATTGCATGGAGGGAGGGGAGGCAAGGGAGAGAAG |
| | ACAGGGGAGAAAGAACAAACTCTCTGATATCTCTCCTGTAAGGGCACTCACCCCATACTG |
| | AGGGCCCCACCCTCACAACTTCATCTAACTCTAACTACTTCCCAAGGGCCTCACCTTTAA |
| | GTACCGTACATTAGGGGTTCGGGCTTCATCATGGGAATTGTTAAGGAAGACAAACATTCA |
| | GTATATAACACCACCAAATTGAGATGGTTATAAATGGTGCAGGACAAATGGGTGGATTTA |
| | TTAGTTAAGTTGCAAGGGAAACTTTATTAAAGAGGAGGAAGAACTTACATCGAAGATGTG |
| | AGAGGCATCCTGGGCAAATGCAATGTGTAGAATTGATCAGGTTAGTGACTAAATGAACAT |
| | AAGGTAAAAAAATTTACAAGACAGTTGAGGAAGCGTAAATGCTGACTGGACATACAGTGA |
| | TACTAAGAAATTATTGTTAAACGATTTTGGTGTGATAGTGACATTGTAATTATATTTTAG |
| | TGTCACAAAGTGAATTATTGATAGATGCATTAAATGCAGTATTTAAAATAATCCATGGGT |
| | GGGAGGAGTGGGTGGGCAATAGAAGAAACAAGTTGACATGACCTGTTTAATTGTCGAACT |
| | TGGGTAATGGACACAGGGGGTTCATTATACTATTTTCTATACTTTTATGTATGCTTAACA |
| | TTTTCCCAACTAGATAGTTAAAAATATAACAGGAACCACATTGGGCACTTAGAGCTTTTT |
| | TTTTTTTTTCCCCACATTTTTATTGGGAGCCGTGGGAGGGGCCTCCTCTGTCATTGGAGG |
| | TGCTCACAGTTTCTTCAGCCACTCCAGGCTGGGGTCCTGGGGGTCTGAGGCTCCCAGTGA |
| | CGAAGGACACGACGAGCACCGGCTCCTTGGCCCAGGCATTCTTGAAGAAGGTGCGGAGTC |
| | TCGCGGCCATCTTGGTCTCGGCGGCGGCGACGGCGGCGAGGACGCGGAGCACTCTGGGAG |
| | TTGTGGTCCCTCTTTTTTTTTTTTTTTTTTTTTTACATTTTAGAAGTGTGTGGAGTTTAG |
| | GGAAATGAAGAGATGAGACTTTTAAGGTTAACGACTCTGACAGAGTTATCTGAAGAGTGC |
| | AACCCAGGCTTTCTATTTGCTTTTACAGGTTGTCTGAAATGTATTTCAATTATGAGACAA |
| | AAAGTGCGTTAGAAACACTTCAAGA |
| 2 | GTTCCTGAGGCTGGCATCTGGATGAGGAAACTGAAGTTGAGGAATAGTGAAGAGTTTGTC |
| | CAATGTCATAGCCCCGTAATCAACGGGACAAAAATTTTCTTGCTGATGGGTCAAGATGGC |
| | ATCGTGAAGTGGTTGTTCACCGTAAACTGTAATACAATCCTGTTTATGGATTTGTTTGCA |
| | TATTTTTCCCTCCATAGGGAAACCTTTCTTCCATGGCTCAGGACACACTCCTGGATCGAG |
| | CCAACAGGAGAACTTTCTGGTAAGCATTTGGCTAACTTTTTTTTTTTTGAGATGGAGTCT |
| | TGCTGTGTCGCCTAGGCTGGAGTGCAGTGGCGTGATCTTGGCTCACTGCAGCCTCCACTT |
| | CCCGGGTTCAATCAATTCTCCTACCTCAACTTCCTGAGTAGCTGGGATTACAGGCGCCCG |
| | CCACCACACCCGGCTCATTTTTGTACTTTTAGTAGAGACACAGTTTTGCCATGTTGGCCA |
| | GGCTGGTCTTGAATTCCTCAGCTCAGGTGATCTGCCTGCCTTGGCCTCTCAAAGTGCTGG |
| | GATTACAGGCGTGAGCCACTGTGCCCGGCCTTGGCTAACTTTTCAAAATTAAAGATTTTG |
| | ACTTGTTACAGTCATGTGACATTTTTTTCTTTCTGTTTGCTGAGTTTTTGATAATTTATA |
| | TCTCTCAAAGTGGAGACTTTAAAAAAGACTCATCCGTGTGCCGTGTTCACTGCCTGGTAT |
| | CTTAGTGTGGACCGAAGCCTAAGGACCCTGAAAACAGCTGCAGATGAAGATGGCAAGCAC |
| | CCGCTGCAAGCTGGCCAGGTACCTGGAGGACCTGGAGGATGTGGACTTGAAGAAATTTAA |
| | GATGCACTTAGAGGACTATCCTCCCCAGAAGGGCTGCATCCCCCTCCCGAGGGGTCAGAC |
| | AGAGAAGGCAGACCATGTGGATCTAGCCACGCTAATGATCGACTTCAATGGGGAGGAGAA |
| | GGCGTGGGCCATGGCCGTGTGGATCTTCGCTGCGATCAACAGGAGAGACCTTTATGAGAA |
| | AGCAAAAAGAGATGAGCCGAAGTGGGGTTCAGATAATGCACGTGTTTCGAATCCCACTGT |
| | GATATGCCAGGAAGACAGCATTGAAGAGGAGTGGATGGGTTTACTGGAGTACCTTTCGAG |
| | AATCTCTATTTGTAAAATGAAGAAAGATTACCGTAAGAAGTACAGAAAGTACGTGAGAAG |
| | CAGATTCCAGTGCATTGAAGACAGGAATGCCCGTCTGGGTGAGAGTGTGAGCCTCAACAA |
| | ACGCTACACACGACTGCGTCTCATCAAGGAGCACCGGAGCCAGCAGGAGAGGGAGCAGGA |
| | GCTTCTGGCCATCGGCAAGACCAAGACGTGTGAGAGCCCCGTGAGTCCCATTAAGATGGA |
| | GTTGCTGTTTGACCCCGATGATGAGCATTCTGAGCCTGTGCACACCGTGGTGTTCCAGGG |
| | GGCGGCAGGGATTGGGAAAACAATCCTGGCCAGGAAGATGATGTTGGACTGGGCGTCGGG |
| | GACACTCTACCAAGACAGGTTTGACTATCTGTTCTATATCCACTGTCGAGAGGTGAGCCT |
| | TGTGACACAGAGGAGCCTGGGGGACCTGATCATGAGCTGCTGCCCCGACCCAAACCCACC |
| | CATCCACAAGATCGTGAGAAAACCCTCCAGAATCCTCTTCCTCATGGACGGCTTCGATGA |
| | GCTGCAAGGTGCCTTTGACGAGCACATAGGACCGCTCTGCACTGACTGGCAGAAGGCCGA |
| | GCGGGGAGACATTCTCCTGAGCAGCCTCATCAGAAAGAAGCTGCTTCCCGAGGCCTCTCT |
| | GCTCATCACCACGAGACCTGTGGCCCTGGAGAAACTGCAGCACTTGCTGGACCATCCTCG |
| | GCATGTGGAGATCCTGGGTTTCTCCGAGGCCAAAAGGAAAGAGTACTTCTTCAAGTACTT |
| | CTCTGATGAGGCCCAAGCCAGGGCAGCCTTCAGTCTGATTCAGGAGAACGAGGTCCTCTT |
| | CACCATGTGCTTCATCCCCCTGGTCTGCTGGATCGTGTGCACTGGACTGAAACAGCAGAT |
| | GGAGAGTGGCAAGAGCCTTGCCCAGACATCCAAGACCACCACCGCGGTGTACGTCTTCTT |
| | CCTTTCCAGTTTGCTGCAGCCCCGGGGAGGGAGCCAGGAGCACGGCCTCTGCGCCCACCT |
| | CTGGGGGCTCTGCTCTTTGGCTGCAGATGGAATCTGGAACCAGAAAATCCTGTTTGAGGA |
| | GTCCGACCTCAGGAATCATGGACTGCAGAAGGCGGATGTGTCTGCTTTCCTGAGGATGAA |
| | CCTGTTCCAAAAGGAAGTGGACTGCGAGAAGTTCTACAGCTTCATCCACATGACTTTCCA |
| | GGAGTTCTTTGCCGCCATGTACTACCTGCTGGAAGAGGAAAAGGAAGGAAGGACGAACGT |
| | TCCAGGGAGTCGTTTGAAGCTTCCCAGCCGAGACGTGACAGTCCTTCTGGAAAACTATGG |
| | CAAATTCGAAAAGGGGTATTTGATTTTTGTTGTACGTTTCCTCTTTGGCCTGGTAAACCA |
| | GGAGAGGACCTCCTACTTGGAGAAGAAATTAAGTTGCAAGATCTCTCAGCAAATCAGGCT |
| | GGAGCTGCTGAAATGGATTGAAGTGAAAGCCAAAGCTAAAAAGCTGCAGATCCAGCCCAG |
| | CCAGCTGGAATTGTTCTACTGTTTGTACGAGATGCAGGAGGAGGACTTCGTGCAAAGGGC |
| | CATGGACTATTTCCCCAAGATTGAGATCAATCTCTCCACCAGAATGGACCACATGGTTTC |
| | TTCCTTTTGCATTGAGAACTGTCATCGGGTGGAGTCACTGTCCCTGGGGTTTCTCCATAA |
| | CATGCCCAAGGAGGAAGAGGAGGAGGAAAAGGAAGGCCGACACCTTGATATGGTGCAGTG |
| | TGTCCTCCCAAGCTCCTCTCATGCTGCCTGTTCTCATGGATTGGTGAACAGCCACCTCAC |
| | TTCCAGTTTTTGCCGGGGCCTCTTTTCAGTTCTGAGCACCAGCCAGAGTCTAACTGAATT |
| | GGACCTCAGTGACAATTCTCTGGGGGACCCAGGGATGAGAGTGTTGTGTGAAACGCTCCA |
| | GCATCCTGGCTGTAACATTCGGAGATTGTGGTTGGGGCGCTGTGGCCTCTCGCATGAGTG |
| | CTGCTTCGACATCTCCTTGGTCCTCAGCAGCAACCAGAAGCTGGTGGAGCTGGACCTGAG |
| | TGACAACGCCCTCGGTGACTTCGGAATCAGACTTCTGTGTGTGGGACTGAAGCACCTGTT |
| | GTGCAATCTGAAGAAGCTCTGGTTGGTCAGCTGCTGCCTCACATCAGCATGTTGTCAGGA |
| | TCTTGCATCAGTATTGAGCACCAGCCATTCCCTGACCAGACTCTATGTGGGGGAGAATGC |
| | CTTGGGAGACTCAGGAGTCGCAATTTTATGTGAAAAAGCCAAGAATCCACAGTGTAACCT |
| | GCAGAAACTGGGGTTGGTGAATTCTGGCCTTACGTCAGTCTGTTGTTCAGCTTTGTCCTC |
| | GGTACTCAGCACTAATCAGAATCTCACGCACCTTTACCTGCGAGGCAACACTCTCGGAGA |
| | CAAGGGGATCAAACTACTCTGTGAGGGACTCTTGCACCCCGACTGCAAGCTTCAGGTGTT |
| | GGAATTAGACAACTGCAACCTCACGTCACACTGCTGCTGGGATCTTTCCACACTTCTGAC |
| | CTCCAGCCAGAGCCTGCGAAAGCTGAGCCTGGGCAACAATGACCTGGGCGACCTGGGGGT |
| | CATGATGTTCTGTGAAGTGCTGAAACAGCAGAGCTGCCTCCTGCAGAACCTGGGGTTGTC |
| | TGAAATGTATTTCAATTATGAGACAAAAAGTGCGTTAGAAACACTTCAAGAAGAAAAGCC |
| | TGAGCTGACCGTCGTCTTTGAGCCTTCTTGGTAGGAGTGGAAACGGGGCTGCCAGACGCC |
| | AGTGTTCTCCGGTCCCTCCAGCTGGGGGCCCTCAGGTGGAGAGAGCTGCGATCCATCCAG |
| | GCCAAGACCACAGCTCTGTGATCCTTCCGGTGGAGTGTCGGAGAAGAGAGCTTGCCGACG |
| | ATGCCTTCCTGTGCAGAGCTTGGGCATCTCCTTTACGCCAGGGTGAGGAAGACACCAGGA |
| | CAATGACAGCATCGGGTGTTGTTGTCATCACAGCGCCTCAGTTAGAGGATGTTCCTCTTG |
| | GTGACCTCATGTAATTAGCTCATTCAATAAAGCACTTTCTTTATTTT |
| 3 | GTTCCTGAGGCTGGCATCTGGATGAGGAAACTGAAGTTGAGGAATAGTGAAGAGTTTGTC |
| | CAATGTCATAGCCCCGTAATCAACGGGACAAAAATTTTCTTGCTGATGGGTCAAGATGGC |
| | ATCGTGAAGTGGTTGTTCACCGTAAACTGTAATACAATCCTGTTTATGGATTTGTTTGCA |
| | TATTTTTCCCTCCATAGGGAAACCTTTCTTCCATGGCTCAGGACACACTCCTGGATCGAG |
| | CCAACAGGAGAACTTTCTGGTAAGCATTTGGCTAACTTTTTTTTTTTTGAGATGGAGTCT |
| | TGCTGTGTCGCCTAGGCTGGAGTGCAGTGGCGTGATCTTGGCTCACTGCAGCCTCCACTT |
| | CCCGGGTTCAATCAATTCTCCTACCTCAACTTCCTGAGTAGCTGGGATTACAGGCGCCCG |
| | CCACCACACCCGGCTCATTTTTGTACTTTTAGTAGAGACACAGTTTTGCCATGTTGGCCA |
| | GGCTGGTCTTGAATTCCTCAGCTCAGGTGATCTGCCTGCCTTGGCCTCTCAAAGTGCTGG |
| | GATTACAGGCGTGAGCCACTGTGCCCGGCCTTGGCTAACTTTTCAAAATTAAAGATTTTG |
| | ACTTGTTACAGTCATGTGACATTTTTTTCTTTCTGTTTGCTGAGTTTTTGATAATTTATA |
| | TCTCTCAAAGTGGAGACTTTAAAAAAGACTCATCCGTGTGCCGTGTTCACTGCCTGGTAT |
| | CTTAGTGTGGACCGAAGCCTAAGGACCCTGAAAACAGCTGCAGATGAAGATGGCAAGCAC |
| | CCGCTGCAAGCTGGCCAGGTACCTGGAGGACCTGGAGGATGTGGACTTGAAGAAATTTAA |
| | GATGCACTTAGAGGACTATCCTCCCCAGAAGGGCTGCATCCCCCTCCCGAGGGGTCAGAC |
| | AGAGAAGGCAGACCATGTGGATCTAGCCACGCTAATGATCGACTTCAATGGGGAGGAGAA |
| | GGCGTGGGCCATGGCCGTGTGGATCTTCGCTGCGATCAACAGGAGAGACCTTTATGAGAA |
| | AGCAAAAAGAGATGAGCCGAAGTGGGGTTCAGATAATGCACGTGTTTCGAATCCCACTGT |
| | GATATGCCAGGAAGACAGCATTGAAGAGGAGTGGATGGGTTTACTGGAGTACCTTTCGAG |
| | AATCTCTATTTGTAAAATGAAGAAAGATTACCGTAAGAAGTACAGAAAGTACGTGAGAAG |
| | CAGATTCCAGTGCATTGAAGACAGGAATGCCCGTCTGGGTGAGAGTGTGAGCCTCAACAA |
| | ACGCTACACACGACTGCGTCTCATCAAGGAGCACCGGAGCCAGCAGGAGAGGGAGCAGGA |
| | GCTTCTGGCCATCGGCAAGACCAAGACGTGTGAGAGCCCCGTGAGTCCCATTAAGATGGA |
| | GTTGCTGTTTGACCCCGATGATGAGCATTCTGAGCCTGTGCACACCGTGGTGTTCCAGGG |
| | GGCGGCAGGGATTGGGAAAACAATCCTGGCCAGGAAGATGATGTTGGACTGGGCGTCGGG |
| | GACACTCTACCAAGACAGGTTTGACTATCTGTTCTATATCCACTGTCGAGAGGTGAGCCT |
| | TGTGACACAGAGGAGCCTGGGGGACCTGATCATGAGCTGCTGCCCCGACCCAAACCCACC |
| | CATCCACAAGATCGTGAGAAAACCCTCCAGAATCCTCTTCCTCATGGACGGCTTCGATGA |
| | GCTGCAAGGTGCCTTTGACGAGCACATAGGACCGCTCTGCACTGACTGGCAGAAGGCCGA |
| | GCGGGGAGACATTCTCCTGAGCAGCCTCATCAGAAAGAAGCTGCTTCCCGAGGCCTCTCT |
| | GCTCATCACCACGAGACCTGTGGCCCTGGAGAAACTGCAGCACTTGCTGGACCATCCTCG |
| | GCATGTGGAGATCCTGGGTTTCTCCGAGGCCAAAAGGAAAGAGTACTTCTTCAAGTACTT |
| | CTCTGATGAGGCCCAAGCCAGGGCAGCCTTCAGTCTGATTCAGGAGAACGAGGTCCTCTT |
| | CACCATGTGCTTCATCCCCCTGGTCTGCTGGATCGTGTGCACTGGACTGAAACAGCAGAT |
| | GGAGAGTGGCAAGAGCCTTGCCCAGACATCCAAGACCACCACCGCGGTGTACGTCTTCTT |
| | CCTTTCCAGTTTGCTGCAGCCCCGGGGAGGGAGCCAGGAGCACGGCCTCTGCGCCCACCT |
| | CTGGGGGCTCTGCTCTTTGGCTGCAGATGGAATCTGGAACCAGAAAATCCTGTTTGAGGA |
| | GTCCGACCTCAGGAATCATGGACTGCAGAAGGCGGATGTGTCTGCTTTCCTGAGGATGAA |
| | CCTGTTCCAAAAGGAAGTGGACTGCGAGAAGTTCTACAGCTTCATCCACATGACTTTCCA |
| | GGAGTTCTTTGCCGCCATGTACTACCTGCTGGAAGAGGAAAAGGAAGGAAGGACGAACGT |
| | TCCAGGGAGTCGTTTGAAGCTTCCCAGCCGAGACGTGACAGTCCTTCTGGAAAACTATGG |
| | CAAATTCGAAAAGGGGTATTTGATTTTTGTTGTACGTTTCCTCTTTGGCCTGGTAAACCA |
| | GGAGAGGACCTCCTACTTGGAGAAGAAATTAAGTTGCAAGATCTCTCAGCAAATCAGGCT |
| | GGAGCTGCTGAAATGGATTGAAGTGAAAGCCAAAGCTAAAAAGCTGCAGATCCAGCCCAG |
| | CCAGCTGGAATTGTTCTACTGTTTGTACGAGATGCAGGAGGAGGACTTCGTGCAAAGGGC |
| | CATGGACTATTTCCCCAAGATTGAGATCAATCTCTCCACCAGAATGGACCACATGGTTTC |
| | TTCCTTTTGCATTGAGAACTGTCATCGGGTGGAGTCACTGTCCCTGGGGTTTCTCCATAA |
| | CATGCCCAAGGAGGAAGAGGAGGAGGAAAAGGAAGGCCGACACCTTGATATGGTGCAGTG |
| | TGTCCTCCCAAGCTCCTCTCATGCTGCCTGTTCTCATGGGTTGGGGCGCTGTGGCCTCTC |
| | GCATGAGTGCTGCTTCGACATCTCCTTGGTCCTCAGCAGCAACCAGAAGCTGGTGGAGCT |
| | GGACCTGAGTGACAACGCCCTCGGTGACTTCGGAATCAGACTTCTGTGTGTGGGACTGAA |
| | GCACCTGTTGTGCAATCTGAAGAAGCTCTGGTTGGTGAATTCTGGCCTTACGTCAGTCTG |
| | TTGTTCAGCTTTGTCCTCGGTACTCAGCACTAATCAGAATCTCACGCACCTTTACCTGCG |
| | AGGCAACACTCTCGGAGACAAGGGGATCAAACTACTCTGTGAGGGACTCTTGCACCCCGA |
| | CTGCAAGCTTCAGGTGTTGGAATTAGACAACTGCAACCTCACGTCACACTGCTGCTGGGA |
| | TCTTTCCACACTTCTGACCTCCAGCCAGAGCCTGCGAAAGCTGAGCCTGGGCAACAATGA |
| | CCTGGGCGACCTGGGGGTCATGATGTTCTGTGAAGTGCTGAAACAGCAGAGCTGCCTCCT |
| | GCAGAACCTGGGGTTGTCTGAAATGTATTTCAATTATGAGACAAAAAGTGCGTTAGAAAC |
| | ACTTCAAGAAGAAAAGCCTGAGCTGACCGTCGTCTTTGAGCCTTCTTGGTAGGAGTGGAA |
| | ACGGGGCTGCCAGACGCCAGTGTTCTCCGGTCCCTCCAGCTGGGGGCCCTCAGGTGGAGA |
| | GAGCTGCGATCCATCCAGGCCAAGACCACAGCTCTGTGATCCTTCCGGTGGAGTGTCGGA |
| | GAAGAGAGCTTGCCGACGATGCCTTCCTGTGCAGAGCTTGGGCATCTCCTTTACGCCAGG |
| | GTGAGGAAGACACCAGGACAATGACAGCATCGGGTGTTGTTGTCATCACAGCGCCTCAGT |
| | TAGAGGATGTTCCTCTTGGTGACCTCATGTAATTAGCTCATTCAATAAAGCACTTTCTTT |
| | ATTTT |
| 4 | GTTCCTGAGGCTGGCATCTGGGGAAACCTTTCTTCCATGGCTCAGGACACACTCCTGGAT |
| | CGAGCCAACAGGAGAACTTTCTGTGTGGACCGAAGCCTAAGGACCCTGAAAACAGCTGCA |
| | GATGAAGATGGCAAGCACCCGCTGCAAGCTGGCCAGGTACCTGGAGGACCTGGAGGATGT |
| | GGACTTGAAGAAATTTAAGATGCACTTAGAGGACTATCCTCCCCAGAAGGGCTGCATCCC |
| | CCTCCCGAGGGGTCAGACAGAGAAGGCAGACCATGTGGATCTAGCCACGCTAATGATCGA |
| | CTTCAATGGGGAGGAGAAGGCGTGGGCCATGGCCGTGTGGATCTTCGCTGCGATCAACAG |
| | GAGAGACCTTTATGAGAAAGCAAAAAGAGATGAGCCGAAGTGGGGTTCAGATAATGCACG |
| | TGTTTCGAATCCCACTGTGATATGCCAGGAAGACAGCATTGAAGAGGAGTGGATGGGTTT |
| | ACTGGAGTACCTTTCGAGAATCTCTATTTGTAAAATGAAGAAAGATTACCGTAAGAAGTA |
| | CAGAAAGTACGTGAGAAGCAGATTCCAGTGCATTGAAGACAGGAATGCCCGTCTGGGTGA |
| | GAGTGTGAGCCTCAACAAACGCTACACACGACTGCGTCTCATCAAGGAGCACCGGAGCCA |
| | GCAGGAGAGGGAGCAGGAGCTTCTGGCCATCGGCAAGACCAAGACGTGTGAGAGCCCCGT |
| | GAGTCCCATTAAGATGGAGTTGCTGTTTGACCCCGATGATGAGCATTCTGAGCCTGTGCA |
| | CACCGTGGTGTTCCAGGGGGCGGCAGGGATTGGGAAAACAATCCTGGCCAGGAAGATGAT |
| | GTTGGACTGGGCGTCGGGGACACTCTACCAAGACAGGTTTGACTATCTGTTCTATATCCA |
| | CTGTCGAGAGGTGAGCCTTGTGACACAGAGGAGCCTGGGGGACCTGATCATGAGCTGCTG |
| | CCCCGACCCAAACCCACCCATCCACAAGATCGTGAGAAAACCCTCCAGAATCCTCTTCCT |
| | CATGGACGGCTTCGATGAGCTGCAAGGTGCCTTTGACGAGCACATAGGACCGCTCTGCAC |
| | TGACTGGCAGAAGGCCGAGCGGGGAGACATTCTCCTGAGCAGCCTCATCAGAAAGAAGCT |
| | GCTTCCCGAGGCCTCTCTGCTCATCACCACGAGACCTGTGGCCCTGGAGAAACTGCAGCA |
| | CTTGCTGGACCATCCTCGGCATGTGGAGATCCTGGGTTTCTCCGAGGCCAAAAGGAAAGA |
| | GTACTTCTTCAAGTACTTCTCTGATGAGGCCCAAGCCAGGGCAGCCTTCAGTCTGATTCA |
| | GGAGAACGAGGTCCTCTTCACCATGTGCTTCATCCCCCTGGTCTGCTGGATCGTGTGCAC |
| | TGGACTGAAACAGCAGATGGAGAGTGGCAAGAGCCTTGCCCAGACATCCAAGACCACCAC |
| | CGCGGTGTACGTCTTCTTCCTTTCCAGTTTGCTGCAGCCCCGGGGAGGGAGCCAGGAGCA |
| | CGGCCTCTGCGCCCACCTCTGGGGGCTCTGCTCTTTGGCTGCAGATGGAATCTGGAACCA |
| | GAAAATCCTGTTTGAGGAGTCCGACCTCAGGAATCATGGACTGCAGAAGGCGGATGTGTC |
| | TGCTTTCCTGAGGATGAACCTGTTCCAAAAGGAAGTGGACTGCGAGAAGTTCTACAGCTT |
| | CATCCACATGACTTTCCAGGAGTTCTTTGCCGCCATGTACTACCTGCTGGAAGAGGAAAA |
| | GGAAGGAAGGACGAACGTTCCAGGGAGTCGTTTGAAGCTTCCCAGCCGAGACGTGACAGT |
| | CCTTCTGGAAAACTATGGCAAATTCGAAAAGGGGTATTTGATTTTTGTTGTACGTTTCCT |
| | CTTTGGCCTGGTAAACCAGGAGAGGACCTCCTACTTGGAGAAGAAATTAAGTTGCAAGAT |
| | CTCTCAGCAAATCAGGCTGGAGCTGCTGAAATGGATTGAAGTGAAAGCCAAAGCTAAAAA |
| | GCTGCAGATCCAGCCCAGCCAGCTGGAATTGTTCTACTGTTTGTACGAGATGCAGGAGGA |
| | GGACTTCGTGCAAAGGGCCATGGACTATTTCCCCAAGATTGAGATCAATCTCTCCACCAG |
| | AATGGACCACATGGTTTCTTCCTTTTGCATTGAGAACTGTCATCGGGTGGAGTCACTGTC |
| | CCTGGGGTTTCTCCATAACATGCCCAAGGAGGAAGAGGAGGAGGAAAAGGAAGGCCGACA |
| | CCTTGATATGGTGCAGTGTGTCCTCCCAAGCTCCTCTCATGCTGCCTGTTCTCATGGATT |
| | GGTGAACAGCCACCTCACTTCCAGTTTTTGCCGGGGCCTCTTTTCAGTTCTGAGCACCAG |
| | CCAGAGTCTAACTGAATTGGACCTCAGTGACAATTCTCTGGGGGACCCAGGGATGAGAGT |
| | GTTGTGTGAAACGCTCCAGCATCCTGGCTGTAACATTCGGAGATTGTGGTTGGGGCGCTG |
| | TGGCCTCTCGCATGAGTGCTGCTTCGACATCTCCTTGGTCCTCAGCAGCAACCAGAAGCT |
| | GGTGGAGCTGGACCTGAGTGACAACGCCCTCGGTGACTTCGGAATCAGACTTCTGTGTGT |
| | GGGACTGAAGCACCTGTTGTGCAATCTGAAGAAGCTCTGGTTGGTCAGCTGCTGCCTCAC |
| | ATCAGCATGTTGTCAGGATCTTGCATCAGTATTGAGCACCAGCCATTCCCTGACCAGACT |
| | CTATGTGGGGGAGAATGCCTTGGGAGACTCAGGAGTCGCAATTTTATGTGAAAAAGCCAA |
| | GAATCCACAGTGTAACCTGCAGAAACTGGGGTTGGTGAATTCTGGCCTTACGTCAGTCTG |
| | TTGTTCAGCTTTGTCCTCGGTACTCAGCACTAATCAGAATCTCACGCACCTTTACCTGCG |
| | AGGCAACACTCTCGGAGACAAGGGGATCAAACTACTCTGTGAGGGACTCTTGCACCCCGA |
| | CTGCAAGCTTCAGGTGTTGGAATTAGACAACTGCAACCTCACGTCACACTGCTGCTGGGA |
| | TCTTTCCACACTTCTGACCTCCAGCCAGAGCCTGCGAAAGCTGAGCCTGGGCAACAATGA |
| | CCTGGGCGACCTGGGGGTCATGATGTTCTGTGAAGTGCTGAAACAGCAGAGCTGCCTCCT |
| | GCAGAACCTGGGGTTGTCTGAAATGTATTTCAATTATGAGACAAAAAGTGCGTTAGAAAC |
| | ACTTCAAGAAGAAAAGCCTGAGCTGACCGTCGTCTTTGAGCCTTCTTGGTAGGAGTGGAA |
| | ACGGGGCTGCCAGACGCCAGTGTTCTCCGGTCCCTCCAGCTGGGGGCCCTCAGGTGGAGA |
| | GAGCTGCGATCCATCCAGGCCAAGACCACAGCTCTGTGATCCTTCCGGTGGAGTGTCGGA |
| | GAAGAGAGCTTGCCGACGATGCCTTCCTGTGCAGAGCTTGGGCATCTCCTTTACGCCAGG |
| | GTGAGGAAGACACCAGGACAATGACAGCATCGGGTGTTGTTGTCATCACAGCGCCTCAGT |
| | TAGAGGATGTTCCTCTTGGTGACCTCATGTAATTAGCTCATTCAATAAAGCACTTTCTTT |
| | ATTTT |
| 5 | GTTCCTGAGGCTGGCATCTGGATGAGGAAACTGAAGTTGAGGAATAGTGAAGAGTTTGTC |
| | CAATGTCATAGCCCCGTAATCAACGGGACAAAAATTTTCTTGCTGATGGGTCAAGATGGC |
| | ATCGTGAAGTGGTTGTTCACCGTAAACTGTAATACAATCCTGTTTATGGATTTGTTTGCA |
| | TATTTTTCCCTCCATAGGGAAACCTTTCTTCCATGGCTCAGGACACACTCCTGGATCGAG |
| | CCAACAGGAGAACTTTCTGGTAAGCATTTGGCTAACTTTTTTTTTTTTGAGATGGAGTCT |
| | TGCTGTGTCGCCTAGGCTGGAGTGCAGTGGCGTGATCTTGGCTCACTGCAGCCTCCACTT |
| | CCCGGGTTCAATCAATTCTCCTACCTCAACTTCCTGAGTAGCTGGGATTACAGGCGCCCG |
| | CCACCACACCCGGCTCATTTTTGTACTTTTAGTAGAGACACAGTTTTGCCATGTTGGCCA |
| | GGCTGGTCTTGAATTCCTCAGCTCAGGTGATCTGCCTGCCTTGGCCTCTCAAAGTGCTGG |
| | GATTACAGGCGTGAGCCACTGTGCCCGGCCTTGGCTAACTTTTCAAAATTAAAGATTTTG |
| | ACTTGTTACAGTCATGTGACATTTTTTTCTTTCTGTTTGCTGAGTTTTTGATAATTTATA |
| | TCTCTCAAAGTGGAGACTTTAAAAAAGACTCATCCGTGTGCCGTGTTCACTGCCTGGTAT |
| | CTTAGTGTGGACCGAAGCCTAAGGACCCTGAAAACAGCTGCAGATGAAGATGGCAAGCAC |
| | CCGCTGCAAGCTGGCCAGGTACCTGGAGGACCTGGAGGATGTGGACTTGAAGAAATTTAA |
| | GATGCACTTAGAGGACTATCCTCCCCAGAAGGGCTGCATCCCCCTCCCGAGGGGTCAGAC |
| | AGAGAAGGCAGACCATGTGGATCTAGCCACGCTAATGATCGACTTCAATGGGGAGGAGAA |
| | GGCGTGGGCCATGGCCGTGTGGATCTTCGCTGCGATCAACAGGAGAGACCTTTATGAGAA |
| | AGCAAAAAGAGATGAGCCGAAGTGGGGTTCAGATAATGCACGTGTTTCGAATCCCACTGT |
| | GATATGCCAGGAAGACAGCATTGAAGAGGAGTGGATGGGTTTACTGGAGTACCTTTCGAG |
| | AATCTCTATTTGTAAAATGAAGAAAGATTACCGTAAGAAGTACAGAAAGTACGTGAGAAG |
| | CAGATTCCAGTGCATTGAAGACAGGAATGCCCGTCTGGGTGAGAGTGTGAGCCTCAACAA |
| | ACGCTACACACGACTGCGTCTCATCAAGGAGCACCGGAGCCAGCAGGAGAGGGAGCAGGA |
| | GCTTCTGGCCATCGGCAAGACCAAGACGTGTGAGAGCCCCGTGAGTCCCATTAAGATGGA |
| | GTTGCTGTTTGACCCCGATGATGAGCATTCTGAGCCTGTGCACACCGTGGTGTTCCAGGG |
| | GGCGGCAGGGATTGGGAAAACAATCCTGGCCAGGAAGATGATGTTGGACTGGGCGTCGGG |
| | GACACTCTACCAAGACAGGTTTGACTATCTGTTCTATATCCACTGTCGAGAGGTGAGCCT |
| | TGTGACACAGAGGAGCCTGGGGGACCTGATCATGAGCTGCTGCCCCGACCCAAACCCACC |
| | CATCCACAAGATCGTGAGAAAACCCTCCAGAATCCTCTTCCTCATGGACGGCTTCGATGA |
| | GCTGCAAGGTGCCTTTGACGAGCACATAGGACCGCTCTGCACTGACTGGCAGAAGGCCGA |
| | GCGGGGAGACATTCTCCTGAGCAGCCTCATCAGAAAGAAGCTGCTTCCCGAGGCCTCTCT |
| | GCTCATCACCACGAGACCTGTGGCCCTGGAGAAACTGCAGCACTTGCTGGACCATCCTCG |
| | GCATGTGGAGATCCTGGGTTTCTCCGAGGCCAAAAGGAAAGAGTACTTCTTCAAGTACTT |
| | CTCTGATGAGGCCCAAGCCAGGGCAGCCTTCAGTCTGATTCAGGAGAACGAGGTCCTCTT |
| | CACCATGTGCTTCATCCCCCTGGTCTGCTGGATCGTGTGCACTGGACTGAAACAGCAGAT |
| | GGAGAGTGGCAAGAGCCTTGCCCAGACATCCAAGACCACCACCGCGGTGTACGTCTTCTT |
| | CCTTTCCAGTTTGCTGCAGCCCCGGGGAGGGAGCCAGGAGCACGGCCTCTGCGCCCACCT |
| | CTGGGGGCTCTGCTCTTTGGCTGCAGATGGAATCTGGAACCAGAAAATCCTGTTTGAGGA |
| | GTCCGACCTCAGGAATCATGGACTGCAGAAGGCGGATGTGTCTGCTTTCCTGAGGATGAA |
| | CCTGTTCCAAAAGGAAGTGGACTGCGAGAAGTTCTACAGCTTCATCCACATGACTTTCCA |
| | GGAGTTCTTTGCCGCCATGTACTACCTGCTGGAAGAGGAAAAGGAAGGAAGGACGAACGT |
| | TCCAGGGAGTCGTTTGAAGCTTCCCAGCCGAGACGTGACAGTCCTTCTGGAAAACTATGG |
| | CAAATTCGAAAAGGGGTATTTGATTTTTGTTGTACGTTTCCTCTTTGGCCTGGTAAACCA |
| | GGAGAGGACCTCCTACTTGGAGAAGAAATTAAGTTGCAAGATCTCTCAGCAAATCAGGCT |
| | GGAGCTGCTGAAATGGATTGAAGTGAAAGCCAAAGCTAAAAAGCTGCAGATCCAGCCCAG |
| | CCAGCTGGAATTGTTCTACTGTTTGTACGAGATGCAGGAGGAGGACTTCGTGCAAAGGGC |
| | CATGGACTATTTCCCCAAGATTGAGATCAATCTCTCCACCAGAATGGACCACATGGTTTC |
| | TTCCTTTTGCATTGAGAACTGTCATCGGGTGGAGTCACTGTCCCTGGGGTTTCTCCATAA |
| | CATGCCCAAGGAGGAAGAGGAGGAGGAAAAGGAAGGCCGACACCTTGATATGGTGCAGTG |
| | TGTCCTCCCAAGCTCCTCTCATGCTGCCTGTTCTCATGGATTGGTGAACAGCCACCTCAC |
| | TTCCAGTTTTTGCCGGGGCCTCTTTTCAGTTCTGAGCACCAGCCAGAGTCTAACTGAATT |
| | GGACCTCAGTGACAATTCTCTGGGGGACCCAGGGATGAGAGTGTTGTGTGAAACGCTCCA |
| | GCATCCTGGCTGTAACATTCGGAGATTGTGGTTGGGGCGCTGTGGCCTCTCGCATGAGTG |
| | CTGCTTCGACATCTCCTTGGTCCTCAGCAGCAACCAGAAGCTGGTGGAGCTGGACCTGAG |
| | TGACAACGCCCTCGGTGACTTCGGAATCAGACTTCTGTGTGTGGGACTGAAGCACCTGTT |
| | GTGCAATCTGAAGAAGCTCTGGTTGGTGAATTCTGGCCTTACGTCAGTCTGTTGTTCAGC |
| | TTTGTCCTCGGTACTCAGCACTAATCAGAATCTCACGCACCTTTACCTGCGAGGCAACAC |
| | TCTCGGAGACAAGGGGATCAAACTACTCTGTGAGGGACTCTTGCACCCCGACTGCAAGCT |
| | TCAGGTGTTGGAATTAGACAACTGCAACCTCACGTCACACTGCTGCTGGGATCTTTCCAC |
| | ACTTCTGACCTCCAGCCAGAGCCTGCGAAAGCTGAGCCTGGGCAACAATGACCTGGGCGA |
| | CCTGGGGGTCATGATGTTCTGTGAAGTGCTGAAACAGCAGAGCTGCCTCCTGCAGAACCT |
| | GGGGTTGTCTGAAATGTATTTCAATTATGAGACAAAAAGTGCGTTAGAAACACTTCAAGA |
| | AGAAAAGCCTGAGCTGACCGTCGTCTTTGAGCCTTCTTGGTAGGAGTGGAAACGGGGCTG |
| | CCAGACGCCAGTGTTCTCCGGTCCCTCCAGCTGGGGGCCCTCAGGTGGAGAGAGCTGCGA |
| | TCCATCCAGGCCAAGACCACAGCTCTGTGATCCTTCCGGTGGAGTGTCGGAGAAGAGAGC |
| | TTGCCGACGATGCCTTCCTGTGCAGAGCTTGGGCATCTCCTTTACGCCAGGGTGAGGAAG |
| | ACACCAGGACAATGACAGCATCGGGTGTTGTTGTCATCACAGCGCCTCAGTTAGAGGATG |
| | TTCCTCTTGGTGACCTCATGTAATTAGCTCATTCAATAAAGCACTTTCTTTATTTT |
| 6 | GTTCCTGAGGCTGGCATCTGGATGAGGAAACTGAAGTTGAGGAATAGTGAAGAGTTTGTC |
| | CAATGTCATAGCCCCGTAATCAACGGGACAAAAATTTTCTTGCTGATGGGTCAAGATGGC |
| | ATCGTGAAGTGGTTGTTCACCGTAAACTGTAATACAATCCTGTTTATGGATTTGTTTGCA |
| | TATTTTTCCCTCCATAGGGAAACCTTTCTTCCATGGCTCAGGACACACTCCTGGATCGAG |
| | CCAACAGGAGAACTTTCTGGTAAGCATTTGGCTAACTTTTTTTTTTTTGAGATGGAGTCT |
| | TGCTGTGTCGCCTAGGCTGGAGTGCAGTGGCGTGATCTTGGCTCACTGCAGCCTCCACTT |
| | CCCGGGTTCAATCAATTCTCCTACCTCAACTTCCTGAGTAGCTGGGATTACAGGCGCCCG |
| | CCACCACACCCGGCTCATTTTTGTACTTTTAGTAGAGACACAGTTTTGCCATGTTGGCCA |
| | GGCTGGTCTTGAATTCCTCAGCTCAGGTGATCTGCCTGCCTTGGCCTCTCAAAGTGCTGG |
| | GATTACAGGCGTGAGCCACTGTGCCCGGCCTTGGCTAACTTTTCAAAATTAAAGATTTTG |
| | ACTTGTTACAGTCATGTGACATTTTTTTCTTTCTGTTTGCTGAGTTTTTGATAATTTATA |
| | TCTCTCAAAGTGGAGACTTTAAAAAAGACTCATCCGTGTGCCGTGTTCACTGCCTGGTAT |
| | CTTAGTGTGGACCGAAGCCTAAGGACCCTGAAAACAGCTGCAGATGAAGATGGCAAGCAC |
| | CCGCTGCAAGCTGGCCAGGTACCTGGAGGACCTGGAGGATGTGGACTTGAAGAAATTTAA |
| | GATGCACTTAGAGGACTATCCTCCCCAGAAGGGCTGCATCCCCCTCCCGAGGGGTCAGAC |
| | AGAGAAGGCAGACCATGTGGATCTAGCCACGCTAATGATCGACTTCAATGGGGAGGAGAA |
| | GGCGTGGGCCATGGCCGTGTGGATCTTCGCTGCGATCAACAGGAGAGACCTTTATGAGAA |
| | AGCAAAAAGAGATGAGCCGAAGTGGGGTTCAGATAATGCACGTGTTTCGAATCCCACTGT |
| | GATATGCCAGGAAGACAGCATTGAAGAGGAGTGGATGGGTTTACTGGAGTACCTTTCGAG |
| | AATCTCTATTTGTAAAATGAAGAAAGATTACCGTAAGAAGTACAGAAAGTACGTGAGAAG |
| | CAGATTCCAGTGCATTGAAGACAGGAATGCCCGTCTGGGTGAGAGTGTGAGCCTCAACAA |
| | ACGCTACACACGACTGCGTCTCATCAAGGAGCACCGGAGCCAGCAGGAGAGGGAGCAGGA |
| | GCTTCTGGCCATCGGCAAGACCAAGACGTGTGAGAGCCCCGTGAGTCCCATTAAGATGGA |
| | GTTGCTGTTTGACCCCGATGATGAGCATTCTGAGCCTGTGCACACCGTGGTGTTCCAGGG |
| | GGCGGCAGGGATTGGGAAAACAATCCTGGCCAGGAAGATGATGTTGGACTGGGCGTCGGG |
| | GACACTCTACCAAGACAGGTTTGACTATCTGTTCTATATCCACTGTCGAGAGGTGAGCCT |
| | TGTGACACAGAGGAGCCTGGGGGACCTGATCATGAGCTGCTGCCCCGACCCAAACCCACC |
| | CATCCACAAGATCGTGAGAAAACCCTCCAGAATCCTCTTCCTCATGGACGGCTTCGATGA |
| | GCTGCAAGGTGCCTTTGACGAGCACATAGGACCGCTCTGCACTGACTGGCAGAAGGCCGA |
| | GCGGGGAGACATTCTCCTGAGCAGCCTCATCAGAAAGAAGCTGCTTCCCGAGGCCTCTCT |
| | GCTCATCACCACGAGACCTGTGGCCCTGGAGAAACTGCAGCACTTGCTGGACCATCCTCG |
| | GCATGTGGAGATCCTGGGTTTCTCCGAGGCCAAAAGGAAAGAGTACTTCTTCAAGTACTT |
| | CTCTGATGAGGCCCAAGCCAGGGCAGCCTTCAGTCTGATTCAGGAGAACGAGGTCCTCTT |
| | CACCATGTGCTTCATCCCCCTGGTCTGCTGGATCGTGTGCACTGGACTGAAACAGCAGAT |
| | GGAGAGTGGCAAGAGCCTTGCCCAGACATCCAAGACCACCACCGCGGTGTACGTCTTCTT |
| | CCTTTCCAGTTTGCTGCAGCCCCGGGGAGGGAGCCAGGAGCACGGCCTCTGCGCCCACCT |
| | CTGGGGGCTCTGCTCTTTGGCTGCAGATGGAATCTGGAACCAGAAAATCCTGTTTGAGGA |
| | GTCCGACCTCAGGAATCATGGACTGCAGAAGGCGGATGTGTCTGCTTTCCTGAGGATGAA |
| | CCTGTTCCAAAAGGAAGTGGACTGCGAGAAGTTCTACAGCTTCATCCACATGACTTTCCA |
| | GGAGTTCTTTGCCGCCATGTACTACCTGCTGGAAGAGGAAAAGGAAGGAAGGACGAACGT |
| | TCCAGGGAGTCGTTTGAAGCTTCCCAGCCGAGACGTGACAGTCCTTCTGGAAAACTATGG |
| | CAAATTCGAAAAGGGGTATTTGATTTTTGTTGTACGTTTCCTCTTTGGCCTGGTAAACCA |
| | GGAGAGGACCTCCTACTTGGAGAAGAAATTAAGTTGCAAGATCTCTCAGCAAATCAGGCT |
| | GGAGCTGCTGAAATGGATTGAAGTGAAAGCCAAAGCTAAAAAGCTGCAGATCCAGCCCAG |
| | CCAGCTGGAATTGTTCTACTGTTTGTACGAGATGCAGGAGGAGGACTTCGTGCAAAGGGC |
| | CATGGACTATTTCCCCAAGATTGAGATCAATCTCTCCACCAGAATGGACCACATGGTTTC |
| | TTCCTTTTGCATTGAGAACTGTCATCGGGTGGAGTCACTGTCCCTGGGGTTTCTCCATAA |
| | CATGCCCAAGGAGGAAGAGGAGGAGGAAAAGGAAGGCCGACACCTTGATATGGTGCAGTG |
| | TGTCCTCCCAAGCTCCTCTCATGCTGCCTGTTCTCATGGGTTGGGGCGCTGTGGCCTCTC |
| | GCATGAGTGCTGCTTCGACATCTCCTTGGTCCTCAGCAGCAACCAGAAGCTGGTGGAGCT |
| | GGACCTGAGTGACAACGCCCTCGGTGACTTCGGAATCAGACTTCTGTGTGTGGGACTGAA |
| | GCACCTGTTGTGCAATCTGAAGAAGCTCTGGTTGGTCAGCTGCTGCCTCACATCAGCATG |
| | TTGTCAGGATCTTGCATCAGTATTGAGCACCAGCCATTCCCTGACCAGACTCTATGTGGG |
| | GGAGAATGCCTTGGGAGACTCAGGAGTCGCAATTTTATGTGAAAAAGCCAAGAATCCACA |
| | GTGTAACCTGCAGAAACTGGGGTTGGTGAATTCTGGCCTTACGTCAGTCTGTTGTTCAGC |
| | TTTGTCCTCGGTACTCAGCACTAATCAGAATCTCACGCACCTTTACCTGCGAGGCAACAC |
| | TCTCGGAGACAAGGGGATCAAACTACTCTGTGAGGGACTCTTGCACCCCGACTGCAAGCT |
| | TCAGGTGTTGGAATTAGACAACTGCAACCTCACGTCACACTGCTGCTGGGATCTTTCCAC |
| | ACTTCTGACCTCCAGCCAGAGCCTGCGAAAGCTGAGCCTGGGCAACAATGACCTGGGCGA |
| | CCTGGGGGTCATGATGTTCTGTGAAGTGCTGAAACAGCAGAGCTGCCTCCTGCAGAACCT |
| | GGGGTTGTCTGAAATGTATTTCAATTATGAGACAAAAAGTGCGTTAGAAACACTTCAAGA |
| | AGAAAAGCCTGAGCTGACCGTCGTCTTTGAGCCTTCTTGGTAGGAGTGGAAACGGGGCTG |
| | CCAGACGCCAGTGTTCTCCGGTCCCTCCAGCTGGGGGCCCTCAGGTGGAGAGAGCTGCGA |
| | TCCATCCAGGCCAAGACCACAGCTCTGTGATCCTTCCGGTGGAGTGTCGGAGAAGAGAGC |
| | TTGCCGACGATGCCTTCCTGTGCAGAGCTTGGGCATCTCCTTTACGCCAGGGTGAGGAAG |
| | ACACCAGGACAATGACAGCATCGGGTGTTGTTGTCATCACAGCGCCTCAGTTAGAGGATG |
| | TTCCTCTTGGTGACCTCATGTAATTAGCTCATTCAATAAAGCACTTTCTTTATTTT |
| 7 | GTTCCTGAGGCTGGCATCTGGATGAGGAAACTGAAGTTGAGGAATAGTGAAGAGTTTGTC |
| | CAATGTCATAGCCCCGTAATCAACGGGACAAAAATTTTCTTGCTGATGGGTCAAGATGGC |
| | ATCGTGAAGTGGTTGTTCACCGTAAACTGTAATACAATCCTGTTTATGGATTTGTTTGCA |
| | TATTTTTCCCTCCATAGGGAAACCTTTCTTCCATGGCTCAGGACACACTCCTGGATCGAG |
| | CCAACAGGAGAACTTTCTGGTAAGCATTTGGCTAACTTTTTTTTTTTTGAGATGGAGTCT |
| | TGCTGTGTCGCCTAGGCTGGAGTGCAGTGGCGTGATCTTGGCTCACTGCAGCCTCCACTT |
| | CCCGGGTTCAATCAATTCTCCTACCTCAACTTCCTGAGTAGCTGGGATTACAGGCGCCCG |
| | CCACCACACCCGGCTCATTTTTGTACTTTTAGTAGAGACACAGTTTTGCCATGTTGGCCA |
| | GGCTGGTCTTGAATTCCTCAGCTCAGGTGATCTGCCTGCCTTGGCCTCTCAAAGTGCTGG |
| | GATTACAGGCGTGAGCCACTGTGCCCGGCCTTGGCTAACTTTTCAAAATTAAAGATTTTG |
| | ACTTGTTACAGTCATGTGACATTTTTTTCTTTCTGTTTGCTGAGTTTTTGATAATTTATA |
| | TCTCTCAAAGTGGAGACTTTAAAAAAGACTCATCCGTGTGCCGTGTTCACTGCCTGGTAT |
| | CTTAGTGTGGACCGAAGCCTAAGGACCCTGAAAACAGCTGCAGATGAAGATGGCAAGCAC |
| | CCGCTGCAAGCTGGCCAGGTACCTGGAGGACCTGGAGGATGTGGACTTGAAGAAATTTAA |
| | GATGCACTTAGAGGACTATCCTCCCCAGAAGGGCTGCATCCCCCTCCCGAGGGGTCAGAC |
| | AGAGAAGGCAGACCATGTGGATCTAGCCACGCTAATGATCGACTTCAATGGGGAGGAGAA |
| | GGCGTGGGCCATGGCCGTGTGGATCTTCGCTGCGATCAACAGGAGAGACCTTTATGAGAA |
| | AGCAAAAAGAGATGAGCCGAAGTGGGGTTCAGATAATGCACGTGTTTCGAATCCCACTGT |
| | GATATGCCAGGAAGACAGCATTGAAGAGGAGTGGATGGGTTTACTGGAGTACCTTTCGAG |
| | AATCTCTATTTGTAAAATGAAGAAAGATTACCGTAAGAAGTACAGAAAGTACGTGAGAAG |
| | CAGATTCCAGTGCATTGAAGACAGGAATGCCCGTCTGGGTGAGAGTGTGAGCCTCAACAA |
| | ACGCTACACACGACTGCGTCTCATCAAGGAGCACCGGAGCCAGCAGGAGAGGGAGCAGGA |
| | GCTTCTGGCCATCGGCAAGACCAAGACGTGTGAGAGCCCCGTGAGTCCCATTAAGATGGA |
| | GTTGCTGTTTGACCCCGATGATGAGCATTCTGAGCCTGTGCACACCGTGGTGTTCCAGGG |
| | GGCGGCAGGGATTGGGAAAACAATCCTGGCCAGGAAGATGATGTTGGACTGGGCGTCGGG |
| | GACACTCTACCAAGACAGGTTTGACTATCTGTTCTATATCCACTGTCGAGAGGTGAGCCT |
| | TGTGACACAGAGGAGCCTGGGGGACCTGATCATGAGCTGCTGCCCCGACCCAAACCCACC |
| | CATCCACAAGATCGTGAGAAAACCCTCCAGAATCCTCTTCCTCATGGACGGCTTCGATGA |
| | GCTGCAAGGTGCCTTTGACGAGCACATAGGACCGCTCTGCACTGACTGGCAGAAGGCCGA |
| | GCGGGGAGACATTCTCCTGAGCAGCCTCATCAGAAAGAAGCTGCTTCCCGAGGCCTCTCT |
| | GCTCATCACCACGAGACCTGTGGCCCTGGAGAAACTGCAGCACTTGCTGGACCATCCTCG |
| | GCATGTGGAGATCCTGGGTTTCTCCGAGGCCAAAAGGAAAGAGTACTTCTTCAAGTACTT |
| | CTCTGATGAGGCCCAAGCCAGGGCAGCCTTCAGTCTGATTCAGGAGAACGAGGTCCTCTT |
| | CACCATGTGCTTCATCCCCCTGGTCTGCTGGATCGTGTGCACTGGACTGAAACAGCAGAT |
| | GGAGAGTGGCAAGAGCCTTGCCCAGACATCCAAGACCACCACCGCGGTGTACGTCTTCTT |
| | CCTTTCCAGTTTGCTGCAGCCCCGGGGAGGGAGCCAGGAGCACGGCCTCTGCGCCCACCT |
| | CTGGGGGCTCTGCTCTTTGGCTGCAGATGGAATCTGGAACCAGAAAATCCTGTTTGAGGA |
| | GTCCGACCTCAGGAATCATGGACTGCAGAAGGCGGATGTGTCTGCTTTCCTGAGGATGAA |
| | CCTGTTCCAAAAGGAAGTGGACTGCGAGAAGTTCTACAGCTTCATCCACATGACTTTCCA |
| | GGAGTTCTTTGCCGCCATGTACTACCTGCTGGAAGAGGAAAAGGAAGGAAGGACGAACGT |
| | TCCAGGGAGTCGTTTGAAGCTTCCCAGCCGAGACGTGACAGTCCTTCTGGAAAACTATGG |
| | CAAATTCGAAAAGGGGTATTTGATTTTTGTTGTACGTTTCCTCTTTGGCCTGGTAAACCA |
| | GGAGAGGACCTCCTACTTGGAGAAGAAATTAAGTTGCAAGATCTCTCAGCAAATCAGGCT |
| | GGAGCTGCTGAAATGGATTGAAGTGAAAGCCAAAGCTAAAAAGCTGCAGATCCAGCCCAG |
| | CCAGCTGGAATTGTTCTACTGTTTGTACGAGATGCAGGAGGAGGACTTCGTGCAAAGGGC |
| | CATGGACTATTTCCCCAAGATTGAGATCAATCTCTCCACCAGAATGGACCACATGGTTTC |
| | TTCCTTTTGCATTGAGAACTGTCATCGGGTGGAGTCACTGTCCCTGGGGTTTCTCCATAA |
| | CATGCCCAAGGAGGAAGAGGAGGAGGAAAAGGAAGGCCGACACCTTGATATGGTGCAGTG |
| | TGTCCTCCCAAGCTCCTCTCATGCTGCCTGTTCTCATGGATTGGTGAACAGCCACCTCAC |
| | TTCCAGTTTTTGCCGGGGCCTCTTTTCAGTTCTGAGCACCAGCCAGAGTCTAACTGAATT |
| | GGACCTCAGTGACAATTCTCTGGGGGACCCAGGGATGAGAGTGTTGTGTGAAACGCTCCA |
| | GCATCCTGGCTGTAACATTCGGAGATTGTGGTTGGGGCGCTGTGGCCTCTCGCATGAGTG |
| | CTGCTTCGACATCTCCTTGGTCCTCAGCAGCAACCAGAAGCTGGTGGAGCTGGACCTGAG |
| | TGACAACGCCCTCGGTGACTTCGGAATCAGACTTCTGTGTGTGGGACTGAAGCACCTGTT |
| | GTGCAATCTGAAGAAGCTCTGGTTGGTCAGCTGCTGCCTCACATCAGCATGTTGTCAGGA |
| | TCTTGCATCAGTATTGAGCACCAGCCATTCCCTGACCAGACTCTATGTGGGGGAGAATGC |
| | CTTGGGAGACTCAGGAGTCGCAATTTTATGTGAAAAAGCCAAGAATCCACAGTGTAACCT |
| | GCAGAAACTGGGGTTGGTGAATTCTGGCCTTACGTCAGTCTGTTGTTCAGCTTTGTCCTC |
| | GGTACTCAGCACTAATCAGAATCTCACGCACCTTTACCTGCGAGGCAACACTCTCGGAGA |
| | CAAGGGGATCAAACTACTCTGTGAGGGACTCTTGCACCCCGACTGCAAGCTTCAGGTGTT |
| | GGAATTAGACAACTGCAACCTCACGTCACACTGCTGCTGGGATCTTTCCACACTTCTGAC |
| | CTCCAGCCAGAGCCTGCGAAAGCTGAGCCTGGGCAACAATGACCTGGGCGACCTGGGGGT |
| | CATGATGTTCTGTGAAGTGCTGAAACAGCAGAGCTGCCTCCTGCAGAACCTGGGGTTGTC |
| | TGAAATGTATTTCAATTATGAGACAAAAAGTGCGTTAGAAACACTTCAAGAAGAAAAGCC |
| | TGAGCTGACCGTCGTCTTTGAGCCTTCTTGGTAGGAGTGGAAACGGGGCTGCCAGACGCC |
| | AGTGTTCTCCGGTCCCTCCAGCTGGGGGCCCTCAGGTGGAGAGAGCTGCGATCCATCCAG |
| | GCCAAGACCACAGCTCTGTGATCCTTCCGGTGGAGTGTCGGAGAAGAGAGCTTGCCGACG |
| | ATGCCTTCCTGTGCAGAGCTTGGGCATCTCCTTTACGCCAGGGTGAGGAAGACACCAGGA |
| | CAATGACAGCATCGGGTGTTGTTGTCATCACAGCGCCTCAGTTAGAGGATGTTCCTCTTG |
| | GTGACCTCATGTAATTAGCTCATTCAATAAAGCACTTTCTTTATTTT |
| 8 | GTTGACTAGGCGCTGTTCTTGCTGGCTGGTGCCCCAGGGCCTGGAGAGGTCTGAAGAAAC |
| | CTGGGAGCCAGCAGCCCGGGGCTCCACTCTGGGTTCTGAAAGCCCATTCCCTGCTCTGCG |
| | GCTCCTCCCACCCCACCTCTTCTCAGCCTTGCAGCTCAAGGGTTGATCTCAGGAGTCCAG |
| | GACCCAGGAGAGGGAAGAATCTGAGGAACACAGAACAGTGAGCGTTGCCCACACCCCATC |
| | TCCCGTCACCACATCTCCCCTCACCCTCACCCTCCCTGCCTGGCCCTGGACCCCATCCCA |
| | GGACCTCCCTATCAGCTGACTTCTTCCAGTGTCTTGCAGGCCCCTCTGGGCTCCTCCCTC |
| | CCCTGGCTTTTCCTACCACTCCCCCTCTATCGGCGTCTATCTGTAGGTGCCCTGGGATTT |
| | ATAAAACTGGGTTCCGAATGCTGAATAAGAGACGGTAAGAGCCAAGGCAAAGGACAGCAC |
| | TGTTCTCTGCCTGCCTGATACCCTCACCACCTGGGAACATCCCCCAGACACCCTCTTAAC |
| | TCCGGGACAGAGATGGCTGGCGGAGCCTGGGGCCGCCTGGCCTGTTACTTGGAGTTCCTG |
| | AAGAAGGAGGAGCTGAAGGAGTTCCAGCTTCTGCTCGCCAATAAAGCGCACTCCAGGAGC |
| | TCTTCGGGTGAGACACCCGCTCAGCCAGAGAAGACGAGTGGCATGGAGGTGGCCTCGTAC |
| | CTGGTGGCTCAGTATGGGGAGCAGCGGGCCTGGGACCTAGCCCTCCATACCTGGGAGCAG |
| | ATGGGGCTGAGGTCACTGTGCGCCCAAGCCCAGGAAGGGGCAGGCCACTCTCCCTCATTC |
| | CCCTACAGCCCAAGTGAACCCCACCTGGGGTCTCCCAGCCAACCCACCTCCACCGCAGTG |
| | CTAATGCCCTGGATCCATGAATTGCCGGCGGGGTGCACCCAGGGCTCAGAGAGAAGGGTT |
| | TTGAGACAGCTGCCTGACACATCTGGACGCCGCTGGAGAGAAATCTCTGCCTCACTCCTC |
| | TACCAAGCTCTTCCAAGCTCCCCAGACCATGAGTCTCCAAGCCAGGAGTCACCCAACGCC |
| | CCCACATCCACAGCAGTGCTGGGGAGCTGGGGATCCCCACCTCAGCCCAGCCTAGCACCC |
| | AGAGAGCAGGAGGCTCCTGGGACCCAATGGCCTCTGGATGAAACGTCAGGAATTTACTAC |
| | ACAGAAATCAGAGAAAGAGAGAGAGAGAAATCAGAGAAAGGCAGGCCCCCATGGGCAGCG |
| | GTGGTAGGAACGCCCCCACAGGCGCACACCAGCCTACAGCCCCACCACCACCCATGGGAG |
| | CCTTCTGTGAGAGAGAGCCTCTGTTCCACATGGCCCTGGAAAAATGAGGATTTTAACCAA |
| | AAATTCACACAGCTGCTACTTCTACAAAGACCTCACCCCAGAAGCCAAGATCCCCTGGTC |
| | AAGAGAAGCTGGCCTGATTATGTGGAGGAGAATCGAGGACATTTAATTGAGATCAGAGAC |
| | TTATTTGGCCCAGGCCTGGATACCCAAGAACCTCGCATAGTCATACTGCAGGGGGCTGCT |
| | GGAATTGGGAAGTCAACACTGGCCAGGCAGGTGAAGGAAGCCTGGGGGAGAGGCCAGCTG |
| | TATGGGGACCGCTTCCAGCATGTCTTCTACTTCAGCTGCAGAGAGCTGGCCCAGTCCAAG |
| | GTGGTGAGTCTCGCTGAGCTCATCGGAAAAGATGGGACAGCCACTCCGGCTCCCATTAGA |
| | CAGATCCTGTCTAGGCCAGAGCGGCTGCTCTTCATCCTCGATGGTGTAGATGAGCCAGGA |
| | TGGGTCTTGCAGGAGCCGAGTTCTGAGCTCTGTCTGCACTGGAGCCAGCCACAGCCGGCG |
| | GATGCACTGCTGGGCAGTTTGCTGGGGAAAACTATACTTCCCGAGGCATCCTTCCTGATC |
| | ACGGCTCGGACCACAGCTCTGCAGAACCTCATTCCTTCTTTGGAGCAGGCACGTTGGGTA |
| | GAGGTCCTGGGGTTCTCTGAGTCCAGCAGGAAGGAATATTTCTACAGATATTTCACAGAT |
| | GAAAGGCAAGCAATTAGAGCCTTTAGGTTGGTCAAATCAAACAAAGAGCTCTGGGCCCTG |
| | TGTCTTGTGCCCTGGGTGTCCTGGCTGGCCTGCACTTGCCTGATGCAGCAGATGAAGCGG |
| | AAGGAAAAACTCACACTGACTTCCAAGACCACCACAACCCTCTGTCTACATTACCTTGCC |
| | CAGGCTCTCCAAGCTCAGCCATTGGGACCCCAGCTCAGAGACCTCTGCTCTCTGGCTGCT |
| | GAGGGCATCTGGCAAAAAAAGACCCTTTTCAGTCCAGATGACCTCAGGAAGCATGGGTTA |
| | GATGGGGCCATCATCTCCACCTTCTTGAAGATGGGTATTCTTCAAGAGCACCCCATCCCT |
| | CTGAGCTACAGCTTCATTCACCTCTGTTTCCAAGAGTTCTTTGCAGCAATGTCCTATGTC |
| | TTGGAGGATGAGAAGGGGAGAGGTAAACATTCTAATTGCATCATAGATTTGGAAAAGACG |
| | CTAGAAGCATATGGAATACATGGCCTGTTTGGGGCATCAACCACACGTTTCCTATTGGGC |
| | CTGTTAAGTGATGAGGGGGAGAGAGAGATGGAGAACATCTTTCACTGCCGGCTGTCTCAG |
| | GGGAGGAACCTGATGCAGTGGGTCCCGTCCCTGCAGCTGCTGCTGCAGCCACACTCTCTG |
| | GAGTCCCTCCACTGCTTGTACGAGACTCGGAACAAAACGTTCCTGACACAAGTGATGGCC |
| | CATTTCGAAGAAATGGGCATGTGTGTAGAAACAGACATGGAGCTCTTAGTGTGCACTTTC |
| | TGCATTAAATTCAGCCGCCACGTGAAGAAGCTTCAGCTGATTGAGGGCAGGCAGCACAGA |
| | TCAACATGGAGCCCCACCATGGTAGTCCTGTTCAGGTGGGTCCCAGTCACAGATGCCTAT |
| | TGGCAGATTCTCTTCTCCGTCCTCAAGGTCACCAGAAACCTGAAGGAGCTGGACCTAAGT |
| | GGAAACTCGCTGAGCCACTCTGCAGTGAAGAGTCTTTGTAAGACCCTGAGACGCCCTCGC |
| | TGCCTCCTGGAGACCCTGCGGTTGGCTGGCTGTGGCCTCACAGCTGAGGACTGCAAGGAC |
| | CTTGCCTTTGGGCTGAGAGCCAACCAGACCCTGACCGAGCTGGACCTGAGCTTCAATGTG |
| | CTCACGGATGCTGGAGCCAAACACCTTTGCCAGAGACTGAGACAGCCGAGCTGCAAGCTA |
| | CAGCGACTGCAGCTGGTCAGCTGTGGCCTCACGTCTGACTGCTGCCAGGACCTGGCCTCT |
| | GTGCTTAGTGCCAGCCCCAGCCTGAAGGAGCTAGACCTGCAGCAGAACAACCTGGATGAC |
| | GTTGGCGTGCGACTGCTCTGTGAGGGGCTCAGGCATCCTGCCTGCAAACTCATACGCCTG |
| | GGGCTGGACCAGACAACTCTGAGTGATGAGATGAGGCAGGAACTGAGGGCCCTGGAGCAG |
| | GAGAAACCTCAGCTGCTCATCTTCAGCAGACGGAAACCAAGTGTGATGACCCCTACTGAG |
| | GGCCTGGATACGGGAGAGATGAGTAATAGCACATCCTCACTCAAGCGGCAGAGACTCGGA |
| | TCAGAGAGGGCGGCTTCCCATGTTGCTCAGGCTAATCTCAAACTCCTGGACGTGAGCAAG |
| | ATCTTCCCAATTGCTGAGATTGCAGAGGAAAGCTCCCCAGAGGTAGTACCGGTGGAACTC |
| | TTGTGCGTGCCTTCTCCTGCCTCTCAAGGGGACCTGCATACGAAGCCTTTGGGGACTGAC |
| | GATGACTTCTGGGGCCCCACGGGGCCTGTGGCTACTGAGGTAGTTGACAAAGAAAAGAAC |
| | TTGTACCGAGTTCACTTCCCTGTAGCTGGCTCCTACCGCTGGCCCAACACGGGTCTCTGC |
| | TTTGTGATGAGAGAAGCGGTGACCGTTGAGATTGAATTCTGTGTGTGGGACCAGTTCCTG |
| | GGTGAGATCAACCCACAGCACAGCTGGATGGTGGCAGGGCCTCTGCTGGACATCAAGGCT |
| | GAGCCTGGAGCTGTGGAAGCTGTGCACCTCCCTCACTTTGTGGCTCTCCAAGGGGGCCAT |
| | GTGGACACATCCCTGTTCCAAATGGCCCACTTTAAAGAGGAGGGGATGCTCCTGGAGAAG |
| | CCAGCCAGGGTGGAGCTGCATCACATAGTTCTGGAAAACCCCAGCTTCTCCCCCTTGGGA |
| | GTCCTCCTGAAAATGATCCATAATGCCCTGCGCTTCATTCCCGTCACCTCTGTGGTGTTG |
| | CTTTACCACCGCGTCCATCCTGAGGAAGTCACCTTCCACCTCTACCTGATCCCAAGTGAC |
| | TGCTCCATTCGGAAGGCCATAGATGATCTAGAAATGAAATTCCAGTTTGTGCGAATCCAC |
| | AAGCCACCCCCGCTGACCCCACTTTATATGGGCTGTCGTTACACTGTGTCTGGGTCTGGT |
| | TCAGGGATGCTGGAAATACTCCCCAAGGAACTGGAGCTCTGCTATCGAAGCCCTGGAGAA |
| | GACCAGCTGTTCTCGGAGTTCTACGTTGGCCACTTGGGATCAGGGATCAGGCTGCAAGTG |
| | AAAGACAAGAAAGATGAGACTCTGGTGTGGGAGGCCTTGGTGAAACCAGGAGATCTCATG |
| | CCTGCAACTACTCTGATCCCTCCAGCCCGCATAGCCGTACCTTCACCTCTGGATGCCCCG |
| | CAGTTGCTGCACTTTGTGGACCAGTATCGAGAGCAGCTGATAGCCCGAGTGACATCGGTG |
| | GAGGTTGTCTTGGACAAACTGCATGGACAGGTGCTGAGCCAGGAGCAGTACGAGAGGGTG |
| | CTGGCTGAGAACACGAGGCCCAGCCAGATGCGGAAGCTGTTCAGCTTGAGCCAGTCCTGG |
| | GACCGGAAGTGCAAAGATGGACTCTACCAAGCCCTGAAGGAGACCCATCCTCACCTCATT |
| | ATGGAACTCTGGGAGAAGGGCAGCAAAAAGGGACTCCTGCCACTCAGCAGCTGAAGTATC |
| | AACACCAGCCCTTGACCCTTGAGTCCTGGCTTTGGCTGACCCTTCTTTGGGTCTCAGTTT |
| | CTTTCTCTGCAAACAAGTTGCCATCTGGTTTGCCTTCCAGCACTAAAGTAATGGAACTTT |
| | GATGATGCCTTTGCTGGGCATTATGTGTCCATGCCAGGGATGCCACAGGGGGCCCCAGTC |
| | CAGGTGGCCTAACAGCATCTCAGGGAATGTCCATCTGGAGCTGGCAAGACCCCTGCAGAC |
| | CTCATAGAGCCTCATCTGGTGGCCACAGCAGCCAAGCCTAGAGCCCTCCGGATCCCATCC |
| | AGGCGCAAAGAGGAATAGGAGGGACATGGAACCATTTGCCTCTGGCTGTGTCACAGGGTG |
| | AGCCCCAAAATTGGGGTTCAGCGTGGGAGGCCACGTGGATTCTTGGCTTTGTACAGGAAG |
| | ATCTACAAGAGCAAGCCAACAGAGTAAAGTGGAAGGAAGTTTATTCAGAAAATAAAGGAG |
| | TATCACAGCTCTTTTAGAATTTGTCTAGCAGGCTTTCCAGTTTTTACCAGAAAACCCCTA |
| | TAAATTAAAAATTTTTTACTTAAATTTAAGAATTAAAAAAATACAAAAAAGAAAAAATGA |
| | AAATAAAGGAATAAGAAGTTACCTACTCCA |
| 9 | GTTGACTAGGCGCTGTTCTTGCTGGCTGGTGCCCCAGGGCCTGGAGAGGTCTGAAGAAAC |
| | CTGGGAGCCAGCAGCCCGGGGCTCCACTCTGGGTTCTGAAAGCCCATTCCCTGCTCTGCG |
| | GCTCCTCCCACCCCACCTCTTCTCAGCCTTGCAGCTCAAGGGTTGATCTCAGGAGTCCAG |
| | GACCCAGGAGAGGGAAGAATCTGAGGAACACAGAACAGTGAGCGTTGCCCACACCCCATC |
| | TCCCGTCACCACATCTCCCCTCACCCTCACCCTCCCTGCCTGGCCCTGGACCCCATCCCA |
| | GGACCTCCCTATCAGCTGACTTCTTCCAGTGTCTTGCAGGCCCCTCTGGGCTCCTCCCTC |
| | CCCTGGCTTTTCCTACCACTCCCCCTCTATCGGCGTCTATCTGTAGGTGCCCTGGGATTT |
| | ATAAAACTGGGTTCCGAATGCTGAATAAGAGACGGTAAGAGCCAAGGCAAAGGACAGCAC |
| | TGTTCTCTGCCTGCCTGATACCCTCACCACCTGGGAACATCCCCCAGACACCCTCTTAAC |
| | TCCGGGACAGAGATGGCTGGCGGAGCCTGGGGCCGCCTGGCCTGTTACTTGGAGTTCCTG |
| | AAGAAGGAGGAGCTGAAGGAGTTCCAGCTTCTGCTCGCCAATAAAGCGCACTCCAGGAGC |
| | TCTTCGGGTGAGACACCCGCTCAGCCAGAGAAGACGAGTGGCATGGAGGTGGCCTCGTAC |
| | CTGGTGGCTCAGTATGGGGAGCAGCGGGCCTGGGACCTAGCCCTCCATACCTGGGAGCAG |
| | ATGGGGCTGAGGTCACTGTGCGCCCAAGCCCAGGAAGGGGCAGGCCACTCTCCCTCATTC |
| | CCCTACAGCCCAAGTGAACCCCACCTGGGGTCTCCCAGCCAACCCACCTCCACCGCAGTG |
| | CTAATGCCCTGGATCCATGAATTGCCGGCGGGGTGCACCCAGGGCTCAGAGAGAAGGGTT |
| | TTGAGACAGCTGCCTGACACATCTGGACGCCGCTGGAGAGAAATCTCTGCCTCACTCCTC |
| | TACCAAGCTCTTCCAAGCTCCCCAGACCATGAGTCTCCAAGCCAGGAGTCACCCAACGCC |
| | CCCACATCCACAGCAGTGCTGGGGAGCTGGGGATCCCCACCTCAGCCCAGCCTAGCACCC |
| | AGAGAGCAGGAGGCTCCTGGGACCCAATGGCCTCTGGATGAAACGTCAGGAATTTACTAC |
| | ACAGAAATCAGAGAAAGAGAGAGAGAGAAATCAGAGAAAGGCAGGCCCCCATGGGCAGCG |
| | GTGGTAGGAACGCCCCCACAGGCGCACACCAGCCTACAGCCCCACCACCACCCATGGGAG |
| | CCTTCTGTGAGAGAGAGCCTCTGTTCCACATGGCCCTGGAAAAATGAGGATTTTAACCAA |
| | AAATTCACACAGCTGCTACTTCTACAAAGACCTCACCCCAGAAGCCAAGATCCCCTGGTC |
| | AAGAGAAGCTGGCCTGATTATGTGGAGGAGAATCGAGGACATTTAATTGAGATCAGAGAC |
| | TTATTTGGCCCAGGCCTGGATACCCAAGAACCTCGCATAGTCATACTGCAGGGGGCTGCT |
| | GGAATTGGGAAGTCAACACTGGCCAGGCAGGTGAAGGAAGCCTGGGGGAGAGGCCAGCTG |
| | TATGGGGACCGCTTCCAGCATGTCTTCTACTTCAGCTGCAGAGAGCTGGCCCAGTCCAAG |
| | GTGGTGAGTCTCGCTGAGCTCATCGGAAAAGATGGGACAGCCACTCCGGCTCCCATTAGA |
| | CAGATCCTGTCTAGGCCAGAGCGGCTGCTCTTCATCCTCGATGGTGTAGATGAGCCAGGA |
| | TGGGTCTTGCAGGAGCCGAGTTCTGAGCTCTGTCTGCACTGGAGCCAGCCACAGCCGGCG |
| | GATGCACTGCTGGGCAGTTTGCTGGGGAAAACTATACTTCCCGAGGCATCCTTCCTGATC |
| | ACGGCTCGGACCACAGCTCTGCAGAACCTCATTCCTTCTTTGGAGCAGGCACGTTGGGTA |
| | GAGGTCCTGGGGTTCTCTGAGTCCAGCAGGAAGGAATATTTCTACAGATATTTCACAGAT |
| | GAAAGGCAAGCAATTAGAGCCTTTAGGTTGGTCAAATCAAACAAAGAGCTCTGGGCCCTG |
| | TGTCTTGTGCCCTGGGTGTCCTGGCTGGCCTGCACTTGCCTGATGCAGCAGATGAAGCGG |
| | AAGGAAAAACTCACACTGACTTCCAAGACCACCACAACCCTCTGTCTACATTACCTTGCC |
| | CAGGCTCTCCAAGCTCAGCCATTGGGACCCCAGCTCAGAGACCTCTGCTCTCTGGCTGCT |
| | GAGGGCATCTGGCAAAAAAAGACCCTTTTCAGTCCAGATGACCTCAGGAAGCATGGGTTA |
| | GATGGGGCCATCATCTCCACCTTCTTGAAGATGGGTATTCTTCAAGAGCACCCCATCCCT |
| | CTGAGCTACAGCTTCATTCACCTCTGTTTCCAAGAGTTCTTTGCAGCAATGTCCTATGTC |
| | TTGGAGGATGAGAAGGGGAGAGGTAAACATTCTAATTGCATCATAGATTTGGAAAAGACG |
| | CTAGAAGCATATGGAATACATGGCCTGTTTGGGGCATCAACCACACGTTTCCTATTGGGC |
| | CTGTTAAGTGATGAGGGGGAGAGAGAGATGGAGAACATCTTTCACTGCCGGCTGTCTCAG |
| | GGGAGGAACCTGATGCAGTGGGTCCCGTCCCTGCAGCTGCTGCTGCAGCCACACTCTCTG |
| | GAGTCCCTCCACTGCTTGTACGAGACTCGGAACAAAACGTTCCTGACACAAGTGATGGCC |
| | CATTTCGAAGAAATGGGCATGTGTGTAGAAACAGACATGGAGCTCTTAGTGTGCACTTTC |
| | TGCATTAAATTCAGCCGCCACGTGAAGAAGCTTCAGCTGATTGAGGGCAGGCAGCACAGA |
| | TCAACATGGAGCCCCACCATGGTAGTCCTGTTCAGGTGGGTCCCAGTCACAGATGCCTAT |
| | TGGCAGATTCTCTTCTCCGTCCTCAAGGTCACCAGAAACCTGAAGGAGCTGGACCTAAGT |
| | GGAAACTCGCTGAGCCACTCTGCAGTGAAGAGTCTTTGTAAGACCCTGAGACGCCCTCGC |
| | TGCCTCCTGGAGACCCTGCGGTTGGCTGGCTGTGGCCTCACAGCTGAGGACTGCAAGGAC |
| | CTTGCCTTTGGGCTGAGAGCCAACCAGACCCTGACCGAGCTGGACCTGAGCTTCAATGTG |
| | CTCACGGATGCTGGAGCCAAACACCTTTGCCAGAGACTGAGACAGCCGAGCTGCAAGCTA |
| | CAGCGACTGCAGCTGGTCAGCTGTGGCCTCACGTCTGACTGCTGCCAGGACCTGGCCTCT |
| | GTGCTTAGTGCCAGCCCCAGCCTGAAGGAGCTAGACCTGCAGCAGAACAACCTGGATGAC |
| | GTTGGCGTGCGACTGCTCTGTGAGGGGCTCAGGCATCCTGCCTGCAAACTCATACGCCTG |
| | GGGCTGGACCAGACAACTCTGAGTGATGAGATGAGGCAGGAACTGAGGGCCCTGGAGCAG |
| | GAGAAACCTCAGCTGCTCATCTTCAGCAGACGGAAACCAAGTGTGATGACCCCTACTGAG |
| | GGCCTGGATACGGGAGAGATGAGTAATAGCACATCCTCACTCAAGCGGCAGAGACTCGGA |
| | TCAGAGAGGGCGGCTTCCCATGTTGCTCAGGCTAATCTCAAACTCCTGGACGTGAGCAAG |
| | ATCTTCCCAATTGCTGAGATTGCAGAGGAAAGCTCCCCAGAGGTAGTACCGGTGGAACTC |
| | TTGTGCGTGCCTTCTCCTGCCTCTCAAGGGGACCTGCATACGAAGCCTTTGGGGACTGAC |
| | GATGACTTCTGGGGCCCCACGGGGCCTGTGGCTACTGAGGTAGTTGACAAAGAAAAGAAC |
| | TTGTACCGAGTTCACTTCCCTGTAGCTGGCTCCTACCGCTGGCCCAACACGGGTCTCTGC |
| | TTTGTGATGAGAGAAGCGGTGACCGTTGAGATTGAATTCTGTGTGTGGGACCAGTTCCTG |
| | GGTGAGATCAACCCACAGCACAGCTGGATGGTGGCAGGGCCTCTGCTGGACATCAAGGCT |
| | GAGCCTGGAGCTGTGGAAGCTGTGCACCTCCCTCACTTTGTGGCTCTCCAAGGGGGCCAT |
| | GTGGACACATCCCTGTTCCAAATGGCCCACTTTAAAGAGGAGGGGATGCTCCTGGAGAAG |
| | CCAGCCAGGGTGGAGCTGCATCACATAGTTCTGGAAAACCCCAGCTTCTCCCCCTTGGGA |
| | GTCCTCCTGAAAATGATCCATAATGCCCTGCGCTTCATTCCCGTCACCTCTGTGGTGTTG |
| | CTTTACCACCGCGTCCATCCTGAGGAAGTCACCTTCCACCTCTACCTGATCCCAAGTGAC |
| | TGCTCCATTCGGAAGGCCATAGATGATCTAGAAATGAAATTCCAGTTTGTGCGAATCCAC |
| | AAGCCACCCCCGCTGACCCCACTTTATATGGGCTGTCGTTACACTGTGTCTGGGTCTGGT |
| | TCAGGGATGCTGGAAATACTCCCCAAGGAACTGGAGCTCTGCTATCGAAGCCCTGGAGAA |
| | GACCAGCTGTTCTCGGAGTTCTACGTTGGCCACTTGGGATCAGGGATCAGGCTGCAAGTG |
| | AAAGACAAGAAAGATGAGACTCTGGTGTGGGAGGCCTTGGTGAAACCAGGAGATCTCATG |
| | CCTGCAACTACTCTGATCCCTCCAGCCCGCATAGCCGTACCTTCACCTCTGGATGCCCCG |
| | CAGTTGCTGCACTTTGTGGACCAGTATCGAGAGCAGCTGATAGCCCGAGTGACATCGGTG |
| | GAGGTTGTCTTGGACAAACTGCATGGACAGGTGCTGAGCCAGGAGCAGTACGAGAGGGTG |
| | CTGGCTGAGAACACGAGGCCCAGCCAGATGCGGAAGCTGTTCAGCTTGAGCCAGTCCTGG |
| | GACCGGAAGTGCAAAGATGGACTCTACCAAGCCCTGAAGGAGACCCATCCTCACCTCATT |
| | ATGGAACTCTGGGAGAAGGGCAGCAAAAAGGGACTCCTGCCACTCAGCAGCTGAAGTATC |
| | AACACCAGCCCTTGACCCTTGAGTCCTGGCTTTGGCTGACCCTTCTTTGGGTCTCAGTTT |
| | CTTTCTCTGCAAACAAGTTGCCATCTGGTTTGCCTTCCAGCACTAAAGTAATGGAACTTT |
| | GATGATGCCTTTGCTGGGCATTATGTGTCCATGCCAGGGATGCCACAGGGGGCCCCAGTC |
| | CAGGTGGCCTAACAGCATCTCAGGGAATGTCCATCTGGAGCTGGCAAGACCCCTGCAGAC |
| | CTCATAGAGCCTCATCTGGTGGCCACAGCAGCCAAGCCTAGAGCCCTCCGGATCCCATCC |
| | AGGCGCAAAGAGGAATAGGAGGGACATGGAACCATTTGCCTCTGGCTGTGTCACAGGGTG |
| | AGCCCCAAAATTGGGGTTCAGCGTGGGAGGCCACGTGGATTCTTGGCTTTGTACAGGAAG |
| | ATCTACAAGAGCAAGCCAACAGAGTAAAGTGGAAGGAAGTTTATTCAGAAAATAAAGGAG |
| | TATCACAGCTCTTTTAGAATTTGTCTAGCAGGCTTTCCAGTTTTTACCAGAAAACCCCTA |
| | TAAATTAAAAATTTTTTACTTAAATTTAAGAATTAAAAAAATACAAAAAAGAAAAAATGA |
| | AAATAAAGGAATAAGAAGTTACCTACTCCA |
| 10 | GTTGACTAGGCGCTGTTCTTGCTGGCTGGTGCCCCAGGGCCTGGAGAGGTCTGAAGAAAC |
| | CTGGGAGCCAGCAGCCCGGGGCTCCACTCTGGGTTCTGAAAGCCCATTCCCTGCTCTGCG |
| | GCTCCTCCCACCCCACCTCTTCTCAGCCTTGCAGCTCAAGGGTTGATCTCAGGAGTCCAG |
| | GACCCAGGAGAGGGAAGAATCTGAGGAACACAGAACAGTGAGCGTTGCCCACACCCCATC |
| | TCCCGTCACCACATCTCCCCTCACCCTCACCCTCCCTGCCTGGCCCTGGACCCCATCCCA |
| | GGACCTCCCTATCAGCTGACTTCTTCCAGTGTCTTGCAGGCCCCTCTGGGCTCCTCCCTC |
| | CCCTGGCTTTTCCTACCACTCCCCCTCTATCGGCGTCTATCTGTAGGTGCCCTGGGATTT |
| | ATAAAACTGGGTTCCGAATGCTGAATAAGAGACGGTAAGAGCCAAGGCAAAGGACAGCAC |
| | TGTTCTCTGCCTGCCTGATACCCTCACCACCTGGGAACATCCCCCAGACACCCTCTTAAC |
| | TCCGGGACAGAGATGGCTGGCGGAGCCTGGGGCCGCCTGGCCTGTTACTTGGAGTTCCTG |
| | AAGAAGGAGGAGCTGAAGGAGTTCCAGCTTCTGCTCGCCAATAAAGCGCACTCCAGGAGC |
| | TCTTCGGGTGAGACACCCGCTCAGCCAGAGAAGACGAGTGGCATGGAGGTGGCCTCGTAC |
| | CTGGTGGCTCAGTATGGGGAGCAGCGGGCCTGGGACCTAGCCCTCCATACCTGGGAGCAG |
| | ATGGGGCTGAGGTCACTGTGCGCCCAAGCCCAGGAAGGGGCAGGCCACTCTCCCTCATTC |
| | CCCTACAGCCCAAGTGAACCCCACCTGGGGTCTCCCAGCCAACCCACCTCCACCGCAGTG |
| | CTAATGCCCTGGATCCATGAATTGCCGGCGGGGTGCACCCAGGGCTCAGAGAGAAGGGTT |
| | TTGAGACAGCTGCCTGACACATCTGGACGCCGCTGGAGAGAAATCTCTGCCTCACTCCTC |
| | TACCAAGCTCTTCCAAGCTCCCCAGACCATGAGTCTCCAAGCCAGGAGTCACCCAACGCC |
| | CCCACATCCACAGCAGTGCTGGGGAGCTGGGGATCCCCACCTCAGCCCAGCCTAGCACCC |
| | AGAGAGCAGGAGGCTCCTGGGACCCAATGGCCTCTGGATGAAACGTCAGGAATTTACTAC |
| | ACAGAAATCAGAGAAAGAGAGAGAGAGAAATCAGAGAAAGGCAGGCCCCCATGGGCAGCG |
| | GTGGTAGGAACGCCCCCACAGGCGCACACCAGCCTACAGCCCCACCACCACCCATGGGAG |
| | CCTTCTGTGAGAGAGAGCCTCTGTTCCACATGGCCCTGGAAAAATGAGGATTTTAACCAA |
| | AAATTCACACAGCTGCTACTTCTACAAAGACCTCACCCCAGAAGCCAAGATCCCCTGGTC |
| | AAGAGAAGCTGGCCTGATTATGTGGAGGAGAATCGAGGACATTTAATTGAGATCAGAGAC |
| | TTATTTGGCCCAGGCCTGGATACCCAAGAACCTCGCATAGTCATACTGCAGGGGGCTGCT |
| | GGAATTGGGAAGTCAACACTGGCCAGGCAGGTGAAGGAAGCCTGGGGGAGAGGCCAGCTG |
| | TATGGGGACCGCTTCCAGCATGTCTTCTACTTCAGCTGCAGAGAGCTGGCCCAGTCCAAG |
| | GTGGTGAGTCTCGCTGAGCTCATCGGAAAAGATGGGACAGCCACTCCGGCTCCCATTAGA |
| | CAGATCCTGTCTAGGCCAGAGCGGCTGCTCTTCATCCTCGATGGTGTAGATGAGCCAGGA |
| | TGGGTCTTGCAGGAGCCGAGTTCTGAGCTCTGTCTGCACTGGAGCCAGCCACAGCCGGCG |
| | GATGCACTGCTGGGCAGTTTGCTGGGGAAAACTATACTTCCCGAGGCATCCTTCCTGATC |
| | ACGGCTCGGACCACAGCTCTGCAGAACCTCATTCCTTCTTTGGAGCAGGCACGTTGGGTA |
| | GAGGTCCTGGGGTTCTCTGAGTCCAGCAGGAAGGAATATTTCTACAGATATTTCACAGAT |
| | GAAAGGCAAGCAATTAGAGCCTTTAGGTTGGTCAAATCAAACAAAGAGCTCTGGGCCCTG |
| | TGTCTTGTGCCCTGGGTGTCCTGGCTGGCCTGCACTTGCCTGATGCAGCAGATGAAGCGG |
| | AAGGAAAAACTCACACTGACTTCCAAGACCACCACAACCCTCTGTCTACATTACCTTGCC |
| | CAGGCTCTCCAAGCTCAGCCATTGGGACCCCAGCTCAGAGACCTCTGCTCTCTGGCTGCT |
| | GAGGGCATCTGGCAAAAAAAGACCCTTTTCAGTCCAGATGACCTCAGGAAGCATGGGTTA |
| | GATGGGGCCATCATCTCCACCTTCTTGAAGATGGGTATTCTTCAAGAGCACCCCATCCCT |
| | CTGAGCTACAGCTTCATTCACCTCTGTTTCCAAGAGTTCTTTGCAGCAATGTCCTATGTC |
| | TTGGAGGATGAGAAGGGGAGAGGTAAACATTCTAATTGCATCATAGATTTGGAAAAGACG |
| | CTAGAAGCATATGGAATACATGGCCTGTTTGGGGCATCAACCACACGTTTCCTATTGGGC |
| | CTGTTAAGTGATGAGGGGGAGAGAGAGATGGAGAACATCTTTCACTGCCGGCTGTCTCAG |
| | GGGAGGAACCTGATGCAGTGGGTCCCGTCCCTGCAGCTGCTGCTGCAGCCACACTCTCTG |
| | GAGTCCCTCCACTGCTTGTACGAGACTCGGAACAAAACGTTCCTGACACAAGTGATGGCC |
| | CATTTCGAAGAAATGGGCATGTGTGTAGAAACAGACATGGAGCTCTTAGTGTGCACTTTC |
| | TGCATTAAATTCAGCCGCCACGTGAAGAAGCTTCAGCTGATTGAGGGCAGGCAGCACAGA |
| | TCAACATGGAGCCCCACCATGGTAGTCCTGTTCAGGTGGGTCCCAGTCACAGATGCCTAT |
| | TGGCAGATTCTCTTCTCCGTCCTCAAGGTCACCAGAAACCTGAAGGAGCTGGACCTAAGT |
| | GGAAACTCGCTGAGCCACTCTGCAGTGAAGAGTCTTTGTAAGACCCTGAGACGCCCTCGC |
| | TGCCTCCTGGAGACCCTGCGGTTGGCTGGCTGTGGCCTCACAGCTGAGGACTGCAAGGAC |
| | CTTGCCTTTGGGCTGAGAGCCAACCAGACCCTGACCGAGCTGGACCTGAGCTTCAATGTG |
| | CTCACGGATGCTGGAGCCAAACACCTTTGCCAGAGACTGAGACAGCCGAGCTGCAAGCTA |
| | CAGCGACTGCAGCTGGTCAGCTGTGGCCTCACGTCTGACTGCTGCCAGGACCTGGCCTCT |
| | GTGCTTAGTGCCAGCCCCAGCCTGAAGGAGCTAGACCTGCAGCAGAACAACCTGGATGAC |
| | GTTGGCGTGCGACTGCTCTGTGAGGGGCTCAGGCATCCTGCCTGCAAACTCATACGCCTG |
| | GGGCTGGACCAGACAACTCTGAGTGATGAGATGAGGCAGGAACTGAGGGCCCTGGAGCAG |
| | GAGAAACCTCAGCTGCTCATCTTCAGCAGACGGAAACCAAGTGTGATGACCCCTACTGAG |
| | GGCCTGGATACGGGAGAGATGAGTAATAGCACATCCTCACTCAAGCGGCAGAGACTCGGA |
| | TCAGAGAGGGCGGCTTCCCATGTTGCTCAGGCTAATCTCAAACTCCTGGACGTGAGCAAG |
| | ATCTTCCCAATTGCTGAGATTGCAGAGGAAAGCTCCCCAGAGGTAGTACCGGTGGAACTC |
| | TTGTGCGTGCCTTCTCCTGCCTCTCAAGGGGACCTGCATACGAAGCCTTTGGGGACTGAC |
| | GATGACTTCTGGGGCCCCACGGGGCCTGTGGCTACTGAGGTAGTTGACAAAGAAAAGAAC |
| | TTGTACCGAGTTCACTTCCCTGTAGCTGGCTCCTACCGCTGGCCCAACACGGGTCTCTGC |
| | TTTGTGATGAGAGAAGCGGTGACCGTTGAGATTGAATTCTGTGTGTGGGACCAGTTCCTG |
| | GGTGAGATCAACCCACAGCACAGCTGGATGGTGGCAGGGCCTCTGCTGGACATCAAGGCT |
| | GAGCCTGGAGCTGTGGAAGCTGTGCACCTCCCTCACTTTGTGGCTCTCCAAGGGGGCCAT |
| | GTGGACACATCCCTGTTCCAAATGGCCCACTTTAAAGAGGAGGGGATGCTCCTGGAGAAG |
| | CCAGCCAGGGTGGAGCTGCATCACATAGTTCTGGAAAACCCCAGCTTCTCCCCCTTGGGA |
| | GTCCTCCTGAAAATGATCCATAATGCCCTGCGCTTCATTCCCGTCACCTCTGTGGTGTTG |
| | CTTTACCACCGCGTCCATCCTGAGGAAGTCACCTTCCACCTCTACCTGATCCCAAGTGAC |
| | TGCTCCATTCGGAAGGAACTGGAGCTCTGCTATCGAAGCCCTGGAGAAGACCAGCTGTTC |
| | TCGGAGTTCTACGTTGGCCACTTGGGATCAGGGATCAGGCTGCAAGTGAAAGACAAGAAA |
| | GATGAGACTCTGGTGTGGGAGGCCTTGGTGAAACCAGGAGATCTCATGCCTGCAACTACT |
| | CTGATCCCTCCAGCCCGCATAGCCGTACCTTCACCTCTGGATGCCCCGCAGTTGCTGCAC |
| | TTTGTGGACCAGTATCGAGAGCAGCTGATAGCCCGAGTGACATCGGTGGAGGTTGTCTTG |
| | GACAAACTGCATGGACAGGTGCTGAGCCAGGAGCAGTACGAGAGGGTGCTGGCTGAGAAC |
| | ACGAGGCCCAGCCAGATGCGGAAGCTGTTCAGCTTGAGCCAGTCCTGGGACCGGAAGTGC |
| | AAAGATGGACTCTACCAAGCCCTGAAGGAGACCCATCCTCACCTCATTATGGAACTCTGG |
| | GAGAAGGGCAGCAAAAAGGGACTCCTGCCACTCAGCAGCTGAAGTATCAACACCAGCCCT |
| | TGACCCTTGAGTCCTGGCTTTGGCTGACCCTTCTTTGGGTCTCAGTTTCTTTCTCTGCAA |
| | ACAAGTTGCCATCTGGTTTGCCTTCCAGCACTAAAGTAATGGAACTTTGATGATGCCTTT |
| | GCTGGGCATTATGTGTCCATGCCAGGGATGCCACAGGGGGCCCCAGTCCAGGTGGCCTAA |
| | CAGCATCTCAGGGAATGTCCATCTGGAGCTGGCAAGACCCCTGCAGACCTCATAGAGCCT |
| | CATCTGGTGGCCACAGCAGCCAAGCCTAGAGCCCTCCGGATCCCATCCAGGCGCAAAGAG |
| | GAATAGGAGGGACATGGAACCATTTGCCTCTGGCTGTGTCACAGGGTGAGCCCCAAAATT |
| | GGGGTTCAGCGTGGGAGGCCACGTGGATTCTTGGCTTTGTACAGGAAGATCTACAAGAGC |
| | AAGCCAACAGAGTAAAGTGGAAGGAAGTTTATTCAGAAAATAAAGGAGTATCACAGCTCT |
| | TTTAGAATTTGTCTAGCAGGCTTTCCAGTTTTTACCAGAAAACCCCTATAAATTAAAAAT |
| | TTTTTACTTAAATTTAAGAATTAAAAAAATACAAAAAAGAAAAAATGAAAATAAAGGAAT |
| | AAGAAGTTACCTACTCCA |
| 11 | GCAGCACAGATCAACATGGAGCCCCACCATGGTAGTCCTGTTCAGGTGGGTCCCAGTCAC |
| | AGATGCCTATTGGCAGATTCTCTTCTCCGTCCTCAAGGTCACCAGAAACCTGAAGGAGCT |
| | GGACCTAAGTGGAAACTCGCTGAGCCACTCTGCAGTGAAGAGTCTTTGTAAGACCCTGAG |
| | ACGCCCTCGCTGCCTCCTGGAGACCCTGCGGTTGGCTGGCTGTGGCCTCACAGCTGAGGA |
| | CTGCAAGGACCTTGCCTTTGGGCTGAGAGCCAACCAGACCCTGACCGAGCTGGACCTGAG |
| | CTTCAATGTGCTCACGGATGCTGGAGCCAAACACCTTTGCCAGAGACTGAGACAGCCGAG |
| | CTGCAAGCTACAGCGACTGCAGCTGGTCAGCTGTGGCCTCACGTCTGACTGCTGCCAGGA |
| | CCTGGCCTCTGTGCTTAGTGCCAGCCCCAGCCTGAAGGAGCTAGACCTGCAGCAGAACAA |
| | CCTGGATGACGTTGGCGTGCGACTGCTCTGTGAGGGGCTCAGGCATCCTGCCTGCAAACT |
| | CATACGCCTGGGGAAACCAAGTGTGATGACCCCTACTGAGGGCCTGGATACGGGAGAGAT |
| | GAGTAATAGCACATCCTCACTCAAGCGGCAGAGACTCGGATCAGAGAGGGCGGCTTCCCA |
| | TGTTGCTCAGGCTAATCTCAAACTCCTGGACGTGAGCAAGATCTTCCCAATTGCTGAGAT |
| | TGCAGAGGAAAGCTCCCCAGAGGTAGTACCGGTGGAACTCTTGTGCGTGCCTTCTCCTGC |
| | CTCTCAAGGGGACCTGCATACGAAGCCTTTGGGGACTGACGATGACTTCTGGGGCCCCAC |
| | GGGGCCTGTGGCTACTGAGGTAGTTGACAAAGAAAAGAACTTGTACCGAGTTCACTTCCC |
| | TGTAGCTGGCTCCTACCGCTGGCCCAACACGGGTCTCTGCTTTGTGATGAGAGAAGCGGT |
| | GACCGTTGAGATTGAATTCTGTGTGTGGGACCAGTTCCTGGGTGAGATCAACCCACAGCA |
| | CAGCTGGATGGTGGCAGGGCCTCTGCTGGACATCAAGGCTGAGCCTGGAGCTGTGGAAGC |
| | TGTGCACCTCCCTCACTTTGTGGCTCTCCAAGGGGGCCATGTGGACACATCCCTGTTCCA |
| | AATGGCCCACTTTAAAGAGGAGGGGATGCTCCTGGAGAAGCCAGCCAGGGTGGAGCTGCA |
| | TCACATAGTTCTGGAAAACCCCAGCTTCTCCCCCTTGGGAGTCCTCCTGAAAATGATCCA |
| | TAATGCCCTGCGCTTCATTCCCGTCACCTCTGTGGTGTTGCTTTACCACCGCGTCCATCC |
| | TGAGGAAGTCACCTTCCACCTCTACCTGATCCCAAGTGACTGCTCCATTCGGAAGGCCAT |
| | AGATGATCTAGAAATGAAATTCCAGTTTGTGCGAATCCACAAGCCACCCCCGCTGACCCC |
| | ACTTTATATGGGCTGTCGTTACACTGTGTCTGGGTCTGGTTCAGGGATGCTGGAAATACT |
| | CCCCAAGGAACTGGAGCTCTGCTATCGAAGCCCTGGAGAAGACCAGCTGTTCTCGGAGTT |
| | CTACGTTGGCCACTTGGGATCAGGGATCAGGCTGCAAGTGAAAGACAAGAAAGATGAGAC |
| | TCTGGTGTGGGAGGCCTTGGTGAAACCAGGAGATCTCATGCCTGCAACTACTCTGATCCC |
| | TCCAGCCCGCATAGCCGTACCTTCACCTCTGGATGCCCCGCAGTTGCTGCACTTTGTGGA |
| | CCAGTATCGAGAGCAGCTGATAGCCCGAGTGACATCGGTGGAGGTTGTCTTGGACAAACT |
| | GCATGGACAGGTGCTGAGCCAGGAGCAGTACGAGAGGGTGCTGGCTGAGAACACGAGGCC |
| | CAGCCAGATGCGGAAGCTGTTCAGCTTGAGCCAGTCCTGGGACCGGAAGTGCAAAGATGG |
| | ACTCTACCAAGCCCTGAAGGAGACCCATCCTCACCTCATTATGGAACTCTGGGAGAAGGG |
| | CAGCAAAAAGGGACTCCTGCCACTCAGCAGCTGAAGTATCAACACCAGCCCTTGACCCTT |
| | GAGTCCTGGCTTTGGCTGACCCTTCTTTGGGTCTCAGTTTCTTTCTCTGCAAACAAGTTG |
| | CCATCTGGTTTGCCTTCCAGCACTAAAGTAATGGAACTTTGATGATGCCTTTGCTGGGCA |
| | TTATGTGTCCATGCCAGGGATGCCACAGGGGGCCCCAGTCCAGGTGGCCTAACAGCATCT |
| | CAGGGAATGTCCATCTGGAGCTGGCAAGACCCCTGCAGACCTCATAGAGCCTCATCTGGT |
| | GGCCACAGCAGCCAAGCCTAGAGCCCTCCGGATCCCATCCAGGCGCAAAGAGGAATAGGA |
| | GGGACATGGAACCATTTGCCTCTGGCTGTGTCACAGGGTGAGCCCCAAAATTGGGGTTCA |
| | GCGTGGGAGGCCACGTGGATTCTTGGCTTTGTACAGGAAGATCTACAAGAGCAAGCCAAC |
| | AGAGTAAAGTGGAAGGAAGTTTATTCAGAAAATAAAGGAGTATCACAGCTCTTTTAGAAT |
| | TTGTCTAGCAGGCTTTCCAGTTTTTACCAGAAAACCCCTATAAATTAAAAATTTTTTACT |
| | TAAATTTAAGAATTAAAAAAATACAAAAAAGAAAAAATGAAAATAAAGGAATAAGAAGTT |
| | ACCTACTCCA |
| 12 | GTTGACTAGGCGCTGTTCTTGCTGGCTGGTGCCCCAGGGCCTGGAGAGGTCTGAAGAAAC |
| | CTGGGAGCCAGCAGCCCGGGGCTCCACTCTGGGTTCTGAAAGCCCATTCCCTGCTCTGCG |
| | GCTCCTCCCACCCCACCTCTTCTCAGCCTTGCAGCTCAAGGGTTGATCTCAGGAGTCCAG |
| | GACCCAGGAGAGGGAAGAATCTGAGGAACACAGAACAGTGAGCGTTGCCCACACCCCATC |
| | TCCCGTCACCACATCTCCCCTCACCCTCACCCTCCCTGCCTGGCCCTGGACCCCATCCCA |
| | GGACCTCCCTATCAGCTGACTTCTTCCAGTGTCTTGCAGGCCCCTCTGGGCTCCTCCCTC |
| | CCCTGGCTTTTCCTACCACTCCCCCTCTATCGGCGTCTATCTGTAGGTGCCCTGGGATTT |
| | ATAAAACTGGGTTCCGAATGCTGAATAAGAGACGGTAAGAGCCAAGGCAAAGGACAGCAC |
| | TGTTCTCTGCCTGCCTGATACCCTCACCACCTGGGAACATCCCCCAGACACCCTCTTAAC |
| | TCCGGGACAGAGATGGCTGGCGGAGCCTGGGGCCGCCTGGCCTGTTACTTGGAGTTCCTG |
| | AAGAAGGAGGAGCTGAAGGAGTTCCAGCTTCTGCTCGCCAATAAAGCGCACTCCAGGAGC |
| | TCTTCGGGTGAGACACCCGCTCAGCCAGAGAAGACGAGTGGCATGGAGGTGGCCTCGTAC |
| | CTGGTGGCTCAGTATGGGGAGCAGCGGGCCTGGGACCTAGCCCTCCATACCTGGGAGCAG |
| | ATGGGGCTGAGGTCACTGTGCGCCCAAGCCCAGGAAGGGGCAGGCCACTCTCCCTCATTC |
| | CCCTACAGCCCAAGTGAACCCCACCTGGGGTCTCCCAGCCAACCCACCTCCACCGCAGTG |
| | CTAATGCCCTGGATCCATGAATTGCCGGCGGGGTGCACCCAGGGCTCAGAGAGAAGGGTT |
| | TTGAGACAGCTGCCTGACACATCTGGACGCCGCTGGAGAGAAATCTCTGCCTCACTCCTC |
| | TACCAAGCTCTTCCAAGCTCCCCAGACCATGAGTCTCCAAGCCAGGAGTCACCCAACGCC |
| | CCCACATCCACAGCAGTGCTGGGGAGCTGGGGATCCCCACCTCAGCCCAGCCTAGCACCC |
| | AGAGAGCAGGAGGCTCCTGGGACCCAATGGCCTCTGGATGAAACGTCAGGAATTTACTAC |
| | ACAGAAATCAGAGAAAGAGAGAGAGAGAAATCAGAGAAAGGCAGGCCCCCATGGGCAGCG |
| | GTGGTAGGAACGCCCCCACAGGCGCACACCAGCCTACAGCCCCACCACCACCCATGGGAG |
| | CCTTCTGTGAGAGAGAGCCTCTGTTCCACATGGCCCTGGAAAAATGAGGATTTTAACCAA |
| | AAATTCACACAGCTGCTACTTCTACAAAGACCTCACCCCAGAAGCCAAGATCCCCTGGTC |
| | AAGAGAAGCTGGCCTGATTATGTGGAGGAGAATCGAGGACATTTAATTGAGATCAGAGAC |
| | TTATTTGGCCCAGGCCTGGATACCCAAGAACCTCGCATAGTCATACTGCAGGGGGCTGCT |
| | GGAATTGGGAAGTCAACACTGGCCAGGCAGGTGAAGGAAGCCTGGGGGAGAGGCCAGCTG |
| | TATGGGGACCGCTTCCAGCATGTCTTCTACTTCAGCTGCAGAGAGCTGGCCCAGTCCAAG |
| | GTGGTGAGTCTCGCTGAGCTCATCGGAAAAGATGGGACAGCCACTCCGGCTCCCATTAGA |
| | CAGATCCTGTCTAGGCCAGAGCGGCTGCTCTTCATCCTCGATGGTGTAGATGAGCCAGGA |
| | TGGGTCTTGCAGGAGCCGAGTTCTGAGCTCTGTCTGCACTGGAGCCAGCCACAGCCGGCG |
| | GATGCACTGCTGGGCAGTTTGCTGGGGAAAACTATACTTCCCGAGGCATCCTTCCTGATC |
| | ACGGCTCGGACCACAGCTCTGCAGAACCTCATTCCTTCTTTGGAGCAGGCACGTTGGGTA |
| | GAGGTCCTGGGGTTCTCTGAGTCCAGCAGGAAGGAATATTTCTACAGATATTTCACAGAT |
| | GAAAGGCAAGCAATTAGAGCCTTTAGGTTGGTCAAATCAAACAAAGAGCTCTGGGCCCTG |
| | TGTCTTGTGCCCTGGGTGTCCTGGCTGGCCTGCACTTGCCTGATGCAGCAGATGAAGCGG |
| | AAGGAAAAACTCACACTGACTTCCAAGACCACCACAACCCTCTGTCTACATTACCTTGCC |
| | CAGGCTCTCCAAGCTCAGCCATTGGGACCCCAGCTCAGAGACCTCTGCTCTCTGGCTGCT |
| | GAGGGCATCTGGCAAAAAAAGACCCTTTTCAGTCCAGATGACCTCAGGAAGCATGGGTTA |
| | GATGGGGCCATCATCTCCACCTTCTTGAAGATGGGTATTCTTCAAGAGCACCCCATCCCT |
| | CTGAGCTACAGCTTCATTCACCTCTGTTTCCAAGAGTTCTTTGCAGCAATGTCCTATGTC |
| | TTGGAGGATGAGAAGGGGAGAGGTAAACATTCTAATTGCATCATAGATTTGGAAAAGACG |
| | CTAGAAGCATATGGAATACATGGCCTGTTTGGGGCATCAACCACACGTTTCCTATTGGGC |
| | CTGTTAAGTGATGAGGGGGAGAGAGAGATGGAGAACATCTTTCACTGCCGGCTGTCTCAG |
| | GGGAGGAACCTGATGCAGTGGGTCCCGTCCCTGCAGCTGCTGCTGCAGCCACACTCTCTG |
| | GAGTCCCTCCACTGCTTGTACGAGACTCGGAACAAAACGTTCCTGACACAAGTGATGGCC |
| | CATTTCGAAGAAATGGGCATGTGTGTAGAAACAGACATGGAGCTCTTAGTGTGCACTTTC |
| | TGCATTAAATTCAGCCGCCACGTGAAGAAGCTTCAGCTGATTGAGGGCAGGCAGCACAGA |
| | TCAACATGGAGCCCCACCATGGTAGTCCTGTTCAGGTGGGTCCCAGTCACAGATGCCTAT |
| | TGGCAGATTCTCTTCTCCGTCCTCAAGGTCACCAGAAACCTGAAGGAGCTGGACCTAAGT |
| | GGAAACTCGCTGAGCCACTCTGCAGTGAAGAGTCTTTGTAAGACCCTGAGACGCCCTCGC |
| | TGCCTCCTGGAGACCCTGCGGTTGGCTGGCTGTGGCCTCACAGCTGAGGACTGCAAGGAC |
| | CTTGCCTTTGGGCTGAGAGCCAACCAGACCCTGACCGAGCTGGACCTGAGCTTCAATGTG |
| | CTCACGGATGCTGGAGCCAAACACCTTTGCCAGAGACTGAGACAGCCGAGCTGCAAGCTA |
| | CAGCGACTGCAGCTGGTCAGCTGTGGCCTCACGTCTGACTGCTGCCAGGACCTGGCCTCT |
| | GTGCTTAGTGCCAGCCCCAGCCTGAAGGAGCTAGACCTGCAGCAGAACAACCTGGATGAC |
| | GTTGGCGTGCGACTGCTCTGTGAGGGGCTCAGGCATCCTGCCTGCAAACTCATACGCCTG |
| | GGGAAACCAAGTGTGATGACCCCTACTGAGGGCCTGGATACGGGAGAGATGAGTAATAGC |
| | ACATCCTCACTCAAGCGGCAGAGACTCGGATCAGAGAGGGCGGCTTCCCATGTTGCTCAG |
| | GCTAATCTCAAACTCCTGGACGTGAGCAAGATCTTCCCAATTGCTGAGATTGCAGAGGAA |
| | AGCTCCCCAGAGGTAGTACCGGTGGAACTCTTGTGCGTGCCTTCTCCTGCCTCTCAAGGG |
| | GACCTGCATACGAAGCCTTTGGGGACTGACGATGACTTCTGGGGCCCCACGGGGCCTGTG |
| | GCTACTGAGGTAGTTGACAAAGAAAAGAACTTGTACCGAGTTCACTTCCCTGTAGCTGGC |
| | TCCTACCGCTGGCCCAACACGGGTCTCTGCTTTGTGATGAGAGAAGCGGTGACCGTTGAG |
| | ATTGAATTCTGTGTGTGGGACCAGTTCCTGGGTGAGATCAACCCACAGCACAGCTGGATG |
| | GTGGCAGGGCCTCTGCTGGACATCAAGGCTGAGCCTGGAGCTGTGGAAGCTGTGCACCTC |
| | CCTCACTTTGTGGCTCTCCAAGGGGGCCATGTGGACACATCCCTGTTCCAAATGGCCCAC |
| | TTTAAAGAGGAGGGGATGCTCCTGGAGAAGCCAGCCAGGGTGGAGCTGCATCACATAGTT |
| | CTGGAAAACCCCAGCTTCTCCCCCTTGGGAGTCCTCCTGAAAATGATCCATAATGCCCTG |
| | CGCTTCATTCCCGTCACCTCTGTGGTGTTGCTTTACCACCGCGTCCATCCTGAGGAAGTC |
| | ACCTTCCACCTCTACCTGATCCCAAGTGACTGCTCCATTCGGAAGGAACTGGAGCTCTGC |
| | TATCGAAGCCCTGGAGAAGACCAGCTGTTCTCGGAGTTCTACGTTGGCCACTTGGGATCA |
| | GGGATCAGGCTGCAAGTGAAAGACAAGAAAGATGAGACTCTGGTGTGGGAGGCCTTGGTG |
| | AAACCAGGAGATCTCATGCCTGCAACTACTCTGATCCCTCCAGCCCGCATAGCCGTACCT |
| | TCACCTCTGGATGCCCCGCAGTTGCTGCACTTTGTGGACCAGTATCGAGAGCAGCTGATA |
| | GCCCGAGTGACATCGGTGGAGGTTGTCTTGGACAAACTGCATGGACAGGTGCTGAGCCAG |
| | GAGCAGTACGAGAGGGTGCTGGCTGAGAACACGAGGCCCAGCCAGATGCGGAAGCTGTTC |
| | AGCTTGAGCCAGTCCTGGGACCGGAAGTGCAAAGATGGACTCTACCAAGCCCTGAAGGAG |
| | ACCCATCCTCACCTCATTATGGAACTCTGGGAGAAGGGCAGCAAAAAGGGACTCCTGCCA |
| | CTCAGCAGCTGAAGTATCAACACCAGCCCTTGACCCTTGAGTCCTGGCTTTGGCTGACCC |
| | TTCTTTGGGTCTCAGTTTCTTTCTCTGCAAACAAGTTGCCATCTGGTTTGCCTTCCAGCA |
| | CTAAAGTAATGGAACTTTGATGATGCCTTTGCTGGGCATTATGTGTCCATGCCAGGGATG |
| | CCACAGGGGGCCCCAGTCCAGGTGGCCTAACAGCATCTCAGGGAATGTCCATCTGGAGCT |
| | GGCAAGACCCCTGCAGACCTCATAGAGCCTCATCTGGTGGCCACAGCAGCCAAGCCTAGA |
| | GCCCTCCGGATCCCATCCAGGCGCAAAGAGGAATAGGAGGGACATGGAACCATTTGCCTC |
| | TGGCTGTGTCACAGGGTGAGCCCCAAAATTGGGGTTCAGCGTGGGAGGCCACGTGGATTC |
| | TTGGCTTTGTACAGGAAGATCTACAAGAGCAAGCCAACAGAGTAAAGTGGAAGGAAGTTT |
| | ATTCAGAAAATAAAGGAGTATCACAGCTCTTTTAGAATTTGTCTAGCAGGCTTTCCAGTT |
| | TTTACCAGAAAACCCCTATAAATTAAAAATTTTTTACTTAAATTTAAGAATTAAAAAAAT |
| | ACAAAAAAGAAAAAATGAAAATAAAGGAATAAGAAGTTACCTACTCCA |
| 13 | GTTGACTAGGCGCTGTTCTTGCTGGCTGGTGCCCCAGGGCCTGGAGAGGTCTGAAGAAAC |
| | CTGGGAGCCAGCAGCCCGGGGCTCCACTCTGGGTTCTGAAAGCCCATTCCCTGCTCTGCG |
| | GCTCCTCCCACCCCACCTCTTCTCAGCCTTGCAGCTCAAGGGTTGATCTCAGGAGTCCAG |
| | GACCCAGGAGAGGGAAGAATCTGAGGAACACAGAACAGTGAGCGTTGCCCACACCCCATC |
| | TCCCGTCACCACATCTCCCCTCACCCTCACCCTCCCTGCCTGGCCCTGGACCCCATCCCA |
| | GGACCTCCCTATCAGCTGACTTCTTCCAGTGTCTTGCAGGCCCCTCTGGGCTCCTCCCTC |
| | CCCTGGCTTTTCCTACCACTCCCCCTCTATCGGCGTCTATCTGTAGGTGCCCTGGGATTT |
| | ATAAAACTGGGTTCCGAATGCTGAATAAGAGACGGTAAGAGCCAAGGCAAAGGACAGCAC |
| | TGTTCTCTGCCTGCCTGATACCCTCACCACCTGGGAACATCCCCCAGACACCCTCTTAAC |
| | TCCGGGACAGAGATGGCTGGCGGAGCCTGGGGCCGCCTGGCCTGTTACTTGGAGTTCCTG |
| | AAGAAGGAGGAGCTGAAGGAGTTCCAGCTTCTGCTCGCCAATAAAGCGCACTCCAGGAGC |
| | TCTTCGGGTGAGACACCCGCTCAGCCAGAGAAGACGAGTGGCATGGAGGTGGCCTCGTAC |
| | CTGGTGGCTCAGTATGGGGAGCAGCGGGCCTGGGACCTAGCCCTCCATACCTGGGAGCAG |
| | ATGGGGCTGAGGTCACTGTGCGCCCAAGCCCAGGAAGGGGCAGGCCACTCTCCCTCATTC |
| | CCCTACAGCCCAAGTGAACCCCACCTGGGGTCTCCCAGCCAACCCACCTCCACCGCAGTG |
| | CTAATGCCCTGGATCCATGAATTGCCGGCGGGGTGCACCCAGGGCTCAGAGAGAAGGGTT |
| | TTGAGACAGCTGCCTGACACATCTGGACGCCGCTGGAGAGAAATCTCTGCCTCACTCCTC |
| | TACCAAGCTCTTCCAAGCTCCCCAGACCATGAGTCTCCAAGCCAGGAGTCACCCAACGCC |
| | CCCACATCCACAGCAGTGCTGGGGAGCTGGGGATCCCCACCTCAGCCCAGCCTAGCACCC |
| | AGAGAGCAGGAGGCTCCTGGGACCCAATGGCCTCTGGATGAAACGTCAGGAATTTACTAC |
| | ACAGAAATCAGAGAAAGAGAGAGAGAGAAATCAGAGAAAGGCAGGCCCCCATGGGCAGCG |
| | GTGGTAGGAACGCCCCCACAGGCGCACACCAGCCTACAGCCCCACCACCACCCATGGGAG |
| | CCTTCTGTGAGAGAGAGCCTCTGTTCCACATGGCCCTGGAAAAATGAGGATTTTAACCAA |
| | AAATTCACACAGCTGCTACTTCTACAAAGACCTCACCCCAGAAGCCAAGATCCCCTGGTC |
| | AAGAGAAGCTGGCCTGATTATGTGGAGGAGAATCGAGGACATTTAATTGAGATCAGAGAC |
| | TTATTTGGCCCAGGCCTGGATACCCAAGAACCTCGCATAGTCATACTGCAGGGGGCTGCT |
| | GGAATTGGGAAGTCAACACTGGCCAGGCAGGTGAAGGAAGCCTGGGGGAGAGGCCAGCTG |
| | TATGGGGACCGCTTCCAGCATGTCTTCTACTTCAGCTGCAGAGAGCTGGCCCAGTCCAAG |
| | GTGGTGAGTCTCGCTGAGCTCATCGGAAAAGATGGGACAGCCACTCCGGCTCCCATTAGA |
| | CAGATCCTGTCTAGGCCAGAGCGGCTGCTCTTCATCCTCGATGGTGTAGATGAGCCAGGA |
| | TGGGTCTTGCAGGAGCCGAGTTCTGAGCTCTGTCTGCACTGGAGCCAGCCACAGCCGGCG |
| | GATGCACTGCTGGGCAGTTTGCTGGGGAAAACTATACTTCCCGAGGCATCCTTCCTGATC |
| | ACGGCTCGGACCACAGCTCTGCAGAACCTCATTCCTTCTTTGGAGCAGGCACGTTGGGTA |
| | GAGGTCCTGGGGTTCTCTGAGTCCAGCAGGAAGGAATATTTCTACAGATATTTCACAGAT |
| | GAAAGGCAAGCAATTAGAGCCTTTAGGTTGGTCAAATCAAACAAAGAGCTCTGGGCCCTG |
| | TGTCTTGTGCCCTGGGTGTCCTGGCTGGCCTGCACTTGCCTGATGCAGCAGATGAAGCGG |
| | AAGGAAAAACTCACACTGACTTCCAAGACCACCACAACCCTCTGTCTACATTACCTTGCC |
| | CAGGCTCTCCAAGCTCAGCCATTGGGACCCCAGCTCAGAGACCTCTGCTCTCTGGCTGCT |
| | GAGGGCATCTGGCAAAAAAAGACCCTTTTCAGTCCAGATGACCTCAGGAAGCATGGGTTA |
| | GATGGGGCCATCATCTCCACCTTCTTGAAGATGGGTATTCTTCAAGAGCACCCCATCCCT |
| | CTGAGCTACAGCTTCATTCACCTCTGTTTCCAAGAGTTCTTTGCAGCAATGTCCTATGTC |
| | TTGGAGGATGAGAAGGGGAGAGGTAAACATTCTAATTGCATCATAGATTTGGAAAAGACG |
| | CTAGAAGCATATGGAATACATGGCCTGTTTGGGGCATCAACCACACGTTTCCTATTGGGC |
| | CTGTTAAGTGATGAGGGGGAGAGAGAGATGGAGAACATCTTTCACTGCCGGCTGTCTCAG |
| | GGGAGGAACCTGATGCAGTGGGTCCCGTCCCTGCAGCTGCTGCTGCAGCCACACTCTCTG |
| | GAGTCCCTCCACTGCTTGTACGAGACTCGGAACAAAACGTTCCTGACACAAGTGATGGCC |
| | CATTTCGAAGAAATGGGCATGTGTGTAGAAACAGACATGGAGCTCTTAGTGTGCACTTTC |
| | TGCATTAAATTCAGCCGCCACGTGAAGAAGCTTCAGCTGATTGAGGGCAGGCAGCACAGA |
| | TCAACATGGAGCCCCACCATGGTAGTCCTGTTCAGGTGGGTCCCAGTCACAGATGCCTAT |
| | TGGCAGATTCTCTTCTCCGTCCTCAAGGTCACCAGAAACCTGAAGGAGCTGGACCTAAGT |
| | GGAAACTCGCTGAGCCACTCTGCAGTGAAGAGTCTTTGTAAGACCCTGAGACGCCCTCGC |
| | TGCCTCCTGGAGACCCTGCGGTTGGCTGGCTGTGGCCTCACAGCTGAGGACTGCAAGGAC |
| | CTTGCCTTTGGGCTGAGAGCCAACCAGACCCTGACCGAGCTGGACCTGAGCTTCAATGTG |
| | CTCACGGATGCTGGAGCCAAACACCTTTGCCAGAGACTGAGACAGCCGAGCTGCAAGCTA |
| | CAGCGACTGCAGCTGGTCAGCTGTGGCCTCACGTCTGACTGCTGCCAGGACCTGGCCTCT |
| | GTGCTTAGTGCCAGCCCCAGCCTGAAGGAGCTAGACCTGCAGCAGAACAACCTGGATGAC |
| | GTTGGCGTGCGACTGCTCTGTGAGGGGCTCAGGCATCCTGCCTGCAAACTCATACGCCTG |
| | GGGCTGGACCAGACAACTCTGAGTGATGAGATGAGGCAGGAACTGAGGGCCCTGGAGCAG |
| | GAGAAACCTCAGCTGCTCATCTTCAGCAGACGGAAACCAAGTGTGATGACCCCTACTGAG |
| | GGCCTGGATACGGGAGAGATGAGTAATAGCACATCCTCACTCAAGCGGCAGAGACTCGGA |
| | TCAGAGAGGGCGGCTTCCCATGTTGCTCAGGCTAATCTCAAACTCCTGGACGTGAGCAAG |
| | ATCTTCCCAATTGCTGAGATTGCAGGCAAGAGCCACGAGGAAAGCTCCCCAGAGGTAGTA |
| | CCGGTGGAACTCTTGTGCGTGCCTTCTCCTGCCTCTCAAGGGGACCTGCATACGAAGCCT |
| | TTGGGGACTGACGATGACTTCTGGGGCCCCACGGGGCCTGTGGCTACTGAGGTAGTTGAC |
| | AAAGAAAAGAACTTGTACCGAGTTCACTTCCCTGTAGCTGGCTCCTACCGCTGGCCCAAC |
| | ACGGGTCTCTGCTTTGTGATGAGAGAAGCGGTGACCGTTGAGATTGAATTCTGTGTGTGG |
| | GACCAGTTCCTGGGTGAGATCAACCCACAGCACAGCTGGATGGTGGCAGGGCCTCTGCTG |
| | GACATCAAGGCTGAGCCTGGAGCTGTGGAAGCTGTGCACCTCCCTCACTTTGTGGCTCTC |
| | CAAGGGGGCCATGTGGACACATCCCTGTTCCAAATGGCCCACTTTAAAGAGGAGGGGATG |
| | CTCCTGGAGAAGCCAGCCAGGGTGGAGCTGCATCACATAGTTCTGGAAAACCCCAGCTTC |
| | TCCCCCTTGGGAGTCCTCCTGAAAATGATCCATAATGCCCTGCGCTTCATTCCCGTCACC |
| | TCTGTGGTGTTGCTTTACCACCGCGTCCATCCTGAGGAAGTCACCTTCCACCTCTACCTG |
| | ATCCCAAGTGACTGCTCCATTCGGAAGGCCATAGATGATCTAGAAATGAAATTCCAGTTT |
| | GTGCGAATCCACAAGCCACCCCCGCTGACCCCACTTTATATGGGCTGTCGTTACACTGTG |
| | TCTGGGTCTGGTTCAGGGATGCTGGAAATACTCCCCAAGGAACTGGAGCTCTGCTATCGA |
| | AGCCCTGGAGAAGACCAGCTGTTCTCGGAGTTCTACGTTGGCCACTTGGGATCAGGGATC |
| | AGGCTGCAAGTGAAAGACAAGAAAGATGAGACTCTGGTGTGGGAGGCCTTGGTGAAACCA |
| | GGAAGGAACACCAGCCAGCCGTGGAACCTCAGGTGCAACAGAGACGCCAGGAGATACTAG |
| | TGCCCAGCAGCCTGCGGCAGTACCAATGAAGCCAGAGAGGGCTTGGTGGATGACAAGGAG |
| | GCCTGAGTAGACCGCAGGTGGGTCTGAGAAATGGGCTTAGGTGAGGCAGGTCTTTGAAGG |
| | ATTTGTTCTTAATCATATGCGAGATGCTCAAAAGGCTGGATGCCTGCTTTTGTGGGTGAA |
| | GAGCAAGAAGAGAAAACAGGTTGTACACATACAGATGCAGATGGAGAGACAGAGAAAAAA |
| | AAGGAAGAAGGCAGAGAAATGCACCAATTCTTGAGCTGTATTATCTCTGGACCTTGGGAT |
| | TGTGGGAGGCTTTATTTTACTACTGATTTTGCCTACACTGTTTTCTCAATTTCTAGTTTT |
| | CTACAAAGATGATGTGTTAGCTTTTTCACGCATTAAGATTAAAATTTAAAACAGACCA |
| 21 | GTCCCACACACAGA |
| 22 | GTGGTGGTCTTGGA |
| 23 | TCCCACACACAGAA |
| 24 | TGGATTCTTGGCTT |
| 25 | GTGGATTCTTGGCT |
| 26 | GGATTCTTGGCTTT |
| 27 | GUCCCACACACAGA |
| 28 | GUGGUGGUCUUGGA |
| 29 | UCCCACACACAGAA |
| 30 | UGGAUUCUUGGCUU |
| 31 | GUGGAUUCUUGGCU |
| 32 | GGAUUCUUGGCUUU |
| 33 | GTCCCACACACAGA |
| 34 | GTGGTGGTCTTGGA |
| 35 | TCCCACACACAGAA |
| 36 | TGGATTCTTGGCTT |
| 37 | GTGGATTCTTGGCT |
| 38 | GGATTCTTGGCTTT |

### EXAMPLES

The following illustrative examples are representative of embodiments of the stimulation, systems, and methods described herein and are not meant to be limiting in any way.

### Example 1. Modulating Expressions of NLRP3 or NLRP1

### Cell Culture Condition and In Vitro Transfection

HMC3 cells are maintained in Eagle's Minimum Essential Media, supplementing with 10% with 10% fetal bovine serum, 100 U/ml penicillin and 100 U/ml streptomycin (Pen-strep, or PS), and the cells are incubated at 37° C. in a humidified incubator containing 5% CO2. For antisense treatment, the cells are plated at 70% confluency the previous day in 96 well plates. On the day of transfection, cells are washed once with OptiMEM medium, and incubated in 80 μL OptiMEM. Transfection mixture are prepared in OptiMEM by mixing the antisense and the Lipofectamine RNAiMax transfection reagents at desired concentration, and 20 μL of the transfection mixture is added into each well and incubated for 2 hours. At the end of the 2 hours, 10 μL of serum is added in to well, and made up the volume to 200 μL with respective culture medium for the cell line. Alternatively, media is replaced 2 hours post transfection or on the next day. The ASO treatment can also be done without transfection reagent. In this case, the oligonucleotide for binding to endogenous nucleic acid encoding NLRP3 or NLRP1 is diluted with OptiMEM and added into the cell culture in a volume less than 5% of the entire volume.

mRNA Knockdown Detection

Cells are harvested by lysis at 48 hours post transfection. The lysis and the follow up mRNA detection are conducted according to the Quantigen assay specified by the manufacturer (ThermoFisher).

### Cell Inflammation Assay

After the antisense treatment, HMC3 cells are monitored at 3- or 5-day post transfection. Cell inflammation is monitored by activation with lipopolysaccharide (LPS ng/mL, Sigma-Aldrich) for 24 hours and adenosine triphosphate (ATP, 5 mM, Sigma-Aldrich) for additional 30 minutes. Cytokines measurements were performed by qualitative ELISA in the media.

### Cell Proliferation Assay

After the antisense treatment, cells are monitored at 3 or 5 days post transfection. Cell proliferation is monitored by CellTiter-Glo assay according to the protocol specified by the manufacturer (Promega).

### Example 2. mRNA Knockdown of NLRP3 Mediated by Chemically Modified Antisense Oligonucleotide

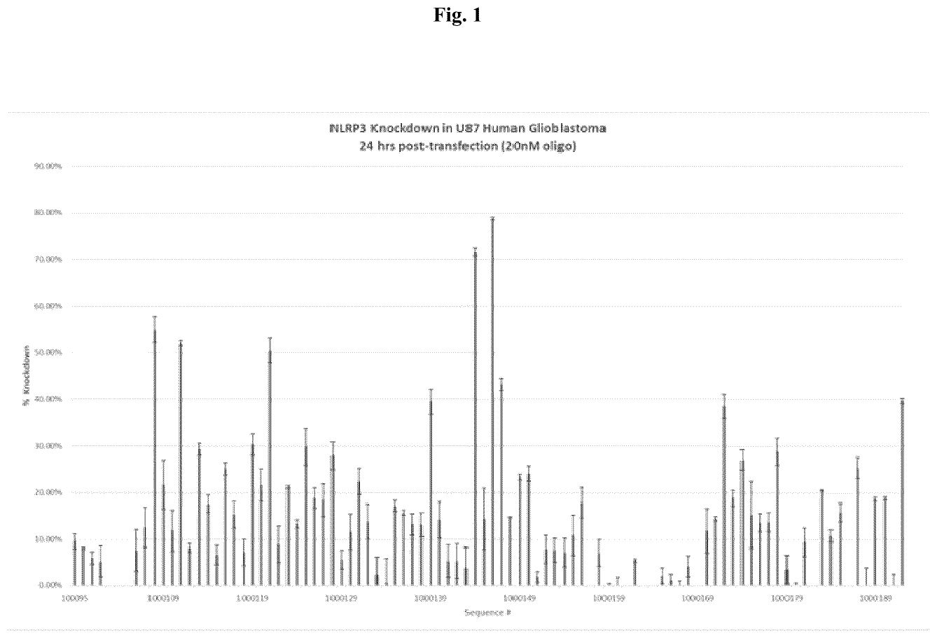

14-mer antisense oligonucleotides containing LNA modifications and fully phosphorothioated backbone were synthesized by Integrated DNA Technologies, Inc. Each oligo was transfected into U87-MG glioblastoma cell line (ATCC) at 20 nM final concentration using 0.3 μL per well of Lipofectamine RNAiMax (Thermo-Fisher) in a 96-well format. After 24 hours, the cells were lysed with 0.05 mL per well of Quantigene lysis mixture (Life Technologies) for gene expression assays. NLRP3 and PPIB housekeeping gene expression were assayed using the Quantigene singleplex assay kit (Life Technologies) according to the manufacturer's protocol for RNA capture, followed by subsequent signal amplification and detection by chemiluminescent reaction. The relative gene expression that was detected per well was quantified in relative light units (RLU) on the Spark luminometer (Tecan). The extend of NLRP3 knockdown was determined by the following formula: % KD sequence X=1-[(RLU NLRP3 sequence X/RLU PPIB sequence X)/(RLU NLRP3 negative control/RLU PPIB negative control)]. Table 2 illustrates the mRNA knockdown mediated by the chemically modified antisense oligonucleotide. FIG. 1 illustrates NLRP3 mRNA knockdown in U87 human glioblastoma cells 24 hours after transfection.

TABLE 2
mRNA knockdown mediated by antisense oligonucleotide
comprising chemical modification

| | Sequence | % KD | % CV | |
|---|---|---|---|---|
| | (“+” denotes LNA, “*” denotes | (@20 | (@20 | SEQ ID |
| Name | phosphorothioate linkage) | nM) | nM) | NO |
| STN-100095 | +G*+T*+C*C*C*A*C*A*C*A*C*+A*+ | 9.57% | 1.62% | SEQ ID |
| | G*+A | | | NO: 95 |
| STN-100096 | +G*+T*+G*G*T*G*G*T*C*T*T*+G*+ | 8.06% | 0.21% | SEQ ID |
| | G*+A | | | NO: 96 |
| STN-100097 | +T*+C*+C*C*A*C*A*C*A*C*A*+G*+ | 5.99% | 1.32% | SEQ ID |
| | A*+A | | | NO: 97 |
| STN-100098 | +T*+G*+G*A*T*T*C*T*T*G*G*+C*+ | 5.17% | 3.36% | SEQ ID |
| | T*+T | | | NO: 98 |
| STN-100099 | +G*+T*+G*G*A*T*T*C*T*T*G*+G*+ | −4.44% | 4.45% | SEQ ID |
| | C*+T | | | NO: 99 |
| STN-100100 | +G*+G*+A*T*T*C*T*T*G*G*C*+T*+ | −2.29% | 1.89% | SEQ ID |
| | T*+T | | | NO: 100 |
| STN-100105 | +G*+T*+G*G*C*T*A*G*A*T*C*+C*+ | −12.79% | 3.08% | SEQ ID |
| | A*+C | | | NO: 105 |
| STN-100106 | +C*+T*+G*T*T*G*A*T*C*G*C*+A*+ | 7.57% | 4.47% | SEQ ID |
| | G*+C | | | NO: 106 |
| STN-100107 | +A*+A*+A*C*C*C*A*T*C*C*A*+C*+ | 12.59% | 4.28% | SEQ ID |
| | T*+C | | | NO: 107 |
| STN-100108 | +G*+T*+G*T*G*C*A*C*A*G*G*+C*+ | 54.93% | 2.70% | SEQ ID |
| | T*+C | | | NO: 108 |
| STN-100109 | +C*+C*+C*T*G*G*A*A*C*A*C*+C*+ | 21.69% | 5.28% | SEQ ID |
| | A*+C | | | NO: 109 |
| STN-100110 | +G*+C*+C*C*A*G*T*C*C*A*A*+C*+ | 11.85% | 4.43% | SEQ ID |
| | A*+T | | | NO: 110 |
| STN-100111 | +A*+G*+G*C*T*C*A*C*C*T*C*+T*+ | 52.06% | 0.57% | SEQ ID |
| | C*+G | | | NO: 111 |
| STN-100112 | +G*+G*+T*G*G*G*T*T*T*G*G*+G*+ | 8.16% | 0.91% | SEQ ID |
| | T*+C | | | NO: 112 |
| STN-100113 | +C*+A*+T*G*A*G*G*A*A*G*A*+G*+ | 29.26% | 1.34% | SEQ ID |
| | G*+A | | | NO: 113 |
| STN-100114 | +G*+T*+G*C*T*C*G*T*C*A*A*+A*+ | 17.58% | 1.83% | SEQ ID |
| | G*+G | | | NO: 114 |
| STN-100115 | +A*+T*+G*T*G*C*T*C*G*T*C*+A*+ | 6.63% | 2.10% | SEQ ID |
| | A*+A | | | NO: 115 |
| STN-100116 | +A*+G*+A*A*T*G*T*C*T*C*C*+C*+ | 25.11% | 1.33% | SEQ ID |
| | C*+G | | | NO: 116 |
| STN-100117 | +A*+T*+G*A*G*C*A*G*A*G*A*+G*+ | 15.28% | 2.84% | SEQ ID |
| | G*+C | | | NO: 117 |
| STN-100118 | +G*+C*+T*G*C*A*G*T*T*T*C*+T*+ | 7.18% | 2.88% | SEQ ID |
| | C*+C | | | NO: 118 |
| STN-100119 | +A*+G*+G*A*T*C*T*C*C*A*C*+A*+ | 30.28% | 2.34% | SEQ ID |
| | T*+G | | | NO: 119 |
| STN-100120 | +T*+T*+C*C*T*T*T*T*G*G*C*+C*+ | 21.62% | 3.48% | SEQ ID |
| | T*+C | | | NO: 120 |
| STN-100121 | +A*+T*+G*A*A*G*C*A*C*A*T*+G*+ | 50.42% | 2.71% | SEQ ID |
| | G*+T | | | NO: 121 |
| STN-100122 | +G*+G*+G*G*G*A*T*G*A*A*G*+C*+ | 8.86% | 3.94% | SEQ ID |
| | A*+C | | | NO: 122 |
| STN-100123 | +C*+A*+G*G*G*G*G*A*T*G*A*+A*+ | 21.22% | 0.29% | SEQ ID |
| | G*+C | | | NO: 123 |
| STN-100124 | +G*+A*+C*C*A*G*G*G*G*G*A*+T*+ | 13.29% | 0.85% | SEQ ID |
| | G*+A | | | NO: 124 |
| STN-100125 | +G*+C*+A*G*A*C*C*A*G*G*G*+G*+ | 29.78% | 3.94% | SEQ ID |
| | G*+A | | | NO: 125 |
| STN-100126 | +C*+A*+G*C*A*G*A*C*C*A*G*+G*+ | 18.80% | 2.18% | SEQ ID |
| | G*+G | | | NO: 126 |
| STN-100127 | +A*+T*+C*C*A*G*C*A*G*A*C*+C*+ | 18.44% | 3.53% | SEQ ID |
| | A*+G | | | NO: 127 |
| STN-100128 | +A*+G*+G*A*A*G*A*A*G*A*C*+G*+ | 27.90% | 2.94% | SEQ ID |
| | T*+A | | | NO: 128 |
| STN-100129 | +G*+A*+A*A*G*G*A*A*G*A*A*+G*+ | 5.63% | 2.03% | SEQ ID |
| | A*+C | | | NO: 129 |
| STN-100130 | +G*+A*+T*T*T*T*C*T*G*G*T*+T*+ | 11.63% | 3.74% | SEQ ID |
| | C*+C | | | NO: 130 |
| STN-100131 | +A*+G*+G*A*T*T*T*T*C*T*G*+G*+ | 22.35% | 2.85% | SEQ ID |
| | T*+T | | | NO: 131 |
| STN-100132 | +A*+T*+C*C*T*C*A*G*G*A*A*+A*+ | 13.76% | 3.77% | SEQ ID |
| | G*+C | | | NO: 132 |
| STN-100133 | +T*+C*+A*T*C*C*T*C*A*G*G*+A*+ | 2.40% | 3.83% | SEQ ID |
| | A*+A | | | NO: 133 |
| STN-100134 | +G*+T*+T*C*A*T*C*C*T*C*A*+G*+ | 0.46% | 5.43% | SEQ ID |
| | G*+A | | | NO: 134 |
| STN-100135 | +T*+C*+G*C*A*G*T*C*C*A*C*+T*+ | 17.15% | 1.11% | SEQ ID |
| | T*+C | | | NO: 135 |
| STN-100136 | +T*+C*+T*C*G*C*A*G*T*C*C*+A*+ | 15.70% | 0.51% | SEQ ID |
| | C*+T | | | NO: 136 |
| STN-100137 | +T*+G*+A*A*G*C*T*G*T*A*G*+A*+ | 13.23% | 2.33% | SEQ ID |
| | A*+C | | | NO: 137 |
| STN-100138 | +T*+G*+G*A*A*A*G*T*C*A*T*+G*+ | 13.14% | 2.55% | SEQ ID |
| | T*+G | | | NO: 138 |
| STN-100139 | +C*+C*+T*G*G*A*A*A*G*T*C*+A*+ | 39.52% | 2.56% | SEQ ID |
| | T*+G | | | NO: 139 |
| STN-100140 | +C*+T*+C*C*T*G*G*A*A*A*G*+T*+ | 14.13% | 3.89% | SEQ ID |
| | C*+A | | | NO: 140 |
| STN-100141 | +A*+A*+C*T*C*C*T*G*G*A*A*+A*+ | 5.27% | 3.51% | SEQ ID |
| | G*+T | | | NO: 141 |
| STN-100142 | +A*+G*+A*A*C*T*C*C*T*G*G*+A*+ | 5.27% | 3.77% | SEQ ID |
| | A*+A | | | NO: 142 |
| STN-100143 | +T*+A*+C*A*A*C*A*A*A*A*A*+T*+ | 3.85% | 4.43% | SEQ ID |
| | C*+A | | | NO: 143 |
| STN-100144 | +A*+G*+G*C*C*A*A*A*G*A*G*+G*+ | 71.60% | 0.88% | SEQ ID |
| | A*+A | | | NO: 144 |
| STN-100145 | +C*+T*+C*T*C*C*T*G*G*T*T*+T*+ | 14.34% | 6.53% | SEQ ID |
| | A*+C | | | NO: 145 |
| STN-100146 | +G*+A*+T*C*T*T*G*C*A*A*C*+T*+ | 78.67% | 0.24% | SEQ ID |
| | T*+A | | | NO: 146 |
| STN-100147 | +G*+A*+G*A*T*C*T*T*G*C*A*+A*+ | 43.25% | 1.33% | SEQ ID |
| | C*+T | | | NO: 147 |
| STN-100148 | +G*+A*+G*A*G*A*T*C*T*T*G*+C*+ | 14.73% | 0.18% | SEQ ID |
| | A*+A | | | NO: 148 |
| STN-100149 | +C*+T*+G*A*G*A*G*A*T*C*T*+T*+ | 23.35% | 0.65% | SEQ ID |
| | G*+C | | | NO: 149 |
| STN-100150 | +T*+G*+C*T*G*A*G*A*G*A*T*+C*+ | 24.10% | 1.66% | SEQ ID |
| | T*+T | | | NO: 150 |
| STN-100151 | +T*+T*+T*C*A*C*T*T*C*A*A*+T*+ | 1.88% | 1.15% | SEQ ID |
| | C*+C | | | NO: 151 |
| STN-100152 | +C*+T*+T*T*C*A*C*T*T*C*A*+A*+ | 7.81% | 3.00% | SEQ ID |
| | T*+C | | | NO: 152 |
| STN-100153 | +C*+T*+G*C*A*T*C*T*C*G*T*+A*+ | 7.67% | 2.59% | SEQ ID |
| | C*+A | | | NO: 153 |
| STN-100154 | +T*+C*+C*T*G*C*A*T*C*T*C*+G*+ | 7.14% | 3.10% | SEQ ID |
| | T*+A | | | NO: 154 |
| STN-100155 | +T*+G*+G*T*C*C*A*T*T*C*T*+G*+ | 10.86% | 4.32% | SEQ ID |
| | G*+T | | | NO: 155 |
| STN-100156 | +G*+T*+C*A*C*T*G*A*G*G*T*+C*+ | 17.82% | 3.28% | SEQ ID |
| | C*+A | | | NO: 156 |
| STN-100157 | +A*+T*+T*G*T*C*A*C*T*G*A*+G*+ | −23.24% | 4.68% | SEQ ID |
| | G*+T | | | NO: 157 |
| STN-100158 | +G*+A*+A*T*G*T*T*A*C*A*G*+C*+ | 7.04% | 2.85% | SEQ ID |
| | C*+A | | | NO: 158 |
| STN-100159 | +G*+C*+G*C*C*C*C*A*A*C*C*+A*+ | −3.56% | 4.00% | SEQ ID |
| | C*+A | | | NO: 159 |
| STN-100160 | +C*+A*+G*C*G*C*C*C*C*A*A*+C*+ | −0.42% | 2.16% | SEQ ID |
| | C*+A | | | NO: 160 |
| STN-100161 | +A*+T*+G*T*C*G*A*A*G*C*A*+G*+ | −2.81% | 0.84% | SEQ ID |
| | C*+A | | | NO: 161 |
| STN-100162 | +G*+A*+G*A*T*G*T*C*G*A*A*+G*+ | 5.44% | 0.38% | SEQ ID |
| | C*+A | | | NO: 162 |
| STN-100163 | +A*+G*+G*A*G*A*T*G*T*C*G*+A*+ | −5.78% | 1.62% | SEQ ID |
| | A*+G | | | NO: 163 |
| STN-100164 | +A*+G*+C*T*C*C*A*C*C*A*G*+C*+ | −4.93% | 2.32% | SEQ ID |
| | T*+T | | | NO: 164 |
| STN-100165 | +C*+A*+G*T*C*C*C*A*C*A*C*+A*+ | 2.10% | 1.70% | SEQ ID |
| | C*+A | | | NO: 165 |
| STN-100166 | +G*+C*+T*T*C*A*G*T*C*C*C*+A*+ | 1.17% | 1.27% | SEQ ID |
| | C*+A | | | NO: 166 |
| STN-100167 | +G*+T*+G*C*T*T*C*A*G*T*C*+C*+ | −0.96% | 1.96% | SEQ ID |
| | C*+A | | | NO: 167 |
| STN-100168 | +A*+G*+G*T*G*C*T*T*C*A*G*+T*+ | 4.12% | 2.24% | SEQ ID |
| | C*C | | | NO: 168 |
| STN-100169 | +G*+T*+C*T*C*C*C*A*A*G*G*+C*+ | −11.24% | 3.03% | SEQ ID |
| | A*+T | | | NO: 169 |
| STN-100170 | +C*+T*+C*C*T*G*A*G*T*C*T*+C*+ | 11.81% | 4.78% | SEQ ID |
| | C*+C | | | NO: 170 |
| STN-100171 | +G*+A*+C*T*C*C*T*G*A*G*T*+C*+ | 14.44% | 0.47% | SEQ ID |
| | T*+C | | | NO: 171 |
| STN-100172 | +A*+T*+T*C*A*C*C*A*A*C*C*+C*+ | 38.52% | 2.56% | SEQ ID |
| | C*+A | | | NO: 172 |
| STN-100173 | +G*+A*+A*T*T*C*A*C*C*A*A*+C*+ | 18.78% | 1.72% | SEQ ID |
| | C*+C | | | NO: 173 |
| STN-100174 | +A*+G*+C*T*G*A*A*C*A*A*C*+A*+ | 27.00% | 2.21% | SEQ ID |
| | G*+A | | | NO: 174 |
| STN-100175 | +G*+T*+T*G*T*C*T*A*A*T*T*+C*+ | 15.19% | 7.12% | SEQ ID |
| | C*+A | | | NO: 175 |
| STN-100176 | +C*+A*+G*T*T*G*T*C*T*A*A*+T*+ | 13.56% | 1.97% | SEQ ID |
| | T*+C | | | NO: 176 |
| STN-100177 | +T*+G*+C*A*G*T*T*G*T*C*T*+A*+ | 13.67% | 2.05% | SEQ ID |
| | A*+T | | | NO: 177 |
| STN-100178 | +C*+A*+T*T*G*T*T*G*C*C*C*+A*+ | 28.70% | 2.90% | SEQ ID |
| | G*+G | | | NO: 178 |
| STN-100179 | +T*+G*+C*T*G*T*T*T*C*A*G*+C*+ | 3.46% | 3.13% | SEQ ID |
| | A*+C | | | NO: 179 |
| STN-100180 | +C*+T*+C*A*G*G*C*T*T*T*T*+C*+ | 3.18% | 3.74% | SEQ ID |
| | T*+T | | | NO: 180 |
| STN-100181 | +A*+G*+C*T*C*A*G*G*C*T*T*+T*+ | 9.36% | 3.04% | SEQ ID |
| | T*+C | | | NO: 181 |
| STN-100182 | +T*+C*+A*G*C*T*C*A*G*G*C*+T*+ | −8.51% | 3.64% | SEQ ID |
| | T*+T | | | NO: 182 |
| STN-100183 | +C*+G*+T*G*G*C*T*A*G*A*T*+C*+ | 20.44% | 0.12% | SEQ ID |
| | C*+A | | | NO: 183 |
| STN-100184 | +T*+C*+C*T*C*T*T*C*C*A*G*+C*+ | 10.68% | 1.34% | SEQ ID |
| | A*+G | | | NO: 184 |
| STN-100185 | +T*+T*+C*C*T*C*T*T*C*C*A*+G*+ | 15.74% | 1.94% | SEQ ID |
| | C*+A | | | NO: 185 |
| STN-100186 | +A*+C*+A*A*C*A*A*A*A*A*T*+C*+ | −6.94% | 4.21% | SEQ ID |
| | A*+A | | | NO: 186 |
| STN-100187 | +A*+G*+A*G*A*T*C*T*T*G*C*+A*+ | 25.25% | 2.22% | SEQ ID |
| | A*+C | | | NO: 187 |
| STN-100188 | +T*+G*+C*T*T*C*A*G*T*C*C*+C*+ | −1.09% | 4.91% | SEQ ID |
| | A*+C | | | NO: 188 |
| STN-100189 | +G*+C*+A*G*T*T*G*T*C*T*A*+A*+ | 18.52% | 0.41% | SEQ ID |
| | T*+T | | | NO: 189 |
| STN-100646 | +C*+T*+A*T*G*T*G*C*T*C*G*+T*+ | 18.71% | 0.32% | SEQ ID |
| | C*+A | | | NO: 646 |
| STN-100101 | +A*+C*+G*T*C*T*A*T*A*C*A*+C*+ | 0% | 2.45% | SEQ ID |
| (negative | C*+A | | | NO: 101 |
| control) | | | | |
| STN-100053 | +C*+C*+T*A*T*G*T*G*C*T*C*G*T* | 39.58% | 0.58% | SEQ ID |
| (16-mer) | +C*+A*+A | | | NO: 3 |

“+” references to a nucleic acid substitution with LNA.
“*” references a chemical modification comprising a phosphorothioate linkage.
“+N” is N nucleotide modified as LNA.
“N*” is phosphothioate linkage coupling with the following nucleoside.

### Example 3. Suppression of Inflammasome Activity in Murine Microglia Following NLRP3 Antisense Oligonucleotide Treatment

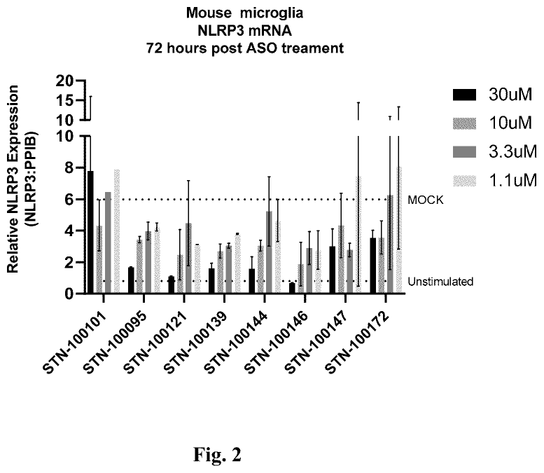

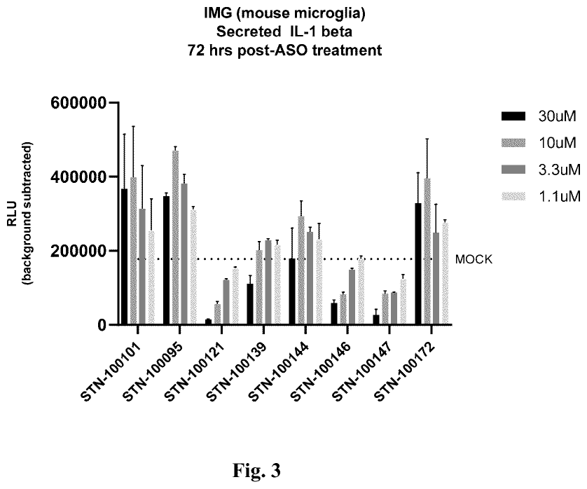

Immortalized mouse microglia (IMG) cells were obtained by Sigma Aldrich and cultured in DMEM (Gibco) supplemented with 10% FBS in 96 well format. NLRP3 targeting antisense oligonucleotides (ASO) were added to cells at varying concentrations for 72 hours and cultured in a 37° C., 5% CO2 incubator. Following ASO treatment, inflammasome stimulation was performed by treatment with 100 ng/mL lipopolysaccharide (LPS, Invivogen) for 90 minutes followed by addition of 5 mM adenosine triphosphate (ATP, Invivogen) for 30 minutes. Secreted IL-1 beta in culture supernatants were measured using the Lumit mouse IL-1 beta assay kit (Promega), and gene expression analyses on cell lysates were conducted by bDNA assay using the Quantigene Singleplex assay system (Life Technologies) according to manufacturers' protocols with chemiluminescent detection for both assay systems performed on the Spark luminometer (Tecan). FIG. 2 illustrates NLRP3 mRNA knockdown in immortalized mouse microglia cells 72 hours after treatment with gymnotically-delivered antisense oligonucleotide and FIG. 3 illustrates the resulting suppression of secreted IL-1 beta cytokine in cell culture supernatants.

### Example 4. Suppression of Inflammasome Activity in Human Peripheral Blood Mononuclear Cells Following NLRP3 Oligonucleotide Treatment

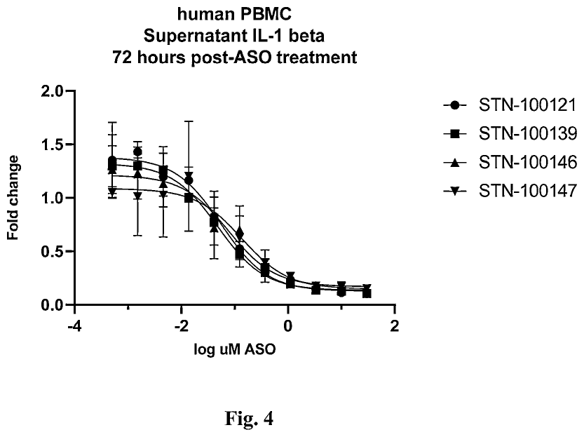

Cryopreserved human peripheral blood mononuclear cells (PBMC) from healthy individuals were procured by iXCells (San Diego, CA) and thawed in a 37° C. water bath for 1 minute and washed in 14 mL of RPMI culture media supplemented with 10% FBS (heat inactivated), sodium pyruvate, nonessential amino acids, L-glutamine, and HEPES. The cells were then plated at 200,000 cells per well (in 0.1 mL) in a 96 well flat bottom cell culture plate and allowed to acclimate for 4 hours prior to antisense oligonucleotide (ASO) treatment. NLRP3-targeting ASOs at varying concentrations were added to cells and placed in a 37° C., 5% CO2 incubator for 72 hours. Following ASO treatment, inflammasome stimulation was performed by treatment with 100 ng/mL lipopolysaccharide (LPS, Invivogen) for 90 minutes followed by addition of 5 mM adenosine triphosphate (ATP, Invivogen) for 30 minutes. Secreted IL-1 beta in culture supernatants were measured using the Lumit human IL-1 beta assay kit (Promega) according to manufacturers' protocols with chemiluminescent detection performed on the Spark luminometer (Tecan). FIG. 4 illustrates the reduction of inflammasome activity via IL-1 beta secretion in cultured human PBMCs following treatment with NLRP3-targeting antisense oligonucleotides.

While the foregoing disclosure has been described in some detail for purposes of clarity and understanding, it will be clear to one skilled in the art from a reading of this disclosure that various changes in form and detail can be made without departing from the true scope of the disclosure. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually and separately indicated to be incorporated by reference for all purposes.

1. A composition comprising an antisense oligonucleotide capable of binding to NACHT, LRR and PYD domains-containing protein 3 (NLRP3) or NLRP1 mRNA.

2. The composition of claim 1, wherein the antisense oligonucleotide specifically binds to the NLRP3 mRNA.

3. (canceled)

4. (canceled)

5. The composition of claim 1, wherein the antisense oligonucleotide specifically binds to the NLRP1 mRNA.

6. (canceled)

7. (canceled)

8. The composition of claim 1, wherein the antisense oligonucleotide specifically binds to the NLRP3 mRNA and NLRP1 mRNA.

9. (canceled)

10. (canceled)

11. The composition of claim 1, wherein the antisense oligonucleotide comprises a nucleic acid sequence that has at least 80%, 85%, 90%, 95%, or 99% sequence similarity to one of the following sequences: SEQ ID NOs: 21-38.

12. The composition of claim 1, wherein the antisense oligonucleotide comprises 12-30 nucleotides in length.

13. The composition of claim 1, wherein the antisense oligonucleotide comprises a gap segment and a wing segment.

14. The composition of claim 13, wherein the antisense oligonucleotide comprises 5′-wing segment and 3′-wing segment.

15. (canceled)

16. The composition of claim 1, wherein the antisense oligonucleotide comprises at least one 2′-modified nucleoside, at least one modified internucleotide linkage, or at least one inverted abasic moiety.

17. The composition of claim 16, wherein the at least one 2′ modified nucleotide comprises 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), 2′-O—N-methylacetamido (2′-O-NMA) modified nucleotide, locked nucleic acid (LNA), constrained ethyl (cEt) sugar, thiomorpholino, ethylene nucleic acid (ENA), or a combination thereof.

18. The composition of claim 17, wherein the at least one modified internucleotide linkage comprises a phosphorothioate linkage or a phosphorodithioate linkage.

19. The composition of claim 1, wherein the antisense oligonucleotide comprises a phosphorodiamidate morpholino oligomer (PMO), thiomorpholino, locked nucleic acid (LNA), or constrained ethyl (cEt) sugar.

20. The composition of claim 1, wherein the antisense oligonucleotide is conjugated with a peptide, antibody, lipid, carbohydrates, or a polymer.

21.-25. (canceled)

26. The composition of claim 1, wherein the antisense oligonucleotide comprises a nucleic acid sequence that is at least 80%, 85%, 90%, 95%, or 99% identical to any one of SEQ ID NOs: 95, 96, 97, 98, 99, 100, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, or 646.

27. The composition of claim 26, wherein the antisense oligonucleotide comprises the nucleic acid sequence that is at least 80%, 85%, 90%, 95%, or 99% identical to any one of SEQ ID NOs: 95, 121, 139, 144, 146, 147, or 172.

28. The composition of claim 1, wherein the antisense oligonucleotide is any one of SEQ ID NOs: 95, 121, 139, 144, 146, 147, or 172.

29. A method of modulating an inflammasome pathway in a cell, comprising: treating the cell with a composition comprising antisense oligonucleotide capable of binding to NLRP3 or NLRP1 mRNA, thereby reducing expression of NLRP3, NLRP1, or inflammasome in the cell.

30. The method of claim 29, wherein the cell is associated with an inflammasome disease or condition.

31. The method of claim 29, wherein the antisense oligonucleotide comprises at least one 2′-modified nucleotide, at least one modified internucleotide linkage, or at least one inverted abasic moiety.

32. The method of claim 29, wherein the at least one 2′ modified nucleotide comprises 2′-O-methyl, 2′-O-methoxyethyl (2′-O-MOE), 2′-O-aminopropyl, 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), 2′-O—N-methylacetamido (2′-O-NMA) modified nucleotide; locked nucleic acid (LNA), locked nucleic acid (LNA), constrained ethyl (cEt) sugar, thiomorpholino, ethylene nucleic acid (ENA), or a combination thereof.

33. The method of claim 29, wherein the expression of NLRP3 or NLRP1 protein or mRNA is reduced at least 30%, at least 40%, at least 50% after the treatment.

34. (canceled)

35. (canceled)

36. A method of treating a disease or condition associated with inflammasome in a subject in need thereof, the method comprising: administering the subject a composition of claim 1, thereby treating the disease or condition associated with inflammasome in the subject.

37.-42. (canceled)
